(19) United States
(12) Patent Application Publication
(10) Pub. No.: US 2020/0138829 A1
(43) Pub. Date: May 7, 2020

(54) METHODS OF CANCER TREATMENT

(71) Applicant: Ferro Therapeutics, Inc., Palo Alto, CA (US)

(72) Inventors: Ruihong Chen, Burlingame, CA (US); Chun Jiang, Hillsborough, CA (US)

(21) Appl. No.: 16/616,384

(22) PCT Filed: May 24, 2018
(86) PCT No.: PCT/US2018/034491

§ 371 Date: Nov. 22, 2019

Related U.S. Application Data
(60) Provisional application No. 62/510,716, filed on May 24, 2017.

(51) Int. Cl. A61K 31/5513; A61K 31/015; A61K 31/497; A61K 31/44; A61K 31/336; A61K 31/53; A61K 31/437; A61K 31/357; A61K 31/17; A61K 31/517; A61K 31/235; A61K 31/439; A61P 35/02; A61K 45/00

(52) U.S. Cl. CPC A61K 31/5513; A61K 31/015; A61K 31/497; A61K 31/44; A61K 31/336; A61K 31/53; A61K 45/00; A61K 31/357; A61K 31/17; A61K 31/517; A61K 31/235; A61K 31/439; A61P 35/02; A61K 31/437

(57) ABSTRACT

The present disclosure relates to a method of treating a subject with cancer with a ferroptosis inducer, including use of the ferroptosis inducer in combination with a second therapeutic agent.

### 1. CROSS-REFERENCE TO RELATED APPLICATIONS

This application claims benefit under 35 U.S.C. § 119(e) of U.S. Provisional application Ser. No. 62/510,716, filed May 24, 2017, the entire contents of which are incorporated herein by reference.

### 2. BACKGROUND

Radiation and drug therapy represent two of the most common types of cancer treatments. Some types of radiation therapy use focused, high-energy photon beams to destroy cancer cells. Photon radiation includes X-rays and gamma rays. Radiation therapy can also employ particle radiation, which includes electron, proton, and neutron beams. Radiation can be used as a curative therapy for a number of cancer types, or used in combination with other treatments, for example prior to surgery or chemotherapy, to reduce initial tumor burden and to destroy any remaining cancer cells after such therapy. Radiation therapy works by damaging the DNA of cancer cells, either by direct or indirect ionization of the atoms that make up the DNA chain Indirect ionization occurs through the generation of reactive oxygen species (ROS), particularly hydroxyl radicals, which then damage the DNA. However, the mechanisms by which DNA damage ultimately leads to cell death appear to be complex, acting through a multitude of cellular signaling pathways that regulate different cell death processes. These processes include apoptosis, mitotic catastrophe, necrosis, senescence, and autophagy. Various genes and intracellular pathways have been reported to be involved in the different types of radiation induced cell death. Apoptosis has been associated with cellular components ATM, p53, Bax, Cytochrome c and Caspases, while mitotic catastrophe appears to implicate cellular components p53, Caspases, and Cytochrome c. Necrosis has been associated with TNF (alpha), PAR, JNK and Caspases while senescence is associated with, among others, cellular components MYC, JNK4A, ARF, p53 and p21. With autophagy, the cellular molecules PI3K, Akt and mTOR may be involved.

Chemotherapy can target different components of the cellular machinery and can have synergistic therapeutic effects when used in combination with radiation therapy. Chemotherapy can be nonspecific, hormonal or targeted. Nonspecific chemotherapeutic agents are generally cytotoxic agents that typically affect cell division, and include, among others, classes of agents such as alkylating agents, antimetabolites, anti-microtubule agents, topoisomerase inhibitors, cytotoxic antibiotics, and platinum-based coordination complexes. Hormone-based cancer therapy is used to treat hormone sensitive cancers (e.g., prostate cancer and breast cancer) by targeting the endocrine system using specific hormones or drugs that inhibit the production or activity of such hormones (hormone antagonists). Hormonal chemotherapeutic agents include, among others, aromatase inhibitors, GnRH analogues, selective estrogen receptor modulators, antiandrogens, estrogens, and progestogens. Targeted chemotherapy attempts to overcome the non-discriminate killing of noncancerous cells by traditional cytotoxic chemotherapeutic agents by acting on specific cellular targets. Types of targeted chemotherapeutic agents include antiangiogenesis agents, apoptosis inducing agents, differentiation agents, and signal transduction inhibitors. Some forms of targeted therapy use traditional non-specific cytotoxic agents but formulated for specific delivery to cancer cells or delivered in such a way to localize the drug to the tumor site. However, most chemotherapy, whether non-specific or targeted, ultimately involve cell death processes that are also implicated in radiation induced killing of cancer cells. Desirable are other chemotherapeutic agents that, either indirectly or directly, effect killing of cancer cells.

### 3. SUMMARY

The present disclosure provides agents functioning through a non-apoptotic pathway, also referred to as ferroptosis, to induce cell death in applications to the treatment of cancer. In one aspect, the ferroptosis inducers are administered to a subject afflicted with cancer in a therapeutically effective amount to treat the cancer. Various ferroptosis inducers can be used, including classes of compounds represented by ferroptosis inducers RSL3, ML162, ML210, erastin, artemisinin and auranofin. Analogs and derivatives of the representative compounds having ferroptosis inducing activity can be used in the methods of treating cancer.

In some embodiments, the ferroptosis inducer is used to treat cancers resistant to or previously treated with a chemotherapeutic agent. In some embodiments, the cancer selected for treatment with the ferroptosis inducer is determined to have or identified as having resistance to a chemotherapeutic agent other than a ferroptosis inducer. In some embodiments, the cancer selected for treatment with the ferroptosis inducer is identified as being previously treated with a chemotherapeutic agent other than a ferroptosis inducer. In some embodiments, the cancer selected for treatment has been previously treated with or is resistant to a chemotherapeutic agent selected from alkylating agents, antibiotic agents, antimetabolic agents (e.g., folate antagonists, purine analogs, pyrimidine analogs, etc.), topoisomerase inhibiting agents, anti-microtubule agents, aromatase inhibitors, antiangiogenic agents, differentiation inducing agents, cell growth arrest inducing agents, apoptosis inducing agents, cytotoxic agents, biologic agents (e.g., monoclonal antibodies), kinase inhibitors and inhibitors of growth factors and their receptors.

In some embodiments, the cancer selected for treatment with the ferroptosis inducer is determined to have or identified as having an activating or oncogenic RAS activity. In some embodiments, the activating or oncogenic RAS activity is an activating or oncogenic RAS mutation. In some embodiments, the activating or oncogenic RAS activity is an activating or oncogenic K-RAS activity, particularly an activating or oncogenic K-RAS mutation. In some embodiments, the activating or oncogenic RAS activity is an activating or oncogenic N-RAS activity, particularly an activating or oncogenic N-RAS mutation. In some embodiments, the activating or oncogenic RAS activity is an activating or oncogenic H-RAS activity, particularly an activating or oncogenic H-RAS mutation.

In some embodiments, the ferroptosis inducer is used in combination with a second therapeutic agent, such as alkylating agents, antibiotic agents, antimetabolic agents (e.g., folate antagonists, purine analogs, pyrimidine analogs, etc.), topoisomerase inhibiting agents, anti-microtubule agents, aromatase inhibitors, antiangiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors. In some embodiments, the second therapeutic agent is a PKC activator, particularly a diterpenoid PKC activator, including among others, tigliane, ingenane, daphnane and lathyrane compounds having PKC activating properties. In some embodiments, the diterpenoid PKC activating compound is a phorbol, deoxyphorbol, ingenol, and lathyrane PKC activating compound, as further described in the detailed description.

In some embodiments in which a ferroptosis inducer is administered in combination with a PCK activating compound, the cancer to be treated can be selected for sensitivity to the PKC activating compound, also referred to as an effective PKC activating potential. In some embodiments, a cancer for treatment with a ferroptosis inducer in combination with a PKC activating compound is determined to have or identified as having an effective PKC activating potential. The presence or absence of an effective PKC activating potential can be assessed in a number of ways, including, assessing PKC activity of the cancer, measuring the phosphorylation of PKC enzymes, and/or determining the presence or absence of inactivating or activity-attenuating mutations in one or more PKC enzymes selected from PKC α, β (e.g., βI or βII,), γ, δ, ε, η, θ, ι/λ, μ, and ζ.

In some embodiments of the combination treatment, the second therapeutic agent can be administered prior to, concurrently with, or subsequent to the administration of the ferroptosis inducer. In some embodiments, the ferroptosis inducer and the second therapeutic agent can be provided as a single composition where appropriate for ease of administration and enhance compliance with the combination treatment regimen.

### 4. BRIEF DESCRIPTION OF THE FIGURES

FIG. 1 shows sensitivity of lung cancer cell line A549 to growth inhibition by prostratin (K101A), and ferroptosis inducers RSL3, Erastin, ML210, Auranofin, and ferroptosis inhibitor Ferrostatin-1.

FIG. 2 shows sensitivity of pancreatic cancer cell line MiaPaCa-2 to growth inhibition by prostratin (K101A), and ferroptosis inducers RSL3, Erastin, ML210, Auranofin, and ferroptosis inhibitor Ferrostatin-1.

FIG. 3 shows sensitivity of pancreatic cancer cell line KP-4 to growth inhibition by prostratin (K101A), and ferroptosis inducers RSL3, Erastin, ML210, Auranofin, and ferroptosis inhibitor Ferrostatin-1.

FIG. 4 shows IC₅₀ values (04) of prostratin (K101A) and ferroptosis inducers RSL3, Erastin, ML210, ML162, Auranofin, Sulfasalazine, Artesunate, Artemisinin, Dihydroatemisinin, Sorafenib, Buthionine sulfoximine (BSO), Altretamine, Almitrine, ferroptosis inhibitors Ferrostatin-1, Liproxstatin-1, PD146176, Deferoxamine mesylate, and apoptosis inhibitor Z-VAD-FMK (ZVAD) in inhibiting growth of cancer cell lines A549, MiaPaCa-2, Panc-1, KP-4, Panc6.03, Panc2.13, PSN, Capan-1, Mino, and Namalwa.

FIG. 5 shows effect on growth of A549 cells by a combination of (i) prostratin (K101A) and RSL3, ML210, or Erastin or (ii) ingenol-3-angelate (K102) and RSL3, ML210, or Erastin at concentrations of RSL3, ML210, or Erastin that do not significantly inhibit cell proliferation individually.

FIG. 6 shows effect on growth of Panc-1 cells by a combination of (i) prostratin (K101A) and RSL3; (ii) ingenol-3-angelate (K102) and RSL3; (iii) prostratin (K101A) and ML210, and (iv) ingenol-3-angelate (K102) and ML210.

FIG. 7 shows effect on growth of Panc2.13 cells by a combination of (i) prostratin (K101A) and RSL3, ML210, or Erastin or (ii) ingenol-3-angelate (K102) and RSL3, ML210, or Erastin at concentrations of RSL3, ML210, or Erastin that do not significantly inhibit cell proliferation individually.

FIG. 8 shows effect on growth of PSN-1 cells by a combination of prostratin (K101A) and RSL3, ML210, or Erastin.

FIG. 9 shows effect on growth of Capan-1 cells by a combination of (i) prostratin (K101A) and RSL3 or ML210, or (ii) ingenol-3-angelate (K102) and RSL3 or ML210 at concentrations that do not significantly inhibit cell proliferation individually.

FIG. 10 shows effect on growth of KP-4 cells by a combination of (i) prostratin (K101A) and Artesunate or Dihydroartemisinin, or (ii) ingenol-3-angelate (K102) and Artesunate or Dihydroartemisinin at concentrations of Artesunate or Dihydroartemisinin that do not significantly inhibit cell proliferation individually.

FIG. 11 shows effect on growth of KP-4 cells by combination of prostratin (K101A) and Erastin or RSL3.

FIG. 12 shows effect on growth of Mino cells by a combination of (i) prostratin (K101A) and Auranofin or ML210 or (ii) ingenol-3-angelate (K102) and Auranofin or ML210 at concentrations of Auranofin or ML210 that do not significantly inhibit cell proliferation individually.

FIG. 13 shows effect on growth of Namalwa cells by a combination of (i) prostratin (K101A) and RSL3 or Auranofin or (ii) ingenol-3-angelate (K102) and RSL3 or Auranofin at concentrations of RSL3 or Auranofin that do not significantly inhibit cell proliferation individually.

FIG. 14 shows effect of ferroptosis inhibitors Ferrostatin-1, Liproxstatin-1, Deferoxamine, and PD146176, and apoptosis inhibitor ZVAD on (i) RSL3-induced or (ii) Erastin-induced cell death in KP-4 cells.

FIG. 15 shows effect of ferroptosis inhibitors Ferrostatin-1, Liproxstatin-1, Deferoxamine, and PD146176 and apoptosis inhibitor ZVAD on (i) RSL3-induced or (ii) Erastin-induced cell death in A549 cells.

### 5. DETAILED DESCRIPTION

As used in this specification and the appended claims, the singular forms “a”, “an” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

### 5.1. Definitions

In reference to the present disclosure, the technical and scientific terms used in the descriptions herein will have the meanings commonly understood by one of ordinary skill in the art, unless specifically defined otherwise. Accordingly, the following terms are intended to have the meanings as described below.

“Polypeptide,” “peptide” and “protein” are used interchangeably herein to denote a polymer of at least two amino acids covalently linked by an amide bond, regardless of length or modification, e.g., post-translational modification such as glycosylation, phosphorylation, lipidation, myristilation, ubiquitination, etc.

“Polynucleotide” or “nucleic acid’ refers to two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. Non-limiting examples of such non-standard linkages include phosphoramidates, phosphorothioates, O-methylphosphodiesters, positively-charged linkages and non-ionic linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. Preferably, such modified or synthetic nucleobases will be encoding nucleobases.

“Ferroptosis” refers to a form of cell death involving generation of reactive oxygen species mediated by iron, and is characterized by in part by lipid peroxidation.

“Ferroptosis Inducer” or “Ferroptosis activator” refers to any agent which induces, promotes or activates ferroptosis.

“K-RAS” refers to Kirsten rat sarcoma viral oncogene homolog, a small GTPase and a member of the RAS family of proteins involved in signal transduction. Exemplary human K-RAS nucleic acid and protein sequences are provided in GenBank Nos. M54968.1 and AAB414942.1, respectively. “K-RAS” as used herein encompasses variants, including orthologs and interspecies homologs, of the human K-RAS protein.

“Mutant K-RAS polypeptide”, “mutant K-RAS protein” and “mutant K-RAS” are used interchangeably and refer to a K-RAS polypeptide comprising at least one K-RAS mutation as compared to the corresponding wild-type K-RAS sequence. Certain exemplary mutant K-RAS polypeptides include, but are not limited to, allelic variants, splice variants, derivative variants, substitution variants, deletion variants, insertion variants, and fusion polypeptides.

“N-RAS” refers to Neuroblastoma RAS Viral (V-RAS) oncogene homolog, a small GTPase and a member of the RAS family of proteins involved in signal transduction. Exemplary human N-RAS nucleic acid and protein sequences are provided in NCBI Accession No. NP_002515 and GenBank Accession No. X02751, respectively. “N-RAS” as used herein encompasses variants, including orthologs and interspecies homologs of the human N-RAS protein.

“Mutant N-RAS polypeptide”, “mutant N-RAS protein” and “mutant N-RAS” are used interchangeably and refer to an N-RAS polypeptide comprising at least one N-RAS mutation as compared to the corresponding wild-type N-RAS sequence. Certain exemplary mutant N-RAS polypeptides include, but are not limited to, allelic variants, splice variants, derivative variants, substitution variants, deletion variants, insertion variants, and fusion polypeptides.

“H-RAS” refers to Harvey Rat Sarcoma viral oncogene homolog, a small GTPase and a member of the RAS family of proteins involved in signal transduction. Exemplary human H-RAS nucleic acid and protein sequences are provided in NCBI Accession No. P01112 and GenBank Accession No. NM_176795, respectively. “H-RAS” as used herein encompasses variants, including orthologs and interspecies homologs of the human H-RAS protein.

“Mutant H-RAS polypeptide”, “mutant H-RAS protein” and “mutant H-RAS” are used interchangeably and refer to an H-RAS polypeptide comprising at least one H-RAS mutation as compared to the corresponding wild-type H-RAS sequence. Certain exemplary mutant H-RAS polypeptides include, but are not limited to, allelic variants, splice variants, derivative variants, substitution variants, deletion variants, insertion variants, and fusion polypeptides.

“Activating K-RAS” refers to a form of K-RAS that has increased activity compared to wild-type K-RAS. The activation of K-RAS activity can result from a mutation or in some embodiments, overexpression of the K-RAS protein.

“Activating N-RAS” refers to a form of N-RAS that has increased activity compared to wild-type N-RAS. The activation of N-RAS activity can result from a mutation, or in some embodiments, overexpression of the N-RAS protein.

“Activating H-RAS” refers to a form of H-RAS that has increased activity compared to wild-type H-RAS. The activation of H-RAS activity can result from a mutation, or in some embodiments, overexpression of the H-RAS protein.

“Mutation” or “mutant” refers to an amino acid or polynucleotide sequence which has been altered by substitution, insertion, and/or deletion. In some embodiments, a mutant or variant sequence can have increased, decreased, or substantially similar activities or properties in comparison to the parental sequence.

“Gain-of-function” refers to enhancement of activity or acquisition of a new or abnormal activity of a nucleic acid or protein. “Gain-of-function mutation” in the context of a protein refers to an altered form of the protein that has enhanced activity or acquires a new or abnormal protein activity.

“Loss-of-function” refers to reduced or abolished activity (e.g., partially or wholly inactivated) of a nucleic acid or protein. “Loss-of-function mutation” in the context of a protein generally refers to an altered form of the protein that has reduced or complete loss of the activity associated with the protein.

“Dominant negative” refers to the effect of an alteration in a gene that results in negation or attenuation of the effect of the normal or wild-type copy of the gene. The dominant negative effect may result from an expression product of the gene, such as an expressed RNA or expressed protein. By way of example and not limitation, a mutated, dominant negative PKC resulting in loss or attenuation of PKC activity can further lead to loss or attenuation of PKC activity of the normal or wild-type PKC, or in some instances, loss or attenuation of PKC activity of other PKC isoforms.

“Dominant negative mutation” refers to a change in an amino acid or polynucleotide sequence which has been altered by substitution, insertion, and/or deletion, and results in the “dominant negative” effect on a biological process, for example a signal transduction pathway.

“Identified” or “determined” refers to analyzing for, detection of, or carrying out a process for the presence or absence of one or more specified characteristics.

“Wild-type” or “naturally occurring” refers to the form found in nature. For example, a naturally occurring or wild-type polypeptide or polynucleotide sequence is a sequence present in an organism that can be isolated from a source in nature and which has not been intentionally modified by human manipulation.

“Control” or “control sample” or “control group” refers to a sample or group that is compared to another sample or group, where generally the control sample or group are the same as a comparison group except for one or more factors being compared.

“Selecting” refers to the process of determining that a subject will receive an agent to treat the occurrence of a condition. Selecting can be based on an individual susceptibility to a particular disease or condition due to, for example, presence of an identifying cellular, physiological or environment factor or factors. In some embodiments, selecting can be based on determining or identifying whether that subject will be responsive to an agent, for example as assessed by identifying the presence of a biomarker and/or drug target marker that makes the subject sensitive, insensitive, responsive, or unresponsive to an agent or treatment.

“Biological sample” refers to any sample including a biomolecule, such as a protein, a peptide, a nucleic acid, a lipid, a carbohydrate or a combination thereof, that is obtained from an organism, particularly a mammal Examples of mammals include humans; veterinary animals like cats, dogs, horses, cattle, and swine; and laboratory animals like mice, rats and primates. In some embodiments, a human subject in the clinical setting is referred to as a patient. Biological samples include tissue samples (such as tissue sections and needle biopsies of tissue), cell samples (for example, cytological smears such as Pap or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, milk, vaginal fluid, saliva, swabs (such as buccal swabs), or any material containing biomolecules that is derived from a first biological sample. In particular embodiments, the biological sample is a “cell free sample”, such as cell free or extracellular polynucleotides, and cell free or extracellular proteins. In some embodiments, cell free DNA or cfDNA refers to extracellular DNA obtained from blood, particularly the serum.

“Subject” as used herein refers to a mammal, for example a dog, a cat, a horse, or a rabbit. In some embodiments, the subject is a non-human primate, for example a monkey, chimpanzee, or gorilla. In some embodiments, the subject is a human, sometimes referred to herein as a patient.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art, particularly in view of the guidance provided in the present disclosure.

“Therapeutically effective amount” refers to that amount which, when administered to an animal for treating a disease, is sufficient to effect such treatment for the disease, disorder, or condition.

“Alkyl” refers to straight or branched chain hydrocarbon groups of 1 to 20 carbon atoms, particularly 1 to 12 carbon atoms, and more particularly 1 to 8 carbon atoms. Exemplary “alkyl” includes, but are not limited to, methyl, ethyl, n-propyl, i-propyl, n-butyl, s-butyl, t-butyl, n-pentyl, and s-pentyl.

“Alkenyl” refers to straight or branched chain hydrocarbon group of 2 to 20 carbon atoms, particularly 2 to 12 carbon atoms, and most particularly 2 to 8 carbon atoms, having at least one double bond. Exemplary “alkenyl” includes, but are not limited to, vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkynyl” refers to a straight or branched chain hydrocarbon group of 2 to 12 carbon atoms, particularly 2 to 8 carbon atoms, containing at least one triple bond. Exemplary “alkynyl” includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkylene”, “alkenylene” and “alkynylene” refers to a straight or branched chain divalent hydrocarbon radical of the corresponding alkyl, alkenyl, and alkynyl, respectively. The “alkylene”, “alkenylene” and “alkynylene” may be optionally substituted, for example with alkyl, alkyloxy, hydroxyl, carbonyl, carboxyl, halo, nitro, and the like.

“Aliphatic” refers to an organic compound characterized by substituted or unsubstituted, straight or branched, and/or cyclic chain arrangements of constituent carbon atoms. Aliphatic compounds do not contain aromatic rings as part of the molecular structure of the compounds. Aliphatic compound can have 1-20 carbon atoms, 1-12 carbon atoms, particularly 1-8 carbon atoms.

“Lower” in reference to substituents refers to a group having between one and six carbon atoms.

“Cycloalkyl” refers to any stable monocyclic or polycyclic system which consists of carbon atoms, any ring of which being saturated. “Cycloalkenyl” refers to any stable monocyclic or polycyclic system which consists of carbon atoms, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, and bicycloalkyls.

“Heterocycloalkyl” or “heterocyclyl” refers to a substituted or unsubstituted 5 to 8 membered, mono- or bicyclic, non-aromatic hydrocarbon, wherein 1 to 3 carbon atoms are replaced by a heteroatom. Heteroatoms and/or heteroatomic groups which can replace the carbon atoms include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or lower alkyl. Examples include oxiranyl, oxetanyl, azetidynyl, oxazolyl, thiazolidinyl, thiazolyl, morpholinyl, pyrrolidinonyl, pyrrolidinyl, piperidinyl, piperazinyl, 2,3-dihydrofuranyl, dihydropyranyl, tetrahydrofuranyl, tetrahydropyranyl, dihydropyridinyl, tetrahydropyridinyl, tetrahydropyrimidinyl, tetrahydrothiophenyl, tetrahydrothiopyranyl, azapanyl, and the like.

“Carbocycle,” “carbocyclyl,” and “carbocyclic,” as used herein, refer to a non-aromatic saturated or unsaturated ring in which each atom of the ring is carbon. The ring may be monocyclic, bicyclic, tricyclic, or even of higher order. Thus, the terms “carbocycle,” “carbocyclyl.” and “carbocyclic,” encompass fused, bridged and spirocyclic systems: Preferably a carbocycle ring contains from 3 to 14 atoms, including 3 to 8 or 5 to 7 atoms, such as for example, 6 atoms. An exemplary bridged carbocycle is adamantyl.

“Aryl” refers to a six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Examples of “aryl” groups include phenyl, naphthyl, biphenyl, phenanthrenyl, naphthacenyl, and the like.

“Heteroaryl” means an aromatic heterocyclic ring, including both monocyclic and bicyclic ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur. In some embodiments, the heteroaryl can be a 5 to 6 membered monocyclic, or 7 to 11 membered bicyclic ring systems. Examples of “heteroaryl” groups include pyrrolyl, pyrazolyl, imidazolyl, pyrazinyl, oxazolyl, isoxazolyl, thiazolyl, furyl, thienyl, pyridyl, pyrimidyl, benzothiazolyl, purinyl, benzimidazolyl, indolyl, isoquinolyl, quinoxalinyl, quinolyl, and the like.

“Fused ring” refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Carbonyl” refers to —C(O)—. The carbonyl group may be further substituted with a variety of substituents to form different carbonyl groups including acids, acid halides, aldehydes, amides, esters, and ketones. For example, an —C(O)R′, wherein R′ is an alkyl is referred to as an alkylcarbonyl. In some embodiments, R′ is selected from an optionally substituted: alkyl, cycloalkyl, cycloalkylalkyl, heterocycloalkyl, heterocycloalkylalkyl, aryl, arylalkyl, heteroaryl, and heteroarylalkyl.

“Halogen” or “halo” refers to fluorine, chlorine, bromine and iodine.

“Hydroxy” refers to —OH.

“Oxy” refer to group —O—, which may have various substituents to form different oxy groups, including ethers and esters. In some embodiments, the oxy group is an —OR′, wherein R′ is selected from an optionally substituted: alkyl, cycloalkyl, cycloalkylalkyl, heterocycloalkyl, heterocycloalkylalkyl, aryl, arylalkyl, heteroaryl, and heteroarylalkyl.

“Acyl” refers to —C(O)R′, where R is hydrogen, or an optionally substituted alkyl, heteroalkyl, cycloalkyl, heterocycloalkyl, heterocycloalkylalkyl, aryl, arylalkyl, heteroaryl, or heteroarylalkyl as defined herein. Exemplary acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to —OR′, wherein R′ is an optionally substituted alkyl.

“Aryloxy” refers to —OR′, wherein R′ is an optionally substituted aryl.

“Carboxy” refers to —COO⁻ or COOM, wherein M is H or a counterion.

“Carbamoyl” refers to —C(O)NR′R′, wherein each R′ is independently selected from H or an optionally substituted alkyl, cycloalkyl, cycloalkylalkyl, heterocycloalkyl, heterocylcoalkylalkyl, aryl, arylalkyl, heteroaryl, or heteroarylalkyl.

“Cyano” refers to —CN.

“Ester” refers to a group such as —C(O)OR′, wherein R′ is selected from an optionally substituted: alkyl, cycloalkyl, cycloalkylalkyl, heterocycloalkyl, heterocyclolalkylalkyl, aryl, arylalkyl, heteroaryl, and heteroarylalkyl.

“Thiol” refers to —SH.

“Sulfanyl” refers to —SR′, wherein R′ is selected from an optionally substituted: alkyl, cycloalkyl, cycloalkylalkyl, heterocyloalkyl, heterocycloalkylalkyl, aryl, arylalkyl, heteroaryl, and heteroarylalkyl. For example, —SR, wherein R is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R′, wherein R′ is an alkyl refers to an alkylsulfonyl. In some embodiments of —S(O)₂R′, R′ is selected from an optionally substituted: alkyl, cycloalkyl, cycloalkylalkyl, heterocyloalkyl, heterocycloalkylalkyl, aryl, arylalkyl, heteroaryl, and heteroarylalkyl.

“Sulfinyl” refers to —S(O)—, which may have various substituents to form different sulfinyl groups including sulfinic acids, sulfinamides, and sulfinyl esters. For example, —S(O)R′, wherein R′ is an alkyl refers to an alkylsulfinyl. In some embodiments of —S(O)R′, R′ is selected from an optionally substituted: alkyl, cycloalkyl, cycloalkylalkyl, heterocyloalkyl, heterocycloalkylalkyl, aryl, arylalkyl, heteroaryl, and heteroarylalkyl.

“Selenide” refers to Se, which may have various substituents, particularly alkyl groups. For example, —SeR′, wherein R′ is an alkyl group refers to an alkylselenide. In some embodiments, R′ is selected from an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphine” refers to —PR′R′R′, wherein each R′ is independently selected from H and an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphate” refers to a group of formula —OP(═O)(OR′)₂, wherein each R′ is independently selected from H and an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphono” refers to a group of formula —P(═O)(OR′)₂, wherein each R′ is independently selected from H and an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphoramide” refers to a group of formula —OP(═O)R′R′, wherein at least one of R′ is an —NR″R″, wherein each R″ is independently selected from H and an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphoramidite” refers to a group of formula —OP(OR′)NR′R′, wherein each R′ is independently selected from an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphoramidate” refers to —OP(═O)(OR′)NR′R, wherein each R′ is independently selected from H and an optionally substituted: alkyl, cycloalkyl, cycloalkylalkyl, heterocyloalkyl, heterocycloalkylalkyl, aryl, arylalkyl, heteroaryl, and heteroarylalkyl.

“Phosphonate” refers to —P(═O)(OR′)₂, wherein each R′ is independently selected from H and an optionally substituted: alkyl, cycloalkyl, cycloalkylalkyl, heterocyloalkyl, heterocycloalkylalkyl, aryl, arylalkyl, heteroaryl and heteroarylalkyl.

“Ureide” refers to a cyclic or acyclic organic molecule of natural or synthetic origin that comprises one or more ureide moieties or derivatives thereof. Exemplary ureides include, among others, urea, uric acid, hydantoin, allantoin, imidazolidinyl urea (1,1′-methylenebis(3-[1-(hydroxymethyl)-2,5-dioxoimidazolidin-4-yl]urea), diazolydinyl urea (1,3-bis(hydroxymethyl)-1-(1,3,4-tris(hydroxymethyl)-2,5-dioxoimidazolidin-4-yl)urea), purines, and derivatives thereof.

“Urea” refers to a group such as —NHC(═O)NR′R′, wherein each R′ is independently selected from H and an optionally substituted: alkyl, cycloalkyl, cycloalkylalkyl, heterocycloalkyl, heterocycloalkylalkyl, aryl, arylalkyl, heteroaryl, and heteroarylalkyl.

“Amino” or “amine” refers to the group —NR′R′ or —NR′R′R′, wherein each R′ is independently selected from H and an optionally substituted: alkyl, cycloalkyl, heterocycloalkyl, alkyloxy, aryl, heteroaryl, heteroarylalkyl, acyl, alkyloxycarbonyl, sulfanyl, sulfinyl, sulfonyl, and the like. Exemplary amino groups include, but are not limited to, dimethylamino, diethylamino, trimethylammonium, triethylammonium, methylysulfonylamino, furanyl-oxy-sulfamino, and the like.

“Amide” refers to a group such as, —C(═O)NR′R′, wherein each R′ is independently selected from H and an optionally substituted: alkyl, cycloalkyl, cycloalkylalkyl, heterocycloalkyl, heterocycloalkylalkyl, aryl, arylalkyl, heteroaryl, and heteroarylalkyl.

“Sulfonamide” refers to —S(O)₂NR′R′, wherein each R′ is independently selected from H and an optionally substituted: alkyl, heteroalkyl, heteroaryl, heterocycle, alkenyl, alkynyl, arylalkyl, heteroarylalkyl, heterocyclylalkyl, -alkylenecarbonyl-, or alkylene-O—C(O)—OR″, where R″ is selected from H, alkyl, heteroalkyl, cyclylalkyl, heterocyclyl, aryl, heteroaryl, alkenyl, alkynyl, arylalkyl, heterocycloalkyl, heteroarylalkyl, amino, and sulfinyl.

“Guanidine” refers to —NR′C(═NR′)NR′R′, wherein each R′ is independently selected from H and an optionally substituted: alkyl, cycloalkyl, cycloalkylalkyl, heterocycloalkyl, heterocycloalkylalkyl, aryl, arylalkyl, heteroaryl, and heteroarylalkyl.

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Optionally substituted” or “substituted” as used herein means one or more hydrogen atoms of the group can each be replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, OR (e.g., hydroxyl, alkyloxy (e.g., methoxy, ethoxy, and propoxy), aryloxy, heteroaryloxy, arylalkyloxy, ether, ester, carbamate, etc.), hydroxyalkyl, alkyloxycarbonyl, alkyloxyalkyloxy, perhaloalkyl, alkyloxyalkyl, SR′ (e.g., thiol, alkylthio, arylthio, heteroarylthio, arylalkylthio, etc.), S⁺R′₂, S(O)R′, SO₂R′, NR′R′ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, thiol, carboxy, aldehyde, keto, carboxylic acid, ester, amide, imine, and imide, including seleno and thio derivatives thereof, wherein each of the substituents can be optionally further substituted. In embodiments in which a functional group with an aromatic carbon ring is substituted, such substitutions will typically number less than about 10 substitutions, more preferably about 1 to 5, with about 1 or 2 substitutions being preferred.

“Prodrug” refers to a derivative of an active compound (e.g., drug) that requires a transformation under the conditions of use, such as within the body or appropriate in vitro conditions, to release the active drug. Prodrugs are frequently, but not necessarily, pharmacologically inactive until converted into the active drug. Prodrugs can be obtained by masking a functional group in the drug believed to be in part required for activity with a progroup to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

Various progroups, as well as the resultant promoieties, suitable for masking functional groups in the active drugs to yield prodrugs can be used. For example, a hydroxyl functional group may be masked as a sulfonate, ester or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed, e.g., in vivo or under appropriate in vitro conditions, to provide the amino group. A carboxyl group may be masked as an ester (including silyl esters and thioesters), amide or hydrazide promoiety, which may be hydrolyzed in vivo to provide the carboxyl group. Included within the scope of prodrugs are, among others, “biohydrolyzable carbonate”, “biohydrolyzable ureide”, “biohydrolyzable carbamate”, “biohydrolyzable ester”, “biohydrolyzable amide”, and “biohydrolyzable phosphate” groups.

“Biohydrolyzable carbonate”, “biohydrolyzable ureide” and “biohydrolyzable carbamate” refers to a carbonate, ureide, or carbamate form, respectively, of a drug substance, such as the PKC activating compound of the disclosure, which (a) does not interfere with the biological activity of the parent substance but confers on that substance advantageous properties in vivo such as duration of action, onset of action, and the like, or (b) is biologically inactive but is readily converted in vivo by the subject to the biologically active principle.

“Biohydrolyzable ester” is an ester of a drug substance, such as the PKC activating compounds of the disclosure, which either (a) does not interfere with the biological activity of the parent substance but confers on that substance advantageous properties in vivo such as duration of action, onset of action, and the like, or (b) is biologically inactive but is readily converted in vivo by the subject to the biologically active principle. Examples include, by way of example, lower alkyl esters, lower acyloxy-alkyl esters, lower alkyloxyacyloxyalkyl esters, alkyloxyacyloxy esters, alkyl acylamino alkyl esters, and choline esters.

“Biohydrolyzable amide” refers to an amide of a drug substance, such as the PKC activating compounds of the disclosure, which either (a) does not interfere with the biological activity of the parent substance but confers on that substance advantageous properties in vivo such as duration of action, onset of action, and the like, or (b) is biologically inactive but is readily converted in vivo by the subject to the biologically active principle.

“Solvate” refers to a complex of variable stoichiometry formed by a solute, such as a PKC activator compound, and a solvent. Such solvents are selected to minimally interfere with the biological activity of the solute. Solvents may be, by way of example and not limitation, water, ethanol, or acetic acid.

“Hydrate” refers to a combination of water with a solute, such as a PKC activator compound, wherein the water retains its molecular state as water and is either absorbed, adsorbed or contained within a form of the solute.

“Pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### 5.2. Treatment of Cancers with Ferrotopsis Inducers

Cell death is crucial for normal development, homeostasis and the prevention of proliferative diseases such as cancer (Fuchs and Steller, 2011, Cell 147(4):742-58; Thompson, C. B., 1995, Science. 1995 267(5203):1456-62). Programmed cell death (PCD) can take different forms, such as apoptosis, mitotic catastrophe, necrosis, senescence, and autophagy. While each of these processes ultimately lead to cell death, the pathways and mechanisms appear to be unique, both at the molecular and cellular level.

Apoptosis is a programmed cell death characterized by morphological alterations in the cell membrane integrity (e.g., permeabilization), rounding up of the cell, reduction in nuclear volume, nuclear fragmentation, chromatin condensation, and mitochondrial membrane permeabilization, thereby leading to the destruction of the cell. Identified molecular components of apoptosis signaling pathways include, among others, ATM, p53, Bax, Cytochrome c and Caspases.

Mitotic catastrophe is cell death occurring during mitosis, such as caused by delayed mitosis or mitotic failure. Mitotic catastrophe can be described as premature or inappropriate entry of cells into mitosis and can be characterized by numerical and structural karyotype abnormalities. It can occur in cells lacking functional apoptotic pathway and can be induced by ionizing radiation and agents affecting stability of microtubule spindles and defective cell cycle checkpoints. Cells with mitotic failure can respond by cell death, necrosis, or senescence. Some identified molecular components implicated in mitotic catastrophe pathways include p53, Caspases and Cytochrome c.

Necrosis has been characterized as uncontrolled and pathological form of cell death but also involving molecular pathways that lead to death of the cell. Necrosis is characterized by cellular energy depletion, damage to membrane lipids, and loss of functioning homeostatic ion pumps/channels. Necrosis can be induced by inhibition of cellular energy production, imbalance of intracellular calcium flux, generation of ROS, and activation of nonapoptotic proteases. Molecular components implicated in regulating necrosis include, among others, TNFalpha, RIP-1, PARP, JNK and Caspases.

Senescence is a cellular response to various types of stress, including telomere uncapping, DNA damage, oxidative stress, and oncogene activity. Senescence can occur following a period of cellular proliferation or a rapid response to acute stress. Once cells have entered senescence, they cease to divide and undergo a series of morphologic and metabolic changes. Cells entering senescence cease to respond to mitogenic stimuli, undergo changes in chromatin structure (e.g., accumulation of heterochromatin foci), and become enlarged and flattened cells having increased adhesion to the extracellular matrix while losing cell-cell contacts. In some instances, cells that have undergone senescence can remain in a viable, non-dividing state for sustained periods of time (e.g., months). Molecular components implicated in regulating senescence include, among others, p53, Rb, INK4A, ARF, p21, and Bcl-2.

Autophagy is evolutionarily conserved and occurs in all eukaryotic cells. It does not strictly lead to cell death but appears to be an adaptive process for responding to metabolic stresses that result in degradation of intracellular proteins and organelles. Autophagy can be activated in response to nutrient starvation, differentiation, and developmental triggers. During autophagy, portions of the cytoplasm are encapsulated in a cellular structure referred to as an autophagosome. Autophagosomes then fuse with lysosomes where the contents are delivered, resulting in their degradation by lysosomal hydrolases. Under normal physiological conditions, autophagy occurs at basal levels in most tissues, contributing to the routine turnover of cytoplasmic components. It can promote cell adaptation and survival during stresses such as starvation, but under some conditions cells can undergo death by excessive autophagy. Some of the molecular components implicated in autophagy mediated cell death include, among others, RIPK1, PI3K, Akt and mTOR.

Ferroptosis has been identified as another cellular pathway that can lead to the death of cells. Ferroptosis does not display the classical features of apoptosis, such as mitochondrial cytochrome c release, caspase activation and chromatin fragmentation (Dolma et al., 2003, Cancer Cell, 2003, 3(3):285-96; Yagoda et al., 2007, Nature. 447(7146):864-8.; Yang and Stockwell, 2008, Chem Biol. 15(3):234-45). Ferroptosis does not appear to be sensitive to inhibitors of caspases, cathepsin or calpain proteases, RIPK1 (necrostatin-1), cyclophilin D (cyclosporin A) or lysosomal function/autophagy that are involved in forms of apoptosis, necrosis and autophagic cell death. Ferroptosis is characterized by increased levels of intracellular reactive oxygen species (ROS) and is prevented by iron chelation or genetic inhibition of cellular iron uptake. Addition of iron, but not by other divalent transition metal ions can potentiate ferroptosis. Cellular components implicated in and regulating ferroptosis include, among others, cysteine-glutamate antiporter (system X⁻_{c}), glutathione peroxidase 4 (GPX4), p53, and cargo receptor NCOA4. The inactivation or inhibition of some of these molecules, for example system X⁻_{c} or GPX4, leads to iron-dependent cell death (see, e.g., Gao et al., 2016, Cell Res. 26:1021-1032). A distinctive morphological feature of ferroptosis is reduction in mitochondrial size and increased membrane density. The prevention of cell death by iron chelation has been suggested to be a rare phenomenon, having only a limited number of triggers that can induce this iron-dependent cell death mechanism (see, e.g., Wolpaw et al., 2011, Proc Natl Acad Sci USA. 108(39):E771-E780). This suggests that ferroptosis might not be subject to the significant selection pressures for accumulation of mutations that inactivate other cell death pathways in cancer cells, thereby affording an alternative pathway for inducing cell death in cancer cells, bypassing mutations that inactivate or attenuate other cell death pathways. The present disclosure shows that ferroptosis inducers can inhibit cell growth of various cancer cells, particularly pancreatic cancer and lymphoma, and that it can have efficacy even in cancer cells made refractory or resistant to other chemotherapeutic agents. Moreover, the ferroptosis inducers are shown to potentiate the activity of cell proliferation inhibitory effects of diterpenoid PKC activators.

In one aspect, the present disclosure provides a method of treating cancer by use of ferroptosis inducers. In some embodiments, a method of treating cancer comprises administering to a subject in need thereof a therapeutically effective amount a ferroptosis inducer.

The ferroptosis inducer can be used as monotherapy, or as further provided below, in a combination therapy with one or more therapeutic treatments, particularly in combination with chemotherapeutic agents, more particularly diterpenoid PKC modulating compounds. In some embodiments, the ferroptosis inducer is used in combination with a second therapeutic agent, where the ferroptosis inducer is used at levels that sensitizes the cancer cell to the second therapeutic agent, particularly at levels of ferroptosis inducer that do not cause significant cell death. In some embodiments, the ferroptosis inducer can be used in combination with radiation therapy, either to sensitize the cells to radiation therapy or as an adjunct to radiation therapy (e.g., at doses sufficient to activate cell death pathway).

In some embodiments, the cancer for treatment with the ferroptosis inducer can be selected from, among others, adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemias and lymphomas), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, vaginal cancer, sarcoma, and soft tissue carcinomas.

In some embodiments, the cancer for treatment with the ferroptosis inducer is pancreatic cancer. In some embodiments, the pancreatic cancer for treatment with the ferroptosis inducer is pancreatic adenocarcinoma or metastatic pancreatic cancer. In some embodiments, the cancer for treatment with the ferroptosis inducer is stage 1, stage II, stage III, or stage IV pancreatic adenocarcinoma.

In some embodiments, the cancer for treatment with a ferroptosis inducer is lung cancer. In some embodiments, the lung cancer for treatment with the ferroptosis inducer is small cell lung cancer or non-small cell lung cancer. In some embodiments, the non-small cell lung cancer for treatment with the ferroptosis inducer is an adenocarcinoma, squamous cell carcinoma, or large cell carcinoma. In some embodiments, the lung cancer for treatment with the ferroptosis inducer is metastatic lung cancer.

In some embodiments, the cancer for treatment with the ferroptosis inducer is a hematologic cancer. In some embodiments, the hematologic cancer is selected from acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), and multiple myeloma.

In some embodiments, the cancer for treatment with the ferroptosis inducer is a leukemia selected from acute lymphoblastic leukemia (ALL), acute myeloid leukemia (AML), chronic lymphocytic leukemia (CLL), chronic myelogenous leukemia (CML), Hairy Cell chronic myelogenous leukemia (CML), and multiple myeloma.

In some embodiments, the cancer for treatment with the ferroptosis inducer is a lymphoma selected from Hodgkin's lymphoma, Non-Hodgkin's lymphoma, and Burkitt's lymphoma.

In some embodiments, the cancer for treatment with the ferroptosis inducer is a cancer characterized by mesenchymal features or mesenchymal phenotype. In many cancers, gain of mesenchymal features is associated with migratory (e.g., intravasation) and invasiveness of cancers. Mesenchymal features can include, among others, enhanced migratory capacity, invasiveness, elevated resistance to apoptosis, and increased production of extracellular matrix (ECM) components. In addition to these physiological characteristics, the mesenchymal features can include expression of certain biomarkers, including among others, E-cadherin, N-cadherin, integrins, FSP-1, α-SMA, vimentin, β-catenin, collagen I, collagen II, collagen III, collagen IV, fibronectin, laminin 5, SNAIL-1, SNAIL-2, Twist-1, Twist-2, and Lef-1. In some embodiments, the cancer selected for treatment with the ferroptosis inducer includes, among others, breast cancer, lung cancer, head and neck cancer, prostate cancer, and colon cancer. In some embodiments, the mesenchymal features can be inherent to the cancer type or induced by or selected for by treatment of cancers with chemo- and/or radiation therapy.

In some embodiments, the cancer for treatment with a ferroptosis inducer is identified as having or determined to have an activating or oncogenic RAS activity. In some embodiments, the RAS is K-RAS, H-RAS or N-RAS. In some embodiments, the activating or oncogenic RAS is an activating or oncogenic RAS mutation.

In some embodiments, the cancer for treatment with a ferroptosis inducer is identified as having or determined to have an activating or oncogenic K-RAS mutation. In some embodiments, the cancer selected for treatment is identified as having or determined to have an activating or oncogenic mutation in human K-RAS at one or more of codon 5, codon 9, codon 12, codon 13, codon 14, codon 18, codon 19, codon 22, codon 23, codon 24, codon 26, codon 33, codon 36, codon 57, codon 59, codon 61, codon 62, codon 63, codon 64, codon 68, codon 74, codon 84, codon 92, codon 35, codon 97, codon 110, codon 115, codon 117, codon 118, codon 119, codon 135, codon 138, codon 140, codon 146, codon 147, codon 153, codon 156, codon 160, codon 164, codon 171, codon 176, codon 185, and codon 188.

In some embodiments, the activating or oncogenic K-RAS mutation can be a mutation in which: codon 5 is K5E; codon 9 is V91; codon 12 is G12A, G12C, G12D, G12F, G12R, G12S, G12V, or G12Y; codon 13 is G13C, G13D, or G13V; codon 14 is V14I or V14L; codon 18 is A18D; codon 19 is L19F; codon 22 is Q22K; codon 23 is L23R; codon 24 is I24N; codon 26 is N26K; codon 33 is D33E; codon 36 is I36L or I36M; codon 57 is D57N; codon 59 is A59E, A59G, or A59T; codon 61 is Q61H, Q61K, Q61L, or Q61R; codon 62 is E62G or E62K; codon 63 is E63K; codon 64 is Y64D, Y64H, or Y64N; codon 68 is R68S; codon 74 is T74P; codon 84 is I84T; codon 92 is D92Y; codon 97 is R97I; codon 110 is P110H or P110S; codon 115 is G115E; codon 117 is K117N; codon 118 is C118S; codon 119 is D119N; codon 135 is R135T; codon 138 is G138V; codon 140 is P140H; codon 146 is A146T or A146V; codon 147 is K147N; codon 153 is D153N; codon 156 is F156L; codon 160 is V160A; codon 164 is R164Q; codon 171 is 1117M; codon 176 is K176Q; codon 185 is C185R or C185S; and codon 188 is M188V.

In particular, the cancer for treatment with the ferroptosis inducer is identified as having or determined to have an oncogenic or activating K-RAS mutation at codon 12, codon 13 and/or codon 61. In some embodiments, the oncogenic or activating K-RAS mutation at codon 12 is G12A, G12C, G12D, G12F, G12R, G12S, G12V, or G12Y; at codon 13 is G13C, G13D, or G13V; and at codon 61 is Q61H, Q61K, Q61L, or Q61R. In some embodiments, the oncogenic or activating K-RAS mutation is a combination of oncogenic or activating K-RAS mutations at codon 12 and codon 13; codon 12 and codon 61; codon 13 and 61; or codon 12, codon 13 and codon 61.

In some embodiments, the cancer for treatment with the ferroptosis inducer is identified as having or determined to have an activating or oncogenic N-RAS mutation. In some embodiments, the cancer is identified as having or determined to have an activating or oncogenic mutation in human N-RAS at one or more of codon 12, codon 13 and codon 61. In some embodiments, the activating or oncogenic N-RAS mutation at codon 12 is G12A, G12C, G12D, G12R, G12S, or G12V. In some embodiments, the activating or oncogenic N-RAS mutation at codon 13 is G13A, G13C, G13D, G13R, G13S, or G13V. In some embodiments, the activating or oncogenic N-RAS mutation at codon 61 is Q61E, Q61H, Q61K, Q61L, Q61P, or Q61R. In some embodiments, the oncogenic or activating N-RAS mutation is a combination of activating or oncogenic N-RAS mutations at codon 12 and codon 13; codon 12 and codon 61; codon 13 and 61; or codon 12, codon 13 and codon 61.

In some embodiments, the cancer for treatment with the ferroptosis inducer is identified as having or determined to have an activating or oncogenic H-RAS mutation. In some embodiments, the cancer selected for treatment is identified as having an activating or oncogenic mutation in human H-RAS at one or more of codon 12, codon 13 and codon 61. In some embodiments, the activating or oncogenic H-RAS mutation at codon 12 is G12A, G12C, G12D, G12R, G12S, or G12V. In some embodiments, the activating or oncogenic H-RAS mutation at codon 13 is G13A, G13C, G13D, G13R, G13S, or G13V. In some embodiments, the activating or oncogenic H-RAS mutation at codon 61 is Q61E, Q61H, Q61K, Q61L, Q61P, or Q61R. In some embodiments, the oncogenic or activating H-RAS mutation is a combination of activating or oncogenic H-RAS mutations at codon 12 and codon 13; codon 12 and codon 61; codon 13 and 61; or codon 12, codon 13 and codon 61.

In some embodiments, the cancer for treatment with the ferroptosis inducer can be a cancer having prevalence (e.g., at least about 10% or more, or about 15% or more of the cancers), of an activating or oncogenic RAS mutation, such as cancer of the biliary tract, cervix, endometrium, pancreas, lung, head and neck, stomach (gastric), colon, small intestine, hematologic (e.g., leukemia, lymphomas, etc.), ovary, prostate, salivary gland, skin, thyroid, aerodigestive tract, and urinary tract.

In some embodiments, the ferroptosis inducer can be used to treat a cancer that is refractory to one or more other chemotherapeutic agents, particularly cytotoxic chemotherapeutic agents; or a cancer resistant to radiation treatment. In particular, the ferroptosis inducers is used to treat cancers that have developed tolerance to chemotherapeutic agents that activate other cell death pathways, such as apoptosis, mitotic catastrophe, necrosis, senescence and/or autophagy.

In some embodiments, the cancer for treatment with the ferroptosis inducer is identified as being refractory or resistant to chemotherapy. In some embodiments, the cancer is refractory or resistant to one or more of alkylating agents, antibiotic agents, antimetabolic agents (e.g., folate antagonists, purine analogs, pyrimidine analogs, etc.), topoisomerase inhibiting agents, anti-microtubule agents (e.g., taxanes, vinca alkaloids), hormonal agents (e.g., aromatase inhibitors), plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors.

In some embodiments, the cancer for treatment with the ferroptosis inducer is a cancer identified as being refractory or resistant to one or more of afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, and vindesine.

In some embodiments, the cancer for treatment with a ferroptosis inducer is identified as being refractory or resistant to one or more chemotherapeutics agents selected from cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and erlotinib.

In some embodiments, the cancer for treatment with a ferroptosis inducer is a cancer resistant to ionizing radiation therapy. The radioresistance of the cancer can be inherent or as a result of radiation therapy. In some embodiments, the cancers for treatment with the ferroptosis inducer is, among others, a radioresistant adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemias and lymphomas), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, and vaginal cancer, particularly breast cancer, glioblastoma, advanced non-small-cell lung cancer, bladder cancer, sarcoma, or soft tissue carcinoma.

In the embodiments herein, the ferroptosis inducer can be selected from agents capable of inducing ferroptosis in cells, particularly agents having ferroptosis inducing activity in cancer cells.

In some embodiments, the ferroptosis inducer is a compound of formula (I):

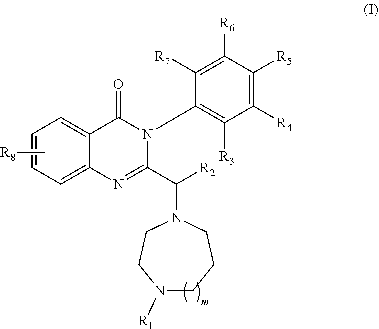

or an N-oxide, hydrate, solvate, or pharmaceutically acceptable salt thereof,

wherein

R₁ selected from the group consisting of H, C₁₋₄ alkyl, arylC₁₋₄ alkyl, aryl, heteroaryl, acyl, alkylsulfonyl, and arylsulfonyl, or R₁ is —NRₐR_{b}, wherein Rₐ and R_{b} are each independently selected from H, arylC₁₋₄alkyl, aryl, heteroaryl, acyl, alkylsulfonyl, and arylsulfonyl;

R₂ is selected from the group consisting of H, C₁₋₈alkyl, —NRₐR_{b}, C₁₋₈alkyl-OR₃, 3- to 8-membered carbocyclic or heterocyclic, aryl, heteroaryl, and arylC₁₋₄alkyl;

R₃, R₄, R₅, R₆ and R₇ are each independently selected from the group consisting of H, halo, C₁₋₈alkyl, C₁₋₈alkylamino, C₁₋₈alkyloxy, C₂₋₈alkenyl, C₂₋₈alkynyl, amide, amine, carbamate, carbonate, carboxy, acyl, ether, heterocycloalkyl, and arylalkyl; and

R₈ is selected from the group consisting of H, halo, NH₂, C₁₋₆ alkyl, C₁₋₄alkyloxy, carbonyl, aryl, heteroaryl, C₃₋₈ cycloalkyl, and C₃₋₈ heterocycloalkyl; and

m is 0 or 1.

In some embodiments, the ferroptosis inducer is a compound of formula (Ia):

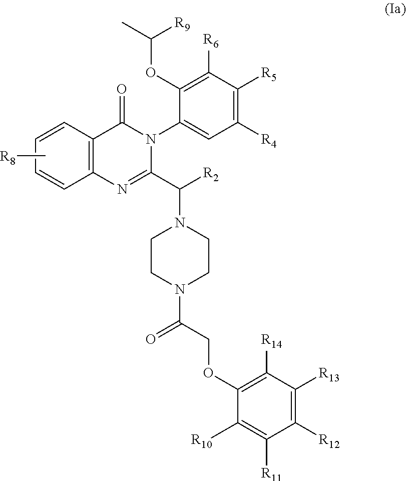

or an N-oxide, hydrate, solvate, or pharmaceutically acceptable salt thereof,

wherein

R₂ is selected from the group consisting of H, C₁₋₈alkyl, —N(Rₐ)(R_{b}), C₁₋₈alkyl-OR₃, 3- to 8-membered carbocyclic or heterocyclic, aryl, heteroaryl, and arylC₁₋₄alkyl;

R₄, R₅, and R₆ are each independently selected from the group consisting of H, halo, C₁₋₈alkyl, C₁₋₈alkylamino, C₁₋₈alkyloxy, C₂₋₈alkenyl, C₂₋₈alkynyl, amide, amine, carbamate, carbonate, carboxy, acyl, ether, heteroalkyl, and arylalkyl;

R₈ is selected from group consisting of H, halo, NH₂, C₁₋₆ alkyl, C₁₋₄ alkyloxy, carbonyl, aryl, heteroaryl, C₃₋₈ cycloalkyl, and C₃₋₈ heterocycloalkyl;

R₉ is H or C₁₋₄alkyl;

R₁₀, R₁₁, R₁₂, R₁₃, and R₁₄ are independently selected from the group consisting of H, halo, C₁₋₄alkyl, C₁₋₄alkylamino, acyl, and alkylsulfonyl; and

Rₐ and R_{b} are each independently selected from the group consisting of H, C₁₋₄ alkyl, arylC₁₋₄alkyl, aryl, heteroaryl, acyl, alkylsulfonyl, and arylsulfonyl.

In some embodiments, the compounds of formula (I) and formula (Ia) and related analogs and derivatives thereof; methods of their synthesis; and dosages are described in patent publication US20070161644, incorporated herein by reference.

In some embodiments, the ferroptosis inducer is a compound of formula (II):

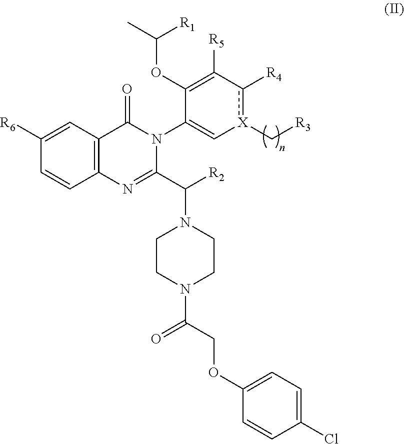

or an N-oxide, hydrate, solvate, or pharmaceutically acceptable salt thereof,

wherein

R₁ is selected from the group consisting of H, C₁₋₄alkyloxy, hydroxy, and halo;

R₂ is selected from the group consisting of H, C₁₋₄alkyloxy, C₃₋₈ cycloalkyl, C₃₋₈ heterocycloalkyl, aryl, heteroaryl, arylC₁₋₄ alkyl;

R₃ is selected from the group consisting of nothing, H, C₁₋₄alkyloxy, carbonyl, C₃₋₈cycloalkyl, and C₃₋₈heterocycloalkyl;

R₄ and R₅ are independently selected from the group consisting of H, C₃₋₈ cycloalkyl, and C₃₋₈heterocycloalkyl;

R₆ is selected from the group consisting of H, NH₂, C₁₋₄ alkyl, C₁₋₄ alkyloxy, carbonyl, aryl, heteroaryl, C₃₋₈ cycloalkyl, and C₃₋₈ heterocycloalkyl;

X is selected from the group consisting of C, N, and O; and

n is an integer from 0-6.

In some embodiments, the ferroptosis inducer is a compound of formula (IIa):

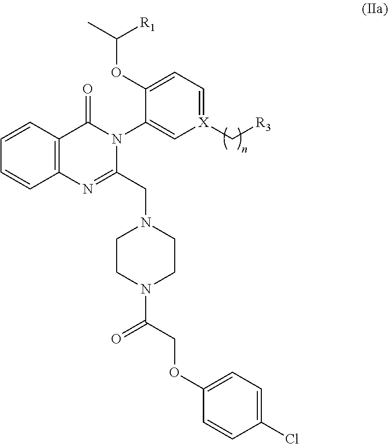

or an N-oxide, hydrate, solvate, or pharmaceutically acceptable salt thereof,

wherein

R₁ is selected from the group consisting of H, C₁₋₄alkyloxy, hydroxy, and halo;

R₃ is selected from the group consisting of nothing, C₁₋₄alkyl, C₁₋₄ alkyloxy, carbonyl, C₃₋₈ cycloalkyl, and C₃₋₈ heterocycloalkyl;

X is selected from the group consisting of C and N; and

n is an integer from 0-6.

In some embodiments, the ferroptosis inducer is a compound of formula (IIb):

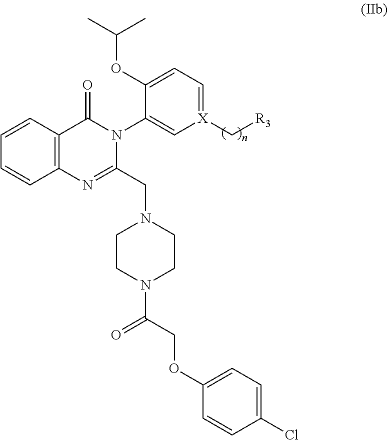

or an N-oxide, hydrate, solvate, or pharmaceutically acceptable salt thereof,

wherein

R₁ is selected from the group consisting of H, C₁₋₄alkyloxy, hydroxy, and halo;

R₃ is selected from the group consisting of nothing, C₁₋₄alkyl, C₁₋₄alkyloxy, carbonyl, C₃₋₈cycloalkyl, and C₃₋₈heterocycloalkyl;

X is selected from the group consisting of C and N; and

n is an integer from 0-6.

In some embodiments, the ferroptosis inducer is selected from:

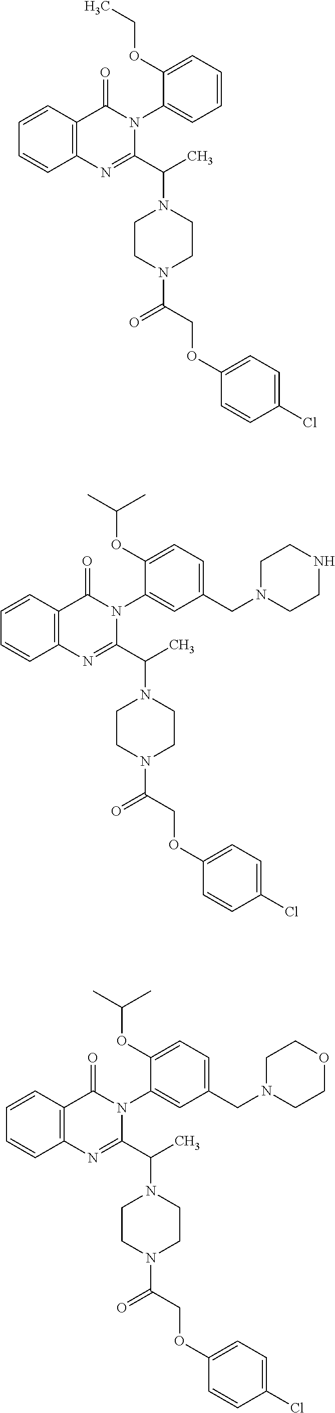 [SMILES: CCOc1ccccc1-n1c(C(C)N2CCN(C(=O)COc3ccc(Cl)cc3)CC2)nc2ccccc2c1=O ; CC(C)Oc1ccc(CN2CCNCC2)cc1-n1c(C(C)N2CCN(C(=O)COc3ccc(Cl)cc3)CC2)nc2ccccc2c1=O ; CC(C)Oc1ccc(CN2CCOCC2)cc1-n1c(C(C)N2CCN(C(=O)COc3ccc(Cl)cc3)CC2)nc2ccccc2c1=O]

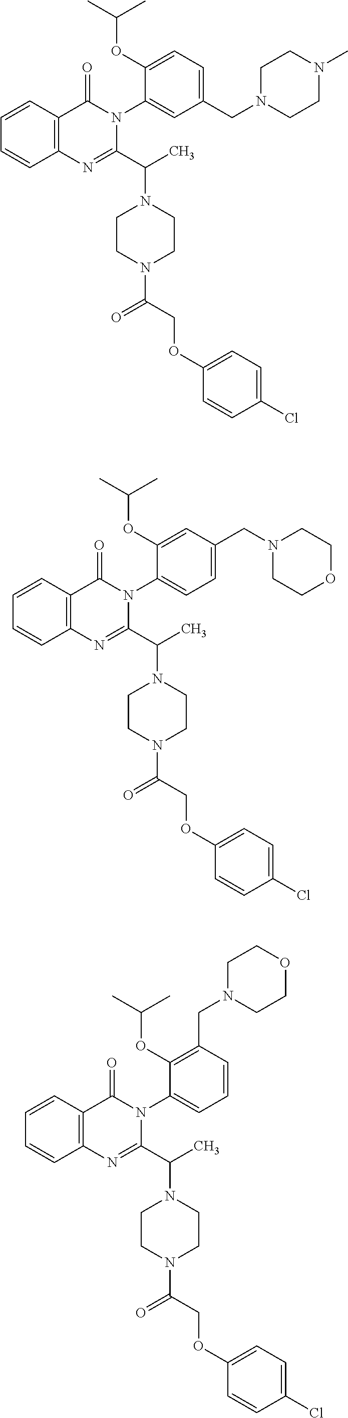 [SMILES: CC(C)Oc1ccc(CN2CCN(C)CC2)cc1-n1c(C(C)N2CCN(C(=O)COc3ccc(Cl)cc3)CC2)nc2ccccc2c1=O ; CC(C)Oc1cc(CN2CCOCC2)ccc1-n1c(C(C)N2CCN(C(=O)COc3ccc(Cl)cc3)CC2)nc2ccccc2c1=O ; CC(C)Oc1c(CN2CCOCC2)cccc1-n1c(C(C)N2CCN(C(=O)COc3ccc(Cl)cc3)CC2)nc2ccccc2c1=O]

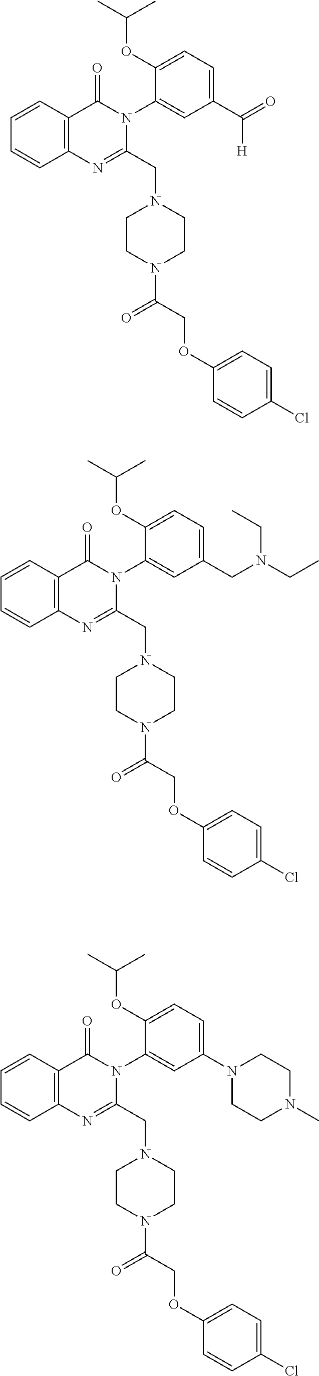 [SMILES: CC(C)Oc1ccc(C=O)cc1-n1c(CN2CCN(C(=O)COc3ccc(Cl)cc3)CC2)nc2ccccc2c1=O ; CCN(CC)Cc1ccc(OC(C)C)c(-n2c(CN3CCN(C(=O)COc4ccc(Cl)cc4)CC3)nc3ccccc3c2=O)c1 ; CC(C)Oc1ccc(N2CCN(C)CC2)cc1-n1c(CN2CCN(C(=O)COc3ccc(Cl)cc3)CC2)nc2ccccc2c1=O]

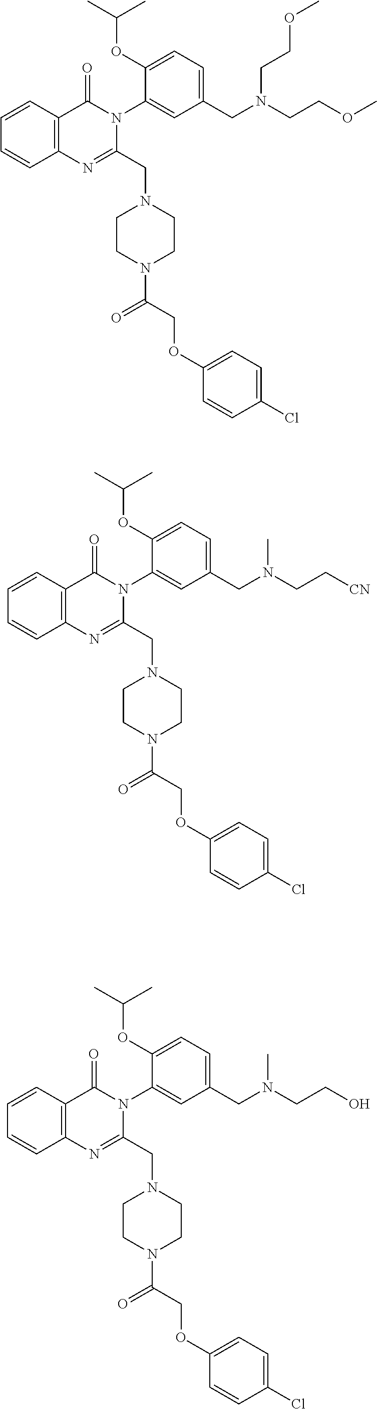 [SMILES: COCCN(CCOC)Cc1ccc(OC(C)C)c(-n2c(CN3CCN(C(=O)COc4ccc(Cl)cc4)CC3)nc3ccccc3c2=O)c1 ; CC(C)Oc1ccc(CN(C)CCC#N)cc1-n1c(CN2CCN(C(=O)COc3ccc(Cl)cc3)CC2)nc2ccccc2c1=O ; CC(C)Oc1ccc(CN(C)CCO)cc1-n1c(CN2CCN(C(=O)COc3ccc(Cl)cc3)CC2)nc2ccccc2c1=O]

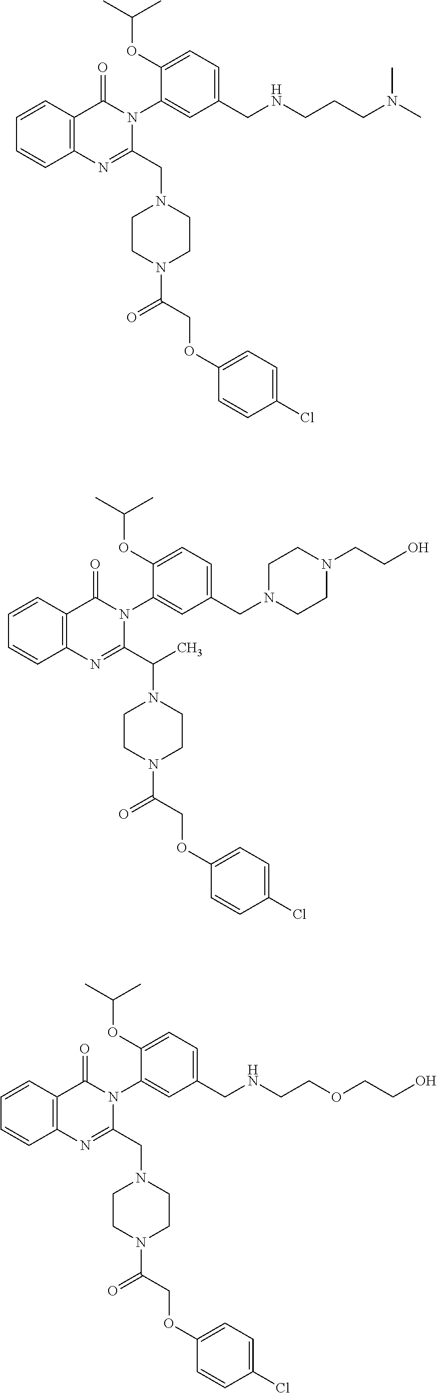 [SMILES: CC(C)Oc1ccc(CNCCCN(C)C)cc1-n1c(CN2CCN(C(=O)COc3ccc(Cl)cc3)CC2)nc2ccccc2c1=O ; CC(C)Oc1ccc(CN2CCN(CCO)CC2)cc1-n1c(C(C)N2CCN(C(=O)COc3ccc(Cl)cc3)CC2)nc2ccccc2c1=O ; CC(C)Oc1ccc(CNCCOCCO)cc1-n1c(CN2CCN(C(=O)COc3ccc(Cl)cc3)CC2)nc2ccccc2c1=O]

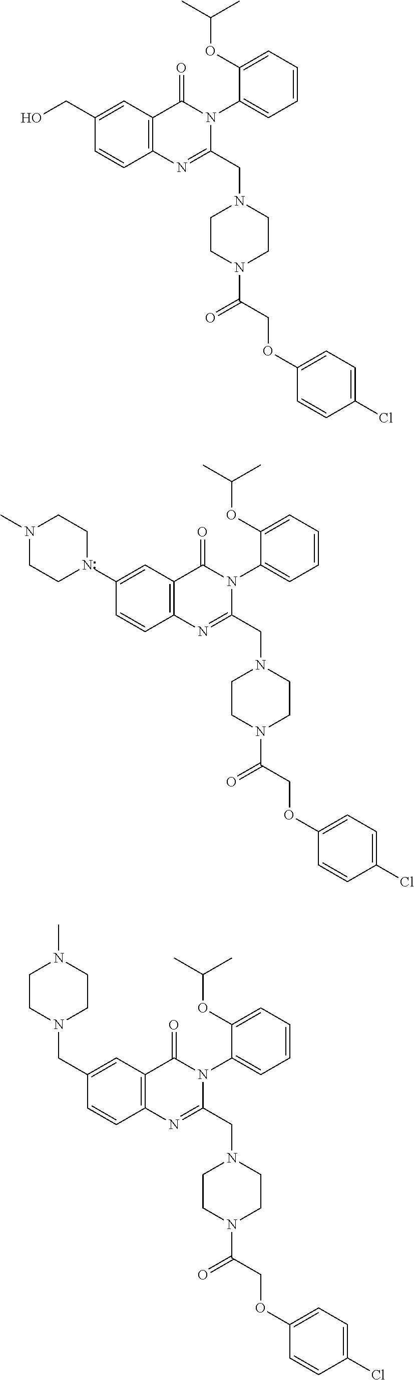 [SMILES: CC(C)Oc1ccccc1-n1c(CN2CCN(C(=O)COc3ccc(Cl)cc3)CC2)nc2ccc(CO)cc2c1=O ; CC(C)Oc1ccccc1-n1c(CN2CCN(C(=O)COc3ccc(Cl)cc3)CC2)nc2ccc(N3CCN(C)CC3)cc2c1=O ; CC(C)Oc1ccccc1-n1c(CN2CCN(C(=O)COc3ccc(Cl)cc3)CC2)nc2ccc(CN3CCN(C)CC3)cc2c1=O]

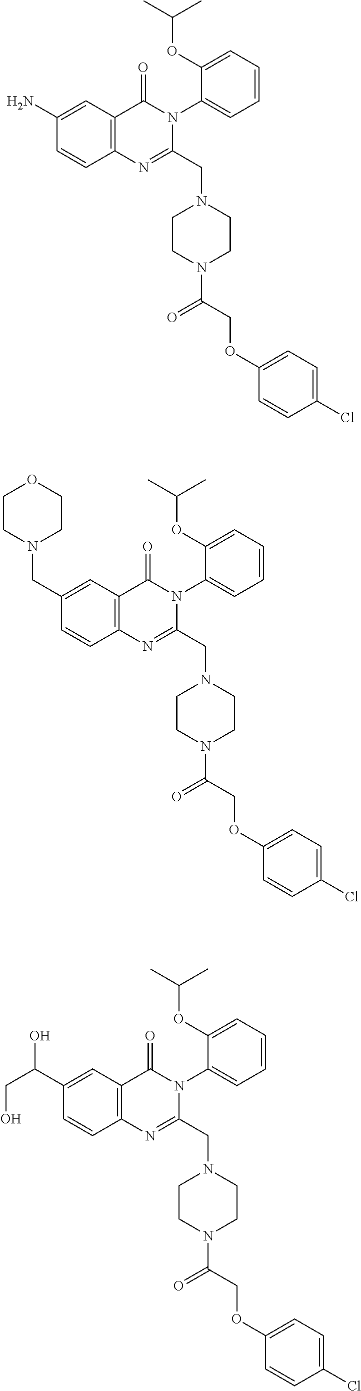 [SMILES: CC(C)Oc1ccccc1-n1c(CN2CCN(C(=O)COc3ccc(Cl)cc3)CC2)nc2ccc(N)cc2c1=O ; CC(C)Oc1ccccc1-n1c(CN2CCN(C(=O)COc3ccc(Cl)cc3)CC2)nc2ccc(CN3CCOCC3)cc2c1=O ; CC(C)Oc1ccccc1-n1c(CN2CCN(C(=O)COc3ccc(Cl)cc3)CC2)nc2ccc(C(O)CO)cc2c1=O]

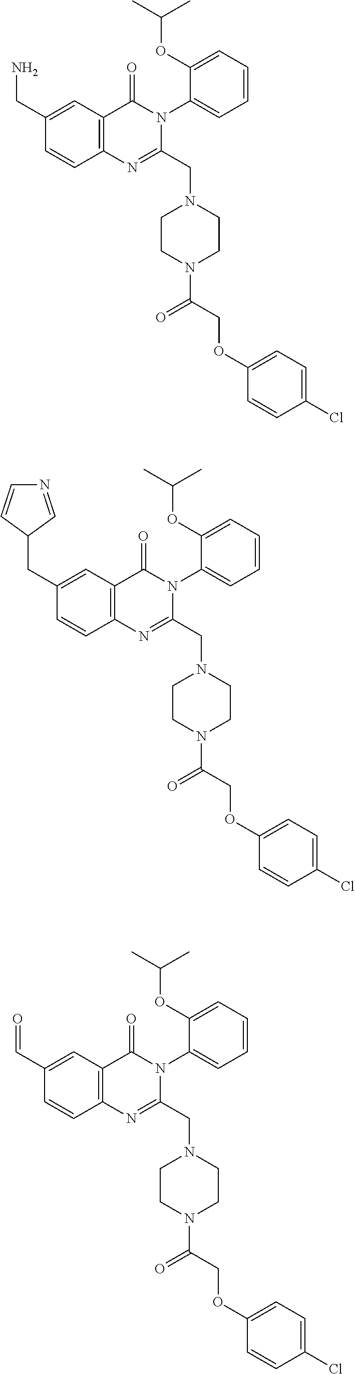 [SMILES: CC(C)Oc1ccccc1-n1c(CN2CCN(C(=O)COc3ccc(Cl)cc3)CC2)nc2ccc(CN)cc2c1=O ; CC(C)Oc1ccccc1-n1c(CN2CCN(C(=O)COc3ccc(Cl)cc3)CC2)nc2ccc(CC3C=CN=C3)cc2c1=O ; CC(C)Oc1ccccc1-n1c(CN2CCN(C(=O)COc3ccc(Cl)cc3)CC2)nc2ccc(C=O)cc2c1=O]

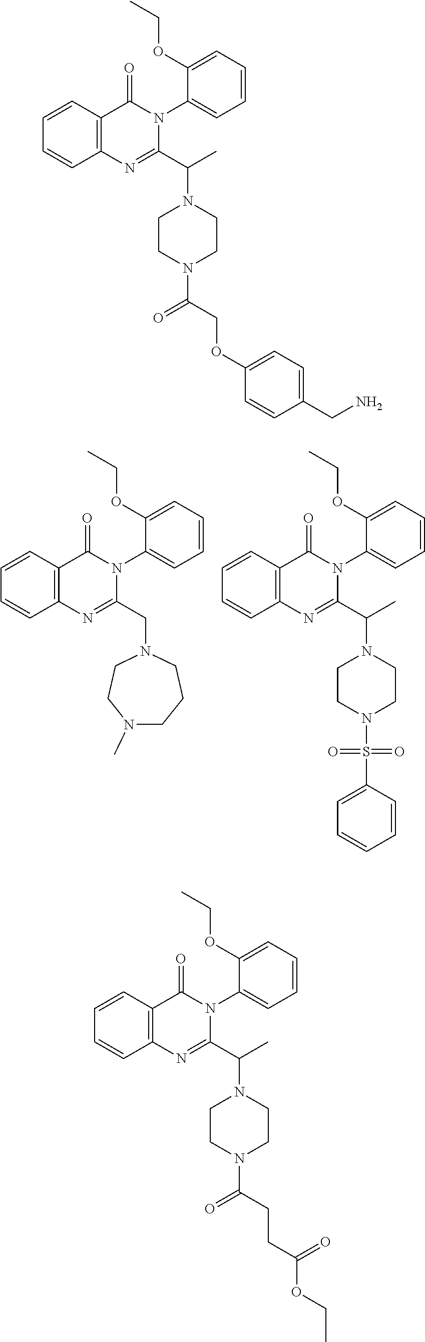

and an N-oxide, hydrate, solvate, and pharmaceutically acceptable salt thereof.

In some embodiments, the compounds of formula (II), formula (IIa), and formula (IIb), for example erastin; methods of their synthesis; and dosages are described in patent publication US20150175558, incorporated herein by reference. Other analogs are described in US20070161644, as noted above.

In some embodiments, the ferroptosis inducer is a compound of formula (III):

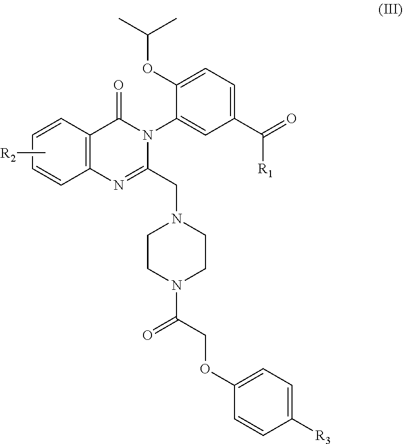

or an N-oxide, hydrate, solvate, or pharmaceutically acceptable salt thereof,

wherein

R₁ is selected from the group consisting of H, C₁₋₆ alkyl, and CF₃, wherein each C₁₋₆ alkyl may be optionally substituted with an atom or a group selected from the group consisting of a halogen, a saturated or unsaturated C₃₋₆-heterocyclyl, and an amine, each heterocyclyl is optionally substituted with an C₁₋₄aliphatic, which C₁₋₄ aliphatic may be optionally substituted with an C₁₋₄ alkylaryl-O—C₁₋₄alkyl;

R₂ is selected from the group consisting of H, halo, and C₁₋₆ aliphatic; and

R₃ is a halo atom.

In some embodiments, the ferroptosis inducer is a compound for formula (Ma):

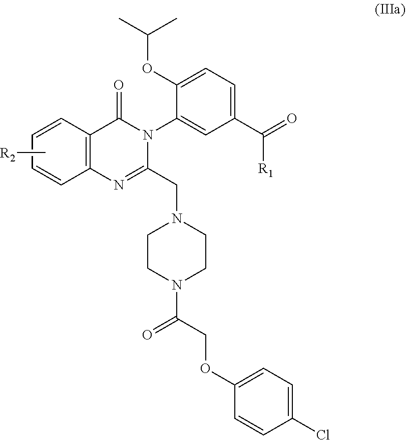

or an N-oxide, solvate, or pharmaceutically acceptable salt thereof, wherein:

R₁ is selected from the group consisting of H, C₁₋₆ alkyl, and CF₃, wherein each C₁₋₆ alkyl may be optionally substituted with an atom or a group selected from the group consisting of a halogen atom, a saturated or unsaturated C₃₋₆-heterocyclyl, and an amine, each heterocyclyl is optionally substituted with an C₁₋₄ aliphatic, wherein the C₁₋₄ aliphatic may be optionally substituted with an C₁₋₄alkylaryl-O—C₁₋₄alkyl; and

R₂ is selected from the group consisting of H, halo, and C₁₋₆ aliphatic.

In some embodiments of the compound of formula (III) or (IIIa), R₂ is H.

In some embodiments, the ferroptosis inducer is selected from:

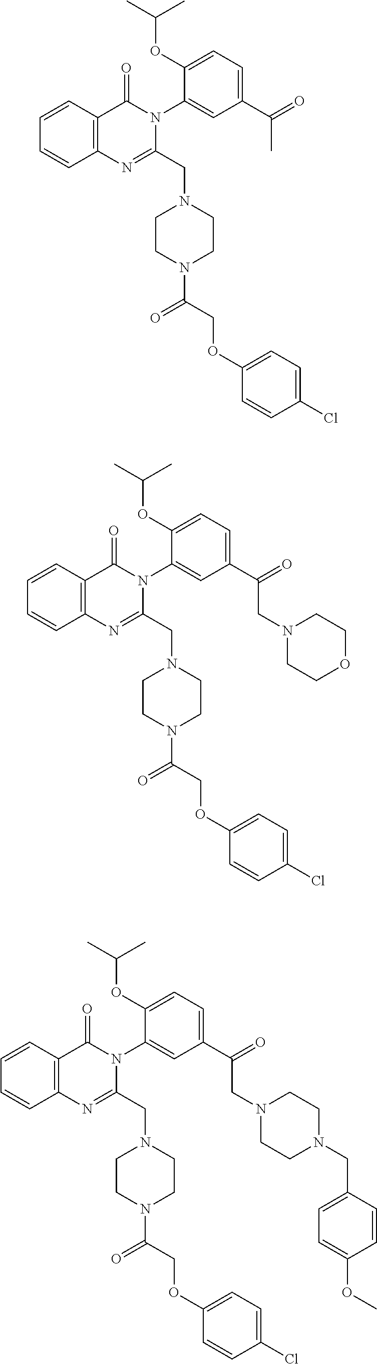 [SMILES: CC(=O)c1ccc(OC(C)C)c(-n2c(CN3CCN(C(=O)COc4ccc(Cl)cc4)CC3)nc3ccccc3c2=O)c1 ; CC(C)Oc1ccc(C(=O)CN2CCOCC2)cc1-n1c(CN2CCN(C(=O)COc3ccc(Cl)cc3)CC2)nc2ccccc2c1=O ; COc1ccc(CN2CCN(CC(=O)c3ccc(OC(C)C)c(-n4c(CN5CCN(C(=O)COc6ccc(Cl)cc6)CC5)nc5ccccc5c4=O)c3)CC2)cc1]

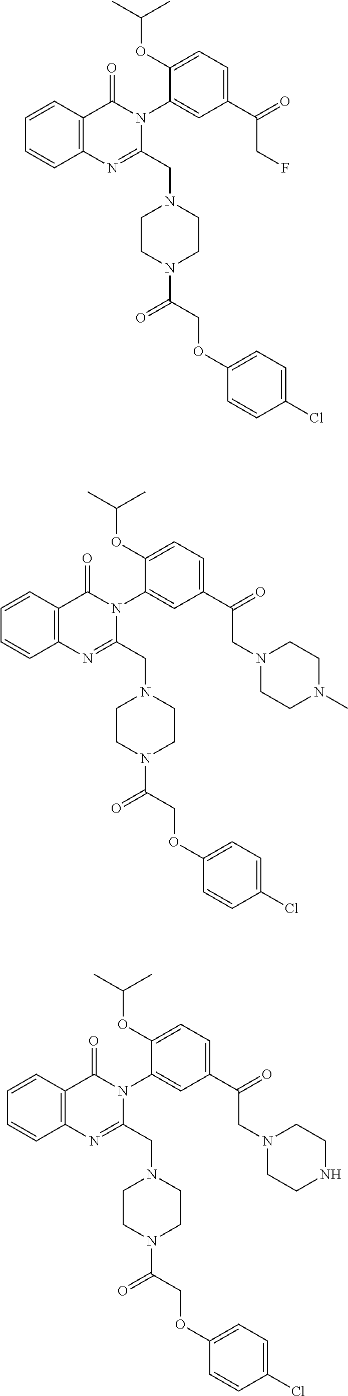 [SMILES: CC(C)Oc1ccc(C(=O)CF)cc1-n1c(CN2CCN(C(=O)COc3ccc(Cl)cc3)CC2)nc2ccccc2c1=O ; CC(C)Oc1ccc(C(=O)CN2CCN(C)CC2)cc1-n1c(CN2CCN(C(=O)COc3ccc(Cl)cc3)CC2)nc2ccccc2c1=O ; CC(C)Oc1ccc(C(=O)CN2CCNCC2)cc1-n1c(CN2CCN(C(=O)COc3ccc(Cl)cc3)CC2)nc2ccccc2c1=O]

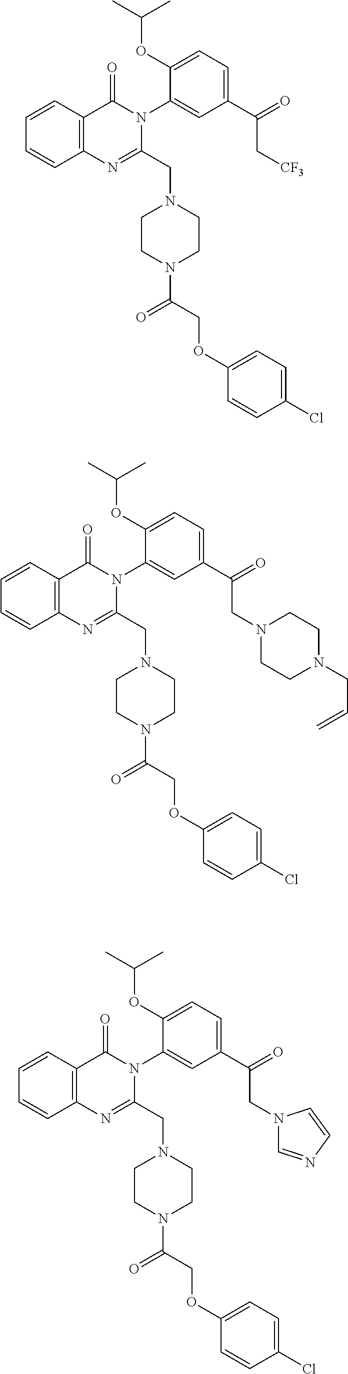 [SMILES: CC(C)Oc1ccc(C(=O)CC(F)(F)F)cc1-n1c(CN2CCN(C(=O)COc3ccc(Cl)cc3)CC2)nc2ccccc2c1=O ; C=CCN1CCN(CC(=O)c2ccc(OC(C)C)c(-n3c(CN4CCN(C(=O)COc5ccc(Cl)cc5)CC4)nc4ccccc4c3=O)c2)CC1 ; CC(C)Oc1ccc(C(=O)Cn2ccnc2)cc1-n1c(CN2CCN(C(=O)COc3ccc(Cl)cc3)CC2)nc2ccccc2c1=O]

and an N-oxide, hydrate, solvate, and pharmaceutically acceptable salt thereof.

In some embodiments, the compounds of formula (III) and formula (Ma); methods of their synthesis; and dosages are described in patent publication WO2015109009, incorporated herein by reference.

In some embodiments, the ferroptosis inducer is a compound of formula (IV):

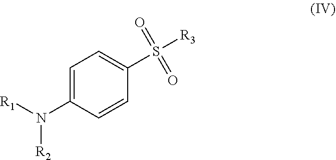

or an N-oxide, hydrate, solvate, or pharmaceutically acceptable salt thereof,

wherein,

R₁ and R₂ are H or together form N=phenyl, wherein the phenyl is substituted with a carboxyl group and/or a hydroxyl; and

R₃ is a —NH-pyridyl.

In some embodiments, the ferroptosis inducer is sulfasalazine:

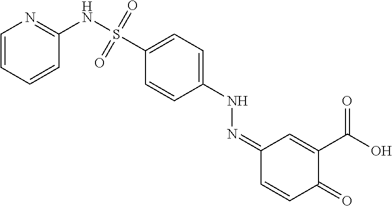 [SMILES: O=C(O)C1=C/C(=N\Nc2ccc(S(=O)(=O)Nc3ccccn3)cc2)C=CC1=O]

or an N-oxide, hydrate, solvate, or pharmaceutically acceptable salt thereof.

Compounds of formula (IV), and analogs and derivatives thereof; methods of their synthesis; and dosages are described in patent publication US20160120884, incorporated herein by reference.

In some embodiments, the ferroptosis inducer is a compound of formula (V):

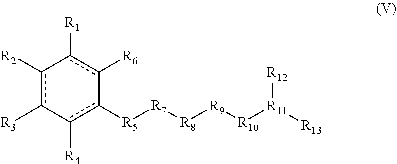

or an N-oxide, hydrate, solvate, or pharmaceutically acceptable salt thereof,

wherein

R₁, R₂, R₃, R₄, and R₆ are independently selected from the group consisting of H, halo, CF₃, OCF₃, C₁₋₄ alkyl, and CN;

R₅ is selected from the group consisting of no atom, NH, O, and C₁₋₄ alkyl;

R₇ is selected from the group consisting of no atom, carbonyl, thiocarbonyl, and sulfonyl;

R₈ is NH or no atom;

R₉ is an aryl or heteroaryl optionally substituted with hydroxy, C₁₋₄ alkyl, halo, and combinations thereof;

R₁₀ is no atom, O, or C₁₋₄ alkyl;

R₁₁ is selected from the group consisting of no atom, aryl, heterocyclyl, and heteroaryl optionally substituted with a halo or a C₁₋₄ alkyl;

R₁₂ is selected from the group consisting of no atom, amide, CN, heteroaryl, O, C₁₋₄ alkyloxy, and amine;

R₁₃ is selected from the group consisting of no atom, C₁₋₄ alkyloxy, amine, CN, carboxy, and carbocyclyl optionally substituted with one or more of C₁₋₄ alkyl and OH; and the dotted line (- - - -) is an optional double bond.

In some embodiments, the compound of formula (V), or an N-oxide, hydrate, or pharmaceutically acceptable salt thereof, has the structure (Va):

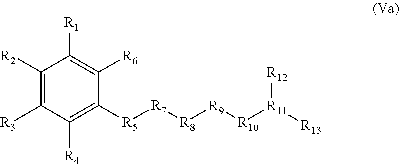

wherein
R₁, R₂, R₃, R₄, R₅, R₆, R₇, R₈, R₉, R₁₀, R₁₁, R₁₂, and R₁₃ are as defined for formula (V).

In some embodiments of the compound of formula (V), or an N-oxide, hydrate, or pharmaceutically acceptable salt thereof,

R₁, R₂, R₃, R₄, and R₆ are independently selected from the group consisting of H, halo, CF₃, OCF₃, C₁₋₄ alkyl, and CN;

R₅ is independently selected from the group consisting of no atom, NH, O, and C₁₋₄ alkyl;

R₇ is selected from the group consisting of no atom, carbonyl, thiocarbonyl, and sulfonyl;

R₈ is NH or no atom;

R₉ is an aryl or heteroaryl optionally substituted with a group selected from the group consisting of H, hydroxy, C₁₋₄ alkyl, halo, and combinations thereof;

R₁₀ is no atom, O, or C₁₋₄ alkyl;

R₁₁ is selected from the group consisting of no atom, aryl, heterocycle, and heteroaryl optionally substituted with a halo or a C₁₋₄ alkyl;

R₁₂ is selected from the group consisting of no atom, amide, CN, heteroaryl, O, C₁₋₄ alkyloxy, and amine;

R₁₃ is selected from the group consisting of no atom, C₁₋₄ alkyloxy, amine, CN, carboxy, carbocyclyl optionally substituted with C₁₋₄alkyl, diol; and the dotted line (- - - - -) is an optional double bond.

In some embodiments, the ferroptosis inducer is a compound of formula (Vb):

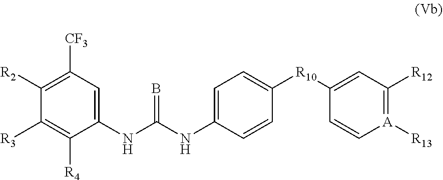

or an N-oxide, hydrate, solvate, or pharmaceutically acceptable salt thereof,

wherein

R₂ and R₃ are independently selected from the group consisting of H, halo, and CF₃;

R₄ is H or halo;

R₁₀ is O or C₁₋₄ alkyl;

A is C or N;

B is O or S;

R₁₂ is selected from the group consisting of no atom, amide, CN, and heteroaryl; and

R₁₃ is selected from the group consisting of no atom and CN.

In some embodiments, the ferroptosis inducer is a compound of formula (Vc):

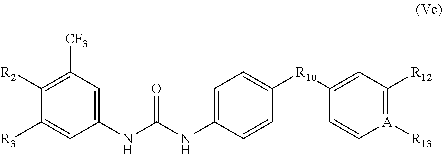

or an N-oxide, hydrate, solvate, or pharmaceutically acceptable salt thereof,

wherein

R₂ and R₃ are independently selected from the group consisting of H, halo, and CF₃;

R₁₀ is O or C₁₋₄alkyl;

A is C or N;

B is O or S;

R₁₂ is selected from the group consisting of no atom, amide, CN, and heteroaryl; and

R₁₃ is selected from the group consisting of no atom or CN.

In some embodiments, the ferroptosis inducer is selected from:

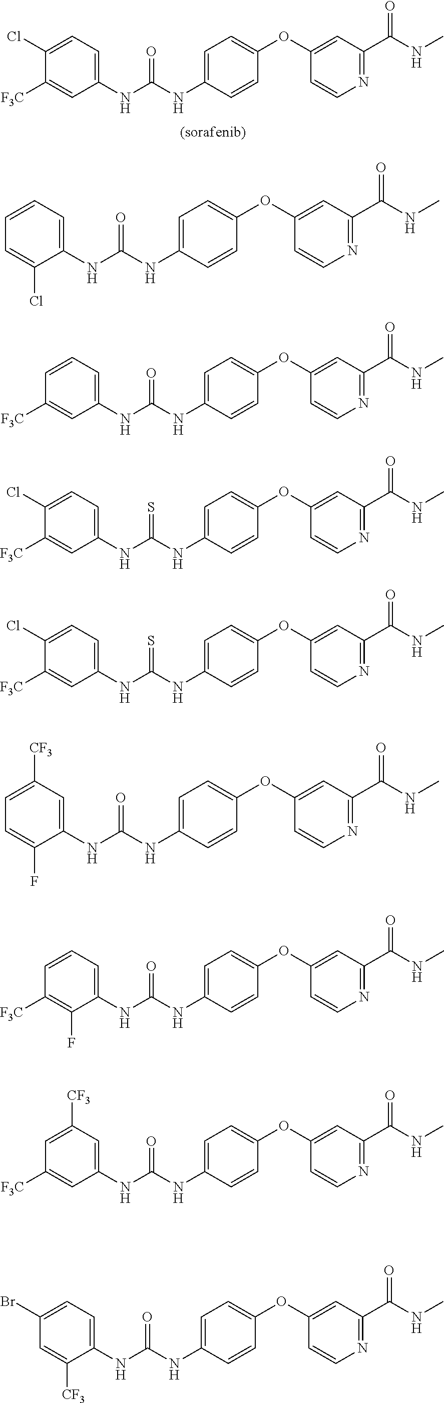

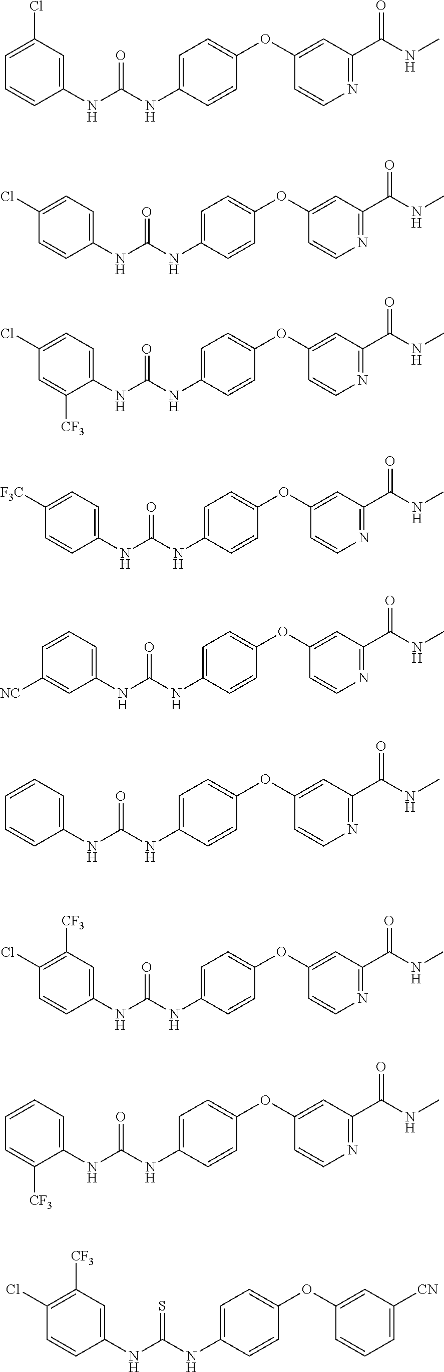

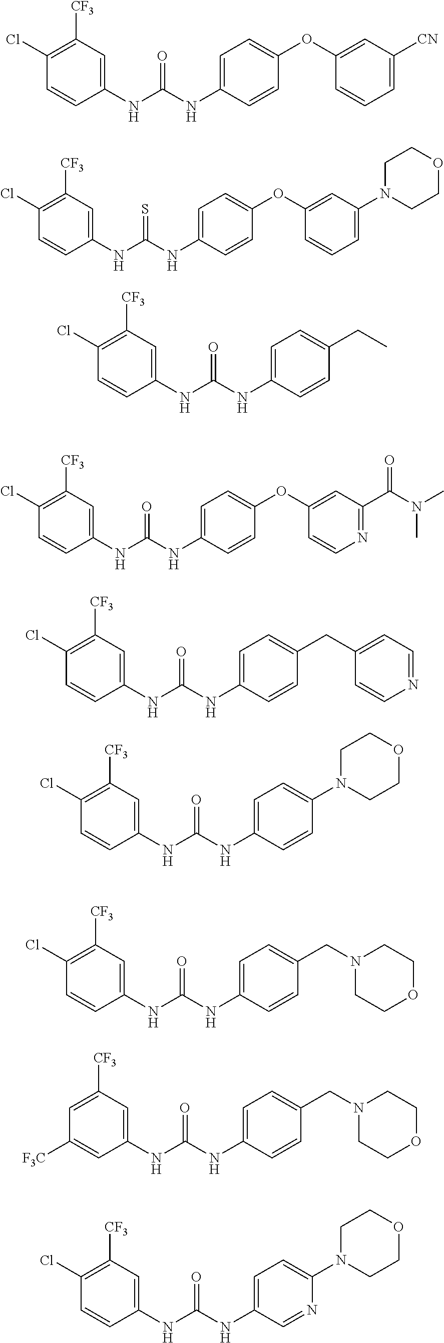

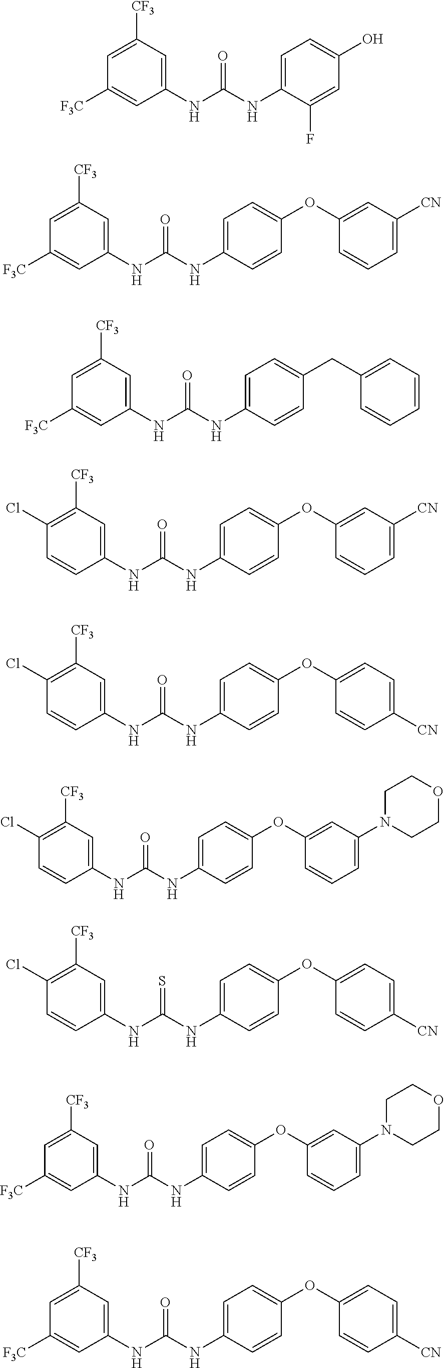

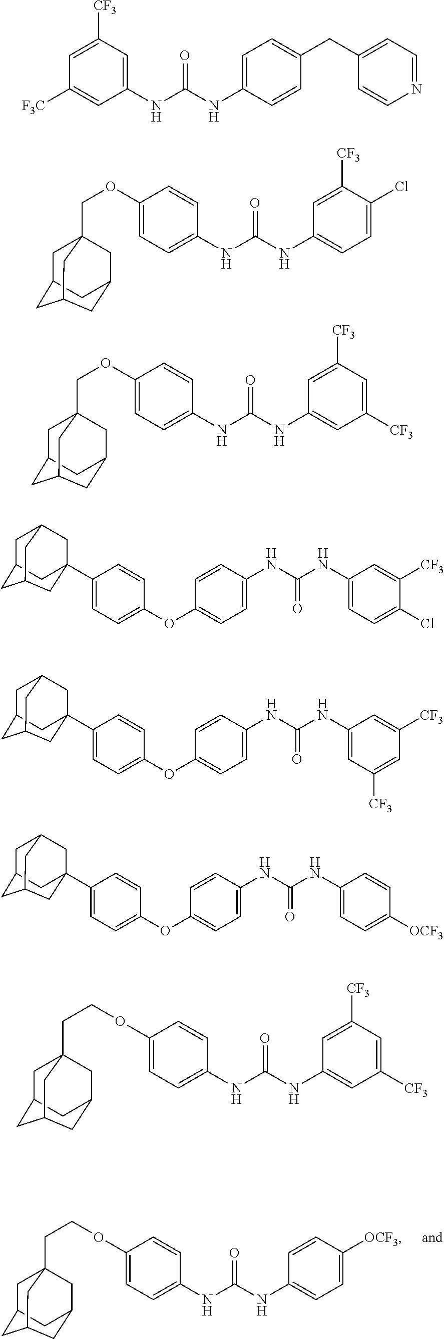

an N-oxide, hydrate, solvate, and pharmaceutically acceptable salt thereof.

In some embodiments, the compounds of formula (V), formula (Va), formula (Vb), and formula (Vc), such as sorafenib; methods of their synthesis; and dosages are described in patent publication WO2015051149, incorporated herein by reference.

In some embodiments, the ferroptosis inducer is a compound of formula (VI):

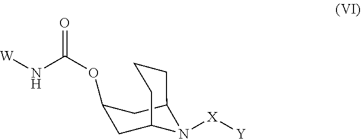

or a hydrate, solvate, or pharmaceutically acceptable salt thereof,

wherein

W is an optionally substituted C₆₋₁₀aryl or C₅₋₁₀heteroaryl;

X is a linking moiety; and

Y is a ferroptosis inducing moiety selected from erastin, erastin-A, erastin-B, desmethyl erastin and a synthetic erastin mimetic. In some embodiments, X is a C₁₋₁₂alkylene, C₁₋₁₂alkyleneamino, C₂₋₁₂alkenylene, or C₂₋₁₂alkenylamino, wherein 1-3 carbon atoms of the alkylene are optionally substituted with O or N.

In some embodiments, the ferroptosis inducer is a compound of formula (VIa),

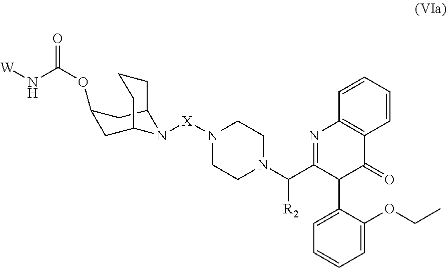

or a hydrate, solvate, or pharmaceutically acceptable salt thereof,

wherein

W is selected from optionally substituted C₆₋₁₀ aryl and C₅₋₁₀heteroaryl;

X is a linking moiety; and

R₂ is selected from hydrogen and methyl.

In some embodiments, X is

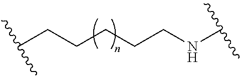

wherein

n is an integer from 1-10. In some embodiments, n is 1, 2, 3, 4, 5, 6, 7, 8, 9 or 10. In some embodiments, n is 1 or 5.

In some embodiments, the ferroptosis inducer is a compound of formula (VIb),

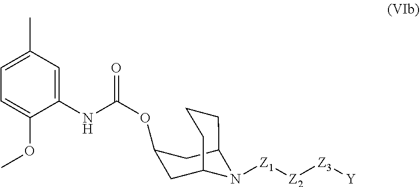

or a hydrate, solvate, or pharmaceutically acceptable salt thereof,

wherein

each of Z¹ and Z³ is independently selected from the group consisting of a bond, C₁₋₁₀alkyl, C₁₋₁₀alkenyl, C₁₋₁₀alkynyl, C₅₋₁₀aryl, C₅₋₁₀heteroaryl, arylalkyl, heteroarylalkyl, alkylaryl, alkylheteroaryl, heterocycloalkylalkyl, —(CH₂)ₐ(OCH₂CH₂)_{b}(CH₂)—, —(CH₂)ₐ(CH₂CH₂O)_{b}(CH₂)_{c}—, —(CH₂)ₐO(CH₂)_{c}—, —(CH₂)ₐS(CH₂)_{c}—, —(CH₂)ₐS(O)₂(CH₂)_{c}—, —(CH₂)ₐS(O)(CH₂)_{c}—, —(CH₂)ₐN(R¹)(CH₂)_{c}—, —(CH₂)ₐN(R¹)C(O)(CH₂)_{c}—, —(CH₂)ₐC(O)N(R¹)(CH₂)_{c}—, —(CH₂)ₐN(R¹)C(O)N(R¹)(CH₂)_{c}—, —(CH₂)ₐS(O)₂N(R¹)(CH₂)_{c}—, —(CH₂)ₐN(R)S(O)₂(CH₂)_{c}—,

Z² is selected from the group consisting of C₁₋₁₀alkyl, C₁₋₁₀alkenyl, C₁₋₁₀alkynyl, C₅₋₁₀aryl, C₅₋₁₀heteroaryl, arylalkyl, heteroarylalkyl, alkylaryl, alkylheteroaryl, heterocycloalkylalkyl, alkylheterocycloalkyl, cycloalkylalkyl, alkylcycloalkyl, —(CH₂)ₐ(OCH₂CH₂)_{b}(CH₂)_{c}—, —(CH₂)ₐ(CH₂CH₂O)_{b}(CH₂)_{c}—, —(CH₂)ₐO(CH₂)_{c}—, —(CH₂)ₐS(CH₂)_{c}—, —(CH₂)ₐS(O)₂(CH₂)_{c}—, —(CH₂)ₐS(O)(CH₂)_{c}—, —(CH₂)ₐN(R¹)(CH₂)_{c}—, —(CH₂)ₐN(R¹)C(O)(CH₂)_{c}—, —(CH₂)ₐC(O)N(R¹)(CH₂)_{c}—, —(CH₂)ₐN(R¹)C(O)N(R¹)(CH₂)_{c}—, —(CH₂)ₐS(O)₂N(R¹)(CH₂)_{c}—, —(CH₂)ₐN(R)S(O)₂(CH₂)_{c}—, wherein each of Z¹, Z² and, Z³ can be optionally substituted with one or more groups chosen from halo, oxo, and C₁₋₁₀alkyl;

each R¹ independently selected from the group consisting of hydrogen, C₁₋₁₀alkyl, and C₁₋₁₀acyl;

each a and c is an integer independently selected from the group consisting of 0, 1, 2, 3, and 4;

each b is an integer independently selected from the group consisting of 1, 2, 3, 4, 5, and 6; and

Y is a ferroptosis inducing moiety selected from the group consisting of erastin, an erastin analog such as erastin-A, erastin-B, or desmethyl-erastin or a simplified synthetic erastin mimetic.

In some embodiments, the ferroptosis inducer is a compound of formula (VIc):

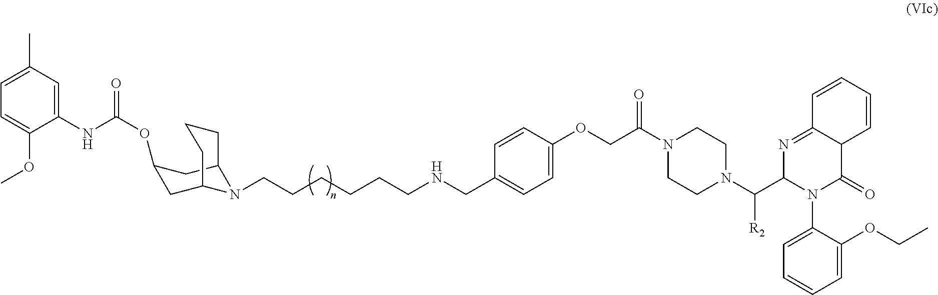

or a hydrate, solvate, or pharmaceutically acceptable salt thereof

wherein

n is selected from 1, 2, 3, 4, and 5; and

R₂ is H or —CH₃.

In some embodiments, the ferroptosis inducer is selected from:

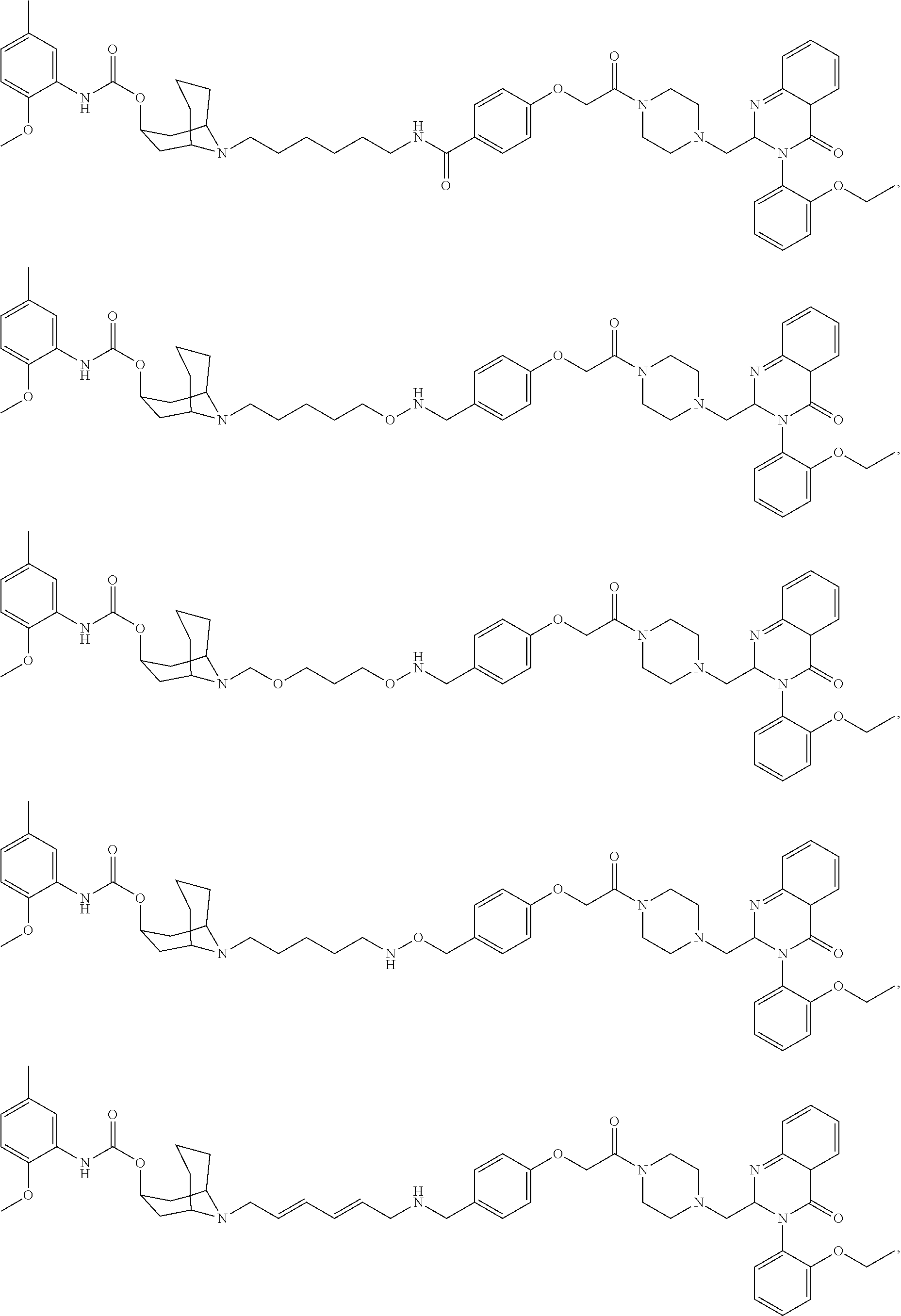

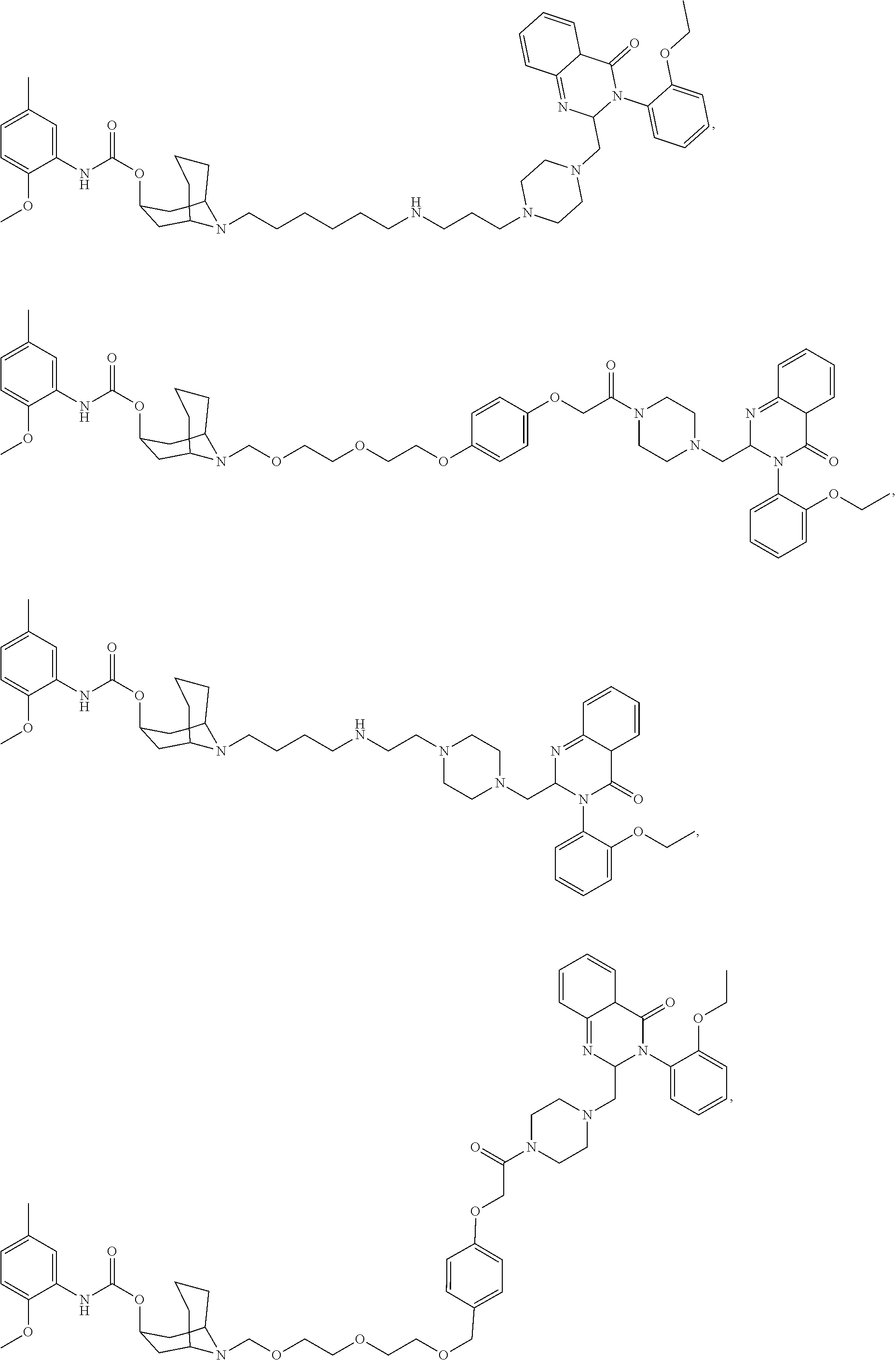 [SMILES: CCOc1ccccc1N1C(=O)C2C=CC=CC2=NC1CN1CCN(CCCNCCCCCCN2C3CCCC34CC(OC(=O)Nc3cc(C)ccc3OC)CC24)CC1 ; CCOc1ccccc1N1C(=O)C2C=CC=CC2=NC1CN1CCN(C(=O)COc2ccc(OCCOCCOCN3C4CCCC45CC(OC(=O)Nc4cc(C)ccc4OC)CC35)cc2)CC1 ; CCOc1ccccc1N1C(=O)C2C=CC=CC2=NC1CN1CCN(CCNCCCCN2C3CCCC34CC(OC(=O)Nc3cc(C)ccc3OC)CC24)CC1 ; CCOc1ccccc1N1C(=O)C2C=CC=CC2=NC1CN1CCN(C(=O)COc2ccc(COCCOCCOCN3C4CCCC45CC(OC(=O)Nc4cc(C)ccc4OC)CC35)cc2)CC1]

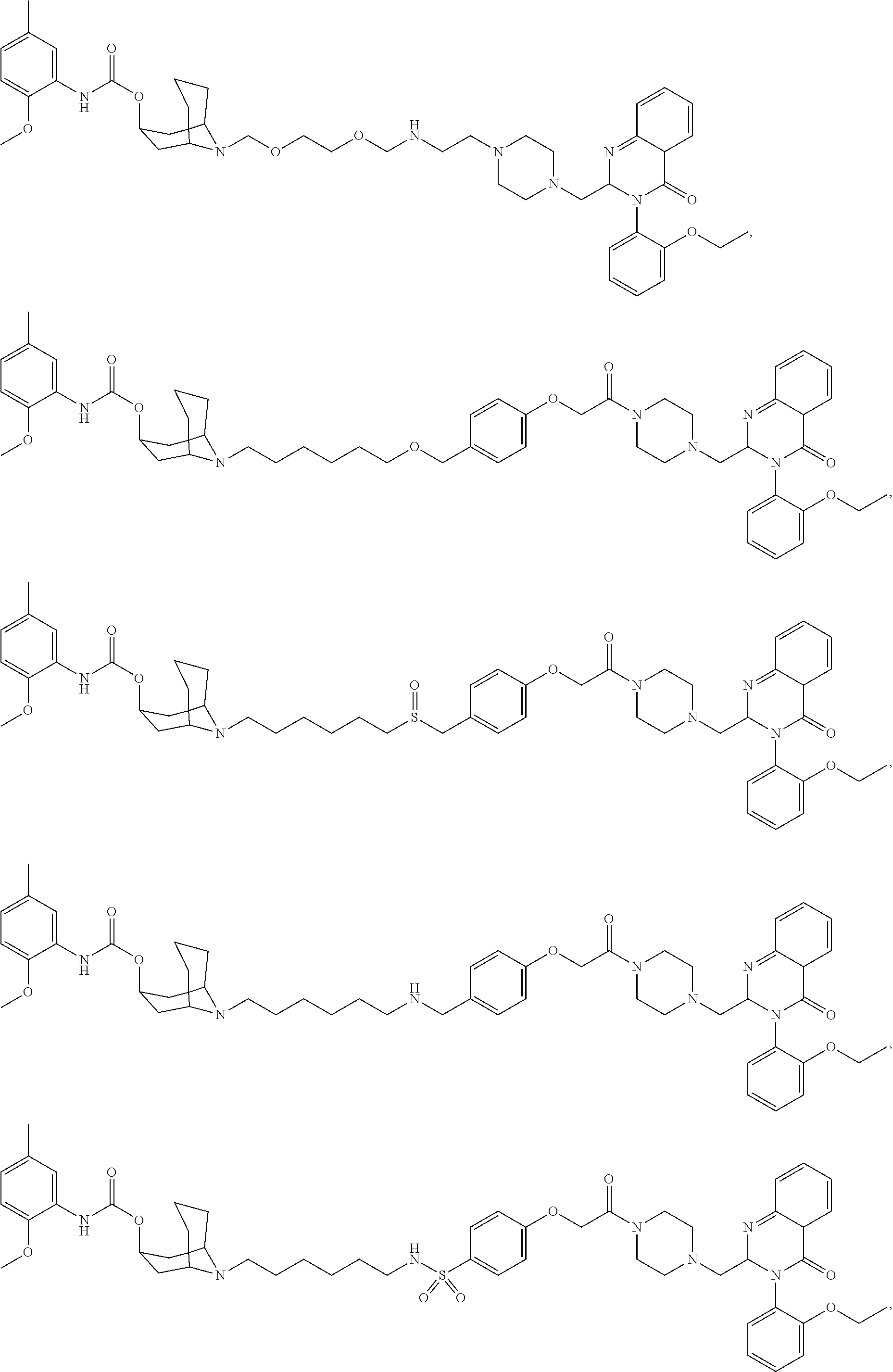

and a hydrate, solvate, and pharmaceutically acceptable salt thereof

In some embodiments, the compounds of formula (VI), formula (VIa), formula (VIb), and formula (VIc); methods of their synthesis; and dosages are described in patent publication WO2015153814 incorporated herein by reference.

In some embodiments, the ferroptosis inducer is a compound of formula (VII):

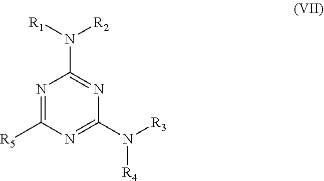

or a hydrate, solvate, or pharmaceutically acceptable salt thereof,

wherein

R₁ and R₂ are each independently H or C₁₋₄alkyl;

R₃ and R₄ are each independently H, C₁₋₄ alkyl, C₂₋₄ alkenyl, optionally substituted aryl or fused aryl; and

R₅ is C₁₋₄alkyl, haloC₁₋₄alkyl; C₁₋₄ alkylamino; or di-C₁₋₄ alkylamino.

In some embodiments, the ferroptosis inducer is selected from:

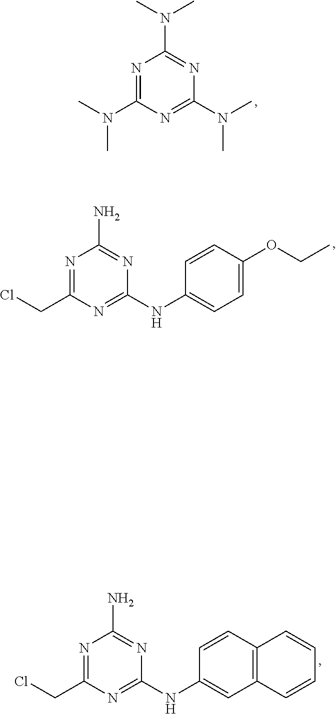

and a hydrate, solvate, and pharmaceutically acceptable salt thereof.

In some embodiments, the compounds of formula (VII) are described in, among others, Woo et al. Cell. 2015, 162(2):441-51, incorporated herein by reference.

In some embodiments, the ferroptosis inducer is a compound of formula (VIII):

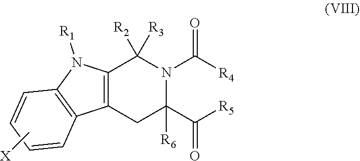

or a hydrate, solvate, or pharmaceutically acceptable salt thereof,

wherein

R₁, R₂, R₃, R₄, R₅, and R₆ are independently selected from the group consisting of H, C₁₋₈alkyloxy, 3- to 8-membered carbocyclic, 3- to 8-membered heterocyclyl, 3- to 8-membered aryl, or 3- to 8-membered heteroaryl, carboxylate, ester, amide, carbohydrate, amino acid, acyl, alkyloxy-substituted acyl, alditol, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether, wherein each alkyl, alkyloxy, carbocyclic, heterocyclyl, aryl, heteroaryl, carboxylate, ester, amide, carbohydrate, amino acid, acyl, alkyloxy-substituted acyl, alditol, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether is optionally substituted with at least one substituent;

R₇ is selected from the group consisting of H, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl may be optionally substituted with at least one substituent;

R₈ is selected from the group consisting of H, C₂₋₈alkenyl, C₂₋₈alkynyl, aryl, carbocycle, heteroaryl, heterocyclyl, alkylaryl, alkylheteroaryl, alkylheterocyclyl, and heteroaryl, wherein each alkyl, alkenyl, alkynyl, aryl, carbocycle, heteroaryl, heterocycle, alkylaryl, alkylheteroaryl, alkylheterocyclyl, and heteroaryl may be optionally substituted with at least one substituent; and

X is 0-4 substituents on the ring to which it is attached, wherein X is selected from the group consisting of H, cyano, oxo, nitro, acyl, acylamino, halo, hydroxy, amino acid, amine, amide, carbamate, ester, ether, carboxylic acid, thio, thioalkyl, thioester, thioether, C₁₋₈ alkyl, C₁₋₈ alkoxy, C₁₋₈alkenyl, arylC₁₋₈alkyl, 3- to 8-membered carbocyclic, 3- to 8-membered heterocyclyl, 3- to 8-membered aryl, 3- to 8-membered heteroaryl, sulfate, sulfonamide, sulfoxide, sulfonate, sulfone, alkylsulfonyl, and arylsulfonyl.

In some embodiments, the ferroptosis inducer is a compound of formula (VIIIa):

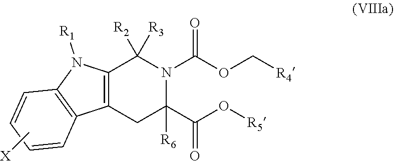

or a hydrate, solvate, or pharmaceutically acceptable salt thereof,

wherein

R₁, R₂, R₃, and R₆ are independently selected from the group consisting of H, C₁₋₈alkyl, C₁₋₈ alkyloxy, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkyloxy, arylalkyl, carbocyclic, heterocyclic, aryl, heteroaryl, acyl, alkylsulfonyl, and arylsulfonyl is optionally substituted with at least one substituent;

R₄′ and R₅′ are independently selected from the group consisting of H, C₁₋₈alkyl, 3- to 8-membered carbocyclic, 3- to 8-membered heterocyclyl, 3- to 8-membered aryl, or 3- to 8-membered heteroaryl, carboxylate, ester, amide, carbohydrate, amino acid, acyl, alkyloxy-substituted acyl, alditol, NR₇R₈, —C(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether, wherein each alkyl, carbocyclic, heterocyclyl, aryl, heteroaryl, carboxylate, ester, amide, carbohydrate, amino acid, acyl, alkyloxy-substituted acyl, alditol, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether is optionally substituted with at least one substituent;

R₇ is selected from the group consisting of H, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl may be optionally substituted with at least one substituent;

R₈ is selected from the group consisting of H, C₁₋₈alkynyl, aryl, carbocycle, heteroaryl, heterocyclyl, alkylaryl, alkylheteroaryl, alkylheterocyclyl, and heteroaryl, wherein each alkyl, alkenyl, alkynyl, aryl, carbocycle, heteroaryl, heterocycle, alkylaryl, alkylheteroaryl, alkylheterocyclyl, and heteroaryl may be optionally substituted with at least one substituent; and

X is 0-4 substituents on the ring to which it is attached, wherein X is selected from the group consisting of H, cyano, oxo, nitro, acyl, acylamino, halo, hydroxy, amino acid, amine, amide, carbamate, ester, ether, carboxylic acid, thio, thioalkyl, thioester, thioether, C₁₋₈alkyl, C₁₋₈alkoxy, C₁₋₈alkenyl, arylC₁₋₈alkyl, 3- to 8-membered carbocyclic, 3- to 8-membered heterocyclyl, 3- to 8-membered aryl, 3- to 8-membered heteroaryl, sulfate, sulfonamide, sulfoxide, sulfonate, sulfone, alkylsulfonyl, and arylsulfonyl.

In some embodiments, the ferroptosis inducer is a compound of formula (VIIIb):

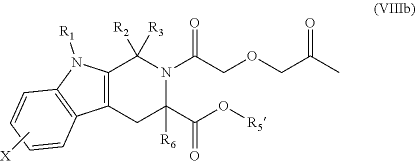

or a hydrate, solvate, or pharmaceutically acceptable salt thereof

wherein

X, R₁, R₂, R₃, R₅′ and R₆ are defined as for the compound of formula (VIIIa).

In some embodiments, the ferroptosis inducer is a compound of formula (VIIIc):

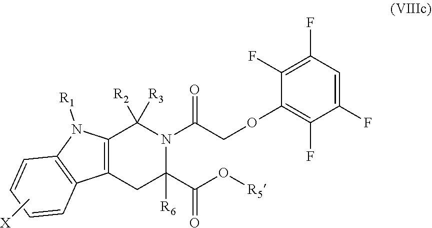

or a hydrate, solvate, or pharmaceutically acceptable salt thereof

wherein

X, R₁, R₂, R₃, R₅′ and R₆ are defined as for the compound of formula (VIIIa).

In some embodiments, the ferroptosis inducer is a compound of formula (VIIId)

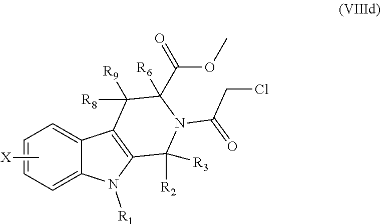

or a hydrate, solvate, or pharmaceutically acceptable salt thereof

wherein

R₂ and R₃ are independently selected from H, methyl, methyl benzoate, propargyl, and phenyl, wherein at least one of R₂ and R₃ is other than H;

R₁, R₆, R₈, and R₉ are independently selected from H, halo, C₁₋₈alkyl, C₁₋₈alkoxy, 3- to 8-membered carbocyclic, 3- to 8-membered heterocyclic, 3- to 8-membered aryl, or 3- to 8-membered heteroaryl, carboxylate, ester, ether, amide, amino acid, acyl, alkoxy-substituted acyl, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, ether, amide, amino acid, acyl, alkoxy-substituted acyl, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether is optionally substituted with at least one substituent;

R₇ is selected from the group consisting of H, C₁₋₈alkyl, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl is optionally substituted with at least one substituent; and

X is 0-4 substituents on the ring to which it is attached, wherein X is selected from the group consisting of H, cyano, oxo, nitro, acyl, acylamino, halo, hydroxy, amino acid, amine, amide, carbamate, ester, ether, carboxylic acid, thio, thioalkyl, thioester, thioether, C₁₋₈alkyl, C₁₋₈alkoxy, C₁₋₈alkenyl, arylC₁₋₈alkyl, 3- to 8-membered carbocyclic, 3- to 8-membered heterocyclyl, 3- to 8-membered aryl, 3- to 8-membered heteroaryl, sulfate, sulfonamide, sulfoxide, sulfonate, sulfone, alkylsulfonyl, and arylsulfonyl.

In some embodiments, the ferroptosis inducer is a compound of formula (IX):

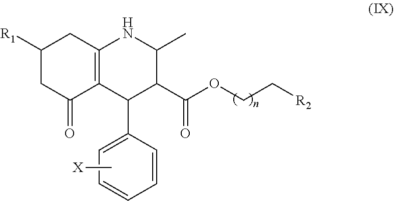

or a hydrate, solvate, or pharmaceutically acceptable salt thereof,

wherein

R₁ and R₂ are independently selected from the group consisting of H, C₁₋₈alkyl, C₁₋₈alkyloxy, 3- to 8-membered carbocyclic, 3- to 8-membered heterocyclyl, 3- to 8-membered aryl, 3- to 8-membered heteroaryl, carboxylate, ester, amide, carbohydrate, amino acid, acyl, alkyloxy-substituted acyl, alditol, NR₃R₄, OC(R₃)₂COOH, SC(R₃)₂COOH, NHCHR₃COOH, COR₄, CO₂R₄, sulfate, sulfonamide, sulfoxide, sulfonate, sulfone, thioalkyl, thioester, and thioether, wherein each alkyl, alkyloxy, carbocyclic, heterocyclic, aryl, heteroaryl, carboxylate, ester, amide, carbohydrate, amino acid, acyl, alkyloxy-substituted acyl, alditol, NR₃R₄, OC(R₃)₂COOH, SC(R₃)₂COOH, NHCHR₃COOH, COR₄, CO₂R₄, sulfate, sulfonamide, sulfoxide, sulfonate, sulfone, thioalkyl, thioester, and thioether is optionally substituted with at least one substituent;

R₃ is selected from the group consisting of H, C₁₋₈alkyl, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl may be optionally substituted with at least one substituent;

R₄ is selected from the group consisting of H, C₁₋₈alkyl, C₁₋₈alkenyl, C₁₋₈alkynyl, aryl, carbocycle, heteroaryl, heterocyclyl, alkylaryl, alkylheteroaryl, alkylheterocyclyl, and heteroaryl, wherein each alkyl, alkenyl, alkynyl, aryl, carbocycle, heteroaryl, heterocyclyl, alkylaryl, alkylheteroaryl, alkylheterocyclyl, and heteroaryl may be optionally substituted with at least one substituent;

X is selected from the group consisting of halo and C₁₋₈alkyl; and

n is 0-8.

In some embodiments, the ferroptosis inducer is a compound of formula (IXa):

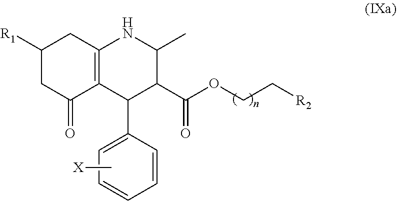

or a hydrate, solvate, or pharmaceutically acceptable salt thereof,

wherein

X, R₁ and R₂ are as defined for formula (IX).

In some embodiments, the ferroptosis inducer is a compound of formula (X):

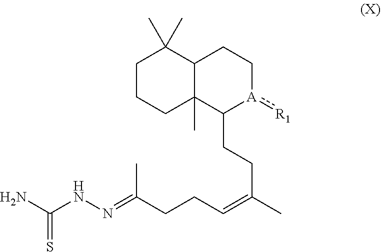

or a hydrate, solvate, or pharmaceutically acceptable salt thereof,

wherein

R₁ is selected from the group consisting of H, C₁₋₈alkyloxy, 3- to 8-membered carbocyclic, 3- to 8-membered heterocyclyl, 3- to 8-membered aryl, or 3- to 8-membered heteroaryl, carboxylate, ester, amide, carbohydrate, amino acid, acyl, alkyloxy-substituted acyl, alditol, NR₂R₃, OC(R₂)₂COOH, SC(R₂)₂COOH, NHCHR₂COOH, COR₃, CO₂R₃, sulfate, sulfonamide, sulfoxide, sulfonate, sulfone, thioalkyl, thioester, and thioether, wherein each alkyl, alkyloxy, carbocyclic, heterocyclyl, aryl, heteroaryl, carboxylate, ester, amide, carbohydrate, amino acid, acyl, alkyloxy-substituted acyl, alditol, NR₂R₃, OC(R₂)₂COOH, SC(R₂)₂COOH, NHCHR₂COOH, COR₃, CO₂R₃, sulfate, sulfonamide, sulfoxide, sulfonate, sulfone, thioalkyl, thioester, and thioether is optionally substituted with at least one substituent;

R₂ is selected from the group consisting of H, C₁₋₈alkyl, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl may be optionally substituted with at least one substituent;

R₃ is selected from the group consisting of H, C₁₋₈alkenyl, C₁₋₈alkynyl, aryl, carbocycle, heteroaryl, heterocyclyl, alkylaryl, alkylheteroaryl, alkylheterocyclyl, and heteroaryl, wherein each alkyl, alkenyl, alkynyl, aryl, carbocycle, heteroaryl, heterocyclyl, alkylaryl, alkylheteroaryl, alkylheterocyclyl, and heteroaryl may be optionally substituted with at least one substituent; and

A is C, N, or S.

In some embodiments, the ferroptosis inducer is a compound of formula (XI):

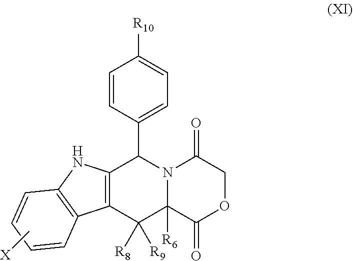

or a hydrate, solvate, or pharmaceutically acceptable salt thereof,

wherein

R₆, R₈, and R₉ are independently selected from the group consisting of H, halo, C₁₋₈alkyl, C₁₋₈alkyloxy, 3- to 8-membered carbocyclic, 3- to 8-membered heterocyclyl, 3- to 8-membered aryl, or 3- to 8-membered heteroaryl, carboxylate, ester, ether, amide, carbohydrate, amino acid, acyl, alkyloxy-substituted acyl, alditol, NR₇R₁₁, OC(R₇)₂COOH, SC(R₇)₂COOH, NHCHR₇COOH, CO₂R₁₁, sulfate, sulfonamide, sulfoxide, sulfonate, sulfone, thioalkyl, thioester, and thioether, wherein each alkyl, alkyloxy, carbocyclic, heterocyclic, aryl, heteroaryl, carboxylate, ester, ether, amide, carbohydrate, amino acid, acyl, alkyloxy-substituted acyl, alditol, NR₇R₁₁, OC(R₇)COOH, SC(R₇)₂COOH, NHCHR₇COOH, COR₁₁, CO₂R₁₁, sulfate, sulfonamide, sulfoxide, sulfonate, sulfone, thioalkyl, thioester, and thioether is optionally substituted with at least one substituent;

R₇ is selected from the group consisting of H, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl may be optionally substituted with at least one substituent;

R₁₁ is selected from the group consisting of H, C₁₋₈alkyl, carbocycle, aryl, heteroaryl, heterocyclyl, alkylaryl, alkylheteroaryl, alkylheterocyclyl, and heteroaryl, wherein each alkyl, alkenyl, alkynyl, carbocycle, aryl, heteroaryl, heterocycle, alkylaryl, alkylheteroaryl, alkylheterocyclyl, and heteroaryl may be optionally substituted with at least one substituent;

X is 0-4 substituents on the ring to which it is attached, wherein X is selected from the group consisting of H, cyano, oxo, nitro, acyl, acylamino, halo, hydroxy, amino acid, amine, amide, carbamate, ester, ether, carboxylic acid, thio, thioalkyl, thioester, thioether, C₁₋₈alkyl, C₁₋₈alkoxy, C₁₋₈alkenyl, arylC₁₋₈alkyl, 3- to 8-membered carbocyclic, 3- to 8-membered heterocyclyl, 3- to 8-membered aryl, 3- to 8-membered heteroaryl, sulfate, sulfonamide, sulfoxide, sulfonate, sulfone, alkylsulfonyl, and arylsulfonyl; and

R₁₀ is selected from the group consisting of a C₁₋₈ ester and an acid, which ester or acid is hydrolyzable in vivo.

In some embodiments, the ferroptosis inducer is selected from:

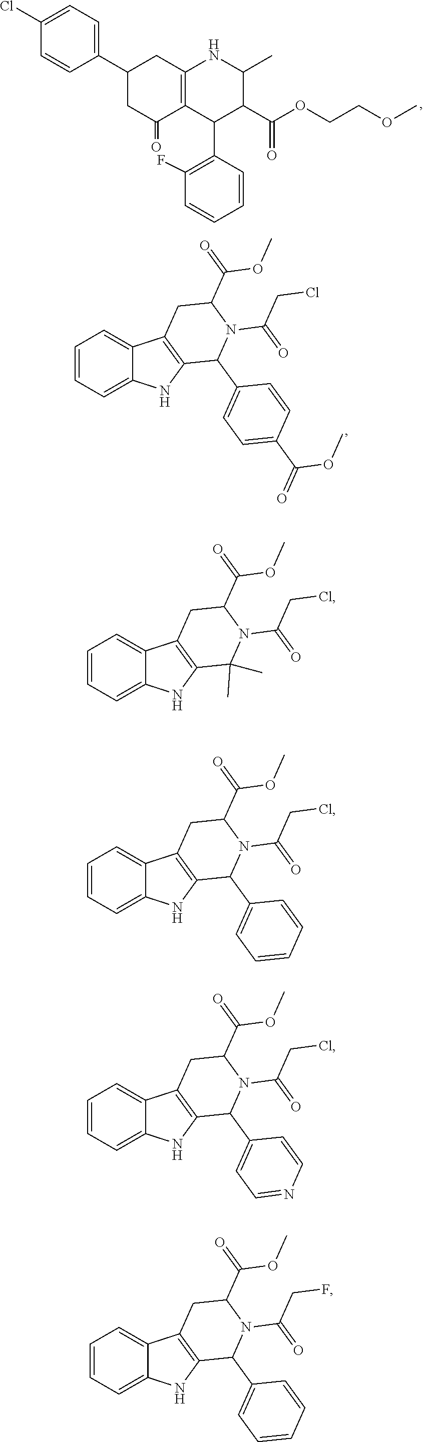

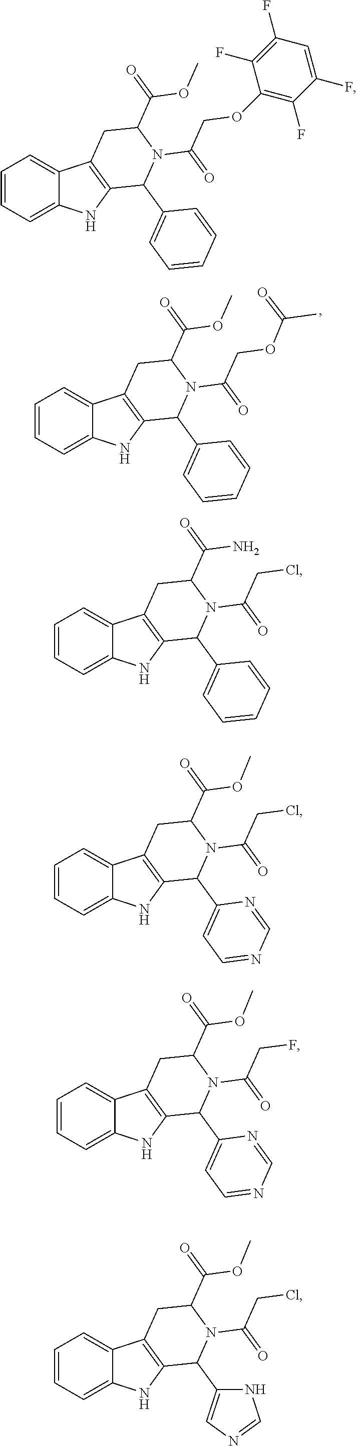

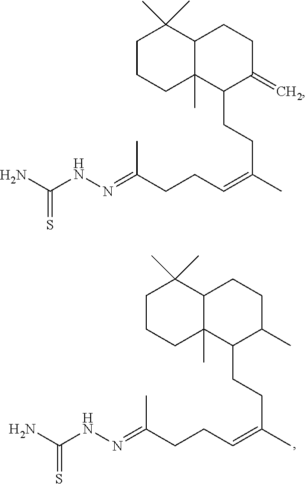 [SMILES: C=C1CCC2C(C)(C)CCCC2(C)C1CC/C(C)=C\CC/C(C)=N/NC(N)=S ; C/C(=C/CC/C(C)=N/NC(N)=S)CCC1C(C)CCC2C(C)(C)CCCC12C]

and a hydrate, solvate, and pharmaceutically acceptable salt thereof.

In some embodiments, the compounds of formula (VIII), formula (VIIIa), formula (VIIIb), formula (VIIIc), formula (VIIId), formula (IX), formula (IXa), formula (X) and formula (XI); methods of their synthesis; and dosages are described in patent publication US20100081654, incorporated herein by reference.

In some embodiments, the ferroptosis inducer is a compound of formula (XII):

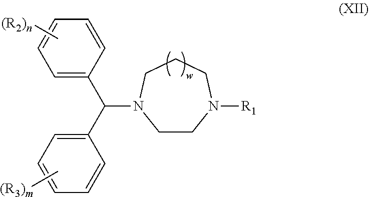

or a hydrate, solvate, or pharmaceutically acceptable salt thereof,

wherein,

R₁ is selected from the group consisting of C₁₋₄ alkyl, C₂₋₄alkenyl, optionally substituted heterocyclyl, optionally substituted aryl, optionally substituted heteroaryl, optionally substituted aryl C₁₋₄alkyl, optionally substituted arylC₂₋₄ alkenyl, optionally substituted heteroarylC₁₋₄ alkyl, optionally substituted heteroarylC₂₋₄ alkenyl, optionally substituted heterocyclylcarbonyl, optionally substituted arylcarbonyl, optionally substituted heteroarylcarbonyl, carboxyC₁₋₆ heteroalkyl, and hydroxyC₁₋₆heteroalkyl, wherein the substitution when present is selected from the group consisting of halo, amino, C₁₋₄ alkylamino, di-C₁₋₄ alkylamino, C₂₋₄ alkenylamino, di-C₂₋₄alkenylamino, nitro, sulfonyl, aminosulfonyl, C₁₋₄ alkyl, heteroaryl, and heterocyclyl;

R₂ and R₃ are independently Cl or F;

n and m are independently 0, 1 or 2; and

w is 0 or 1.

In some embodiments of the compounds of formula (XII), R₁ is

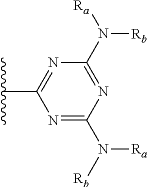

wherein,

Rₐ and R_{b} are each independently selected from the group consisting of H, C₁₋₄ alkyl, C₂₋₄alkenyl, optionally substituted aryl, and fused aryl.

In some embodiments of the compounds of formula (XII), R₁ is

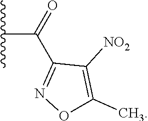 [SMILES: *C(=O)c1noc(C)c1[N+](=O)[O-]]

In some embodiments, the ferroptosis inducer is selected from:

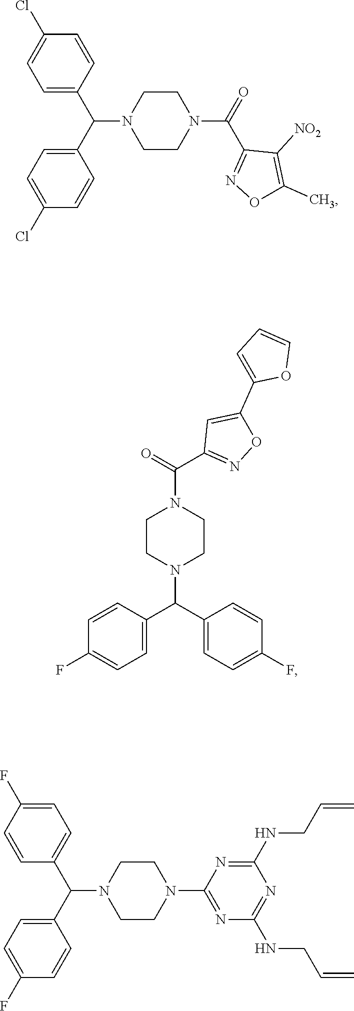 [SMILES: Cc1onc(C(=O)N2CCN(C(c3ccc(Cl)cc3)c3ccc(Cl)cc3)CC2)c1[N+](=O)[O-] ; O=C(c1cc(-c2ccco2)on1)N1CCN(C(c2ccc(F)cc2)c2ccc(F)cc2)CC1 ; C=CCNc1nc(NCC=C)nc(N2CCN(C(c3ccc(F)cc3)c3ccc(F)cc3)CC2)n1]

and a hydrate, solvate, and pharmaceutically acceptable salt thereof.

Exemplary compounds of formula (XII) are described in, among others, Weiwer et al., 2012, Bioorg Med Chem Lett. 22(4):1822-1826; and Bittker et al., “Screen for RAS-Selective Lethal Compounds and VDAC Ligands—Probe 2” Probe Reports from the NIH Molecular Libraries Program [Internet]. Bethesda (Md.): National Center for Biotechnology Information (US); 2010-, 2011 Feb. 10 [Updated 2011 Dec. 12], PMID: 22834036; all publications incorporated herein by reference.

In some embodiments, the ferroptosis inducer is a compound of formula (XIII):

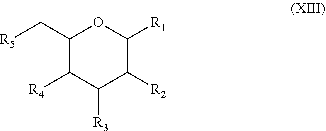

or a hydrate, solvate, or pharmaceutically acceptable salt thereof

wherein,

R₁, R₂, R₃, R₄ and R₅ are each independently selected from the group consisting of H, OH, C₁₋₄alkyloxy, C₁₋₄alkylcarbonyloxy, —NRₐR_{b}, —S—Au═P(R₆)₃, and —S—Au—R₇; wherein

each R₆ is independently an optionally substituted C₁₋₄ alkyl;

R₇ is

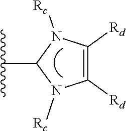

wherein

each R_{c} is independently selected from the group consisting of H, C₂₋₄alkenyl, optionally substituted phenylC₁₋₄ alkyl, and optionally substituted phenyl, wherein the optional substitution on the phenyl is —C₁₋₄ alkyl on one or more of the carbon atoms of the phenyl;

each R_{d} is H, C₁₋₄alkyl, or the two R_{d} together form an optionally substituted phenyl;

Rₐ and R_{b} are each independently selected from the group consisting of H and C₁₋₄alkylcarbonyl; and

at least one of R₁, R₂, R₃, R₄ and R₅ is —S—Au═P(R₆)₃ or —S—Au—R₇.

In some embodiments, the compound of formula (XIII) is selected from:

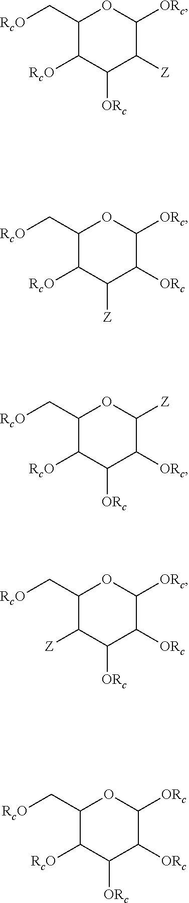

and a hydrate, solvate, and pharmaceutically acceptable salt thereof

wherein,

each R_{c} is independently selected from the group consisting of H, C₁₋₄alkyl, or C₁₋₄alkylcarbonyl and

Z is —S—Au═P(R₆)₃ or —S—Au—R₇.

In some embodiments, the compound of formula (XIII) is selected from

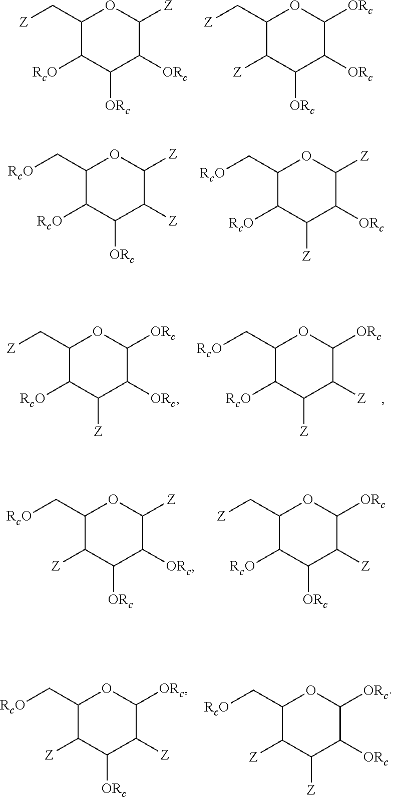

and a hydrate, solvate, and pharmaceutically acceptable salt thereof

wherein,

each R_{c} is independently selected from the group consisting of H, C₁₋₄alkyl, or C₁₋₄alkylcarbonyl; and

Z is —S—Au═P(R₆)₃ or —S—Au—R₇.

In some embodiments, the ferroptosis inducer is a compound is selected from:

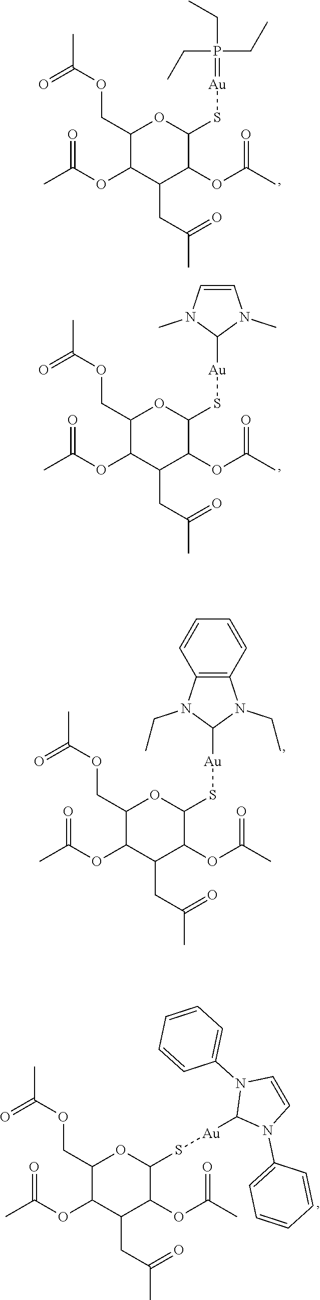

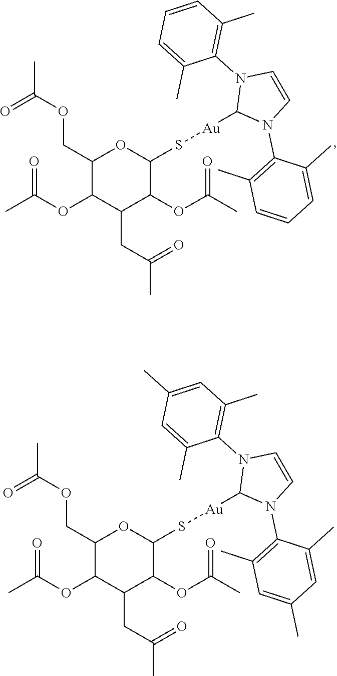

and a hydrate, solvate, and pharmaceutically acceptable salt thereof.

Various compounds and derivatives of formula (XIII); methods of their synthesis; and dosages are described in patent publication WO2012142615, incorporated herein by reference.

In some embodiments, the ferroptosis inducer is a compound of formula (XIV):

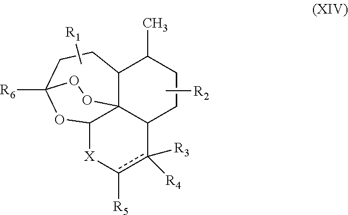

or a hydrate, solvate, or pharmaceutically acceptable salt thereof

wherein

[symbol] is a single or double bond;

R₁, R₂, R₃, R₄, and R₆ are each independently selected from the group consisting of H, halo, —CF₃, ═CH₂, —OR₇, —NR₇R₈, —(CH₂)ₙCOOR₇, —(CH₂)ₙC(O)R₇, —(CH₂)ₙCONR₇R₇, optionally substituted C₁₋₆ alkyl, optionally substituted C₂₋₆ alkenyl, optionally substituted C₂₋₆ alkynyl, optionally substituted C₃₋₇ cycloalkyl, optionally substituted C₃₋₇ heterocycloalkyl, optionally substituted aryl and optionally substituted heteroaryl;

R₅ is selected from the group consisting of H, halo, ═O, —OR₇, —NR₇R₈, —(CH₂)ₙCF₃, —(CH₂)ₙCHF₂, —(CH₂). C(O)R₇, —O(CH₂)ₙCOOR₇, —OC(O)(CH₂)ₙCOOR₇, —OC(O)R₇, optionally substituted C₁₋₆alkyl, optionally substituted C₂₋₆ alkenyl, optionally substituted C₂₋₆ alkynyl, optionally substituted C₃₋₇ cycloalkyl, optionally substituted C₃₋₇ heterocycloalkyl, optionally substituted aryl and optionally substituted heteroaryl;

X is O or —NR₇;

wherein

R₇ is selected from the group consisting of H, optionally substituted C₁₋₆alkyl, optionally substituted, optionally substituted C₂₋₆ alkenyl, and optionally substituted C₂₋₆ alkynyl; and

R₈ is selected from the group consisting of H or an optionally substituted C₁₋₆ alkyl, optionally substituted C₂₋₆ alkenyl, optionally substituted C₂₋₆ alkynyl, optionally substituted cycloalkyl, optionally substituted aryl, optionally substituted heteroaryl, and optionally substituted arylalkyl; or

R₇ and R₈ together with the nitrogen atom to which R₇ and R₈ are attached form an optionally substituted heterocyclic ring, wherein the heterocyclic atom is N, O or S; and

n is 0, 1, 2 or 3.

In some embodiments, the compounds of formula (XIV) are selected from artelinic acid, artemether, artemotil (arteether, 13-arteether), artenimol (dihydroartemisinin, 13-dihydroartemisinin) and artesunate, or a pharmaceutically acceptable salt thereof, or derivatives or analogs thereof. In some embodiments, the compound of formula (XIV) is selected from artemisone, dihydroartemisinin hemisuccinate, dihydrodroartemisinin succinate, dihydro-artemisinin glucuronide, sodium artesunate, 20 dihydroartemisitene dimers, 11-aza-artemisinin derivatives, amino-functionalized 1,2,4-trioxanes, artemisinin endoperoxides, deoxy-artemisinins, spiro and dispiro 1,2,4-trioxolane, mixed steroidal 1,2,4,5-tetraoxane compounds, substituted 1,2,4-trioxanes, trioxane derivatives based on artemisinin, seco-acrtemisinins, trioxane dimer compounds, conjugates of artelinic acid arteethers from dihydroartemisinin, artemisinine or artemisinene derivatives, C-10 carbon substituted artemisinin-like trioxane compounds, water-soluble trioxanes alpha arteether, artemisinin dimers, (+)-deoxoarteminisinin and analogs of (+)-deoxoartemisinin, and 10-substituted derivatives of dihydroartemisinin, as well as its salts or other derivatives thereof. Compounds of formula (XIV) and analogs and derivatives thereof are described in, among others, patent publications U.S. Pat. Nos. 6,984,640, 6,586,464, 7,910,750, US20050119232, EP0974354, and WO2015155303; all publications incorporated herein by reference.

In some embodiments, the compound of formula (XIV) is selected from:

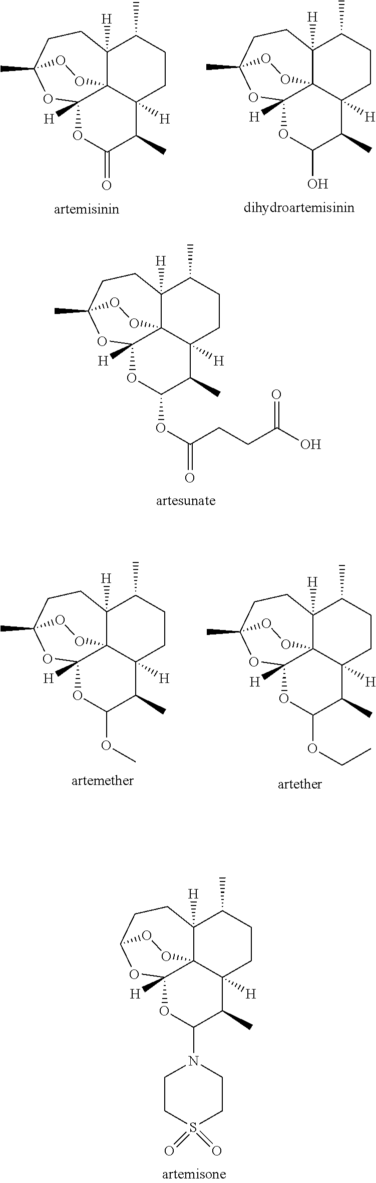

and a hydrate, solvate, and pharmaceutically acceptable salt thereof.

In some embodiments, the compounds of formula (XIV), such as artensunate, are used in the treatment of pancreatic cancer.

In some embodiments, the ferroptosis inducer is a compound of formula (XV):

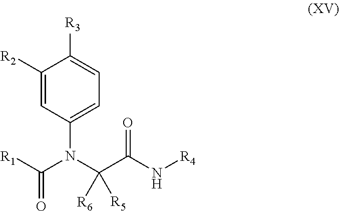

or a hydrate, solvate, or pharmaceutically acceptable salt thereof,

wherein

R₁ is an optionally substituted C₁₋₄ alkyl, optionally substituted C₁₋₄ alkyl-O—, optionally substituted C₁₋₄ alkyl-O—C₁₋₄ alkyl, or optionally substituted heteroaryl, wherein the substitution when present is selected from OH, halo, and thio;

R₂ is H, halo, or C₁₋₄ alkyl-O—;

R₃ is H or C₁₋₄ alkyl-O—;

R₄ is optionally substituted cycloalkylC₁₋₄ alkyl, optionally substituted arylC₁₋₄ alkyl, optionally substituted heteroarylC₁₋₄ alkyl, or optionally substituted aryl-S(O)₂C₁₋₄ alkyl, wherein the substitution when present is OH, halo or C₁₋₄ alkyl;

R₅ is H or C₁₋₄ alkyl; and

R₆ is H or optionally substituted cycloalkyl, heterocycloalkyl, C₁₋₄ alkyl, wherein the substitution when present is selected from halo or C₁₋₄ alkyl.

In some embodiments of the compound of formula (XV), or a hydrate, solvate, or pharmaceutically acceptable salt thereof,

R₁ is HOCH₂—, ClCH₂—, C₁₋₄ alkyl, CH₃O—, CH₃—O—CH₂—, or thiopyrimidine;

R₂ is H, Cl, or CH₃O—;

R₃ is H or CH₃O—;

R₄ is C₃₋₆ cycloalkylC₁₋₄ alkyl, phenylC₁₋₄ alkyl, heteroarylC₁₋₄ alkyl, or 4-methylphenyl-S(O)₂C₁₋₄ alkyl;

R₅ is H or CH₃—; and

R₆ is H, CH₃—, or

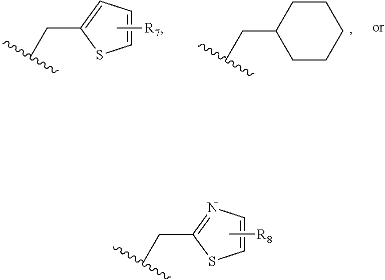

wherein

R₇ is H or halo; and

R₈ is H or C₁₋₄ alkyl.

In some embodiments, the ferroptosis inducer is selected from:

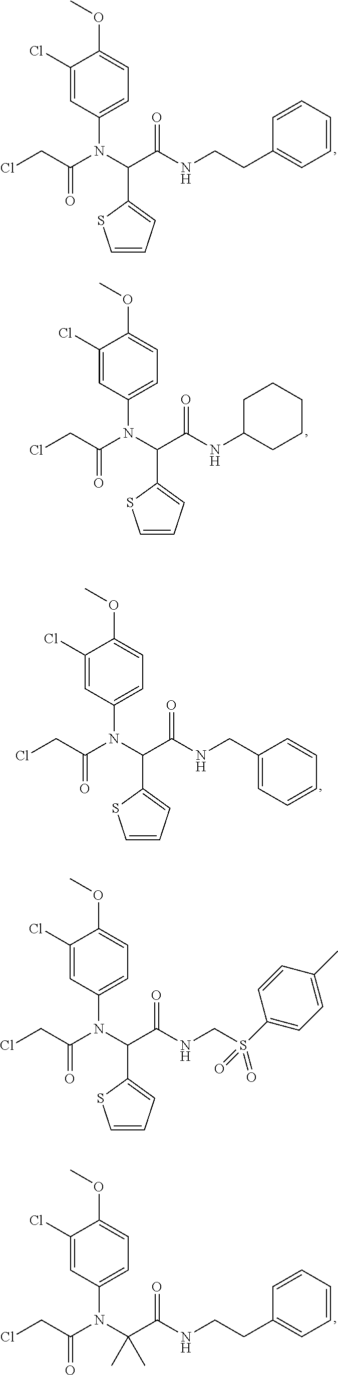

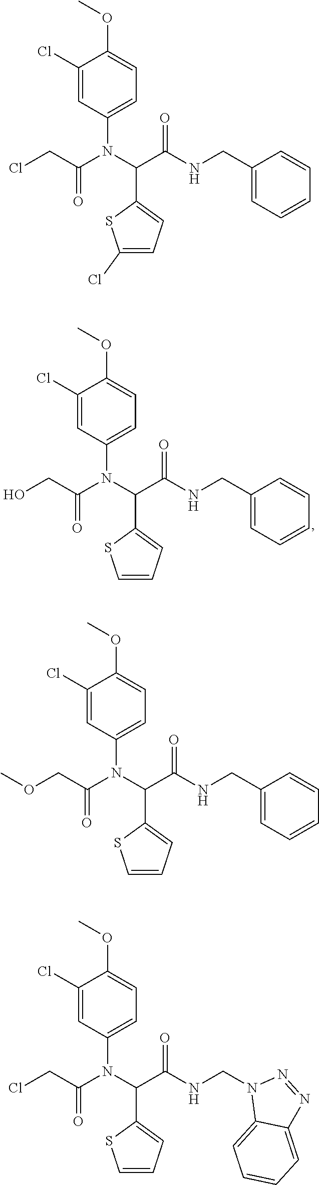 [SMILES: COc1ccc(N(C(=O)CCl)C(C(=O)NCc2ccccc2)c2ccc(Cl)s2)cc1Cl ; COc1ccc(N(C(=O)CO)C(C(=O)NCc2ccccc2)c2cccs2)cc1Cl ; COCC(=O)N(c1ccc(OC)c(Cl)c1)C(C(=O)NCc1ccccc1)c1cccs1 ; COc1ccc(N(C(=O)CCl)C(C(=O)NCn2nnc3ccccc32)c2cccs2)cc1Cl]

and a hydrate, solvate, and pharmaceutically acceptable salt thereof.

Ferroptosis inducer compounds of formula (XV) are described in, among others, Weiwer et al, 2012, Bioorg Med Chem Lett. 22(4):1822-1826; and Bittker et al., “Screen for RAS-Selective Lethal Compounds and VDAC Ligands—Probe 1,” Probe Reports from the NIH Molecular Libraries Program [Internet], Bethesda (Md.), National Center for Biotechnology Information (US); 2010-. 2010 Mar. 26 [updated 2011 Feb. 10], PMID: 21634081; all publications incorporated herein by reference.

In some embodiments, the ferroptosis inducer, such as those described herein by chemical structure, include RSL3, erastin and erastin derivatives (MEII, PE, AE), ML210 (DPI10), ML162 (DPI7), auranofin, sulfasalazine, artesunate, artemisinin, dihydroatemisinin, artemether, and other artemisinin derivatives, sorafenib and sorafenib analogs (SRS13-45 and SRS13-60), buthionine sulfoximine (BSO), altretamine, almitrine, lanperisone, DPI2, DPI12, DPI13, DPI17, DPI18, DPI19, 31MEW44, and ML160.

In some embodiments, the ferroptosis inducer can be an inhibitory nucleic acid targeting expression of a cellular molecule involved in regulating ferroptosis. Nucleic acids, such as shRNA and anti-sense nucleic acids can target cellular components, such as cysteine-glutamate antiporter (system X⁻_{c}), glutathione peroxidase 4 (GPX4), NOX, and ALOX15. shRNAs and inhibitory RNAs for such cellular targets are described in patent publications US20150079035, US20150175558, US20100081654, WO2015051149, WO2015084749, WO2013152039 and WO2015109009; all publications incorporated herein by reference. Other cellular targets and corresponding inhibitory nucleic acids are described in Gao et al., 2016, Cell Res. 26:1021-1032, incorporated herein by reference.

### 5.3. Combination Treatments

In another aspect, the ferroptosis inducer is used in combination with one or more of other therapeutic treatments for cancer.

In some embodiments, a subject with cancer is treated with a combination of a ferroptosis inducer and radiation therapy. In some embodiments, the method comprises administering to a subject with cancer a therapeutically effective amount of a ferroptosis inducer, and adjunctively treating the subject with an effective amount of radiation therapy. In some embodiments, the ferroptosis inducer is administered to the subject in need thereof prior to, concurrently with, or subsequent to the treatment with radiation.

In some embodiments, the method comprises administering an effective amount of a ferroptosis inducer to a subject with cancer to sensitize the cancer to radiation treatment, and administering a therapeutically effective amount of radiation therapy to treat the cancer. In some embodiments, an effective amount of X-ray and gamma ray is administered to the subject. In some embodiments, an effective amount of particle radiation is administered to the subject, where the particle radiation is selected from electron beam, proton beam, and neutron beam radiation. In some embodiments, the radiation therapy is fractionated.

In some embodiments, a subject with cancer is treated with a therapeutically effective amount of a ferroptosis inducer and a therapeutically effective amount of a second chemotherapeutic agent. In some embodiments, the second chemotherapeutic agent is selected from an alkylating agent, antibiotic agent, antimetabolic agent (e.g., folate antagonists, purine analogs, pyrimidine analogs, etc.), topoisomerase inhibiting agent, antimicrotubule agent (e.g., taxanes, vinca alkaloids), hormonal agent (e.g., aromatase inhibitors), plant-derived agent and synthetic derivatives thereof, anti-angiogenic agent, differentiation inducing agent, cell growth arrest inducing agent, apoptosis inducing agent, cytotoxic agent, agent affecting cell bioenergetics, i.e., affecting cellular ATP levels and molecules/activities regulating these levels, anti-cancer biologic agent (e.g., monoclonal antibodies), kinase inhibitors and inhibitors of growth factors and their receptors.

In some embodiments, the second chemotherapeutic agent is selected from afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, idelalisib, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, olaparib, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, vindesine, and the like. In some embodiments, the ferroptosis inducer is administered prior to, concurrently with, or subsequent to the treatment with the chemotherapeutic agent.

In some embodiments, the method of treating a cancer comprises administering a therapeutically effective amount of a ferroptosis inducer and a therapeutically effective amount a biologic agent used to treat cancer. In some embodiments, the biologic agent is selected from anti-BAFF (e.g., belimumab); anti-CCR₄ (e.g., mogamulizumab); anti-CD19/CD3 (e.g., blinatumomab); anti-CD20 (e.g., obinutuzumab, rituximab, ibritumomab tiuxetan, ofatumumab, tositumomab); anti-CD22 (e.g., moxetumomab pasudotox); anti-CD30 (e.g., brentuximab vedotin); anti-CD33 (e.g., gemtuzumab); anti-CD37 (e.g., otlertuzumab); anti-CD38 (e.g., daratumumab); anti-CD52 (e.g., alemtuzumab); anti-CD56 (e.g., lorvotuzumab mertansine); anti-CD74 (e.g., milatuzumab); anti-CD105; anti-CD248 (TEM1) (e.g., ontuxizumab); anti-CTLA4 (e.g., tremelimumab, ipilimumab); anti-EGFL7 (e.g., parsatuzumab); anti-EGFR (HER₁/ERBB1) (e.g., panitumumab, nimotuzumab, necitumumab, cetuximab, imgatuzumab, futuximab); anti-FZD7 (e.g., vantictumab); anti-HER₂ (ERBB2/neu) (e.g., margetuximab, pertuzumab, ado-trastuzumab emtansine, trastuzumab); anti-HER₃ (ERBB3); anti-HGF (e.g., rilotumumab, ficlatuzumab); anti-IGF-1R (e.g., ganitumab, figitumumab, cixutumumab, dalotuzumab); anti-IGF-2R; anti-KIR (e.g., lirilumab, onartuzumab); anti-MMP9; anti-PD-1 (e.g., nivolumab, pidilizumab, lambrolizumab); anti-PD-L1 (e.g. Atezolizumab); anti-PDGFRa (e.g., ramucirumab, tovetumab); anti-PD-L2; anti-PIGF (e.g., ziv-aflibercept); anti-RANKL (e.g., denosumab); anti-TNFRSF 9 (CD 137/4-1 BB) (e.g., urelumab); anti-TRAIL-RI/DR4, R2/D5 (e.g., dulanermin); anti-TRAIL-R1/D4 (e.g., mapatumumab); anti-TRAIL-R2/D5 (e.g., conatumumab, lexatumumab, apomab); anti-VEGFA (e.g., bevacizumab, ziv-aflibercept); anti-VEGFB (e.g., ziv-aflibercept); and anti-VEGFR₂ (e.g., ramucirumab).

In some embodiments, the ferroptosis inducer is used in combination with a PKC modulating compound, particularly a diterpenoid PKC activator compound. In some embodiments, the method of treating cancer comprises administering to a subject in need thereof a therapeutically effective amount of a ferroptosis inducer and a therapeutically effective amount of a diterpenoid PKC modulator, particularly a PKC activator. Classes of diterpenoid compounds capable of modulating PKC activity include tigliane (e.g., phorbol, deoxyphorbol, etc.), ingenane (e.g., ingenol), daphnane and lathyrane diterpenoids. In some embodiments, the PKC activator for use in the methods herein include PKC activating phorbol, deoxyphorbol, ingenol, daphnane and lathyrane compounds, including enantiomers, derivatives, analogs, and prodrugs thereof, and salts, hydrates, and solvates thereof.

In some embodiments, the ferroptosis inducer is used in combination with a tigliane or phorbol class of PKC activating compounds. These compounds comprise a partial structure of formula A:

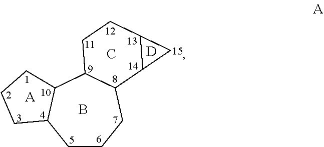

In some embodiments, the bond between carbon atoms 5 and 6, carbon atoms 6 and 7, and carbon atoms 1 and 2, are each independently a double bond, as illustrated in formula A1 and A2, below. In some embodiments, carbon atoms 5 and 6 or carbon atoms 6 and 7 are bonded to a common oxygen atom to form an epoxide, as illustrated in formula A3 and A4.

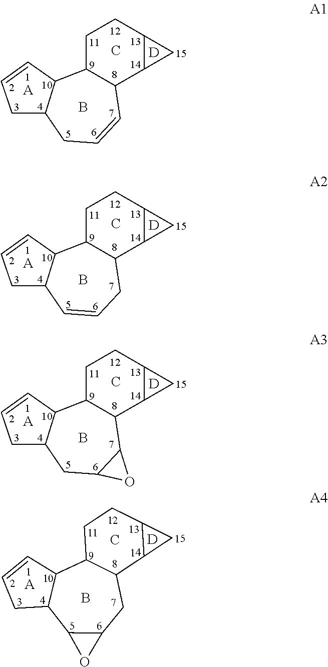

In various embodiments, substituents can be present on one or more of carbon atoms 2, 3, 4, 5, 6, 7, 9, 11, 12, 13, 14, and 15 of formula A, particularly of formula A1, A2, A3 or A4. PKC activating phorbol compounds and derivatives, analogs, and prodrugs thereof, and methods of their synthesis are described in, among others, U.S. Pat. Nos. 4,716,179; 5,145,842; 6,268,395; Kawamura et al., 2016, “Nineteen-step total synthesis of (+)-phorbol,” Nature 532:90; Duran-Pena et al., 2014, Natural Product Reports 31:940-952; Shi et al., 2008, Chem. Rev. 108:4295-4327; all of which are incorporated herein by reference.

Deoxyphorbols comprise a partial structure of formula A, particularly the partial structures of formula A1, A2, A3 or A4, except that the carbon atom at position 12 of the structure formula is unsubstituted (i.e., H). In some embodiments, substituents can be present on one or more of carbon atoms 2, 3, 4, 5, 6, 7, 9, 11, 13, 14, and 15 of formula A, particularly of formula A1, A2, A3 or A4 where the PKC activating compound is deoxy at carbon atom 12. PKC activating deoxyphorbol compounds and derivatives, analogs, and prodrugs thereof, and methods of their synthesis are described in among others, U.S. Pat. Nos. 6,432,452; 8,022,103, 8,067,632; 8,431,612; 8,536,378; 8,816,122; US 20090187046; US 20110014699; US 20120101283; Wender, et al., 2008, “Practical Synthesis of Prostratin, DPP, and Their Analogs, Adjuvant Leads Against Latent HIV,” Science. 320(5876):649-652; Beans et al., 2013, “Highly potent, synthetically accessible prostratin analogs induce latent HIV expression in vitro and ex vivo,” Proc Natl Acad Sci USA 110(29):11698-11703; Tsai et al., 2016, “Isolation of Phorbol Esters from Euphorbia grandicornis and Evaluation of Protein Kinase C- and Human Platelet-Activating Effects of Euphorbiaceae Diterpenes,” J Nat Prod. 79(10):2658-2666; Duran-Pena et al., 2014, Natural Product Reports 31:940-952; Shi et al., 2008, Chem. Rev. 108:4295-4327; all publications incorporated herein by reference.

In some embodiments, the tigliane class of PKC activating compounds (e.g., phorbol and deoxyphorbol) have an alkyl (e.g., methyl) at carbon atoms 2, 11, and 15, and an optionally substituted alkyl, e.g., methyl or methylene at carbon atom 6. As will be understood by the skilled artisan, the numbering of the carbon atoms for such structures can use the following:

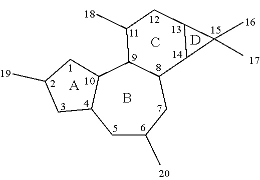

In some embodiments, the ferroptosis inducer is used in combination with an ingenane or ingenol class of PKC modulating compounds. These compounds comprise a partial structure of formula B:

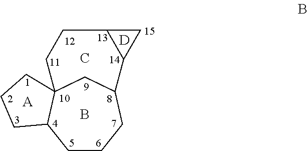

In some embodiments, the bond between carbon atoms 6 and 7 and carbon atoms 1 and 2 are each independently a double bond, as illustrated in formula B1 below. In some embodiments, carbon atom 9 is bonded to an oxygen atom to form a carbonyl, as illustrated in formula B2. In some embodiments, carbon atoms 6 and 7 are bonded to a common oxygen atom to form an epoxide, as illustrated in formula B3. In some embodiments, substituents can be present on one or more carbon atoms 2, 3, 4, 5, 6, 7, 9, 11, 12, 13, 14 and 15 of formula B, particularly of formula B1, B2 and B3.

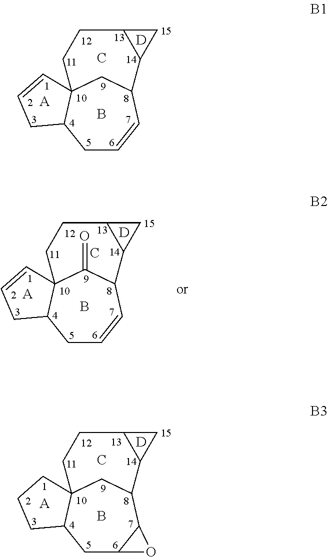

Ingenol compounds and derivatives, analogs, and prodrugs thereof, and methods of their synthesis are described in among others, U.S. Pat. Nos. 6,432,452; 8,022,103, 8,106,092; 8,431,612; 8,901,356; 9,102,687; US 20080069809; US 2010204318; US 20130324600; US 20130331446; US 20140371311; US 20150175622; WO20130182688; WO2014066967; Jorgensen et al., 2013, “14-Step Synthesis of (+)-Ingenol from (+)-3-Carene,” Science 341(6148):878-882; McKerral et al., 2014, “Development of a Concise Synthesis of (+)-Ingenol,” J. Am Chem Soc. 136 (15):5799-5810; Liang et al., 2013, Bioorg Med Chem Lett. 23:5624-5629; Grue-Sorensen et al., 2014, “Synthesis, biological evaluation and SAR of 3-benzoates of ingenol for treatment of actinic keratosis and non-melanoma skin cancer,” Bioorg Med Chem Lett. 24:54-60; Duran-Pena et al., 2014, Natural Product Reports 31:940-952; Shi et al., 2008, Chem. Rev. 108:4295-4327; all of which are incorporated herein by reference.

In some embodiments, the ingenane class of PKC activating compounds (e.g., ingenols) have an alkyl (e.g., methyl) at carbon atoms 2, 11, and 15, and an optionally substituted alkyl, e.g., methyl or methylene at carbon atom 6. As will be understood by the skilled artisan, the numbering of the carbon atoms for such structures can use the following:

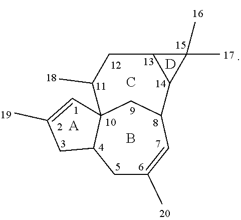

In some embodiments, the ferroptosis inducer is used in combination with a daphnane class of PKC modulating compounds. These compounds comprise a partial structure of formula C:

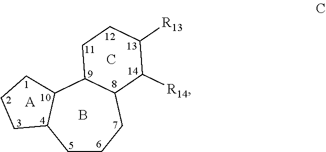

wherein one of R₁₃ and R₁₄ is an optionally substituted lower alkenyl of structure:

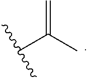 [SMILES: *C(=C)C]

The daphnane class of diterpenoid PKC modulators constitutes a diverse group of compounds. In some embodiments, the bond between carbon atoms 6 and 7 and the bond between carbon atoms 1 and 2 are each independently a double bond, as illustrated in formula C1 and C3 below. In some embodiments, the carbon atoms 6 and 7 are bonded to a common oxygen atom to form an epoxide, as illustrated in formula C2 and C4.

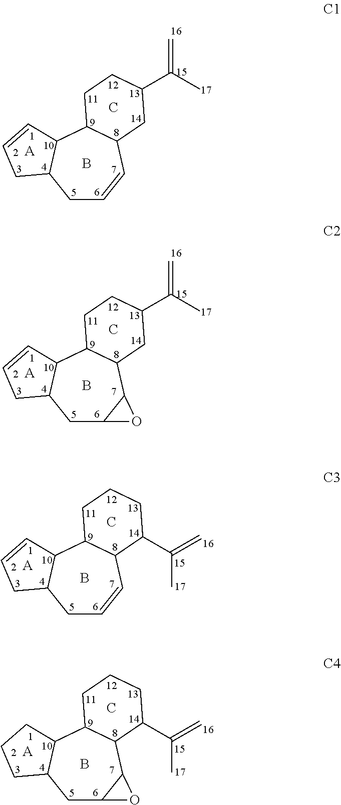

In some embodiments, substituents can be present on one or more carbon atoms 1, 2, 3, 4, 5, 6, 7, 9, 12, 13, and 14 of formula C, and additionally at carbon atom 17 for compounds of formula C1, C2, C3 and C4. Exemplary daphnane diterpenoid PKC activators include, among others, GD-1, yuanhuacine, mezerein, sapintoxin D, thymeleatoxin A, simplexin, gnidimacrin, pimelea factor S7, genididin, geniditrin and gnidilatin. Daphnane PKC activating compounds, and derivatives and analogs thereof, are described in among others, U.S. Pat. No. 5,145,842; Wender et al., 2011, Nat Chem. 3(8):615-619; Yoshida et al., 1996, Int J Cancer 66(2):268-73; and Brooks et al., 1989, Carcinogenesis 10(2):283-8; all publications incorporated herein by reference.

In some embodiments, the ferroptosis inducer is used in combination with a lathyrane class of PKC modulating compounds. These compounds comprise a partial structure of formula D:

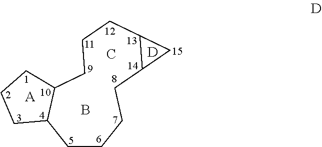

In some embodiments, the bond between carbon atoms 1 and 2, carbon atoms 4 and 10, carbon atoms 4 and 5, carbon atoms 5 and 6, carbon atoms 6 and 7, and carbon atoms 11 and 12, can independently be a double bond, where compatible with substituents on the structure. In some embodiments, carbon atoms 1 and 2, carbon atoms 4 and 10, or carbon atoms 3 and 6 are bonded to a common oxygen atom to form an epoxide ring. In some embodiments, the lathyrane compound contains an epoxide ring and a double bond, where compatible with substituents on the compound. In the some embodiments, the lathyrane compound has the partial structures D1 to D6 illustrated below.

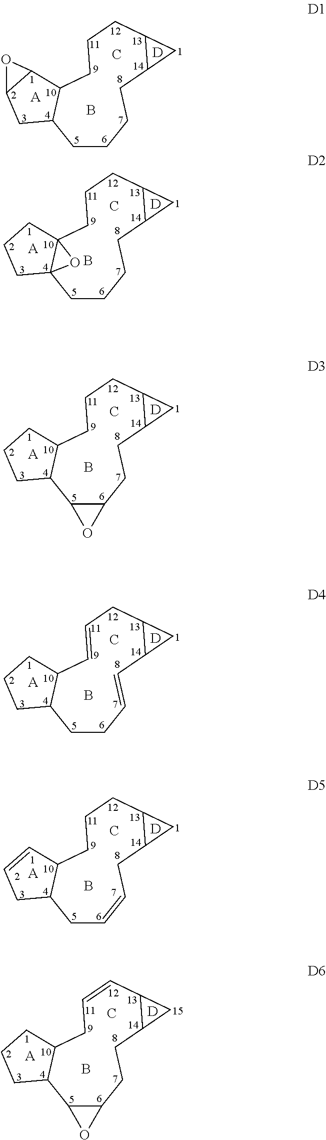

In various embodiments, substituents can be present on or more of the carbon atoms 1 to 15 of formula D, particularly lathyranes of formula D1, D2, D3, D4, D5 and D6. PKC activating lathyrane compounds, and derivatives and analogs, and method of their synthesis are described in, among others, Appendino et al., 1999, J Nat Prod. 62(10):1399-404; Ferreira et al., 2002, Phytochemistry 61(4):373-7; Valente et al., 2004, Planta Med. 70(3):244-9; Pusztai et al., 2007, Anticancer Res. 27(1A):201-5; Duran-Pena et al., 2014, Natural Product Reports 31:940-952; Shi et al., 2008, Chem. Rev. 108:4295-4327; Li et al., 2009, J Nat Prod. 72(6):1001-5; Zhang et al., 2011, Molecules. April 16(4):3222-31; Huang et al., 2014, Nat Prod Bioprospect. 4(2):91-100; Reis et al., 2014, Planta Med. 80(18):1739-45; Lin et al., 2017, Acta Pharm Sin B. 7(1):59-64; all publications incorporated herein by reference.

In some embodiments, the lathyrane class of PKC activating compounds have an alkyl (e.g., methyl) at carbon atom 15 (e.g., dimethyl), and optionally alkyl groups (e.g., methyl) at one or more of carbon atom 2, 6 and 11. As will be understood by the skilled artisan, in some embodiments, the numbering of the carbon atoms for such structures can use the following:

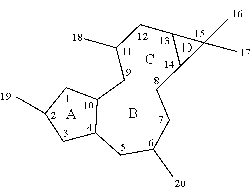

In some embodiments, the PKC activating tigliane, ingenane, daphnane or lathyrane compound for use in the methods herein is a non-tumor promoting tigliane, ingenane, daphnane or lathyrane diterpenoid compound. “Tumor promoting” refers to the ability of a compound to promote tumorigenesis, while a “non-tumor promoting” characteristic refers to the absence or insignificant activity in promoting tumorigenesis.

In some embodiments, the PKC activating tigliane, ingenane, daphnane or lathyrane compound for use in the methods does not significantly down-regulate expression of PKC protein. While many tigliane, ingenane and daphnane diterpenoids have PKC activating activity, some of the compounds also down-regulate expression of PKC protein. In some instances, this down-regulation could reduce or negate the advantageous effects of PKC activation. For example, PKC activating compounds that have tumor-promoting properties, such as 12-O-Tetradecanoylphorbol-13-acetate (TPA), also known as phorbol 12-myristate 13-acetate (PMA), have been shown to down-regulate PKC expression following extended exposure of cells to compounds (see, e.g., Lu et al., Mol Cell Biol., 17(6):3418-3428). In some embodiments, PKC activating tigliane, ingenane, daphnane or lathyrane compound can be selected for low or minimal PKC down-regulating characteristics. In some embodiments, PKC activating compounds are selected which does not downregulate PKC activity by more than 20%, 30%, 40%, 50%, 60%, or 70% of activity present in the absence of the PKC activating compound. In some embodiments, the down-regulation (or absence of down-regulation) is for global PKC expression. In some embodiments, the down-regulation is with respect to one or more of PKC isoforms selected from PKC α, β, γ, δ, ε, η, θ, ι/λ, μ, and ζ. Exemplary non-tumor promoting diterpenoid PKC activating compounds are based on 12-deoxyphorbol compounds, such as prostratin.

In some embodiments, the PKC activator is a compound of structural formula (PI):

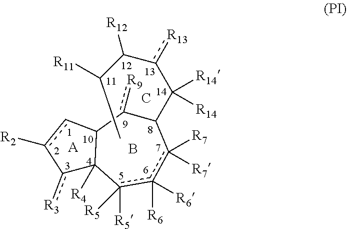

or an enantiomer, hydrate, solvate, or pharmaceutically acceptable salt thereof

wherein

Ring C is attached to Ring B at carbon atom 9 or 10;

R₂ is selected from H or lower alkyl;

R₃ is H, or O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl, —S(O)₂R_{b}, —S(O)₂OR_{b}, or —P(O)(OR_{b})₂;

R₄ and R₅ are independently H, halo, cyano, or R₄ is —OR_{c}, wherein R_{c} is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, —S(O)₂R_{b}, —S(O)₂OR_{b}, and —P(O)(OR_{b})₂;

R₅′ and R₆′ are H, or R₅′ and R₆′ together form a bond or are bonded to a common oxygen atom to form an epoxide;

R₆ is —NR_{b}R_{b}, —NHC(O)R_{b}, —SR_{b}, SOR_{b}, —S(O)₂R_{b}, —S(O)₂OR_{b}, —P(O)(OR_{b})₂, —SeR_{b}, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —C₁₋₄ alkyl-O—R_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, —S(O)₂R_{b}, —S(O)₂OR_{b}, —P(O)(OR_{b}), or R_{d} is a promoiety which is hydrolyzable under biological conditions to yield an -alkyl-OH.

R₆′ and R₇′ are H, or R₆′ and R₇′ together form a bond or are bonded to a common oxygen atom to form an epoxide;

R₇ is H or OH;

R₉ is H, oxo, or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl, or R₉′, is an O atom which is bonded to an optionally substituted common C atom bonded to R₁₃′ and R₁₄′, wherein R₁₃′ and R₁₄′ each is an O atom;

R₁₁ is lower alkyl;

R₁₂ is H, halo, —NR_{b}R_{b}, —NHC(O)R_{b}, —SR_{b}, SOR_{B}, —S(O)₂R_{b}, —S(O)₂OR_{b}, —P(O)(OR_{b})₂, —SeR_{b}, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or R₁₂ is —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, —S(O)₂R_{b}, —S(O)₂OR_{b}, and —P(O)(OR_{b})₂;

R₁₃ is H, halo, oxo, —NR_{b}R_{b}, —NHC(O)R_{b}, —SR_{b}, SOR_{B}, —S(O)₂R_{b}, —S(O)₂OR_{b}, —P(O)(OR_{b})₂, —SeR_{b}, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, —S(O)₂R_{b}, —S(O)₂OR_{b}, and —P(O)(OR_{b})₂;

R₁₃′ and R₁₄′ are independently H, OH, or are bonded to a common carbon atom to form a cyclopropyl ring, wherein the cyclopropyl ring is optionally mono- or disubstituted with OH, halo, —NR_{b}R_{b}, —NHC(O)R_{b}, —SR_{b}, SOR_{b}, —S(O)₂R_{b}, —S(O)₂OR_{b}, and —OP(O)(OR_{b})₂, —SeR_{b}, optionally substituted alkyl, optionally substituted cycloalkyl, optionally substituted heteroalkyl, optionally substituted aryl, optionally substituted heteroaryl, optionally substituted alkyloxy, optionally substituted alkenyloxy, optionally substituted alkynyloxy, optionally substituted cycloalkyloxy, optionally substituted cycloalkenyloxy, optionally substituted heterocycloalkyloxy, optionally substituted aryloxy, optionally substituted heteroaryloxy, optionally substituted arylalkyloxy, optionally substituted arylalkenyloxy, optionally substituted heteroarylalkyloxy, optionally substituted heteroarylalkenyloxy, optionally substituted alkylcarbonyloxy, optionally substituted alkenylcarbonyloxy, optionally substituted alkynylcarbonyloxy, optionally substituted arylcarbonyloxy, optionally substituted heteroarylcarbonyloxy, optionally substituted arylalkylcarbonyloxy, optionally substituted arylalkenylcarbonyloxy, optionally substituted heteroarylalkylcarbonyloxy, optionally substituted heteroarylalkenylcarbonyloxy, optionally substituted carboxyalkylcarbonyloxy, optionally substituted amino acid carbonyloxy, carbamate, phosphine, phosphoramide, phosphoramidite, phosphoramidate, phosphonate, sulfonamide, amide, guanidine, urea; or a progroup which is hydrolysable under biological conditions to yield an -alkyl-OH group, or R₁₃′ and R₁₄′ are each an O atom which is bonded to an optionally substituted common C atom bonded to R₉, wherein R₉ is an O atom;

R₁₄ is H, OH or optionally substituted alkenyl;

wherein each R_{b} is independently H, optionally substituted alkyl, optionally substituted alkenyl, alkynyl, optionally substituted aryl, optionally substituted heteroaryl, optionally substituted arylalkyl, or optionally substituted heteroarylalkyl; and

the dashed line (- - - - -) represents an optional bond.

In some embodiments of structural formula (PI), R₆ is —CH₂Rₕ, wherein

Rₕ is —O—C(O)—Rᵢ, wherein Rᵢ is a moiety which bears a permanent charge or which is ionizable at a pH in the range of about 2 to 8, and wherein the —O—C(O)—Rᵢ is hydrolyzable under biological conditions to yield an —OH group. In some embodiments, R, is an optionally substituted carboxyalkyl, wherein the carboxy is COOM, and wherein M is an H or a counterion. In some embodiments, the alkyl of Rᵢ is a C₁₋₆ alkyl. In some embodiments, Rⱼ is an amino acid of structure —(CH₂)ₙ—CH(CH₂)ₙ—NH₂)—(CH₂)ₙ—C(O)OM or —(CH₂)ₙ—CHNH₂—(CH₂)ₙ—C(O)OM, wherein n is 0, 1, 2, 3 or 4. In some embodiments, R₁ is an aminoalkyl, wherein the amino group is —NRⱼRⱼ or —NRₖRₖRₖ, wherein each Rⱼ and Rₖ is independently H, lower alkyl, lower alkyloxyalkyl, heteroalkyl, or two Rⱼ taken together with the nitrogen atom to which they are bonded form a 5-7 membered heteroatomic ring. In some embodiments, the alkyl of the aminoalkyl is a C₁₋₆ alkyl. In some embodiments, —NRⱼRⱼ is N-morpholinyl, piperazinyl, 1-piperazinyl, 1-methyl-piperazinyl, or 1-methyl-4-piperazinyl. In some embodiments, the progroup and promoieties are as described in, for example, patent publication US2011/0224297, paragraphs [0036] to [0045], incorporated herein by reference.

In some embodiments, the PKC activator is a compound of structural formula (PII):

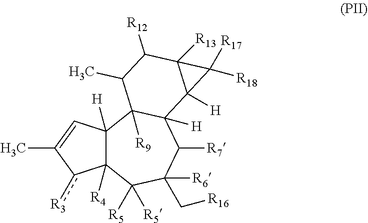

or an enantiomer, hydrate, solvate, or pharmaceutically acceptable salt thereof,

wherein

R₃ is O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;

R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₅′ and R₆′ are H, or R₅′ and R₆′ together form a bond or are bonded to a common oxygen atom to form an epoxide;

R₆′ and R₇′ are H, or R₆′ and R₇′ together form a bond or are bonded to a common oxygen atom to form an epoxide;

R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl;

R₁₂ is H, halo, or —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₁₃ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₁₆ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group at R₁₆; and

R₁₇ and R₁₈ are each independently H, OH, amino, thiol, sulfanyl, sulfinyl, sulfonyl, optionally substituted alkyl, optionally substituted cycloalkyl, optionally substituted heteroalkyl, optionally substituted aryl, optionally substituted heteroaryl, optionally substituted alkyloxy, optionally substituted alkenyloxy, optionally substituted aryloxy, optionally substituted arylalkyloxy, optionally substituted alkylcarbonyloxy, optionally substituted alkenylcarbonyloxy, optionally substituted arylcarbonyloxy, optionally substituted arylalkylcarbonyloxy, phosphine, phosphate, phosphoramide, phosphoramidite, phosphoramidate, phosphonate, sulfate, sulfonate, sulfonamide, sulfone, sulfite, amide, guanidine, or urea.

In some embodiments of structural formula (PII), Rib is —O—C(O)—Rᵢ, wherein Rᵢ is a moiety which bears a permanent charge or which is ionizable at a pH in the range of about 2 to 8, and wherein the —O—C(O)—R; is hydrolyzable under biological conditions to yield an —OH group. In some embodiments, Rᵢ is an optionally substituted carboxyalkyl, wherein the carboxy is COOM, and wherein M is an H or a counterion. In some embodiments, the alkyl of Rᵢ is a C₁₋₆ alkyl. In some embodiments, Rᵢ is an amino acid of structure —(CH₂)ₙ—CH(CH₂)ₙ—NH₂)—(CH₂)ₙ—C(O)OM or —(CH₂)ₙ—CHNH₂—(CH₂)ₙ—C(O)OM, wherein n is 0, 1, 2, 3 or 4. In some embodiments, R₁ is an aminoalkyl, wherein the amino group is —NRⱼRⱼ or —NRₖRₖRₖ, wherein each Rⱼ and Rₖ is independently H, lower alkyl, lower alkyloxyalkyl, heteroalkyl, or two Rⱼ taken together with the nitrogen atom to which they are bonded form a 5-7 membered heteroatomic ring. In some embodiments, the alkyl of the aminoalkyl is a C₁₋₆ alkyl. In some embodiments, —NRⱼRⱼ is N-morpholinyl, piperazinyl, 1-piperazinyl, 1-methyl-piperazinyl, or 1-methyl-4-piperazinyl. In some embodiments, the progroup and promoieties are as described in, for example, patent publication US2011/0224297, paragraphs [0036] to [0045], incorporated herein by reference.

In some embodiments, the PKC activator is the compound of formula (PIIa):

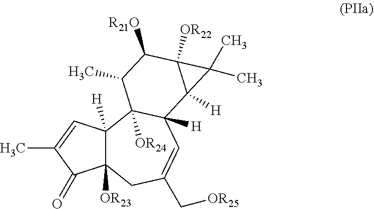

or an enantiomer, hydrate, solvate, or pharmaceutically acceptable salt thereof,

wherein

R₂₁, R₂₂, R₂₃, and R₂₄ are each independently H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and

R₂₅ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R₂₅ is a promoiety which is hydrolyzable under biological conditions to yield an —OH group at the C20 carbon atom.

In some embodiments, the PKC activator is a compound of formula (PIIb):

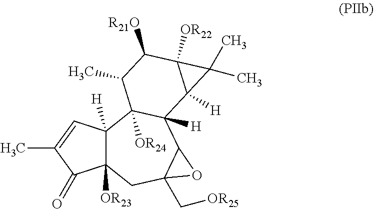

or an enantiomer, hydrate, solvate, or pharmaceutically acceptable salt thereof,
wherein,

R₂₁, R₂₂, R₂₃, R₂₄ and R₂₅ are as defined for formula (PIIa).

In some embodiments, the PKC activator is a compound of formula (PIIc):

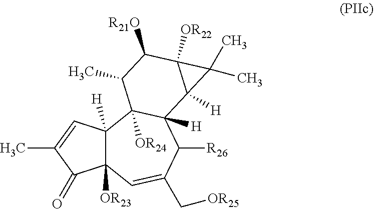

or an enantiomer hydrate, solvate, or pharmaceutically acceptable salt thereof,

wherein,

R₂₁, R₂₂, R₂₃, R₂₄ and R₂₅ are as defined for formula (PIIa), and

R₂₆ is H or OH.

In some embodiments, the PKC activator is a compound of formula (PIId):

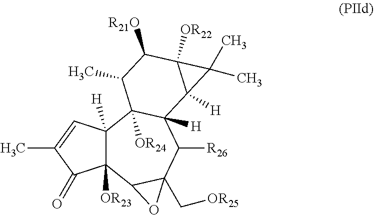

or an enantiomer, hydrate, solvate, or pharmaceutically acceptable salt thereof,

wherein,

R₂₁, R₂₂, R₂₃, R₂₄ and R₂₅ are as defined for formula (PIIa), and

R₂₆ is H or OH.

In some embodiments of structural formula (PIIa), (PIIb), (PIIc) or (PIId), the aryl is an optionally substituted phenyl.

In some embodiments of structural formula (PIIa), (PIIb), (PIIc) and (PIId), R₂₅ forms a promoiety as described in formula (PIT) above. In some embodiments of structural formula (PIIa), (PIIb), (PIIc) and (PIId), R₂₅ is an optionally substituted carboxyalkylcarbonyl, wherein the carboxy is COOM, wherein M is an H or a counterion. In some embodiments, the alkyl is a C₁₋₆ alkyl. In some embodiments, R₂₅ is an amino acid carbonyl, where the amino acid portion has the structure —(CH₂)ₙ—CH(CH₂)ₙ—NH₂)—(CH₂)ₙ—C(O)OM or —(CH₂)ₙ—CHNH₂—(CH₂)ₙ—C(O)OM, wherein n is 0, 1, 2, 3, or 4. In some embodiments, R₂₅ is an aminoalkylcarbonyl, wherein the alkyl is a C₁₋₆ alkyl and the amino group is —NRⱼRⱼ or —NRₖRₖRₖ, wherein each Rⱼ and Rₖ is independently H, lower alkyl, lower alkyloxyalkyl, heteroalkyl, or two Rⱼ taken together with the nitrogen atom to which they are bonded form a 5-7 membered heteroatomic ring. In some embodiments, —NRⱼRⱼ is N-morpholinyl, piperazinyl, 1-piperazinyl, 1-methyl-piperazinyl, or 1-methyl-4-piperazinyl. In some embodiments, the progroup and promoieties are as described in, for example, patent publication US2011/0224297, paragraphs [0036] to [0045], incorporated herein by reference.

In some embodiments, the PKC activator is selected from the exemplary phorbol compounds presented below, including, among others, phorbol 13-butyrate; phorbol 12-decanoate; phorbol 13-decanoate; phorbol 12,13-diacetate, phorbol 13,20-diacetate, phorbol 12,13-dibenzoate, phorbol 12,13 dibutyrate, phorbol 12,13 didecanoate; phorbol 12,13-dihexanoate; phorbol 12,13 dipropionate, phorbol 12-myristate; phorbol 13-myristate, phorbol 12-myristate-13-acetate (TPA), phorbol 12,13,20-triacetate; phorbol 12-acetate, phorbol 13-acetate, phorbol-12-tigliate 13-decanoate, or salts, hydrates, solvates, or prodrugs thereof. In some embodiments, the prodrugs for the specified phorbol compounds contain a biohydrolyzable carbonate, biohydrolyzable ureide, biohydrolyzable carbamate, biohydrolyzable ester, biohydrolyzable amide, or biohydrolyzable phosphate group. In particular, the prodrug for the specified compound contains a biohydrolyzable ester, more particularly at the C20 carbon.

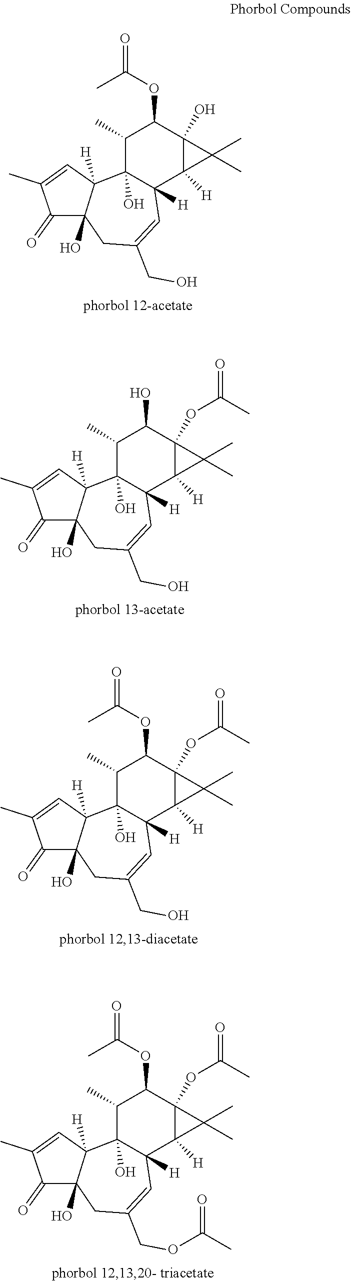

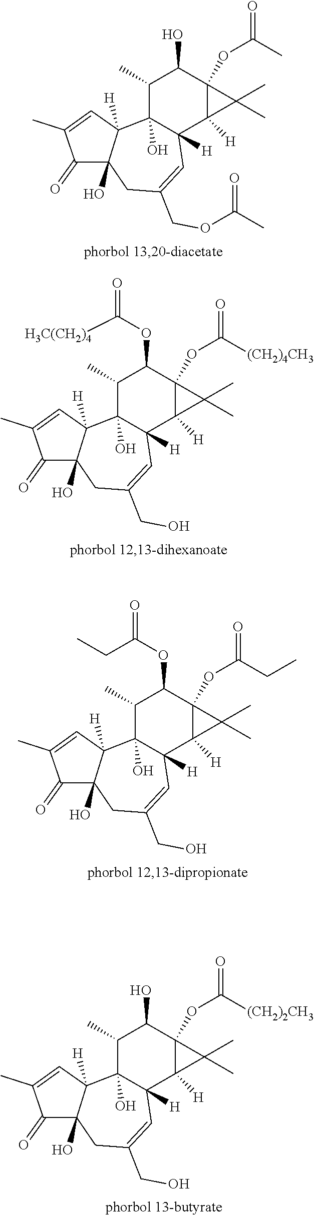

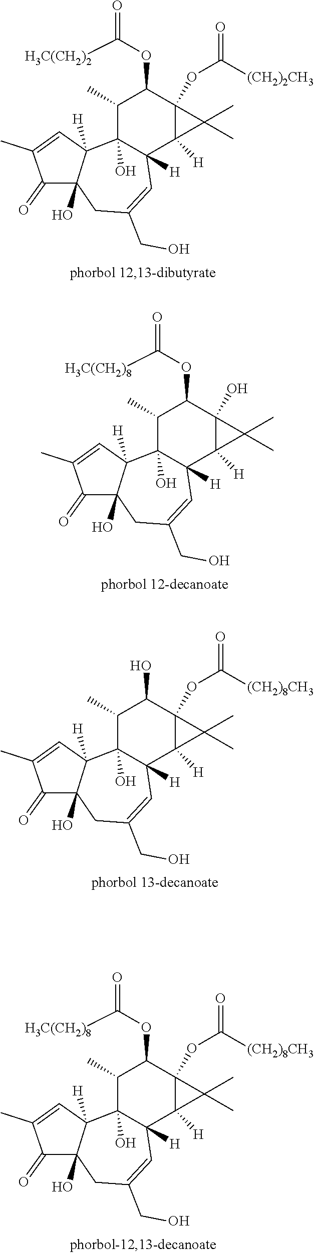

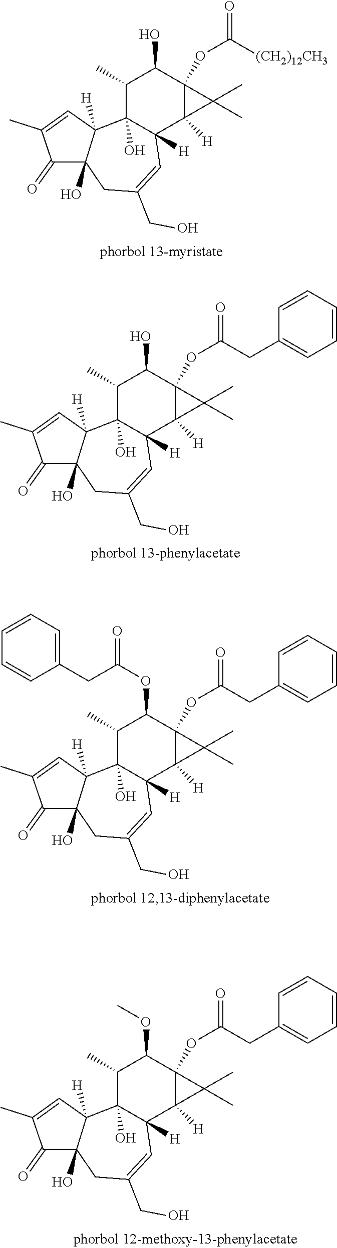

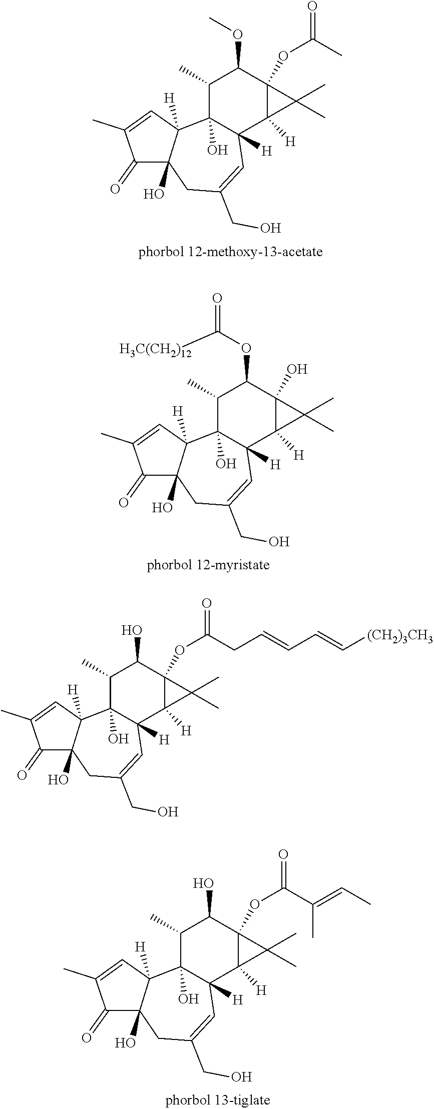

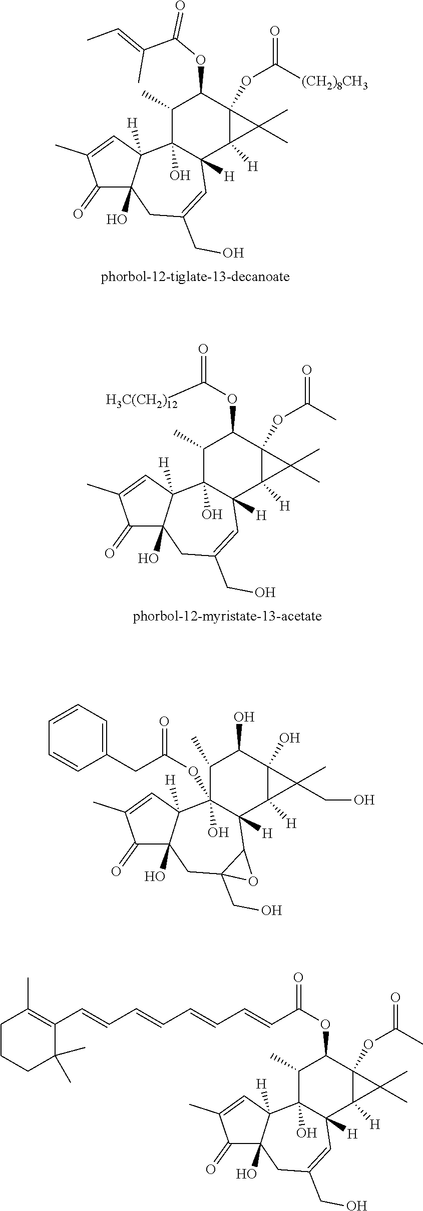

In some embodiments, the PKC activator is compound of structural formula (PIII):

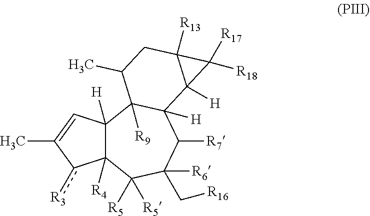

or an enantiomer, hydrate, solvate, or pharmaceutically acceptable salt thereof,

wherein

R₃ is O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;

R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₅′ and R₆′ are H, or R₅′ and R₆′ together form a bond or are bonded to a common oxygen atom to form an epoxide;

R₆′ and R₇′ are H, or R₆′ and R₇′ together form a bond or are bonded to a common oxygen atom to form an epoxide;

R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl;

R₁₃ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₁₆ is H, halo, or —O—R_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group—at the C20 carbon atom; and

R₁₇ and R₁₈ are each independently H, OH, amino, thiol, sulfanyl, sulfinyl, sulfonyl, optionally substituted alkyl, optionally substituted cycloalkyl, optionally substituted heteroalkyl, optionally substituted aryl, optionally substituted heteroaryl, optionally substituted alkyloxy, optionally substituted alkenyloxy, optionally substituted alkynyloxy, optionally substituted cycloalkyloxy, optionally substituted cycloalkenyloxy, optionally substituted heterocycloalkyloxy, optionally substituted aryloxy, optionally substituted heteroaryloxy, optionally substituted arylalkyloxy, optionally substituted arylalkenyloxy, optionally substituted heteroarylalkyloxy, optionally substituted heteroarylalkenyloxy, optionally substituted alkylcarbonyloxy, optionally substituted alkenylcarbonyloxy, optionally substituted alkynylcarbonyloxy, optionally substituted arylcarbonyloxy, optionally substituted heteroarylcarbonyloxy, optionally substituted arylalkylcarbonyloxy, optionally substituted arylalkenylcarbonyloxy, optionally substituted heteroarylalkylcarbonyloxy, optionally substituted heteroarylalkenylcarbonyloxy, optionally substituted carboxyalkylcarbonyloxy, optionally substituted amino acid carbonyloxy, phosphine, phosphate, phosphoramide, phosphoramidite, phosphoramidate, phosphonate, sulfate, sulfonate, sulfonamide, sulfone, sulfite, amide, guanidine, urea, or a progroup which is hydrolyzable under biological conditions to yield an -alkyl-OH group.

In some embodiments of structural formula (PIII), R₁₆ is —O—C(O)—Rᵢ, wherein Rᵢ is a moiety which bears a permanent charge or which is ionizable at a pH in the range of about 2 to 8, and wherein the —O—C(O)—Rᵢ is hydrolyzable under biological conditions to yield an —OH group. In some embodiments, Rᵢ is an optionally substituted carboxyalkyl, wherein the carboxy is COOM, and wherein M is an H or a counterion. In some embodiments, the alkyl of Rᵢ is a lower alkylene. In some embodiments, Rᵢ is an amino acid of structure —(CH₂)ₙ—CH(CH₂)ₙ—NH₂)—(CH₂)ₙ—C(O)OM or —(CH₂)ₙ—CHNH₂—(CH₂)ₙ—C(O)OM, wherein n is 0, 1, 2, 3 or 4. In some embodiments, R₁ is an aminoalkyl, wherein the amino group is —NRⱼRⱼ or —NRₖRₖRₖ, wherein each Rⱼ and Rₖ is independently H, lower alkyl, lower alkyloxyalkyl, heteroalkyl, or two Rⱼ taken together with the nitrogen atom to which they are bonded form a 5-7 membered heteroatomic ring. In some embodiments, the alkylene of the aminoalkyl is a lower alkyl. In some embodiments, —NRⱼRⱼ is N-morpholinyl, piperazinyl, 1-piperazinyl, 1-methyl-piperazinyl, or 1-methyl-4-piperazinyl. In some embodiments, the progroup and promoieties are as described in, for example, patent publication US2011/0224297, paragraphs [0036] to [0045], incorporated herein by reference.

In some embodiments, the PKC activator is a compound of formula (PIIIa) or (PIIIb):

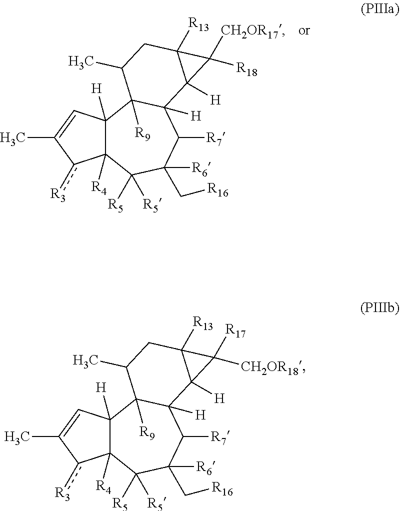

or an enantiomer, hydrate, solvate, or pharmaceutically acceptable salt thereof,

wherein

R₃, R₄, R₅, R₅′ R₆′, R₇′, R₉, R₁₃, and R₁₆ are as defined for formula (PIII);

R₁₇ or R₁₈ is H, OH, amino, thiol, sulfanyl, sulfinyl, sulfonyl, optionally substituted alkyl, optionally substituted cycloalkyl, optionally substituted heteroalkyl, optionally substituted aryl, optionally substituted heteroaryl, optionally substituted alkyloxy, optionally substituted aryloxy, optionally substituted arylalkyloxy, phosphine, phosphate, phosphoramide, phosphoramidite, phosphoramidate, phosphonate, sulfate, sulfonate, sulfonamide, sulfone, sulfite, amide, guanidine, or urea; and

R₁₇′ or R₁₈′ is H, optionally substituted alkyl, optionally substituted cycloalkyl, optionally substituted heteroalkyl, optionally substituted aryl, optionally substituted heteroaryl, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a progroup which is hydrolyzable under biological conditions to yield an —OH group.

In some embodiments, the PKC activator is a compound of formula (PIIIc):

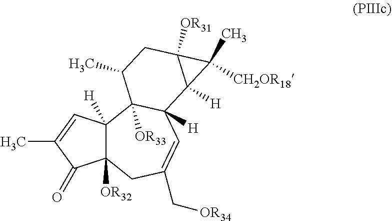

or an enantiomer, hydrate, solvate, or pharmaceutically acceptable salt thereof,

wherein,

R₁₈′ is H, optionally substituted alkyl, optionally substituted cycloalkyl, optionally substituted heteroalkyl, optionally substituted aryl, optionally substituted heteroaryl, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a promoiety which is hydrolyzable under biological conditions to yield an —OH group;

R₃₁, R₃₂, and R₃₃ are each independently H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and

R₃₄ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R₃₄ is a promoiety which is hydrolyzable under biological conditions to yield an —OH group at the C20 carbon atom.

In some embodiments, the PKC activator is a compound of formula (PIIId):

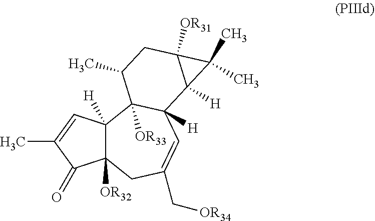

or an enantiomer, hydrate, solvate, or pharmaceutically acceptable salt thereof,

wherein

R₃₁, R₃₂, and R₃₃ are each independently H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and

R₃₄ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R₃₄ is a promoiety which is hydrolyzable under biological conditions to yield an —OH group at the C20 carbon atom.

In some embodiments, the PKC activator is a compound of formula (PIIIe):

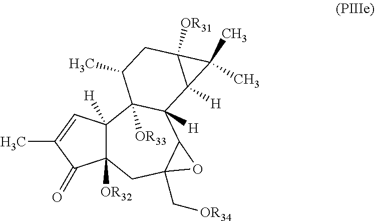

or an enantiomer, hydrate, solvate, or pharmaceutically acceptable salt thereof,

wherein,

R₃₁, R₃₂, R₃₃, and R₃₄ are as defined for formula (PIIIc).

In some embodiments of the compound of formula (PIIIc), (PIIId) and (PIIIe), R₃₄ is an optionally substituted carboxyalkylcarbonyl, wherein the carboxy is —COOM, wherein M is an H or a counterion. In some embodiments, the alkyl is a C₁₋₆ alkyl. In some embodiments, R₃₄ is an amino acid carbonyl of structure —C(O)—(CH₂)ₙ—CH(CH₂)ₙ—NH₂)—(CH₂)ₙ—C(O)OM or —C(O)—CH₂)ₙ—CHNH₂—(CH₂)ₙ—C(O)OM, wherein n is 0, 1, 2, 3, or 4. In some embodiments, R₃₄ is an aminoalkyl, wherein the amino group is —NRⱼRⱼ or —NRₖRₖRₖ, wherein each Rⱼ and Rₖ are independently H, lower alkyl, lower alkyloxyalkyl, heteroalkyl, or two Rⱼ taken together with the nitrogen atom to which they are bonded form a 5-7 membered heteroatomic ring. In some embodiments, the alkyl of R₃₄ is a C₁₋₆ alkyl. In some embodiments, the —NRⱼRⱼ is N-morpholinyl, piperazinyl, 1-piperazinyl, 1-methyl-piperazinyl, or 1-methyl-4-piperazinyl. In some embodiments, the progroup and promoieties are as described in, for example, patent publication US2011/0224297, paragraphs [0036] to [0045], incorporated herein by reference.

In some embodiments, the PKC activator is selected from the exemplary deoxyphorbol compounds presented below, including, among others, 12-deoxyphorbol 13-angelate, 12-deoxyphorbol 13-angelate 20-acetate, 12-deoxyphorbol 13-isobutyrate, 12-deoxyphorbol 13-isobutyrate 20-acetate, 12-deoxyphorbol 13-phenylacetate, 12-deoxyphorbol 13-phenylacetate 20-acetate, 12-deoxyphorbol 13-tetradecanoate, 12-deoxyphorbol 13-acetate (prostratin), or salts, hydrates, solvates, or prodrugs thereof. In some embodiments, the prodrugs for the specified deoxyphorbol compounds contain a biohydrolyzable carbonate, biohydrolyzable ureide, biohydrolyzable carbamate, biohydrolyzable ester, biohydrolyzable amide, or biohydrolyzable phosphate group. In particular, the prodrug for the specified deoxyphorbol compound contains a biohydrolyzable ester, more particularly at the C20 carbon.

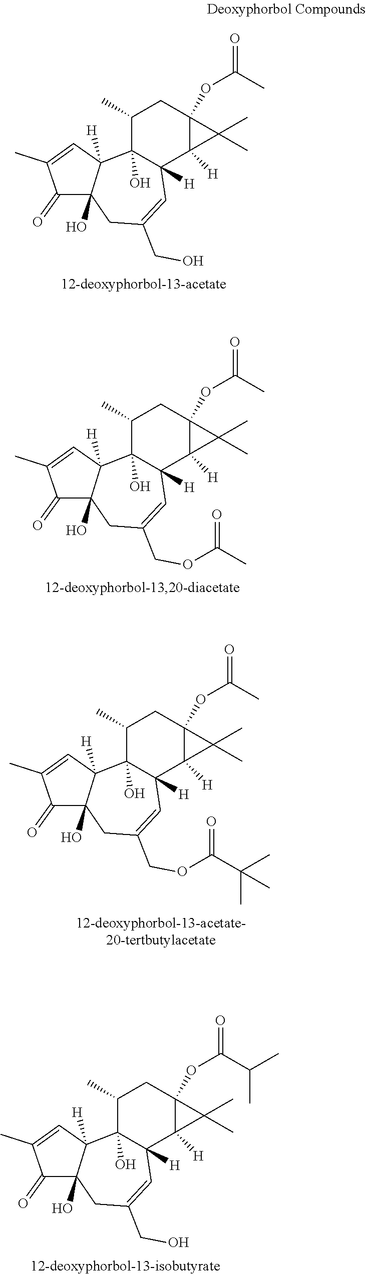

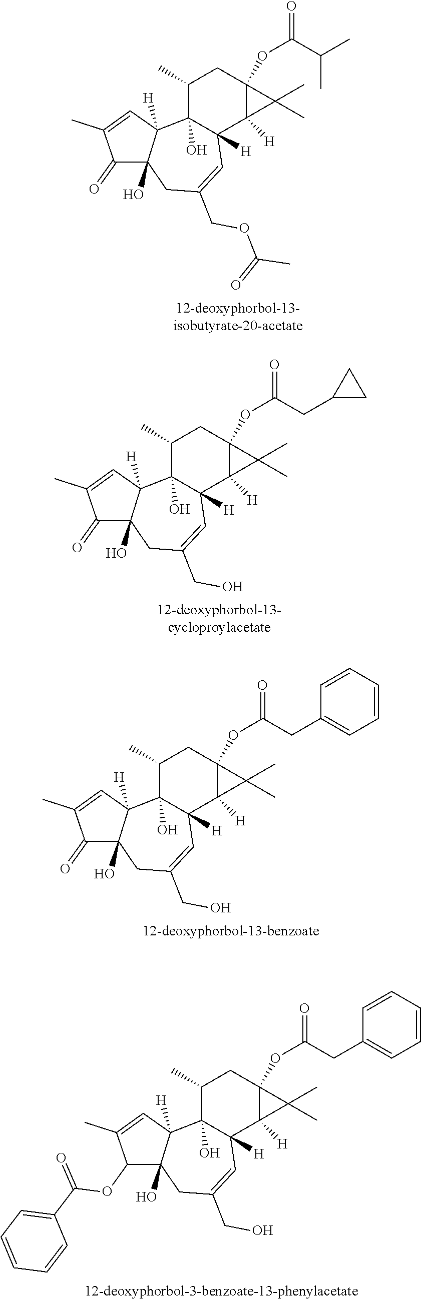

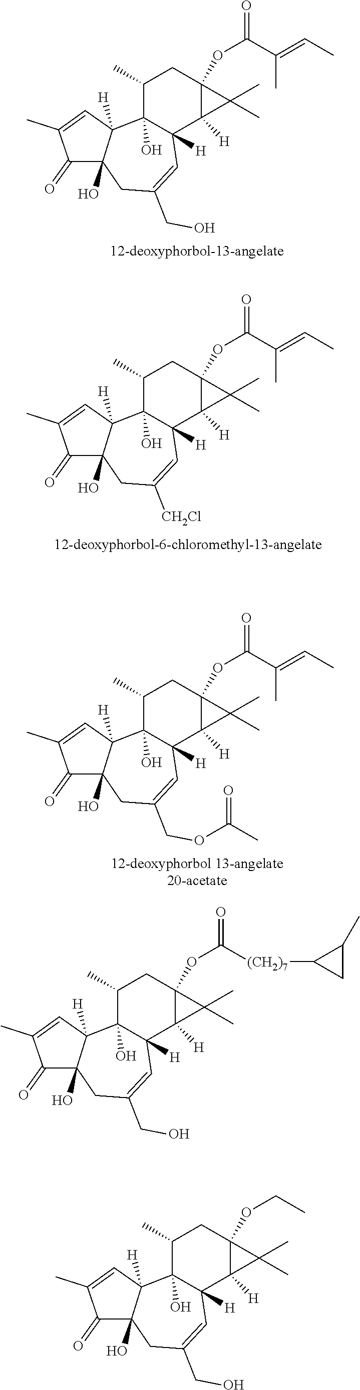

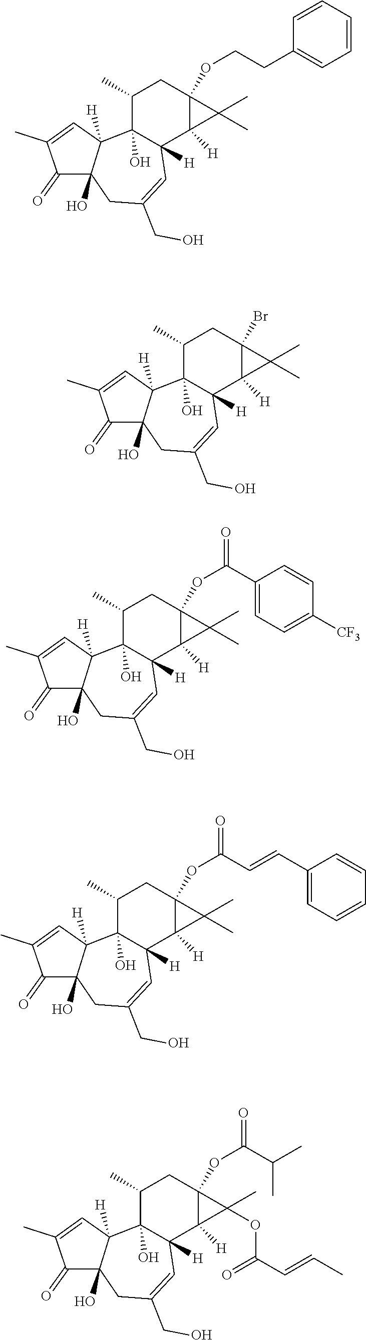

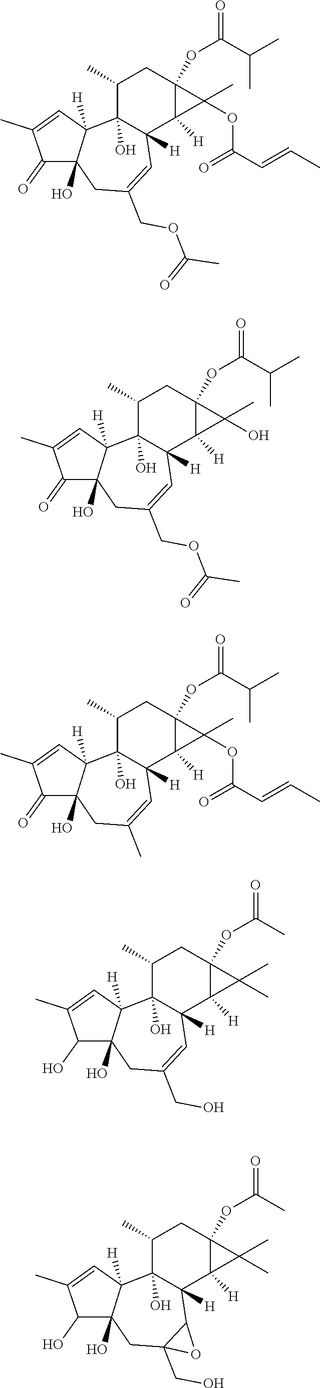

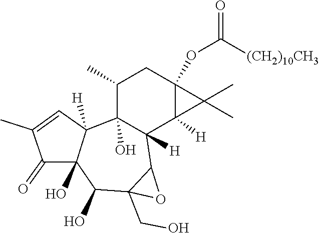

In some embodiments, the PKC activator is a compound of formula (PIV):

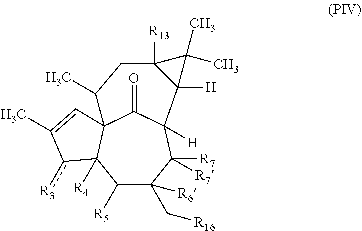

or an enantiomer, hydrate, solvate, or pharmaceutically acceptable salt thereof,

wherein

R₃ is O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;

R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₆′ and R₇′ are H, or R₆′ and R₇′ together form a bond or are bonded to a common oxygen atom to form an epoxide;

R₇ is H or OH;

R₁₃ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and

R₁₆ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group at R₁₆. In some embodiments, the progroup and promoieties are as described in, for example, patent publication US2011/0224297, paragraphs [0036] to [0045], incorporated herein by reference.

In some embodiments, the PKC activator comprises a compound of formula (PIVa):

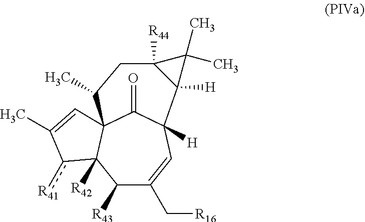

or an enantiomer, hydrate, solvate, or pharmaceutically acceptable salt thereof,

wherein

R₄₁ is O double bonded to the ring carbon, or R₄₁ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;

R₄₂ and R₄₃ are independently H, halo, or —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₄₄ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and

R₁₆ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a biohydrolyzable promoiety which is hydrolyzable under biological conditions to yield an —OH group at R₁₆.

In some embodiments, the PKC activator comprises a compound of formula (PIVb):

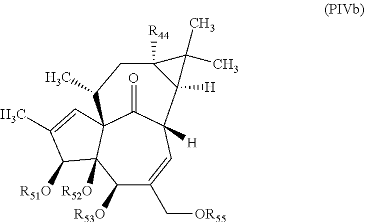

or an enantiomer, hydrate, solvate, or pharmaceutically acceptable salt thereof,

wherein

R₄₄ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₅₁ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl; and

R₅₂ and R₅₃ are independently H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and

R₅₅ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a promoiety which is hydrolyzable under biological conditions to yield an —OH group at the C20 carbon atom.

In some embodiments, the PKC activator comprises a compound of formula (PIVc):

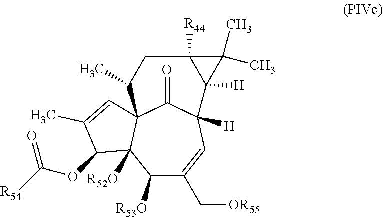

or an enantiomer, hydrate, solvate, or pharmaceutically acceptable salt thereof,

wherein

R₄₄ is H or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₅₂ and R₅₃ are independently H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₅₄ is H, an optionally substituted alkyl, optionally substituted alkenyl, optionally substituted cycloalkyl, optionally substituted aryl, optionally substituted heteroaryl, optionally substituted arylalkyl, optionally substituted arylalkenyl, optionally substituted heteroarylalkyl, optionally substituted heteroarylalkenyl; and

R₅₅ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a promoiety which is hydrolyzable under biological conditions to yield an —OH group at the C20 carbon atom.

In some embodiments of the compound of formula (PIVb) and (PIVc), R₅₅ is an optionally substituted carboxyalkylcarbonyl, wherein the carboxy is —COOM, wherein M is an H or a counterion. In some embodiments, the alkyl is a lower alkyl. In some embodiments, R₅₅ is an amino acid carbonyl of structure —C(O)—(CH₂)ₙ—CH(CH₂)ₙ—NH₂)—(CH₂)ₙ—C(O)OM or —C(O)—CH₂)ₙ—CHNH₂—(CH₂)ₙ—C(O)OM, wherein n is 0, 1, 2, 3, or 4. In some embodiments, R₅₅ is an aminoalkyl, wherein the amino group is —NRⱼRⱼ or —NRₖRₖRₖ, wherein each Rⱼ and Rₖ are independently H, lower alkyl, lower alkyloxyalkyl, heteroalkyl, or two Rⱼ taken together with the nitrogen atom to which they are bonded form a 5-7 membered heteroatomic ring. In some embodiments, the alkyl of R₅₅ is a lower alkyl. In some embodiments, the —NRⱼRⱼ is N-morpholinyl, piperazinyl, 1-piperazinyl, 1-methyl-piperazinyl, or 1-methyl-4-piperazinyl. In some embodiments, the progroup and promoieties are as described in, for example, patent publication US2011/0224297, paragraphs [0036] to [0045], incorporated herein by reference.

In some embodiments, the PKC activator comprises a compound of formula (PIVd):

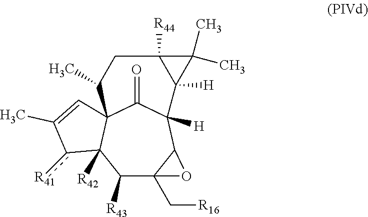

or an enantiomer, hydrate, solvate, or pharmaceutically acceptable salt thereof,
wherein

R₄₁, R₄₂, R₄₃, R₄₄ and R₁₆ are as defined for formula (PIVa).

In some embodiments, the PKC activator is selected from the exemplary ingenane compounds presented below, including, among others, ingenol-3-angelate, ingenol-5-angelate, ingenol-3,20-dibenzoate, 20-O-acetyl-ingenol-3-angelate, ingenol-3O-(3,5-diethyl-4-isoxazolecarboxylate), or 20-deoxy-ingenol-3-angelate, ingenol-20-benzoate, or solvates, hydrates, and prodrugs thereof. In some embodiments, the prodrugs for the specified ingenol compounds contain a biohydrolyzable carbonate, biohydrolyzable ureide, biohydrolyzable carbamate, biohydrolyzable ester, biohydrolyzable amide, or biohydrolyzable phosphate group. In particular, the prodrug for the specified ingenane compounds contains a biohydrolyzable ester, more particularly at the C20 carbon atom.

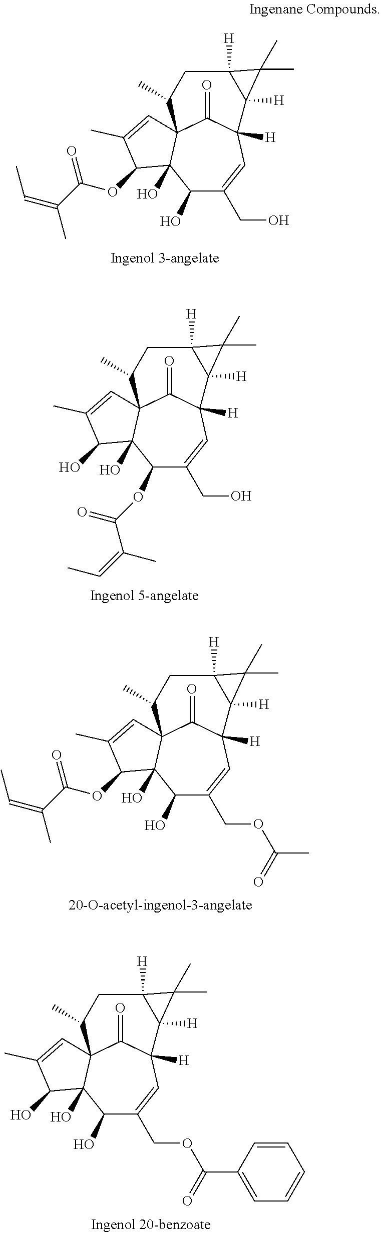

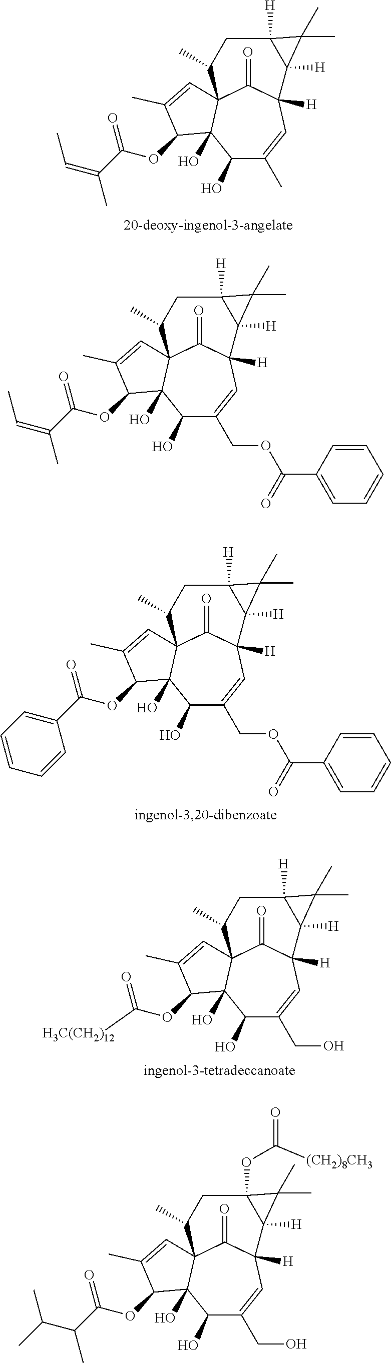

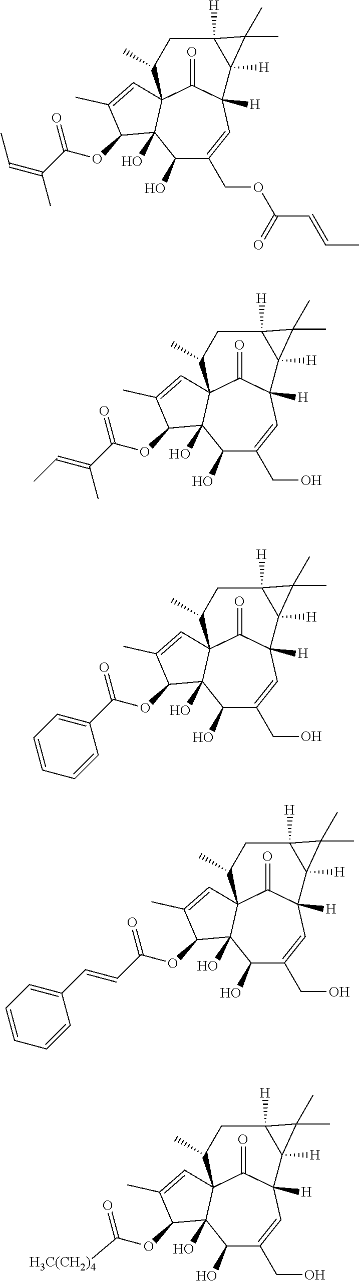

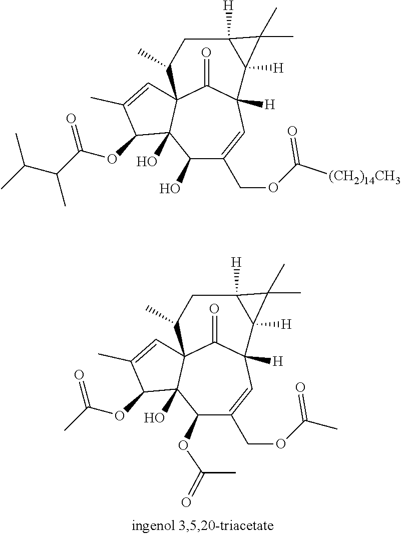

In some embodiments, the PKC activator comprises a compound of formula (PV):

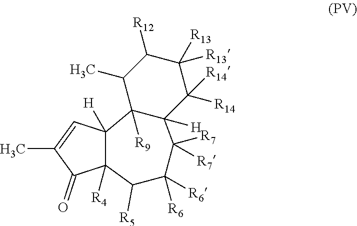

or an enantiomer, hydrate, solvate, or pharmaceutically acceptable salt thereof,

wherein,

R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, or R₉′ is an O atom which is bonded to an optionally substituted common C atom bonded to R₁₃′ and R₁₄′, wherein R₁₃′ and R₁₄′ each is an O atom;

R₆ is —NR_{b}R_{b}, —NHC(O)R_{b}, —SR_{b}, —SOR_{B}, —S(O)₂R_{b}, —S(O)₂OR_{b}, —P(O)(OR_{b})₂, —SeR_{b}, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or -alkyl-O—R_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, —S(O)₂R_{b}, —S(O)₂OR_{b}, —P(O)(OR_{b}), or R_{d} is a promoiety which is hydrolyzable under biological conditions to yield an -alkyl OH;

R₆′ and R₇′ are H, or R₆′ and R₇′ together form a bond or are bonded to a common oxygen atom to form an epoxide;

R₇ is H or OH;

R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl;

R₁₂ is H, halo, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or R₁₂ is —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₁₃ is H, halo, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, or optionally substituted heteroarylalkenylcarbonyl;

R₁₃′ and R₁₄′ are independently H or OH, or R₁₃′ and R₁₄′ are each an O atom which is bonded to an optionally substituted common C atom which is bonded to R₉, wherein R₉ is an O atom; and

R₁₄ is H, OH or optionally substituted alkenyl;

wherein one of R₁₃ and R₁₄ is an alkenyl of structure

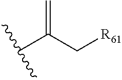

wherein R₆₁ is H or OH.

In some embodiments, the PKC activator comprises a compound of formula (PVa):

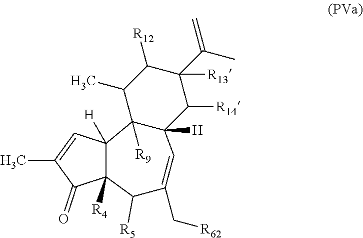

or an enantiomer, hydrate, solvate, or pharmaceutically acceptable salt thereof,

wherein,

R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, or R₉′ is an O atom which is bonded to an optionally substituted common C atom bonded to R₁₃′ and R₁₄′, wherein R₁₃′ and R₁₄′ each is an O atom;

R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, optionally substituted arylalkyloxycarbonyl, or R₉ is an O atom which is bonded to an optionally substituted common C atom bonded to R₁₃′ and R₁₄′, wherein R₁₃′ and R₁₄′ each is an O atom;

R₁₂ is H, halo, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or R₁₂ is —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₁₃′ and R₁₄′ are independently H or OH, or R₁₃′ and R₁₄′ are each an O atom which is bonded to an optionally substituted common C atom which is bonded to R₉, wherein R₉ is an O atom; and

R₆₂ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group at R₆₂.

In some embodiments, the PKC activator comprises a compound of formula (PVb):

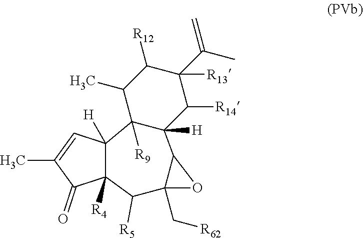

or an enantiomer, hydrate, solvate, or pharmaceutically acceptable salt thereof,

wherein

R₄, R₅, R₉, R₁₂, R₁₃′, R₁₄′, and R₆₂ are as defined for the compound of formula (PVa).

In some embodiments, the PKC activator comprises a compound of formula (PVc):

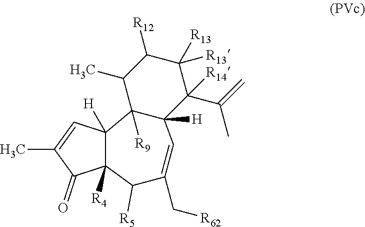

or an enantiomer, hydrate, solvate, or pharmaceutically acceptable salt thereof

wherein,

R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, or R₉′ is an O atom which is bonded to an optionally substituted common C atom bonded to R₁₃′ and R₁₄′, wherein R₁₃′ and R₁₄′ each is an O atom;

R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, optionally substituted arylalkyloxycarbonyl, or R₉ is an O atom which is bonded to an optionally substituted common C atom bonded to R₁₃′ and R₁₄′, wherein R₁₃′ and R₁₄′ each is an O atom;

R₁₂ is H, halo, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or R₁₂ is —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;

R₁₃ is H, halo, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, or optionally substituted heteroarylalkenylcarbonyl;

R₁₃′ and R₁₄′ are independently H or OH, or R₁₃′ and R₁₄′ are each an O atom which is bonded to an optionally substituted common C atom which is bonded to R₉, wherein R₉ is an O atom; and

R₆₂ is H, halo, or —O—R_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group at R₆₂.

In some embodiments, the PKC activator comprises a compound of formula (PVd):

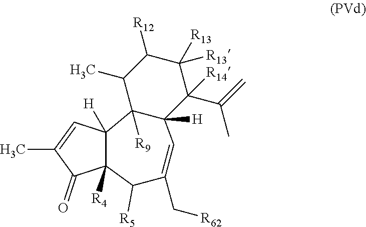

or an enantiomer, hydrate, solvate, or pharmaceutically acceptable salt thereof,

wherein

R₄, R₅, R₉, R₁₂, R₁₃, R₁₃′, R₁₄′ and R₆₂ are as defined for formula (PVc).

In some embodiments, the PKC activator comprises a compound of formula (PVe):

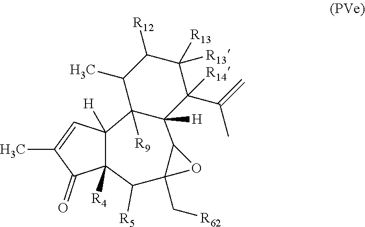

or an enantiomer, hydrate, solvate, or pharmaceutically acceptable salt thereof,

wherein

R₄, R₅, R₉, R₁₂, R₁₃, R₁₃′, R₁₄′ and R₆₂ are as defined for formula (PVc).

In some embodiments, the daphnane PKC activator is a compound selected from GD-1, yuanhuacine, sapintoxin D, thymeleatoxin A, simplexin, gnidimacrin, pimelea factor S7, genididin, geniditrin and gnidilatin. In some embodiments, the prodrugs for the specified daphnane compounds contain a biohydrolyzable carbonate, biohydrolyzable ureide, biohydrolyzable carbamate, biohydrolyzable ester, biohydrolyzable amide, or biohydrolyzable phosphate group. In particular, the prodrug for the specified daphnane compounds contains a biohydrolyzable ester, more particularly at the C20 carbon of formula (PV).

In some embodiments, the diterpenoid PKC activator is a lathyrane compound having PKC activating activity. In some embodiments, the PKC activator comprises a compound of formula (PVI):

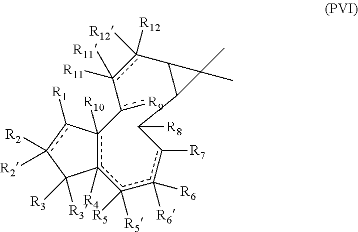

or an enantiomer, hydrate, solvate, or pharmaceutically acceptable salt thereof,
wherein

R₁ is H, or R₁ with R₂′ is a bond, or is bonded to a common O atom with R₂′ to form a epoxide ring;

R₂ is CH₃—, HOCH₂—, CH₃(O)COCH₂—; CH₃(O)COCH₂—, or phenyl(O)COCH₂—;

R₂′ is H, OH, CH₃, or R₂′ with R₁ is a bond, or is bonded to a common O atom with R₁ to form an epoxide ring;

R₃ is OH, C₁₋₆alkyl (O)CO—; C₂₋₆ alkenyl(O)CO—, phenyl(O)CO—; phenylC₁₋₄ alkyl(O)CO—, phenylC₂₋₄alkenyl(O)CO—; pyrrolidinyl(O)CO—; or R₃ with R₃′ is ═O,

R₃′ is H, or R₃′ with R₃ is ═O;

R₄ is H, or R₄ with R₁₀ or with R₅′ is a bond, or is bonded to a common O atom with R₁₀ to form an epoxide ring;

R₅ is H, —CH₃, OH, CH₃(O)CO—, C₁₋₄alkyl-O—, or phenylC₂₋₄ alkenyl(O)CO—,

R₅′ is H, or R₅′ with R₄ or R₆′ is a bond, or is bonded to a common O atom with R₆′ to form an epoxide ring;

R₆ is H, CH₃—, CH₂— which is bonded to a common O atom with R₆′ to form an epoxide ring, or R₆ with R₆′ is ═CH₂;

R₆′; is H, OH, CH₃(O)CO—, or R₆′ with R₅′ or R₇ is a bond, or is bonded to a common O atom with R₅′ to form the epoxide ring;

R₇ is H, OH, CH₃(O)CO—, phenyl(O)CO—, C₂₋₈ alkenyl-(O)CO—, or phenylC₁₋₄ alkyl-(O)CO—;

R₈ is H, OH, C₁₋₆ alkyl-O—, C₁₋₄alkyl(O)CO—, C₂₋₆ alkenyl(O)CO—, or phenyl(O)CO—;

R₉ is OH, ═O or ═CH₂;

R₁₀ is H, OH, CH₃(O)CO—, phenylC₂₋₄ alkenyl(O)CO—, or R₁₀ with R₄ is a bond, or is bonded to a common O atom with R₄ to form the epoxide ring;

R₁₁ is H, CH₃—, CH₂OH, or R₁₁ with R₁₁′ is ═CH₂,

R₁₁′ is H, or R₁₁′ with R₁₂′ is a bond;

R₁₂ is H, OH, C₁₋₆ alkyl(O)CO—, or C₂₋₆ alkenyl(O)CO—; and

R₁₂′ is H, OH, or R₁₂′ with R₁₁′ is a bond; and

[symbol] represents an optional double bond, with the proviso that

when a double bond is present between C4 and C10 atoms, there is no double bond between the C4 and C5 atoms;

when a double bond is present between C4 and C5 atoms, there is no double bond between the C4 and C10 atoms and the C5 and C6 atoms;

when a double bond is present between C5 and C6 atoms, there is no double bond between the C4 and C5 atoms and C6 and C7 atoms; and

when a double bond is present between C6 and C7 atoms, there is no double bond between the C5 and C6 atoms.

In some embodiments of the compound of formula (PVI), the phenylC₂₋₄ alkenyl(O)CO— is a cinnamate group. In some embodiments of the compound of formula (PVI), the C₂₋₆ alkenyl(O)CO— is selected from tiglate and angelate groups.

In some embodiments, the PKC activator comprises a compound of formula (PVIa):

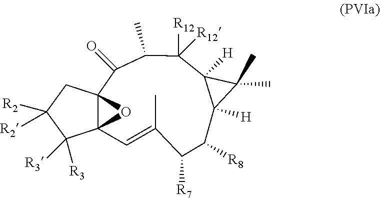

or an enantiomer, hydrate, solvate, or pharmaceutically acceptable salt thereof,

wherein

R₂ is CH₃—, HOCH₂—, CH₃(O)CO—, CH₃(O)COCH₂—, or phenyl(O)COCH₂—;

R₂′ is H, OH, or CH₃—;

R₃ is OH, C₁₋₆alkyl(O)CO—; C₂₋₆alkenyl(O)CO—, phenyl(O)CO—; phenylC₁₋₄alkyl-(O)CO—, phenylC₂₋₄alkenyl(O)CO—; pyrrolidinyl-(O)CO—; or R₃ with R₃′ is ═O;

R₃′ is H, or R₃′ with R₃ is ═O;

R₇ is H, OH, CH₃(O)CO—, phenyl(O)CO—, C₂₋₈ alkenyl-(O)CO—, or phenylC₁₋₄ alkyl-(O)CO—

R₈ is H, OH, C₁₋₆ alkyl-O—, C₁₋₄alkyl(O)CO—, C₂₋₆ alkenyl-(O)CO—, or phenyl(O)CO—,

R₁₂ is H, OH, C₁₋₆ alkyl-(O)CO—, or C₂₋₆ alkenyl-(O)CO—; and

R₁₂′ is H or OH.

In some embodiments, the PKC activator comprises a compound of formula (PVIb):

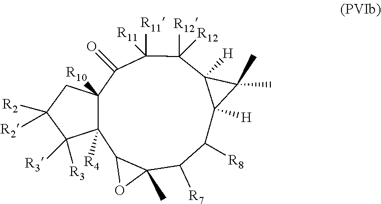

or an enantiomer, hydrate, solvate, or pharmaceutically acceptable salt thereof,

wherein

R₂ is CH₃—, HOCH₂—, CH₃(O)CO—, CH₃(O)COCH₂—, or phenyl(O)COCH₂—;

R₂′ is H, OH, CH₃, or R₂′ with R₁ is a bond;

R₃ is OH, C₁₋₆ alkyl-(O)CO—; C₂₋₆ alkenyl-(O)CO—, phenyl(O)CO—; phenylC₁₋₄alkyl-(O)CO—, phenylC₂₋₄alkenyl(O)CO—; pyrrolidinyl(O)CO—; or R₃ with R₃′ is ═O,

R₃′ is H, or R₃′ with R₃ is ═O;

R₄ is H or R₄ with R₁₀ is a bond;

R₇ is H, OH, CH₃(O)CO—, phenyl(O)CO—, C₂₋₈ alkenyl-(O)CO—, or phenylC₁₋₄alkyl-(O)CO—;

R₈ is H, OH, C₁₋₆alkyl-O—, C₁₋₄alkyl(O)CO—, C₂₋₆ alkenyl-(O)CO—, or phenyl(O)CO—;

R₁₀ is H, OH, CH₃(O)CO—, phenylC₂₋₄ alkenyl(O)CO—, or R₁₀ with R₄ is a bond;

R₁₁ is CH₃—, HOCH₂—, or R₁₁ with R₁₁′ is ═CH₂,

R₁₁′ is H, or R₁₁′ with R₁₂′ is a bond;

R₁₂ is H, OH, C₁₋₆ alkyl-(O)CO—, or C₂₋₆ alkenyl-(O)CO—; and

R₁₂′ is H, OH, or R₁₂′ with R₁₁′ is a bond.

In some embodiments, the PKC activator comprises a compound of formula (PVIc):

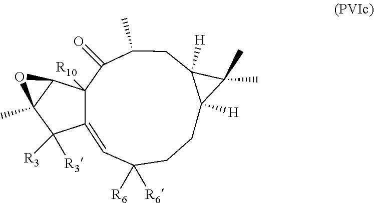

or an enantiomer, hydrate, solvate, or pharmaceutically acceptable salt thereof,

wherein

R₃ is OH, C₁₋₆alkyl-(O)CO—; C₂₋₆ alkenyl-(O)CO—, phenyl(O)CO—; phenylC₁₋₄ alkyl-(O)CO—, phenylC₂₋₄alkenyl(O)CO—; pyrrolidinyl-(O)CO—; or R₃ with R₃′ is ═O;

R₃′ is H, or R₃′ with R₃ is ═O;

R₆ is H, —CH₃, —CH₂ which is bonded to a common O atom with R₆′ to form an epoxide ring, or R₆ with R₆′ is ═CH₂;

R₆′; is H, OH, or CH₃(O)CO—; and

R₁₀ is H, OH, CH₃(O)CO— or phenylC₂₋₄ alkenyl(O)CO—.

In some embodiments, the PKC activator comprises a compound of formula (PVId):

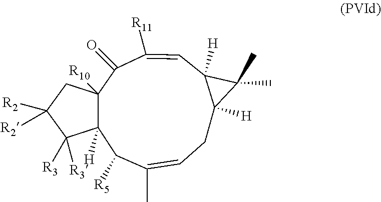

or an enantiomer, hydrate, solvate, or pharmaceutically acceptable salt thereof,

wherein

R₂ is —CH₃, HOCH₂—, CH₃(O)CO—, CH₂(O)COCH₂— or phenyl(O)COCH₂—;

R₂′ is H, OH or CH₃;

R₃ is OH, C₁₋₆alkyl-(O)CO—, C₂₋₆ alkenyl-(O)CO—, phenyl(O)CO—; phenylC₁₋₄alkyl-(O)CO—, phenylC₂₋₄alkenyl(O)CO—; pyrrolidinyl(O)CO—; or R₃ with R₃′ is ═O;

R₃′ is H, or R₃′ with R₃ is ═O;

R₆ is H, CH₃—, —CH₂— which is bonded to a common O atom with R₆′ to form an epoxide ring, or R₆ with R₆′ is ═CH₂;

R₆′; is H, OH, or CH₃(O)CO—;

R₁₀ is H, OH, CH₃(O)CO—, or phenylC₂₋₄ alkenyl(O)CO—; and

R₁₁ is H, CH₃—, or HOCH₂—.

In some embodiments, the lathyrane PKC activating compound is selected from the exemplary lathyrane compounds including, among others, 7,8,12-O-triacetyl-3-O-(2-methylbutanoyl)ingol, 3,12-O-diacteyl-7-O-(2-methylbutanoyl)-8-methylingol, 3,7,12-O-triacetyl-8-Obenzoyl-2-epi-ingol, 3,12-O-diacetyl-7-O-angeloyl8-methoxyingol, 7,12-O-diacetyl-3-Ophenylacetyl-8-methoxyingol, euphorbia factor L1, euphorbia factor L10, deoxy euphorbia factor L1, euphorbia factor L2, euphorbia factor L3, euphorbia factor L8, euphorbia factor L11, euphorbia factor L9, jolkinol B, jolkinol A, jolkinol C, jolkinol D, latilagascene A, latilagascene B, latilagascene C, latilagascene D, latilagascene E, japodagrol, japodagrin, latazienone, jatrowedione, euphohelioscopin A, euphohelioscopin B, euphohelioscopin C, jatrowediol, curculathyrane A, and curculathyrane B, or solvates, hydrates, and prodrugs thereof. In some embodiments, the lathyrane PKC activating compound is selected from the exemplary lathyrane compounds shown below.

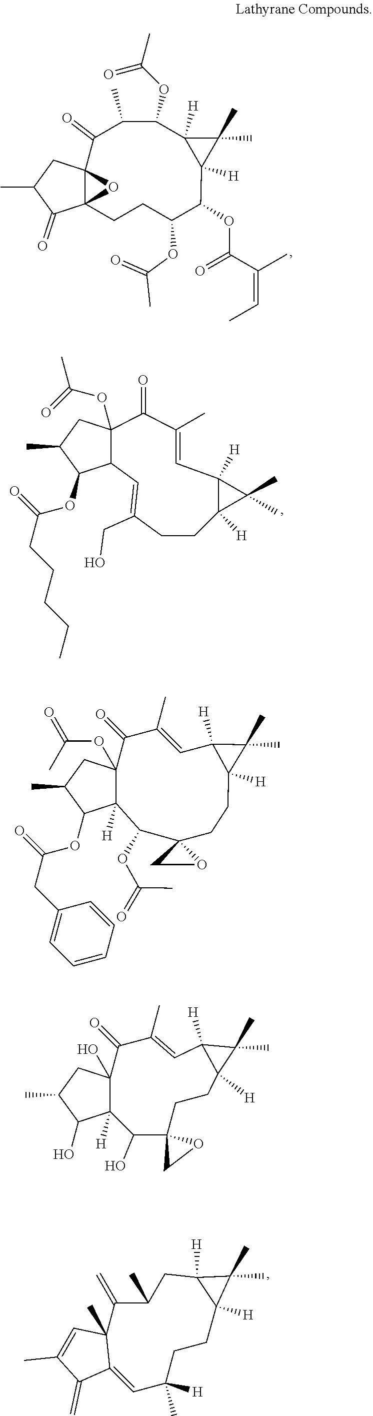

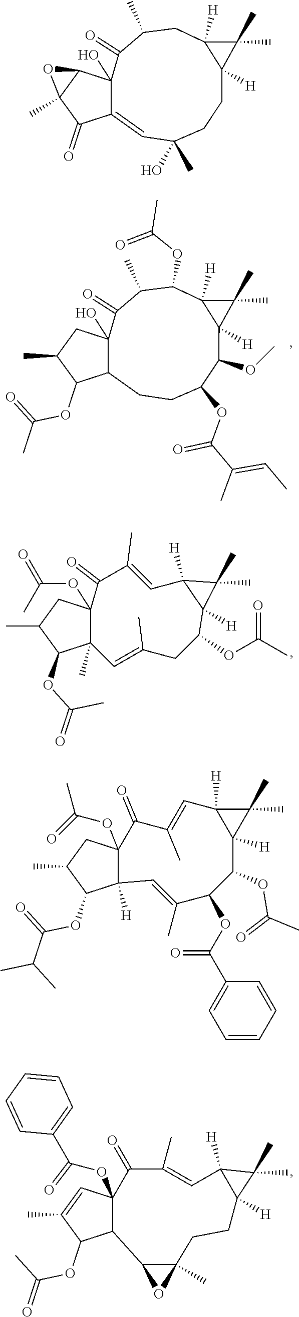

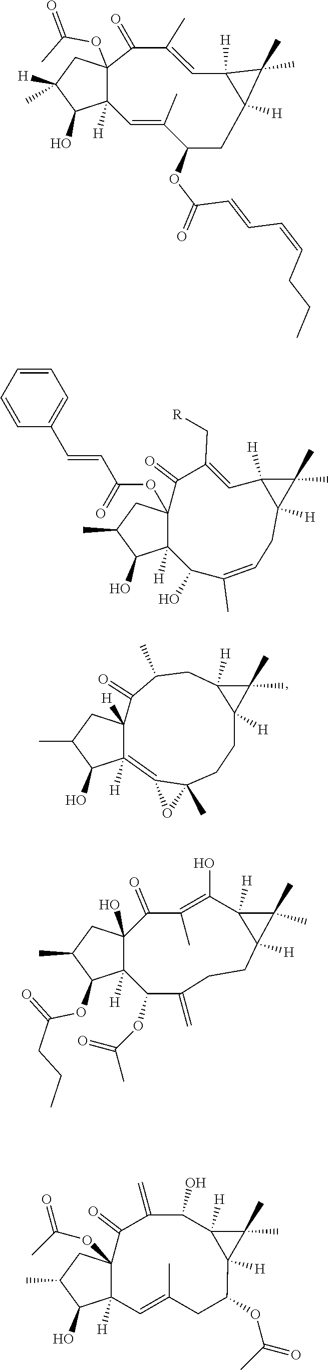

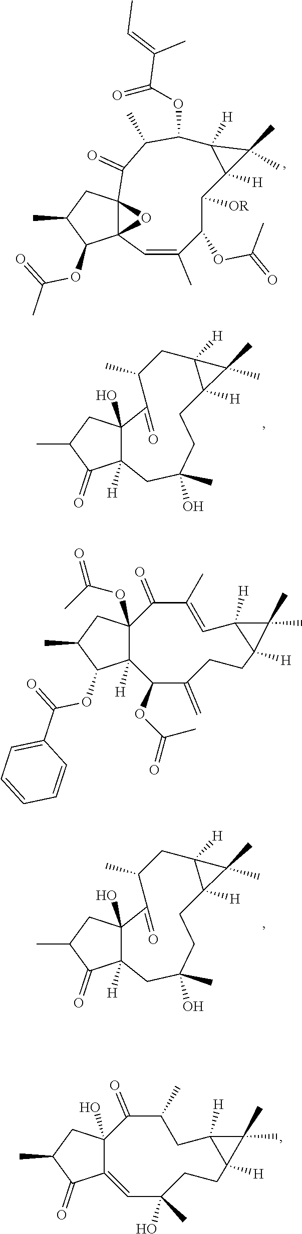

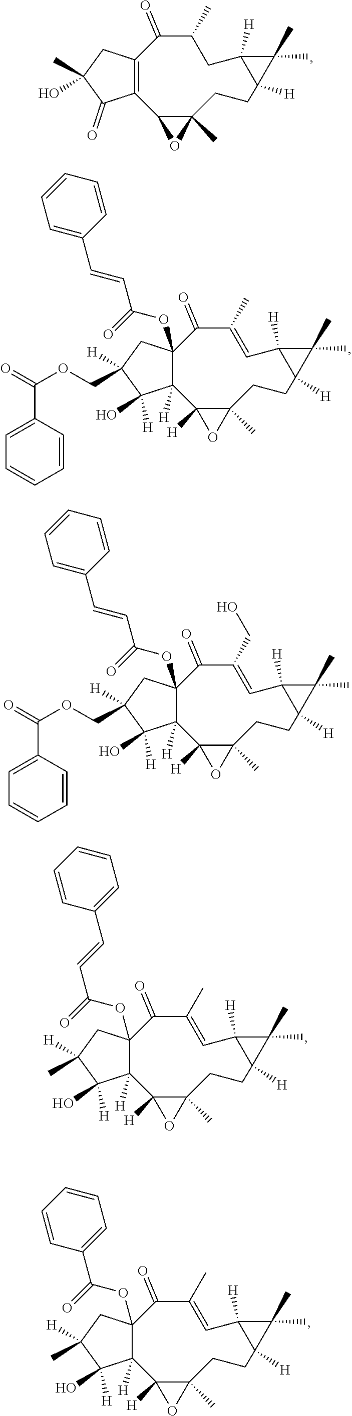

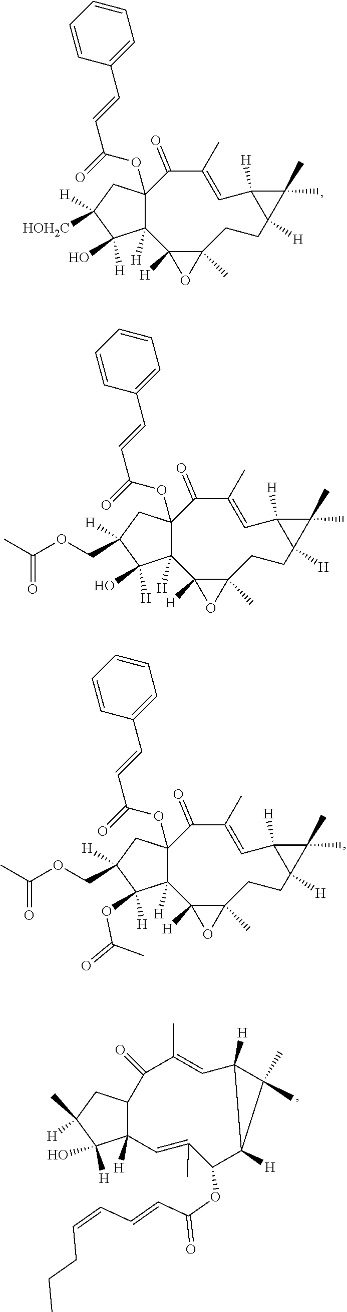

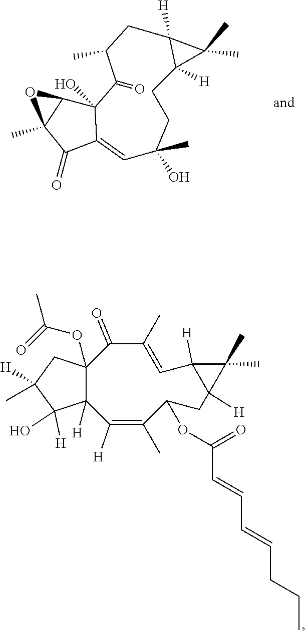

and pharmaceutically acceptable salts thereof.

In some embodiments where the cancer is to be treated with a combination of a ferroptosis inducer and a diterpenoid PKC activator, the cancer for treatment can be selected for sensitivity to the diterpenoid PKC activator, particularly by assessing the PKC activation potential of the cancer (see, e.g., International application PCT/US2016/61711, incorporated herein by reference). In some embodiments, a method of treating a subject with cancer comprises determining or identifying a PKC activation potential of the cancer for a PKC activator, and administering to the subject having a cancer determined to have an effective PKC activation potential a therapeutically effective amount of a ferroptosis inducer and a PKC activator, particularly a diterpenoid PKC activator compound. In some embodiments, a method of treating a subject with cancer comprises administering to a subject in need thereof a therapeutically effective amount of a ferroptosis inducer and a PKC activator, wherein the cancer has been determined or identified as having an effective PKC activation potential for the PKC activator.

In some embodiments, the PKC activation potential can take into account (a) the basal level of PKC activity present in the cancer cell, and/or (b) the increase in PKC activity upon contacting the cancer cell or upon treatment of the cancer with the PKC activator. In some embodiments, the level of PKC activity can be assessed for total PKC activity or activity of one or more specific PKC isoforms.

The presence of an effective PKC activation potential for a PKC activator can be determined by various methods. In some embodiments, the effective PKC activation potential can be determined by measuring the level of PKC activation in cancer cells sensitive to the PKC activator, e.g., based on inhibition of cell proliferation. For example, the level of PKC activation associated with 50% inhibition of cell proliferation (IC₅₀) by a PKC activator can be used as an effective PKC activation potential for the PKC activator. In some embodiments, a cancer cell insensitive or resistant to the PKC activator, e.g., insignificant effect on cell proliferation at concentration of PKC activator sufficient to inhibit proliferation of PKC-activator sensitive cells (e.g., IC₅₀), can be used to identify the PKC enzyme activated by the PKC activator in sensitive cancer cells. In some embodiments, the basal level of PKC activity in PKC activator sensitive cells as compared to level of PKC activity in PKC activator insensitive cells can be used to determine a basal level of PKC activity, either as total PKC activity or activity of one or more specific PKC isoforms associated with sensitivity to the PKC activator.

In some embodiments, the effective PKC activation potential for a PKC activator can be determined by the use of a PKC inhibitor. The PKC inhibitor can be a broad spectrum inhibitor or a specific inhibitor targeting one or a limited set of the PKC isoforms. In some embodiments, a cancer cell sensitive to a PKC activator can be treated with different concentrations of a PKC inhibitor and then treated with the PKC activator. The reduction in PKC activator-mediated inhibition of cell proliferation by treatment with the PKC inhibitor and the associated level of PKC activation can provide a measure of the level of effective PKC activation sufficient for inhibiting cell proliferation. In some embodiments, the PKC inhibitor used is an inhibitor specific to a PKC isoform or specific to a limited set of PKC isoforms.

In some embodiments, a cancer with a basal level of total PKC activity of at least 20%, 30%, 40%, 50%, 60%, 70%, 80%, 90% or more of the basal PKC activity present in a suitable control, for example non-cancerous cells or tissues or normal cells or tissues, can provide an indication of sensitivity to a PKC activator, and thus a basis for selection of the cancer for treatment with a combination of the ferroptosis inducer and PKC activator.

In some embodiments, a cancer which displays an increase in total PKC activity of at least 30%, 40% 50%, 60%, 70%, 80%, 90%, 100%, 150%, 200% or more above the PKC activity of untreated cancer cells, in presence of or following treatment with the PKC activator indicates sensitivity to the PKC activator and thus a basis for selection of the cancer for treatment with a combination of the ferroptosis inducer and PKC activator.

In some embodiments, a cancer which has increased total PKC activity upon treatment with the PKC activator, such as in the foregoing, and in which the total PKC activity following treatment is at least 30%, 40%, 50%, 60%, 70%, 80%, 90%, 100%, 150%, 200% or more of the PKC activity of control non-cancerous cells or tissue indicates sensitivity to the PKC activator and thus a basis for selection of the cancer for treatment with a combination of the ferroptosis inducer and PKC activator.

In some embodiments, the selection of a cancer for treatment with a combination of the ferroptosis inducer and PKC activator is based on the PKC activation potential for one or more of PKC isoforms. In some embodiments, the PKC activation potential is determined or measured for one or more of PKC isoforms selected from PKC α, β, γ, δ, ε, η, θ, ι/λ, μ and ζ. In some embodiments, the PKC activation potential is determined or measured for one or more classical PKCs. Exemplary classical PKCs include PKC α, β (e.g., βI, βII), and γ. In some embodiments, the PKC activation potential is determined or measured for one or more novel PKCs. Exemplary novel PKCs include δ, ε, η, and θ. In some embodiments, the PKC activation potential is determined or measured for one or more atypical PKCs. Exemplary atypical PKCs include ι/λ, and ζ. In some embodiments, the PKC activation potential is determined or measured for PKCμ, which is a member of the protein kinase D (PKD) family.

In some embodiments, a cancer with a basal level of PKC activity of at least 20%, 30%, 40%, 50%, 60%, 70%, 80%, 90% or more of the basal PKC activity for one or more of PKC isoforms selected from PKC α, β, γ, δ, ε, η, θ, ι/λ, μ and ζ as compared to a suitable control level, for example the basal level in non-cancerous cells or tissues (e.g., normal cells or tissues), can provide an indication of sensitivity to a PKC activator, and thus a basis for selection of the cancer for treatment with a combination of the ferroptosis inducer and PKC activator. In some embodiments, the determination of a basal level of PKC activity can be useful where the PKC activity is known to be expressed in the control cells or tissues in the absence of treatment with the PKC activator.

In some embodiments, a cancer which displays or is capable of an increase in one or more of PKC α, β, γ, δ, ε, θ, ι/λ, μ and ζ activity of at least 30%, 40%, 50%, 60%, 70%, 80%, 90%, 100%, 150%, 200% or more above the PKC α, β, γ, δ, ε, η, θ, ι/λ, μ and ζ activity, respectively, of a control level, e.g., untreated cancer cells, in presence of or following treatment with the PKC activator is selected for treatment with a combination of the ferroptosis inducer and PKC activator.

In some embodiments, the PKC activation potential is measured for one or more of PKC α, β, and γ. In some embodiments, a cancer with a basal level of PKC α, β, or γ activity of at least 20%, 30%, 40%, 50%, 60%, 70%, 80%, 90% or more of the basal PKC α, β, or γ activity, respectively, of a control level, e.g., normal cells or normal tissue, is indicated for treatment with a combination of the ferroptosis inducer and PKC activator. In some embodiments, a cancer which displays or is capable of an increase in one or more of PKC α, β, and γ activity of at least 30%, 40%, 50%, 60%, 70%, 80%, 90%, 100%, 150%, 200% or more above the PKC α, β, or γ activity, respectively, of a control level, e.g., untreated cancer cells, in presence of or following treatment with the PKC activator is selected for treatment with a combination of the ferroptosis inducer and PKC activator. In some embodiments, a cancer which displays or is capable of an increase in PKC α, β, or γ activity upon treatment with the PKC activator, such as in the foregoing, and in which the total PKC α, β, or γ activity is at least 30%, 40%, 50%, 60%, 70%, 80%, 90%, 100%, 150%, 200% or more of the PKC activity of a control level, e.g., untreated cells, in presence of or following treatment with the PKC activator is selected for treatment with a combination of the ferroptosis inducer and PKC activator.

In some embodiments, the PKC activation potential is measured for one or more of PKC δ, ε, η, or θ. In some embodiments, a cancer with a basal level of PKC δ, ε, η, or θ activity of at least 20%, 30%, 40%, 50%, 60%, 70%, 80%, 90% or more of the basal PKC δ, ε, η, or θ activity, respectively, of a control level, e.g., normal cells or normal tissue, is indicated for treatment with a combination of the ferroptosis inducer and PKC activator. In some embodiments, a cancer which displays or is capable of an increase in one or more of PKC δ, ε, η, or θ activity of at least 30%, 40%, 50%, 60%, 70%, 80%, 90%, 100%, 150%, 200% or more above the PKC δ, ε, η, or θ activity, respectively, of a control level, e.g., untreated cancer cells, in presence of or following treatment with the PKC activator is selected for treatment with a combination of the ferroptosis inducer and PKC activator. In some embodiments, a cancer which displays or is capable of an increase in PKC δ, ε, η, or θ activity upon treatment with the PKC activator, such as in the foregoing, and in which the total PKC δ, ε, η, or θ activity is at least 30%, 40%, 50%, 60%, 70%, 80%, 90%, 100%, 150%, 200% or more of the PKC activity of a control level, e.g., untreated cells, in presence of or following treatment with the PKC activator is selected for treatment with a combination of the ferroptosis inducer and PKC activator.

In some embodiments, the PKC activation potential is measured for one or more of PKC ι/λ, μ or ζ. In some embodiments, a cancer with a basal level of PKC ι/λ, μ or ζ activity of at least 20%, 30%, 40%, 50%, 60%, 70%, 80%, 90% or more of the basal PKC ι/λ, μ or ζ activity, respectively, of a control level, e.g., normal cells or normal tissue, is indicated for treatment with a combination of the ferroptosis inducer and PKC activator. In some embodiments, a cancer which displays or is capable of an increase in one or more of PKC ι/λ, μ or ζ activity of at least 30%, 40%, 50%, 60%, 70%, 80%, 90%, 100%, 150%, 200% or more above the PKC ι/λ, μ or ζ activity, respectively, of a control level, e.g., untreated cancer cells, in presence of or following treatment with the PKC activator is selected for treatment with a combination of the ferroptosis inducer and PKC activator. In some embodiments, a cancer which displays or is capable of an increase in PKC ι/λ, μ or ζ activity upon treatment with the PKC activator, such as in the foregoing, and in which the total PKC ι/λ, μ or ζ activity is at least 30%, 40%, 50%, 60%, 70%, 80%, 90%, 100%, 150%, 200% or more of the PKC activity of a control level, e.g., untreated cells, in presence of or following treatment with the PKC activator is selected for treatment with a combination of the ferroptosis inducer and PKC activator.

In some embodiments, the PKC activation potential can be assessed by detecting or measuring one or more phosphorylated amino acid sequences in the PKC enzyme, particularly phosphorylation associated with activation or activity of the PKC enzyme. In some embodiments, the phosphorylated amino acid sequence detected has (i) increased phosphorylation induced by the PKC activator in a control, e.g., normal cells, and/or (ii) increased phosphorylation in control PKC activator-sensitive cancer cells but not in control PKC activator insensitive cancer cells. Determining the PKC activation potential can be based on identified phosphorylated amino acid sequences in one or more of PKC α, β (e.g., βI or βII,), γ, δ, ε, η, θ, ι/λ, μ, and ζ, particularly phosphorylated amino acid sequences localized in the protein kinase domain and carboxy terminal tail of the PKC, also referred to as the C3 and C4 domains (see., e.g., Newton, A. C., 2010, Am J Physiol Endocrinol Metab. 298:E395-E402; Steinberg, S. F., 2008, Physiol Rev. 88(4):1341-1378; incorporated herein by reference). Compilation of phosphorylated sites in each of the PKC enzymes is available at PhosphoSitePlus® at world wide web (www) at phosphosite.org. While the amino acid positions of the phosphorylation sited are indicated for human PKC enzymes, the equivalent sites can be identified in other mammals. For example, equivalent amino acid positions in mouse are provided in parenthesis.

In some embodiments, determining PKC activation potential for human PKCα can measure phosphorylation at one or more of S226, T228, T497, T638, S657 and Y658, particularly T497, T638, S657 and Y658.

In some embodiments, determining PKC activation potential for human PKCβ (βI/βII) can measure phosphorylation at one or more of Y368, T500, T504, Y507, Y515, Y518, T635, T642(641), and S661(660), particularly T500, T642(641), and S661(660).

In some embodiments, determining PKC activation potential for human PKCγ can measure phosphorylation at one or more of T514, T518, Y521, Y529, Y532, T655, T674 and S687, particularly T514 and T674.

In some embodiments, determining PKC activation potential for human PKCδ can measure phosphorylation at one or more of Y64, T141, Y187, T295, S299, Y313 (Y131), Y374, S503, T505, S506, T507, T511, Y514, Y567, Y630, S643, S645, Y646, S647, S658, and S664, particularly Y64, Y187, Y313, T505, T507 (T505), T511, Y514, Y567, Y630, S643, S645, Y646, S647, S658, and S664.

In some embodiments, determining PKC activation potential for human PKCε can measure phosphorylation at one or more of Y250, T309, S329, S337, S346, S350, S368, S388, T566, T710, and S729, particularly T566, T710, and S729.

In some embodiments, determining PKC activation potential for human PKCη can measure phosphorylation at one or more of S28, S32, Y94, S317, S327, Y381, T656, S676, S685, S695, and S675, particularly S327, Y381, T656, and S675.

In some embodiments, determining PKC activation potential for human PKCθ. can measure phosphorylation at one or more of Y90, T219, T307, T536, T538, Y545, S676, S685, and S695, particularly Y90, T538, S676, S685 and S695.

In some embodiments, determining PKC activation potential for human PKCι/λ can measure phosphorylation at one or more of Y136, T403, T409, T410, S411, T412, S459, T555, T557, T564, Y584, and S591, particularly T403, T409, T410, S411, T412, S459, T555, T557, T564, Y584, and S591.

In some embodiments, determining PKC activation potential for human PKCμ can measure phosphorylation at one or more of Y95, S205, S208, S219, S223, Y463, S738, S742, T746, S748, and S910, particularly Y95, Y463, S738, S742, S744, T746, S748, and S910, more particularly S910. The S910, Ser738, and Ser742 in human PKC are equivalent to Ser916, Ser744, and Ser748, respectively, in mouse PKCμ.

In some embodiments, determining PKC activation potential for human PKCζ can measure phosphorylation at one or more of S262, Y263, S375, T410, Y417, Y428, S520, T560, and S591, particularly T410, Y417, Y428, S520, T560, and S591.

In some embodiments, the PKC activation potential is determined by measuring phosphorylation at the kinase domain activation loop, the turn motif, and/or the hydrophobic motif of the PKC. In some embodiments, the PKC activation potential is determined by detecting phosphorylation at the kinase domain activation loop. Exemplary phosphorylations occurring at the activation loop of human PKCs include T497 for PKCα, T500 for PKCβ, T514 for PKCγ, T505 for PKCδ, T538 for PKCθ, T566 for PKCε, T512 for PKCη, T410 for PKCζ, T403 for PKCι/λ, and S738/S742 for PKCμ.

In some embodiments, the PKC activation potential is determined by measuring phosphorylation at the kinase domain turn motif. Exemplary phosphorylations occurring at the turn motif of PKCs include S638 for PKCα, T641 for PKCβ(βII and βII), T655 for PKCγ, T643 for PKCδ, S676 for PKCθ, T710 for PKCε, T645 for PKCη, and T560 for PKCζ.

In some embodiments, the PKC activation potential is determined by measuring phosphorylation at the kinase domain hydrophobic motif and/or carboxy terminal domain. Exemplary phosphorylations occurring at the hydrophobic motif and/or carboxy terminal domain include S657 for PKCα, S660 for PKCβ (βII and βII), S674 for PKCγ, S662 for PKCδ, S695 for PKCθ, S729 for PKCε, S664 for PKCη, and S910 for PKCμ.

In some embodiments, the PKC activation potential is determined by measuring phosphorylation at one or more autophosphorylation sites in the PKC enzyme. Exemplary autophosphorylation sites include: S638 for PKCα, T641 for PKCβ, T141/T295/T514 for PKCγ, T295/T505 for PKCδ, T219/T538/S676/S695 for PKCθ, S729 for PKCε, T655 for PKCη, T560 for PKCζ, and S738/S742/S910 for PKCμ.

In some embodiments, the PKC activation potential is determined for phosphorylation of PKCμ at Ser910, which is equivalent to Ser916 in mouse. In some embodiments, a method of determining the sensitivity of a cancer or selecting a cancer for treatment with a combination of the ferroptosis inducer and PKC activator includes determining the level of phosphorylated PKCμ at Ser910 in the cancer, wherein an elevated level of phosphorylated PKCμ at Ser910 upon treatment with the PKC activator indicates sensitivity of the cancer to the PKC activator. In some embodiments, a cancer or a subject with cancer is selected for treatment with a combination of the ferroptosis inducer and PKC activator if the cancer is determined to have (i) an elevated level of phosphorylated PKCμ at Ser910 upon treatment of the cancer with the PKC activator, or (ii) an elevated level of phosphorylated PKCμ at Ser910 upon treatment of the cancer with the PKC activator as compared to a control level, e.g., basal level in untreated cancer or normal cells or tissues. In some embodiments, a method of treating a subject with cancer comprises administering to a subject in need thereof a therapeutically effective amount of a ferroptosis inducer and a PKC activator, e.g., a diterpenoid PKC activator, wherein the cancer is determined to have an elevated level of phosphorylated PKCμ at Ser910 upon treatment of the cancer with the PKC activator.

It is to be understood that in some embodiments, phosphorylation of a PKC can be correlated with insensitivity of a cancer to a PKC activator, in contrast to phosphorylation of a PKC that is correlated with sensitivity to the PKC activator. In some embodiments, the phosphorylated PKC can be present endogenously in the absence of treatment with a PKC activator, where presence of the phosphorylated PKC correlates with insensitivity to the PKC activator. In some embodiments, the phosphorylation of the PKC correlated with insensitivity to the PKC activator occurs in response to treatment of the cancer with a PKC activator. In some embodiments, the phosphorylation correlated with insensitivity to a PKC activator is phosphorylation of PKCδ, particularly phosphorylation of PKCδ at Tyr311. In some embodiments, a method of determining the sensitivity of a cancer or selecting a cancer for treatment with a combination of the ferroptosis inducer and PKC activator includes determining the level of phosphorylated PKCδ at Tyr311 in the cancer, wherein (i) an absence of phosphorylated PKCδ at Tyr311, or (ii) a basal level of phosphorylated PKCδ at Tyr311 as compared to a control level, e.g., basal level in control PKC activator sensitive cancer, indicates sensitivity of the cancer to the PKC activator. A basal level as used in this context refers to the level of phosphorylated PKCδ at Tyr311 in control PKC activator sensitive cancer cells, with or without treatment with the PKC activator. In some embodiments, a cancer or a subject with a cancer is selected for treatment with a combination of the ferroptosis inducer and PKC activator if the cancer has: (i) an absence of phosphorylated PKCδ at Tyr311, or (ii) a basal level of phosphorylated PKCδ at Tyr311 as compared to a control level. In some embodiments, a method of treating a subject with cancer comprises administering to the subject in need thereof a therapeutically effective amount of a ferroptosis inducer and a PKC activator, e.g., a diterpenoid PKC activator, wherein the cancer is determined to have: (i) an absence of phosphorylated PKCδ at Tyr311, and/or (ii) a basal level of phosphorylated PKCδ at Tyr311 as compared to a control level, e.g., basal level in control PKC activator sensitive cancer cells or tissues.

In some embodiments, a cancer or a subject with cancer is not selected for treatment with a ferroptosis inducer and PKC activator if the cancer is determined to have (i) phosphorylated PKCδ at Tyr311, and/or (ii) an elevated level of phosphorylated PKCδ at Tyr311 as compared to a basal control level, e.g., level in control PKC activator sensitive cancer cells or tissues, or normal cells or tissue. In some embodiments, a cancer or a subject with cancer is not selected for treatment with a ferroptosis inducer and PKC activator when the level of phosphorylated PKCδ at Tyr311 is elevated compared to a control basal level, e.g., basal level in PKC activator sensitive cancer cells or tissues, or normal cells or tissues. In some embodiments, the sensitivity or insensitivity of a cancer to a PKC activator can be based on assessment of the level of phosphorylated PKCμ at Ser910 (Ser916), and the level of phosphorylated PKCδ at Tyr311.

In some embodiments, a cancer or a subject with a cancer is selected for treatment with a ferroptosis inducer and PKC activator if the cancer is determined to have (i) an elevated level of phosphorylated PKCμ at Ser910 upon treatment with the PKC inhibitor, and (ii) an absence or a basal level of phosphorylated PKCδ at Tyr311 as compared to a control level. In some embodiments, a method of treating a subject with cancer comprises administering to the subject in need thereof a therapeutically effective amount of a ferroptosis inducer and PKC activator, e.g., a diterpenoid PKC activator, wherein the cancer is determined to have: (i) an elevated level of phosphorylated PKCμ at Ser910 upon treatment with the PKC inhibitor, and (ii) an absence or a basal level of phosphorylated PKCδ at Tyr311 as compared to a control level.

In some embodiments, the PKC activation potential can be assessed by determining the presence or absence of mutations in the gene encoding a PKC enzyme, where the mutations result in inactivation or attenuation of PKC activity, such as gene deletions and other loss-of-function mutations. The presence of such mutations in the PKC gene may result in low or no basal level of PKC activity and also display ineffective PKC activation upon treatment with the PKC activator. Accordingly, in some embodiments, the PKC activation potential is assessed by identifying or determining in the cancer the presence or absence of one or more loss-of-function mutations (e.g., inactivating or activity-attenuating) in the gene encoding the PKC enzyme. In various embodiments, a cancer determined or identified as being negative for loss-of-function mutations in one or more of PKC enzymes is selected for treatment with the combination of a ferroptosis inducer and PKC activator. In some embodiments, cancer determined or identified as being negative for two or more, three or more, four or more, or five or more loss-of-function mutations is selected for treatment with the combination. In some embodiments, a cancer is not selected for treatment if it is determined or identified as having loss-of-function mutations in one or more PKC enzymes. In some embodiments, a cancer is not selected for treatment if it is determined or identified as having two or more, three or more, four or more, or five or more loss-of-function mutations. In view of the presence of various PKC isoforms, in some embodiments, the cancer is not selected for treatment with the combination if two or more, three or more, four or more, or five or more PKC isoforms are determined or identified as having a loss-of-function mutation. In some embodiments, a cancer assessed for presence of a loss-of-function PKC mutation and measured for activation potential identifies the basis for selecting the cancer for treatment with the combination. In some embodiments, assessment based on identification of or absence of a loss-of-function mutation alone is used as the basis for selecting or not selecting the cancer for treatment with the combination.

In some embodiments, the loss-of-function mutation is assessed for one or more of PKC isoforms selected from PKC α, β, γ, δ, ε, η, θ, ι/λ, μ and ζ. In some embodiments, the loss-of-function mutation is assessed for one or more classical PKCs, including PKC α, β (e.g., βI, βII), and γ. In some embodiments, the loss-of-function mutation is assessed for one or more novel PKCs, including PKC δ, ε, η, and θ. In some embodiments, the loss-of-function mutation is assessed for one or more atypical PKCs, including PKC ι/λ and ζ. In some embodiments, the loss-of-function mutation is assessed for PKCμ.

In some embodiments, the loss-of-function mutation is assessed for one or more PKC isoforms selected from PKC α, β, and γ.

In some embodiments, the PKC is PKCα, and the loss-of-function mutation is an inactivating or activity-attenuating deletion or partial deletion of the gene encoding PKCα, or a loss-of-function mutation at one or more of codon 58, codon 61, codon 63, codon 75, codon 257, codon 435, codon 444, codon 481, codon 506, and codon 508. In some embodiments, the loss-of-function mutation in PKCα is one or more of αW58L, αG61W, αQ63H, αH75Q, αG257V, αF435C, αA444V, αD481E, αA506V, αA506T, and αE508K.

In some embodiments, the PKC is PKCβ, and the loss-of-function mutation is an inactivating or activity-attenuating deletion or partial deletion of the gene encoding PKCβ, or a loss-of-function mutation at one or more of codon 61, codon 353, codon 417, codon 484, codon 509, codon 523, codon 561, codon 585, and codon 619. In some embodiments, the loss-of-function mutation in PKCβ is one or more of βG61W, βF353L, βY417H, βD484N, βA509V, βA509T, βD523N, βP561H, βG585S, and βP619Q.

In some embodiments, the PKC is PKCγ, and the loss-of-function mutation is an inactivating or activity-attenuating deletion or partial deletion of the gene encoding PKCγ, or a loss-of-function mutation at one or more of codon 23, codon 57, codon 193, codon 218, codon 254, codon 362, codon 431, codon 450, codon 461, codon 498, codon 524, codon 537, and codon 575. In some embodiments, the loss-of-function mutation in PKCγ is one or more of γG23E, γG23W, γW57splice, γD193N, yT218M, γT218R, γD254N, γF362fs, γF362L, γG450C, γY431F, γA461T, γA461V, γD498N, yP524L, γP524R, γD537G, γD537Y, and γP575H.

In some embodiments, the loss-of-function mutation is assessed for one or more PKC isoforms selected from PKC δ, ε, η, or θ.

In some embodiments, the PKC is PKCδ, and the loss-of-function mutation is an inactivating or activity-attenuating deletion or partial deletion of the gene encoding PKCδ, or a loss-of-function mutation at one or more of codon 146, codon 454, codon 517, codon 530, and codon 568. In some embodiments, the loss-of-function mutation in PKCδ is one or more of δG146R, δA454V, δP517S, δD530G, δP568A, and δP568S.

In some embodiments, the PKC is PKCε, and the loss-of-function mutation is an inactivating or activity-attenuating deletion or partial deletion of the gene encoding PKCε, or a loss-of-function mutation at one or more of codon 162, codon 197, codon 502, and codon 576. In some embodiments, the loss-of-function mutation in PKCε is one or more of εR162H, εQ197P, εR502X, and εP576S.

In some embodiments, the PKC is PKCη, and the loss-of-function mutation is an inactivating or activity-attenuating deletion or partial deletion of the gene encoding PKCη, or a loss-of-function mutation at one or more of codon 284, codon 591, codon 596, and codon 598. In some embodiments, the loss-of-function mutation in PKCη is one or more of ηH1284Y, ηK591E, ηK591N, ηR596H, and ηG598V.

In some embodiments, the PKC is PKCθ, and the loss-of-function mutation is an inactivating or activity-attenuating deletion or partial deletion of the gene encoding PKC θ, or a loss-of-function mutation at one or more of codon 171, codon 485, codon 548, and codon 616. In some embodiments, the loss-of-function mutation in PKCθ is one or more of θW171X, θA485T, θP548S, and θR616Q.

In some embodiments, the loss-of-function mutation is assessed for one or more PKC isoforms selected from PKCι/λ, μ and ζ.

In some embodiments, the PKC is PKCι/λ, and the loss-of-function mutation is an inactivating or activity-attenuating deletion or partial deletion of the gene encoding PKCι/λ, or a loss-of-function mutation at one or more of codon 179, codon 359, codon 396, and codon 423. In some embodiments, the loss-of-function mutation in PKCι/λ is one or more of ιH179Y, ιS359, ιD396E, and ιE423D.

In some embodiments, the PKC is PKCμ, and the loss-of-function mutation is an inactivating or activity-attenuating deletion or partial deletion of the gene encoding PKCμ, or a loss-of-function mutation at one or more of the mutations found in breast and colon cancer (see, e.g., Kan et al., 2010, Nature 466:869-873).

In some embodiments, the PKC is PKCζ, and the loss-of-function mutation is an inactivating or activity-attenuating deletion or partial deletion of the gene encoding PKCζ, or a loss-of-function mutation at codon 421. In some embodiments, the loss-of-function mutation in PKCζ is E421K.

In some embodiments, the PKC loss-of-function mutation is in the kinase domain of PKC, which sequence is conserved in eukaryotic PKCs (see, e.g., Kornev et al., 2006, Proc Natl Acad Sci. USA 103:17783-17788, incorporated herein by reference). In some embodiments, the PKC loss-of-function mutation is a loss-of-function mutation in the activation loop, the turn motif, and/or the hydrophobic motif of the PKC kinase domain.

In some embodiments, the PKC mutations are dominant negative mutations, particularly dominant negative mutations which result in attenuated global PKC activity in the cancer cell and which can attenuate activation by PKC activators. In some embodiments, the dominant negative mutation is one or more of PKCα (e.g., H75Q), PKCγ (e.g., P524R), and PKCβ (e.g., A509V). In some embodiments, a subject with a cancer which is determined or identified as having one or more dominant negative PKC mutations is not selected for treatment with the combination. In some embodiments, a subject with a cancer which is determined or identified as being negative for at least one, at least two or more, at least three or more, or at least for or more dominant negative mutations in PKC are selected for treatment with the combination.

In some embodiments, the PKC activation potential can be assessed by determining or identifying in the cancer the presence or absence of mutations affecting interaction of the PKC enzyme with the PKC activator, particularly a diterpenoid PKC activator. In some embodiments, a cancer with identified mutations occurring in the C1 domain of PKC and affecting interaction with a diterpenoid PKC activator with the PKC is not selected for treatment with the combination. For example, exemplary mutations affecting the interaction of PKC with phorbol PKC activator are described in, for example, Wang et al., 2001, J Biol Chem. 276:19580-19587; and Kazanietz et al, 1995, J Biol Chem. 270:21852-21859; incorporated herein by reference. In some embodiments, a cancer determined or identified as negative for mutations affecting interaction of a PKC activator with the PKC protein is indicated for treatment with the combination.

In some embodiments, the assessment of the PKC activation potential of the cancer can also include determining or identifying the expression level of the PKC enzyme during or following treatment with the PKC activator. In some embodiments, the determining or identifying the expression level of the PKC enzyme is carried out as an adjunct to assessment of the PKC activation potential based on PKC activity, e.g., PKC phosphorylation. In some embodiments, the expression level of the PKC enzyme is determined for one or more PKC isoforms α, β (e.g., βI or βII,), γ, δ, ε, η, θ, ι/λ, μ, and ζ. In some embodiments, an assessment of the PKC activation potential includes determining or identifying the expression level of one or more of PKC isoforms α, β (e.g., βI or βII,), and γ. In some embodiments, an assessment of the PKC activation potential includes determining or identifying the expression level of one or more of PKC isoforms δ, ε, η, and θ. In some embodiments, an assessment of the PKC activation potential includes determining or identifying the expression level of one or more of PKC isoforms ι/λ, Ξ, and ζ. In various embodiments, the measured expression level of the PKC enzyme is compared to a control or reference level, such as the level of PKC in the cancer prior to treatment with the PKC activator and/or the level of PKC in non-cancerous cell or tissue, e.g., normal cell or tissue. In some embodiments, the measured expression level of PKC enzyme is compared to the level in the cancer prior to treatment with the PKC activator. In some embodiments, the expression level of the PKC enzyme is determined at the protein level or at the level of mRNA. In some embodiments, a cancer having an effective PKC activation potential and elevated expression of PKC enzymes is selected for treatment with the PKC activator.

In some embodiments, cancers for selection and treatment based on its PKC activation potential includes, among others, cancer of the pancreas, lung, colon, head and neck, stomach (gastric), biliary tract, endometrium, ovary, small intestine, urinary tract, liver, cervix, breast, brain, renal, skin, bone, and kidney, and hematologic cancers, such as lymphomas and leukemias. In some embodiments, the PKC activation potential in the cancer is determined for one or more of PKC isoforms selected from PKC α, β, γ, δ, ε, η, θ, ι/λ, μ and ζ.

In some embodiments, the cancer selected based on PKC activation potential is pancreatic cancer. In some embodiments, the pancreatic cancer is pancreatic adenocarcinoma or metastatic pancreatic cancer. In some embodiments, the PKC activation potential in the pancreatic cancer is determined for one or more of PKC isoforms selected from PKC α, β, γ, δ, ε, η, θ, ι/λ, μ and ζ. In some embodiments, the pancreatic cancer is selected for treatment with the combination if the cancer is identified as being negative for loss-of-function mutations in one or more of PKC γ, δ, ε, μ, and θ. In some embodiments, the pancreatic cancer is not selected for treatment with the combination if the cancer is determined or identified as having a loss-of-function mutations in one or more of PKC γ, δ, ε, μ and θ.

In some embodiments, the cancer selected based on PKC activation potential is colon cancer. In some embodiments, the colon cancer is a colon adenocarcinoma or a metastatic colon cancer. In some embodiments, the PKC activation potential in the colon cancer is determined for one or more of PKC isoforms selected from PKC α, β, γ, δ, ε, η, θ, ι/λ, μ and ζ. In some embodiments, the colon cancer is selected for treatment with the combination if the cancer is determined or identified as being negative for loss-of-function mutations in one or more of PKC α, β, γ, δ, η, μ and ι/λ. In some embodiments, the colon cancer is not selected for treatment with the combination if the cancer is determined or identified as having loss-of-function mutations in one or more of PKC α, β, γ, δ, η, μ and ι/λ.

In some embodiments, the cancer selected based on PKC activation potential is lung cancer. In some embodiments, the lung cancer is small cell lung cancer. In some embodiments, the lung cancer is non-small cell lung cancer. In some embodiments, the non-small cell lung cancer is an adenocarcinoma, squamous cell carcinoma, or large cell carcinoma. In some embodiments, the lung cancer is metastatic lung cancer. In some embodiments, the PKC activation potential in the lung cancer is determined for one or more of PKC isoforms selected from PKC α, β, γ, δ, ε, η, θ, ι/λ, μ and ζ. In some embodiments, the lung cancer is selected for treatment with the combination if the cancer is determined or identified as being negative for loss-of-function mutations in one or more of PKC γ, β, α, δ, ε, μ and η. In some embodiments, the lung cancer is not selected for treatment with the combination if the cancer is determined or identified as having loss-of-function mutations in one or more of PKC γ, β, α, δ, ε, μ and η.

In some embodiments, the cancer selected based on PKC activation potential is stomach or gastric cancer. In some embodiments, the PKC activation potential in the stomach or gastric cancer is determined for one or more of PKC isoforms selected from PKC α, β, γ, δ, ε, η, θ, ι/λ, μ and ζ. In some embodiments, the stomach or gastric cancer is selected for treatment with the combination if the cancer is determined or identified as being negative for loss-of-function mutations in one or more of PKC γ, δ and μ. In some embodiments, the stomach or gastric cancer is not selected for treatment with the combination if the cancer is determined or identified as having loss-of-function mutations in one or more of PKC γ, δ and μ.

In some embodiments, the cancer selected based on PKC activation potential is endometrial or ovarian cancer. In some embodiments, the PKC activation potential in the endometrial or ovarian cancer is determined for one or more of PKC isoforms selected from PKC α, β, γ, δ, ε, η, θ, ι/λ, μ and ζ. In some embodiments, the endometrial or ovarian cancer is selected for treatment with the combination if the cancer is determined or identified as being negative for loss-of-function mutations in one or more of PKC α, β, γ, δ, ε, η, ι/λ, μ and ζ. In some embodiments, the endometrial or ovarian cancer is not selected for treatment with the combination if the cancer is determined or identified as having loss-of-function mutations in one or more of PKC α, β, γ, δ, ε, η, ι/λ, μ and ζ.

In some embodiments, the cancer selected based on PKC activation potential is breast cancer. In some embodiments, the breast cancer is metastatic breast cancer. In some embodiments, the breast cancer is estrogen receptor negative breast cancer. In some embodiments, the breast cancer is Her2 negative breast cancer. In some embodiments, the breast cancer is estrogen receptor positive breast cancer. In some embodiments, the breast cancer is Her2 positive breast cancer. In some embodiments, the breast cancer is selected for treatment with the combination if the cancer is determined or identified as being negative for loss-of-function mutations in one or more of PKC α, β, γ, δ, ε, η, ι/λ, μ and ζ. In some embodiments, the breast cancer is not selected for treatment with the combination if the cancer is determined or identified as having loss-of-function mutations in one or more of PKC α, β, γ, δ, ε, η, ι/λ, μ and θ.

In some embodiments, the cancer selected based on PKC activation potential is head and neck cancer. In some embodiments, the head and neck cancer is selected for treatment with the combination if the cancer is determined or identified as being negative for loss-of-function mutations in one or more of PKC α, β, γ, δ, η, μ and ι/λ. In some embodiments, the head and neck cancer is not selected for treatment with the combination if the cancer is determined or identified as having loss-of-function mutations in one or more of PKC α, β, γ, δ, η, μ and ι/λ.

For the methods herein, mutations in K-RAS, N-RAS, H-RAS and PKC enzymes can be identified using various techniques available to the skilled artisan. In various embodiments, the presence or absence of a mutation can be determined by known DNA or RNA detection methods, for example, DNA sequencing, oligonucleotide hybridization, polymerase chain reaction (PCR) amplification with primers specific to the mutation, or protein detection methods, for example, immunoassays or biochemical assays to identify a mutated protein, such as mutated K-RAS, N-RAS and PKCs. In some embodiments, the nucleic acid or RNA in a sample can be detected by any suitable methods or techniques of detecting gene sequences. Such methods include, but are not limited to, PCR, reverse transcriptase-PCR (RT-PCR), in situ PCR, in situ hybridization, Southern blot, Northern blot, sequence analysis, microarray analysis, or other DNA/RNA hybridization platforms (see, e.g., Taso et al., 2010, Lung Cancer 68(1):51-7). In particular, detection of mutations can use samples obtained non-invasively, such as cell free nucleic acid (e.g., cfDNA) from blood.

In some embodiments, mutations can be detected using various Next-Gen sequencing (NGS) techniques, particularly high-throughput NGS techniques. Exemplary NGS techniques include, among others, Polony sequencing (see, e.g., Shendure et al., 2005, Science 309(5741):1728-32), IonTorrent sequencing (see, e.g., Rusk, N., 2011, Nat Meth 8(1):44-44), pyrosequencing (see, e.g., Marguiles et al., 2005, Nature 437(7057):376-380), reversible dye sequencing with colony sequencing (Bentley et al., 2008, Nature 456(7218):53-59; Illumina, CA, USA), sequencing by ligation (e.g., SOLid systems of Applied Biosystems; Valouev et al., 2008, Genome Res. 18(7):1051-1063), high throughput rolling circle “nanoball” sequencing (see, e.g., Drmanac et al., 2010, Science 327 (5961):78-81; Porreca, G. J., 2010, Nature Biotech. 28 (1):43-44), and zero-mode wave guide based sequencing (see, e.g., Chin et al., 2013, Nat Methods 10(6):563-569); all publications incorporated herein by reference. In some embodiments, massively parallel sequencing of target genes, such as genes encoding K-RAS, N-RAS, H-RAS, or PKCs can be carried out to detect or identify presence or absence of mutations in the cancer being assessed for treatment with a PKC activator.

In some embodiments, detection of point mutations in target nucleic acids can be accomplished by molecular cloning of the target nucleic acid molecules and sequencing the nucleic acid molecules using available techniques. Alternatively, amplification techniques such as PCR can be used to amplify target nucleic acid sequences directly from a genomic DNA preparation from a tumor tissue, cell sample, or cell free sample (e.g., cell free plasma from blood). The nucleic acid sequence of the amplified molecules can then be determined to identify mutations. Design and selection of appropriate primers are within the abilities of one of ordinary skill in the art. Other methods of detecting mutations that can be used include, among others, ligase chain reaction, allele-specific PCR restriction fragment length polymorphism, single stranded conformation polymorphism analysis, mismatch detection proteins (e.g., GRIN2A or TRRAP), RNase protection (e.g., Winter et al., 1985, Proc. Natl. Acad. Sci. USA 82:7575-7579), enzymatic or chemical cleavage (Cotton et al., 1988, Proc. Natl. Acad. Sci. USA 85: 4397; Shenk et al., 1975, Proc. Natl. Acad. Sci. USA 72:989).

In some embodiments, mutations in nucleic acid molecules can also be detected by screening for alterations of the corresponding protein. For example, monoclonal antibodies immunoreactive with a target gene product can be used to screen a tissue, for example an antibody that is known to bind to a particular mutated position of the gene product (protein). For example, a suitable antibody may be one that binds to a deleted exon or that binds to a conformational epitope comprising a deleted portion of the target protein. Lack of cognate antigen would indicate a mutation. Such immunological assays can be accomplished using any convenient format known in the art, such as Western blot, immunohistochemical assay and ELISA. For example, antibody-based detection of K-ras mutations is described in Elisabah et al., 2013, J Egypt Natl Cancer Inst. 25(1):51-6).

The expression of mRNA or proteins, such as expression of PKC or downstream elements, such as Frizzled, can use standard techniques available to the skilled artisan, including some of the methods described above. For example, the mRNA encoding a protein of interest can be detected by hybridization with nucleic acid probes, reverse transcription, polymerase chain reaction, and combinations thereof (e.g., RT-qPCR). In some embodiments, chip-based or bead-based microarrays containing nucleic acid probes hybridizing to the target sequence can be used. In some embodiments, mRNA expression can be detected directly in the target cells, such as by in-situ hybridization.

In some embodiments, the protein products can be detected directly. Direct detection can use a binding agent that binds specifically to the protein, such as antibodies or target-interacting proteins or small molecule reagents that bind specifically with the protein target of interest (see, e.g., Current Protocols in Immunology, Coligan et al., eds., John Wiley & Sons (updates to 2015); Immunoassays: A Practical Approach, Gosling, ed., Oxford University Press (2000)). In some embodiments, the protein product can be detected by immunological methods, including, by way of example, enzyme immunoassays, enzyme-linked immunoassays, fluorescence polarization immunoassay, and chemiluminescence assay.

For determining PKC activation potential, general methods for detecting PKC activity can be used, such as described in Protein Kinase C Protocols, Newton, A. C. ed., Humana Press, Totowa, N.J. USA (2003), incorporated herein by reference. In some embodiments, the assays for detecting kinase activity can use synthetic substrates or natural substrates that are the target of the PKC enzymes and detecting the phosphorylated substrate, for example by transfer of detectable phospho group (e.g., ³²P-labeled or ligand labeled ATP) or detection of the phosphorylated product, such as with an antibody that binds the phosphorylated product (PegTag®, Promega, USA). In some embodiments, PKC activity can be detected in situ (see, e.g., Iori et al., 2003, Diabetologia. 46(4):524-30). Samples for examining PKC activity includes cells and tissues obtained from a patient, and/or circulating cancer cells obtained from the peripheral blood or lymph of patients (see, e.g., Karabacak et al., 2014, Nat Protoc. 9(3):694-710; van de Stolpe et al., 2011, Cancer Res. 71:5955-5960; Yu et al., 2011, J Cell Biol. 192(3):373-382; and Stott et al., 2010, Proc Natl Acad Sci. USA 107(43):18392-18397; all publications incorporated herein by reference). In some embodiments, the PKC activity can be measured by use of synthetic peptide substrates. These synthetic peptide substrates can be based on amino acid sequences known to be phosphorylated naturally in a PKC enzyme. Substrates for PKCα, β and γ are described in Toomik et al., 1997, Biochem J. 322:455-460; substrates for PKCα, β, δ, ζ, and μ are described in Nishikawa et al., J Biol Chem. 272(2):952-960; Chen et al., 1993, Biochem. 32(4):1032-1039; and Wang et al., 2012, Structure 20(6):791-801; incorporated herein by reference. PKC substrates are also available commercially (see, e.g., Abcam, MA, USA; Perkin Elmer, USA; ImmuneChem, BC, Canada; and Promega, USA).

Detection of phosphorylated PKC enzymes can use standard techniques, such as antibodies that distinguish phosphorylated protein from non-phosphorylated protein or by detection of a labeled phosphate group (e.g., ³²P) (see, e.g., Barcelo et al., 2014, Cancer Res. 74:1190-1190; Vila Petroff et al., 2010, J Mol Cell Cardiol. 9(1):106-112; Zhang et al., 2002, J Biol Chem. 277(42):39379-39387; Dissanayake et al., 2008, Methods Mol Biol. 468:187; all publications incorporated herein by reference). In some embodiments, antibodies that detect phosphorylated target proteins can be obtained commercially (see, e.g., Abcam, USA; Cell Signaling Technology, USA). In some embodiments, detecting or measuring phosphorylated proteins by use of anti-phospho antibodies can comprise: affinity isolating the PKC protein; and detecting phosphorylated protein with an anti-phospho antibody. In some embodiments, the affinity isolated PKC protein can be separated, such as by gel electrophoresis, the separated proteins bound onto a membrane substrate; and the membrane probed with an anti-phospho antibody. The binding of the anti-phospho antibody to phosphorylated protein can be detected with anti-phospho antibodies containing a detectable label, or by use of a secondary antibody directed against the primary anti-phospho antibody, where the secondary antibody contains a detectable label. The detectable label can be, by way of example and not limitation, a radioactive label, detectable enzyme (e.g., horseradish peroxidase); or fluorescent molecule. Exemplary antibodies for detecting phosphorylated sequences in PKC enzymes are provided below on Table A. Also provided is an antibody against phosphorylated CaMKii (Thr286). The level of phospho-CaMKii (Thr286) serves as a marker of K-Ras driven stemness pathways.

TABLE A

| Antibody Name | Vendor | Cat No. | Species | Dilution |
|---|---|---|---|---|
| GAPDH (loading control) | Millipore | MAB374 | Mouse | 1:10000 |
| β-Actin (loading control) | Sigma | A5441 | Mouse | 1:10000 |
| Vinculin (loading control) | Sigma | V9131 | Mouse | 1:20000 |
| Phospho-CaMKii (Thr286) | Abcam | ab32678 | Rabbit | 1:1000 |
| Phospho-PKC substrate | Cell Signaling | 6967 | Rabbit | 1:1000 |
| Motif [(R/KXpSX(R/K)] MultiMab ™ | | | | |
| Phospho(Ser)-PKC substrate | Cell Signaling | 2261 | Rabbit | 1:500 |
| Phospho-PKC(pan)(βII Ser660) | Cell Signaling | 9371 | Rabbit | 1:1000 |
| Phospho-PKCα/β (Thr638/641) | Cell Signaling | 9375 | Rabbit | 1:1000 |
| Phospho-PKCδ/θ (Ser643/676) | Cell Signaling | 9376 | Rabbit | 1:1000 |
| Phospho-PKD/PKCμ (Ser744/748) | Cell Signaling | 2054 | Rabbit | 1:1000 |
| Phospho-PKD/PKCμ (Ser916) | Cell Signaling | 2051 | Rabbit | 1:1000 |
| Phospho-PKCδ (Thr505) | Cell Signaling | 9374 | Rabbit | 1:1000 |
| Phospho- PKCδ (Ser299) | Abcam | Ab133456 | Rabbit | 1:1000 |
| Phospho-PKCδ (Tyr311) | Cell Signaling | 2055 | Rabbit | 1:1000 |
| Phospho-PKCζ/λ (Thr410/403) | Cell Signaling | 9378 | Rabbit | 1:1000 |
| PKD/PKCμ | Cell Signaling | 2052 | Rabbit | 1:1000 |
| P44/42 Erk1/2 | Cell Signaling | 9102 | Rabbit | 1:1000 |
| Phospho-p44/42 Erk1/2 | Cell Signaling | 9106 | Mouse | 1:1000 |
| (Thr202/Tyr204) | | | | |
| Phospho-c-Raf (Ser338) | Cell Signaling | 9427 | Rabbit | 1:1000 |

In some embodiments, phosphorylation can be detected in situ in a cell, for example, using an antibody directed against the phosphorylated protein. In some embodiments, the technique of in situ proximity ligation assay can be used to detect phosphorylated proteins in situ (see, e.g., Soderberg et al., 2006, Nat Methods 3:995-1000; Jarvious et al., 2007, Method Mol Cell Proteomics 6:1500-1509). Other methods of in situ detection of phosphorylated proteins are described in, for example, Roche et al., “Detection of Protein Phosphorylation in Tissues and Cells,” in Current Protocols in Neuroscience, John Wiley & Sons (2001); incorporated herein by reference.

Biological sample for the method herein include any samples are amenable to analysis herein, such as tissue or biopsy samples containing cancer cells, or any biological fluids that contain the material of interests (e.g., DNA), such as blood, plasma, saliva, tissue swabs, and intestinal fluids. In some embodiments, exosomes extruded by cancer cells and obtained from blood or other body fluids can be used to detect nucleic acids and proteins produced by the cancer cells.

General biological, biochemical, immunological and molecular biological methods applicable to the present disclosure are described in Sambrook et al., Molecular Cloning: A Laboratory Manual 2^{nd} Ed. (1989) Cold Spring Harbor Laboratory Press, Cold Spring Harbor, N.Y.; Current Protocols in Molecular Biology, Ausubel et al., ed., John Wiley & Sons (2015); Current Protocols in Immunology, Coligan, J E ed., John Wiley & Sons (2015); and Methods in Enzymology, Vol. 200, Abelson et al., ed., Academic Press (1991). All publications are incorporated herein by reference.

### 5.4. Formulations and Administration

In some embodiments, pharmaceutical compositions of the therapeutic agents can be formulated by standard techniques using one or more physiologically acceptable carriers or excipients. Suitable pharmaceutical carriers are described herein and in Remington: The Science and Practice of Pharmacy, 21^{st} Ed. (2005). The therapeutic compounds and their physiologically acceptable salts, hydrates and solvates can be formulated for administration by any suitable route, including, among others, topically, nasally, orally, parenterally, rectally or by inhalation. In some embodiments, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets, capsules, and solutions can be administered orally, rectally or vaginally.

For oral administration, a pharmaceutical composition can take the form of, for example, a tablet or a capsule prepared by conventional means with a pharmaceutically acceptable excipient. Tablets and capsules comprising the active ingredient can be prepared together with excipients such as: (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; (c) binders, e.g., magnesium aluminum silicate, starch paste, gelatin, tragacanth, methylcellulose, sodium carboxymethylcellulose, polyvinylpyrrolidone and/or hydroxypropyl methylcellulose; (d) disintegrants, e.g., starches (including potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. The compositions are prepared according to conventional mixing, granulating or coating methods.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The therapeutic agents can be formulated for parenteral administration, for example by bolus injection or continuous infusion. Formulations for injection can be presented in unit dosage form, for example, in ampoules or in multi-dose containers, with an optionally added preservative. Injectable compositions can be aqueous isotonic solutions or suspensions. In some embodiments for parenteral administration, the therapeutic agents can be prepared with a surfactant, such as Cremaphor, or lipophilic solvents, such as triglycerides or liposomes. The compositions may be sterilized and/or contain adjuvants, such as preserving, stabilizing, wetting or emulsifying agents, solution promoters, salts for regulating the osmotic pressure and/or buffers. Alternatively, the therapeutic agent can be in powder form for reconstitution with a suitable vehicle, for example, sterile pyrogen-free water, before use. In addition, they may also contain other therapeutically effective substances.

For administration by inhalation, the therapeutic agent may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a therapeutic agent with a carrier. Preferred carriers include absorbable pharmacologically acceptable solvents to assist passage through the skin of the host. For example, transdermal devices are in the form of a bandage or patch comprising a backing member, a reservoir containing the therapeutic agent optionally with carriers, optionally a rate controlling barrier to deliver the compound to the skin of the host at a controlled and predetermined rate over a prolonged period of time, and a means to secure the device to the skin. Matrix transdermal formulations may also be used.

Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

The therapeutic agent can also be formulated as a rectal composition, for example, suppositories or retention enemas, for example, containing conventional suppository bases, for example, cocoa butter or other glycerides, or gel forming agents, such as carbomers.

In some embodiments, the therapeutic agent can be formulated as a depot preparation. Such long-acting formulations can be administered by implantation (for example, subcutaneously or intramuscularly) or by intramuscular injection. The therapeutic agent can be formulated with suitable polymeric or hydrophobic materials (for example as an emulsion in an acceptable oil), ion exchange resins, biodegradable polymers, or as sparingly soluble derivatives, for example, as a sparingly soluble salt.

The pharmaceutical compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

### 5.5. Therapeutically Effective Amount and Dosing

In some embodiments, a pharmaceutical composition of the therapeutic agent is administered to a subject, preferably a human, at a therapeutically effective dose to prevent, treat, or control a condition or disease as described herein. The pharmaceutical composition is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose” or “therapeutically effective amount.”

The dosage of therapeutic agents can take into consideration, among others, the species of warm-blooded animal (mammal), the body weight, age, condition being treated, the severity of the condition being treated, the form of administration, route of administration. The size of the dose also will be determined by the existence, nature, and extent of any adverse effects that accompany the administration of a particular therapeutic compound in a particular subject.

In some embodiments, a suitable dosage of a ferroptosis inducer compound or a composition thereof is from about 1 ng/kg to about 1000 mg/kg, from 0.01 mg/kg to 900 mg/kg, 0.1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg. In some embodiments, the suitable dosages of the ferroptosis inducer can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In some embodiments, the daily dose of the ferroptosis inducer can be administered once per day or divided into subdoses and administered in multiple doses, e.g., twice, three times, or four times per day.

In some embodiments, the ferroptosis inducer can be administered with one or more of a second therapeutic agent, sequentially or concurrently, either by the same route or by different routes of administration. When administered sequentially, the time between administrations is selected to benefit, among others, the therapeutic efficacy and/or safety of the combination treatment. In some embodiments, the ferroptosis inducer can be administered first followed by a second therapeutic agent, or alternatively, the second therapeutic agent can be administered first followed by the ferroptosis inducer. By way of example and not limitation, the time between administrations is about 1 hr, about 2 hr, about 4 hr, about 6 hr, about 12 hr, about 16 hr or about 20 hr. In some embodiments, the time between administrations is about 1, about 2, about 3, about 4, about 5, about 6, or about 7 more days. In some embodiments, the time between administrations is about 1 week, 2 weeks, 3 weeks, or 4 weeks or more. In some embodiments, the time between administrations is about 1 month or 2 months or more.

When administered concurrently, the ferroptosis inducer can be administered separately at the same time as the second therapeutic agent, by the same or different routes, or administered in a single composition by the same route. In some embodiments, the amount and frequency of administration of the second therapeutic agent can used standard dosages and standard administration frequencies used for the particular therapeutic agent. See, e.g., Physicians' Desk Reference, 70th Ed., PDR Network, 2015; incorporated herein by reference.

In some embodiments where the ferroptosis inducer is administered in combination with a second therapeutic agent, the dose of the second therapeutic agent is administered at a therapeutically effective dose. In some embodiments, a suitable dose can be from about 1 ng/kg to about 1000 mg/kg, from about 0.01 mg/kg to about 900 mg/kg, from about 0.1 mg/kg to about 800 mg/kg, from about 1 mg/kg to about 700 mg/kg, from about 2 mg/kg to about 500 mg/kg, from about 3 mg/kg to about 400 mg/kg, from about 4 mg/kg to about 300 mg/kg, or from about 5 mg/kg to about 200 mg/kg. In some embodiments, the suitable dosages of the ferroptosis inducer can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In some embodiments, guidance for dosages of the second therapeutic agent is provided in Physicians' Desk Reference, 70^{th} Ed, PDR Network (2015), incorporated herein by reference.

In some embodiments where second therapeutic agent is a diterpenoid PKC activating compound, the compound can be administered in a daily dose in the range from about 0.01 mg per kg of subject weight (0.01 mg/kg) to about 1000 mg/kg. In some embodiments, the daily dose is a dose in the range of about 0.1 mg/kg to about 500 mg/kg. In some embodiments, the daily dose is a dose in the range of about 1 mg/kg to about 500 mg/kg. In some embodiments, the daily dose is about 2 mg/kg to about 250 mg/kg. In another embodiment, the daily dose is about 5 mg/kg to about 100 mg/kg. In another embodiment, the daily dose is about 5 mg/kg to about 100 mg/kg. In some embodiments, the daily dose is about 0.01 mg/kg, 0.05 mg/kg, 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/kg, 10 mg/kg, 20 mg/kg, 40 mg/kg, 50 mg/kg, 100 mg/kg, 200 mg/kg or 500 mg/kg. In some embodiments, the daily dose can be administered once per day or divided into subdoses and administered in multiple doses, e.g., twice, three times, or four times per day.

It to be understood that optimum dosages, toxicity, and therapeutic efficacy of such therapeutic agents may vary depending on the relative potency of individual therapeutic agent and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Therapeutic agents or combinations thereof that exhibit large therapeutic indices are preferred. While certain agents that exhibit toxic side effects can be used, care should be used to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

The following examples are provided to further illustrate the methods of the present disclosure, and the compounds and compositions for use in the methods. The examples described are illustrative only and are not intended to limit the scope of the invention in any way.

### 6. EXAMPLES

### Example 1: Effect of Ferroptosis Inducers on Proliferation of Different Cancer Cell Lines

Erastin, RSL3, and ML210 were initially identified as molecules inducing cell death preferentially in RAS mutant cells in comparison to Ras wild-type cells (Dixon et al., 2012, Cell. 149(5):1060-72; Dolma et al., 2003, Cancer Cell. 3(3):285-96; Yang and Stockwell., 2008, Chem Biol. 15(3):234-45; Bittker et al., Screen for RAS-Selective Lethal Compounds and VDAC Ligands—Probe 2. Probe Reports from the NIH Molecular Libraries Program. Bethesda (Md.): National Center for Biotechnology Information (US); 2010-2011)). Therefore, cells with RAS activating mutations may be sensitive and have similar range of sensitivity to these compounds. However, a number of previous studies or screens utilized engineered cell lines expressing wild-type or mutant H-RAS. Although sharing many common downstream effector pathways, K-RAS and H-RAS are different in their ability to activate cancer stemness pathways (Wang et al., 2015, Cell 163(5):1237-51). Recent literature also suggests that sensitivity to the initially identified Ras synthetic lethal compounds is not entirely dependent on Ras mutational status of a particular cell line. Cancer cell lines harboring mutant Ras are no more sensitive to these compounds than those expressing wild-type Ras (Yang et al., 2014, Cell. 156:317-331; Cao and Dixon, 2016, Cell. Mol. Life Sci. 73:2195-2209). Thus, experiments were conducted to determine sensitivity of these compounds in cancer cell lines harboring K-RAS mutations since K-RAS is the RAS isoform frequently mutated in many cancer cells. Studies were also conducted on cells that did not harbor K-RAS mutations.

Various ferroptosis inducer compounds were tested in a panel of cancer cell lines including pancreatic, lung, and lymphoma cell lines, which are listed in Table 1.

TABLE 1
Cancer Cell Lines

| Cell Line Name | Tumor Type |
|---|---|
| A549 | Lung, adenocarcinoma |
| MiaPaCa-2 | Pancreatic, carcinoma |
| Panc1 | Pancreatic, epithelioid carcinoma |
| KP-4 | Pancreatic, carcinoma |
| Panc6.03 | Pancreatic, ductal adenocarcinoma |
| Panc2.13 | Pancreatic, adenocarcinoma |
| PSN-1 | Pancreatic, adenocarcinoma |
| Capan-1 | Pancreatic, adenocarcinoma, liver metastasis |
| Mino | Lymphoma, mantle cell lymphoma |
| | (B cell non-Hodgkin's lymphoma) |
| Namalwa | Lymphoma, Burkitt's lymphoma |

The ferroptosis inducer compounds, including RSL3, Erastin, ML210, Auranofin, Sulfasalazine, Artesunate, Artemisinin, Dihydroartemisinin, Sorafenib, Buthionine sulfoximine (BSO), Altretamine, Almitrine, and ML162 tested on the cell lines are listed in Table 2.

TABLE 2
Compound Structures

| Compound | Structure |
|---|---|
| RSL3 | 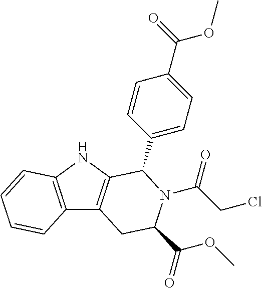 |
| ML210 | 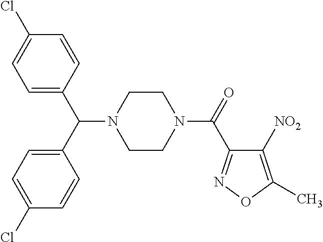 [SMILES: Cc1onc(C(=O)N2CCN(C(c3ccc(Cl)cc3)c3ccc(Cl)cc3)CC2)c1[N+](=O)[O-]] |
| Erastin | 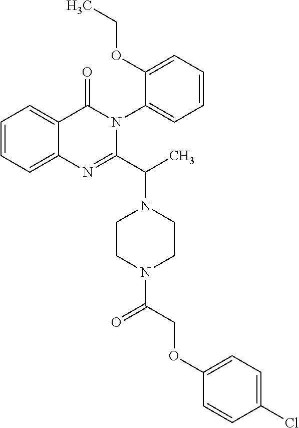 [SMILES: CCOc1ccccc1-n1c(C(C)N2CCN(C(=O)COc3ccc(Cl)cc3)CC2)nc2ccccc2c1=O] |
| Sulfasalazine | 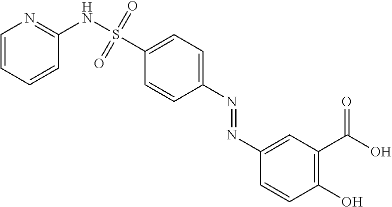 [SMILES: O=C(O)c1cc(/N=N/c2ccc(S(=O)(=O)Nc3ccccn3)cc2)ccc1O] |
| Artemisinin | 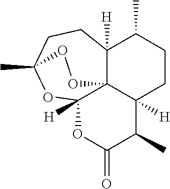 [SMILES: C[C@@H]1CC[C@H]2[C@@H](C)C(=O)O[C@@H]3O[C@]4(C)CC[C@@H]1[C@]32OO4] |
| Sorafenib | 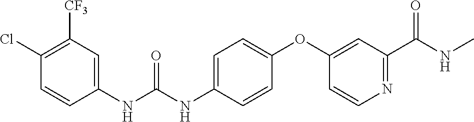 [SMILES: CNC(=O)c1cc(Oc2ccc(NC(=O)Nc3ccc(Cl)c(C(F)(F)F)c3)cc2)ccn1] |
| Altretamine | 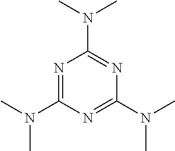 |
| Auranofin | 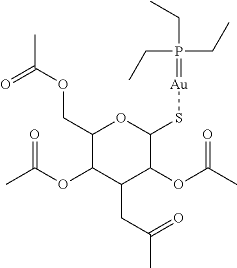 |
| Artesunate | 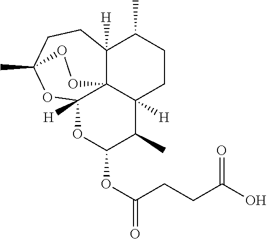 [SMILES: C[C@H]1[C@H](OC(=O)CCC(=O)O)O[C@@H]2O[C@]3(C)CC[C@H]4[C@H](C)CC[C@@H]1[C@@]24OO3] |
| Dihydro- artemisinin | 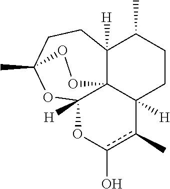 |
| BSO | 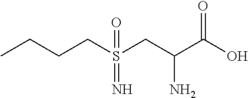 |
| Almitrine | 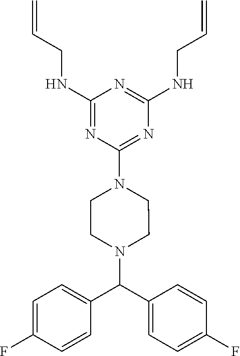 [SMILES: C=CCNc1nc(NCC=C)nc(N2CCN(C(c3ccc(F)cc3)c3ccc(F)cc3)CC2)n1] |
| Artemether | 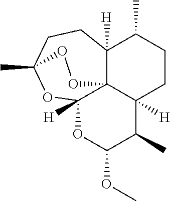 [SMILES: CO[C@@H]1O[C@@H]2O[C@]3(C)CC[C@H]4[C@H](C)CC[C@@H]([C@H]1C)[C@@]24OO3] |
| ML162 | 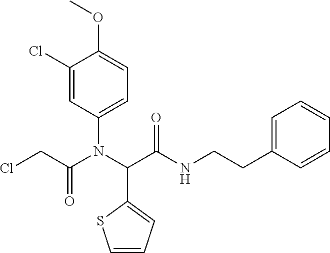 [SMILES: COc1ccc(N(C(=O)CCl)C(C(=O)NCCc2ccccc2)c2cccs2)cc1Cl] |

Cell viability assay was performed to assess the potency of the ferroptosis inducer compounds in various cancer cell lines with K-Ras mutations. Cells at a density of 1,000-10,000 cells/well were seeded in 96-well plates and incubated at 37° C. overnight. A series of 9 different concentrations of compound stocks (500×) were created by 3-fold serial dilution in DMSO. These compounds were further diluted in culture media and then added to cells so that the final DMSO concentration was equal to 0.25% or less. After 96 hours of incubation, 50 μL of CellTiter Glo reagent (Promega) was added to each well and luminescence was measured after 10 minutes using EnVision (PerkinElmer). K101A (a PKC activator) was used as the reference compound titrated from top concentration of 30 μM. All compounds were tested initially from 30 μM as the top concentration in duplicates (range of 4.6 nM-30 μM). The top concentration was then adjusted to higher (from up to 1000 μM) or lower (from as low as 0.1 μM) for compounds that showed potency out of the initial range Luminescence from cells treated with DMSO alone was set as Max and % of inhibition was calculated as follows: Inhibition %=(Max-Sample value)/Max*100. Data was analyzed using XL-fit software (ID Business Solutions Ltd.). IC50, relative IC50, or % of top inhibition was calculated.

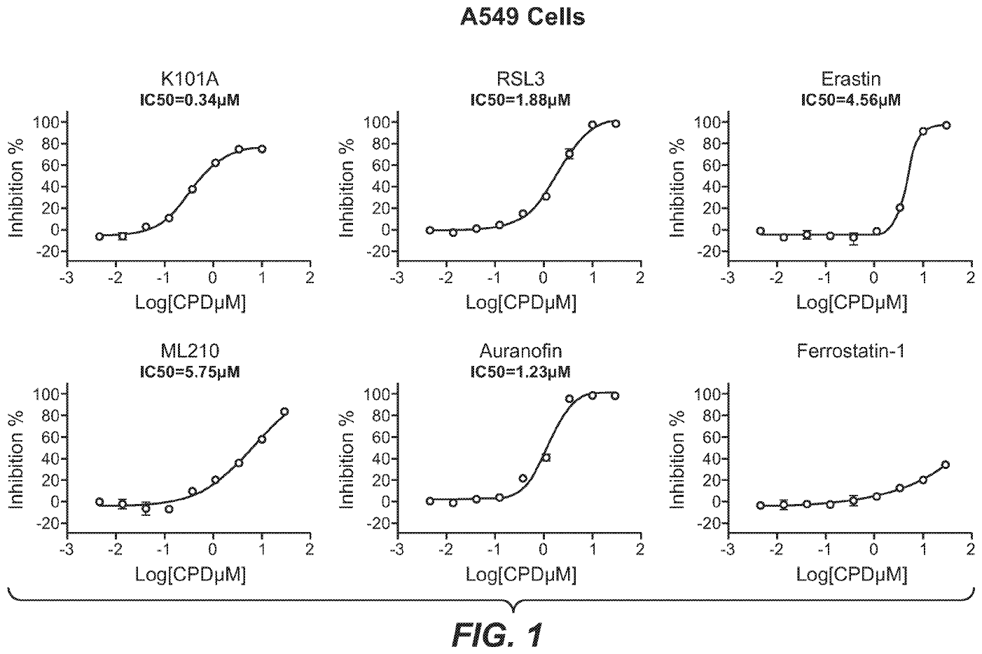

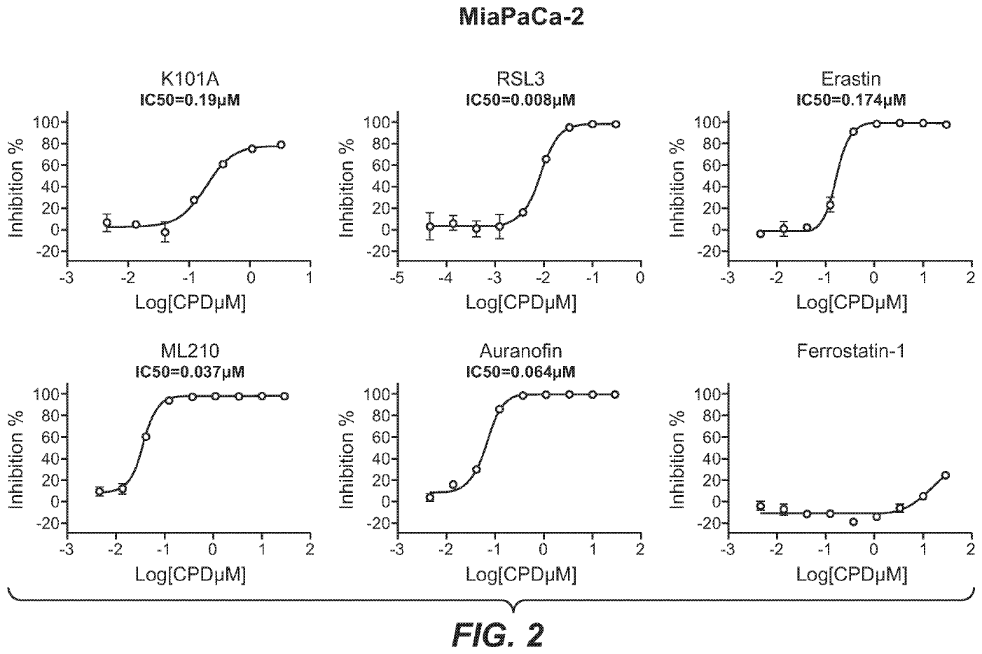

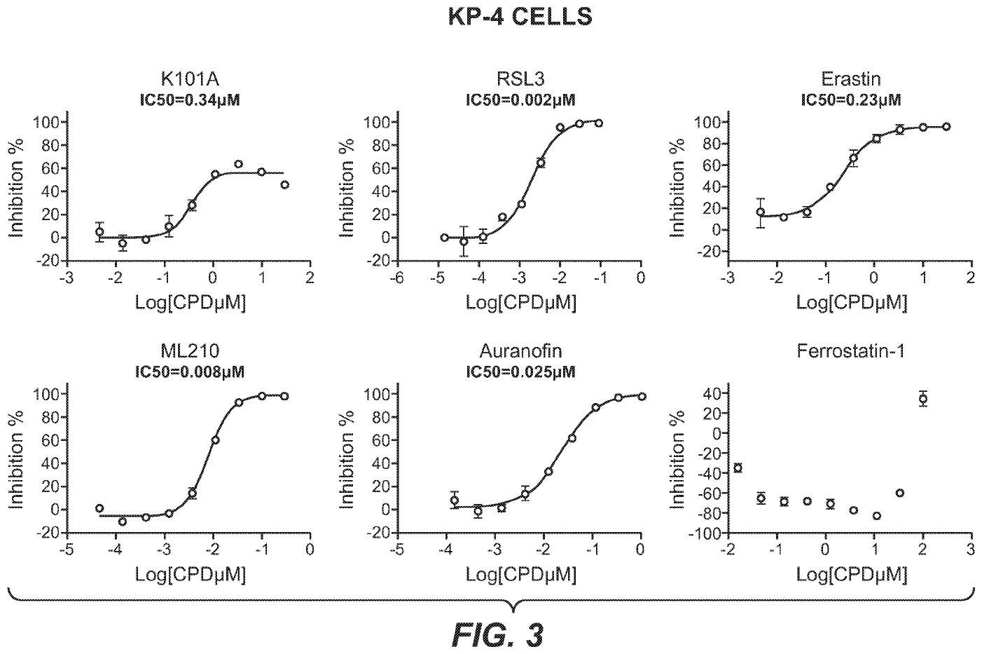

IC50s of PKC activator K101A (K101A), ferroptosis inducers RSL3, Erastin, ML210, Auranofin, and ferroptosis inhibitor Ferrostatin-1 are shown in FIG. 1 for A549 lung cancer cell line, FIG. 2 for MiaPaCa-2 pancreatic cancer cell line, FIG. 3 for KP-4 pancreatic cancer cell line. While K101A had similar potency inhibiting cell proliferation of A549, MiaPaCa-2, and KP-4, ferroptosis inducers showed dramatically different potency in different cell lines. For example, RSL3 was two to three orders of magnitude more potent in MiaPaCa-2 (IC50=0.008 μM) and KP-4 (IC50=0.002 μM) pancreatic cell lines than in A549 (IC50=1.88 μM) lung cancer cell line. Similarly, another GPX4 inhibitor ML210 was also at least two orders of magnitude more potent in the pancreatic cell lines. To a lesser extent, Erastin or Auranofin was roughly 20-50 fold more potent in the pancreatic cell lines than in A549. Interestingly, the same ferroptosis inducers have very different potency in these three K-Ras mutant cell lines. A recent report indicates that Ras mutation status does not predict sensitivity to Erastin-induced ferroptosis after testing over a hundred cancer cell lines from different tissues with or without Ras mutations (Yang et al., 2014, Cell 156(1-2):317-31). Cell lines from B cell lymphoma and renal cell carcinoma, which do not have K-Ras mutations, are the most sensitive cell lines to Erastin. However, the magnitude of differential sensitivity towards ferroptosis inducers in this study is much bigger than those reported in the literature.

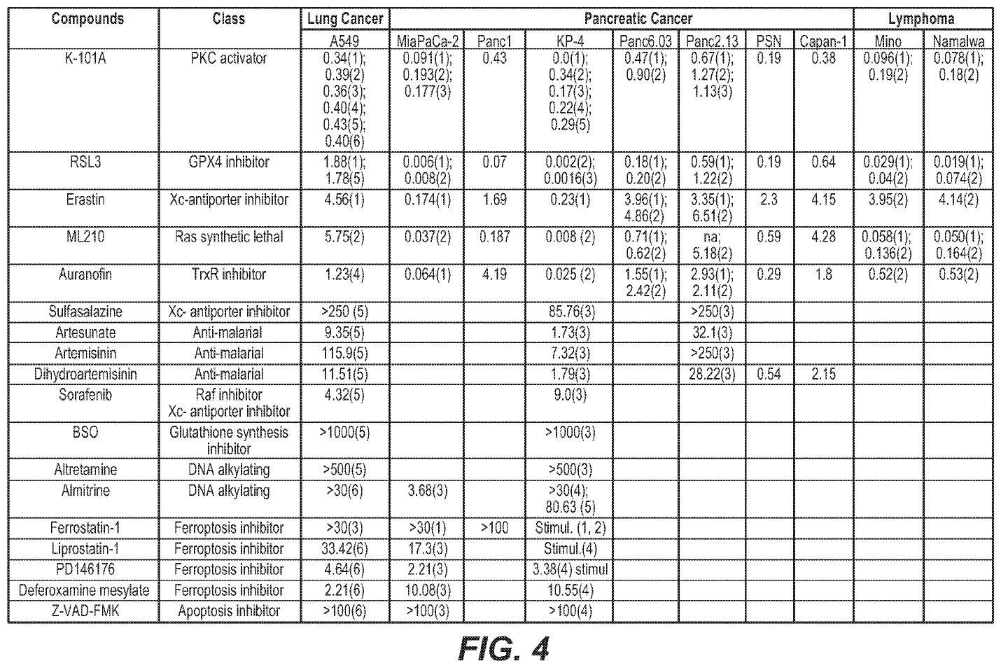

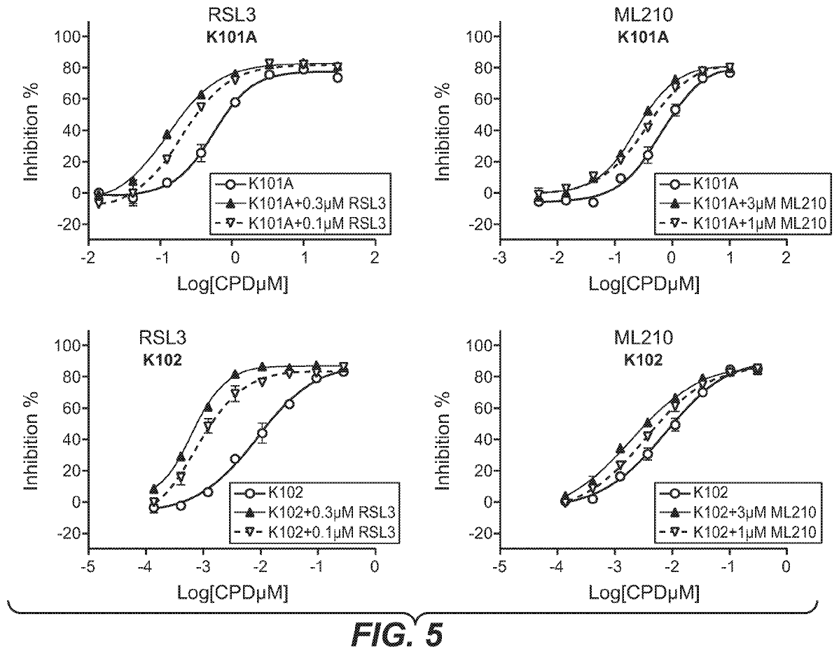

Next, RSL3, ML210, Erastin, and Auranofin were tested on additional K-RAS mutant pancreatic cell lines (Panc1, Panc6.03, Panc2.13, PSN-1, and Capan-1) in Table 1. The IC50s are listed in FIG. 4. Although not as sensitive as MiaPaCa-2 or KP-4, these pancreatic cancer cell lines showed a range of selectivity to cell death induced by different ferroptosis inducers in comparison to A549: All five pancreatic cell lines showed selective cell death, ranging 2-26 fold, induced by RSL3; While no selectivity was demonstrated for Panc2.13 or Capan-1, three pancreatic cell lines Panc1, Panc6.03, and PSN-1 showed 8-30 fold sensitivity for ML210; Panc1 and PSN-1 showed ˜2 fold selectivity for Erastin-induced cell death; Only PSN-1 showed ˜4 fold sensitivity for Auranofin-induced cell death.

RSL3, ML210, Erastin, and Auranofin were also tested in B cell lymphoma, a tumor type that has been shown to be one of the most sensitive to Erastin-induced ferroptosis (Yang et al., 2014, Cell 156(1-2):317-31). Surprisingly, two B cell lymphoma cell lines Mino and Namalwa were not more sensitive to Erastin-induced cell death in comparison to A549. However, other ferroptosis inducers did show selective killing of lymphoma cells, for example, 30-50 fold selectivity for RSL3 and ML210 and ˜2 fold for Auranofin in comparison to A549.

Based on the results above, pancreatic cancer seems to be have high sensitivity to ferroptosis inducers in general with a subset of pancreatic cancer cell lines much more sensitive than B cell lymphoma, a cell type identified as the most sensitive in the literature. Thus, in addition to K-Ras mutations, other factors, such as tumor origin, genetic context, stemness and mesenchymal phenotypes may also contribute to the sensitivity.

Because A549 and KP-4 cells had shown significant differential sensitivity towards a few well-known ferroptosis inducers, these two cell lines were used to test additional compounds that may inhibit targets in the ferroptosis pathway (Cao and Dixon, 2016, Cell Mol Life Sci. 73(11-12):2195-209; Xie et al., 2016, Cell Death Differ. 23(3):369-79). These compounds are listed in Table 2 and the results are listed in FIG. 4. ML162, another irreversible GPX4 inhibitor, inhibited KP-4 cells at low nM concentrations, showing >80 fold selectivity over its activity against A549 cells. Like Erastin, Sulfasalazine and Sorafenib have been suggested to act as inhibitors of cystine/glutamate antiporter system Xc⁻ activity. As shown in FIG. 4, while Sorfenib did not show selective killing of KP-4 cells as Erastin, Sulfasalazine showed selectivity although the potency was very weak in the high μM range, a concentration difficult to achieve in vivo. Artesunate, Dihydroartemisinin, and Artemisinin, the anti-malarial drugs, had been reported to induce ROS and ferroptotic cell death in a few cancers (Eling et al., 2015, Oncoscience 2(5):517-32; Roh et al., 2016, Redox Biol. 11:254-62). Concentration of Artesunate used in vitro is in the high μM range (e.g., 50 μM), which is also a concentration/AUC difficult to generate in vivo. In the assays used herein, the compounds showed 5-15 fold selective killing of KP-4 over A549 at μM range. A different analog, Artemether, also showed >5 fold potency towards KP-4 pancreatic cells when compared with A549. Dihydroartemisinin inhibited PSN-1 at IC50 of 0.54 μM and Capan-1 at 2.15 μM. Thus, in a subset of pancreatic cancers, Artemisinin related compounds demonstrated low μM potency and could be useful in vivo. BSO, a glutathione synthesis inhibitor, acts to deplete intracellular GSH leading to ferroptosis in certain cell lines. However, it had minimal effects on both A549 or KP-4 cells at very high concentration (>1 mM). Two other chemotherapeutic drugs, Altretamine and Almitrine, were very weak inhibitors of A549 and KP-4 cell lines. Thus, the Artemisinin related compounds showed a level of selective toxicity in a few pancreatic cancer cell lines at concentrations that may be achievable in vivo.

A number of ferroptosis inhibitors, such as Ferrostatin-1, Liproxstatin-1, Deferoxamine mesylate (Deferoxamine), and PD146176, and an apoptosis inhibitor Z-VAD-FMK (ZVAD) were also tested in A549, MiaPaCa-2, and KP-4 and the IC50s are shown in FIG. 4. In general, these compounds are not potent in blocking cell proliferation and do not show differential activity among three cell lines.

Based on the data, some ferroptosis inducers, such as GPX4 inhibitors RSL3, ML210, and their improved analogs, cystine/glutamate antiporter system Xc⁻ inhibitor Erastin or Sulfasalazine, thioredoxin reductase inhibitor Auranofin, Malaria drugs Artemisinin, Dihydroartemisinin, Artesunate, and their analogs Artemether, can work as monotherapy in a subset of pancreatic cancer cells.

### Example 2: Effect of Combination of Ferroptosis Inducer and PKC Activator on Proliferation of A549 Cancer Cells

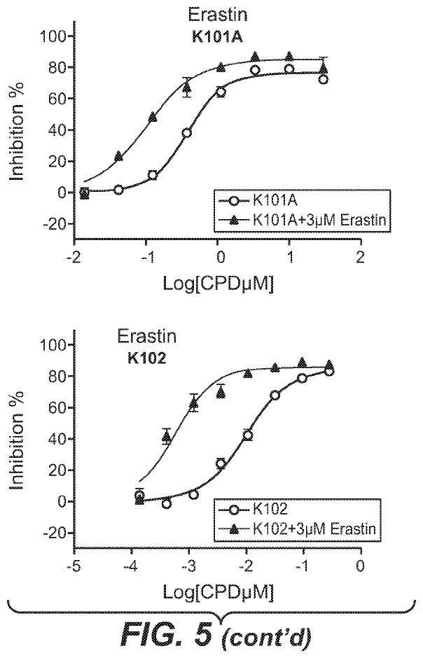

Since ferroptosis inducers are identified via Ras synthetic lethal screens and these compounds by themselves are not very potent inducers of cell death in A549 cells, we sought to determine if they can facilitate anti-proliferative potential of the PKC activators, which could block preferentially K-Ras driven stemness (Wang et al., 2015, Cell 163(5):1237-51). K101A or K102 were tested in 9-point titration proliferation assays in the presence of DMSO or ferroptosis inducers that showed differential activities in Example 1. In the combination experiment, ferroptosis inducers were used at concentrations where they induced minimal or no effect on proliferation of A549 alone. As shown in FIG. 5, both RSL3 and Erastin synergized strongly with K101A or K102 to block proliferation, whereas ML210 showed minor synergy. No synergy was observed for ferroptosis inducers Auranofin, Sulfasalazine, and Artemisinin related compounds. It has been suggested that PKC is a negative regulator of ferroptosis by phosphorylating HSPB1 which blocks iron uptake and ROS production (Xie et al., 2016, Cell Death Differ. 23(3):369-79). PKC activators then should act antagonistically with ferroptosis inducers. However, the data suggest that the opposite is true. Indeed, literature suggested that PKC activation may initiate ferroptosis in neuronal tissues (DoVan et al., 2016, Neurobiol Dis. 94:169-78) and PKC can phosphorylate iron-response element-binding protein, a positive regulator of ferroptosis (Eisenstein et al., 1993, J Biol Chem. 268(36):27363-70). Therefore, whether PKC activators synergize or antagonize with ferroptosis inducers can be tested empirically.

### Example 3: Effect of Combination of Ferroptosis Inducer and PKC Activator on Proliferation of Pancreatic Cancer Cells

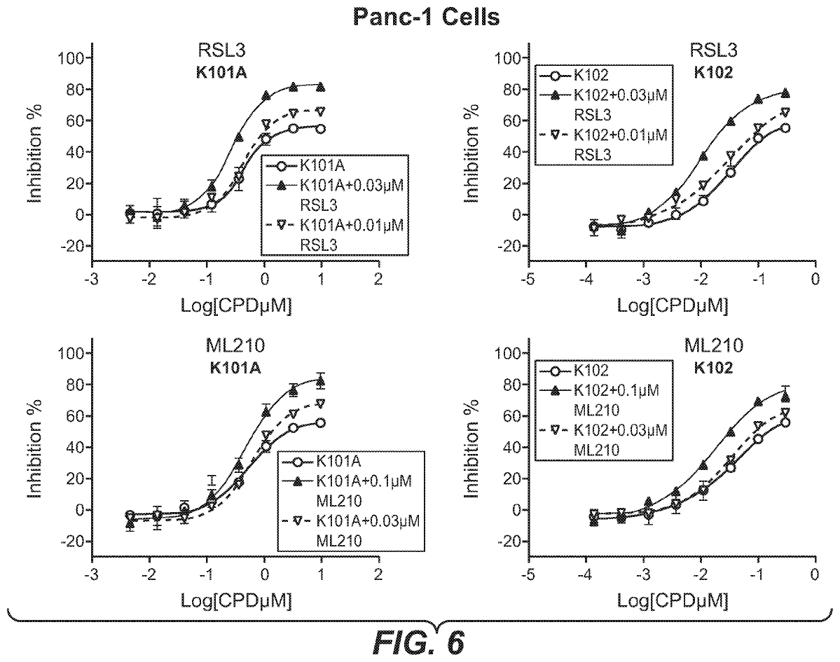

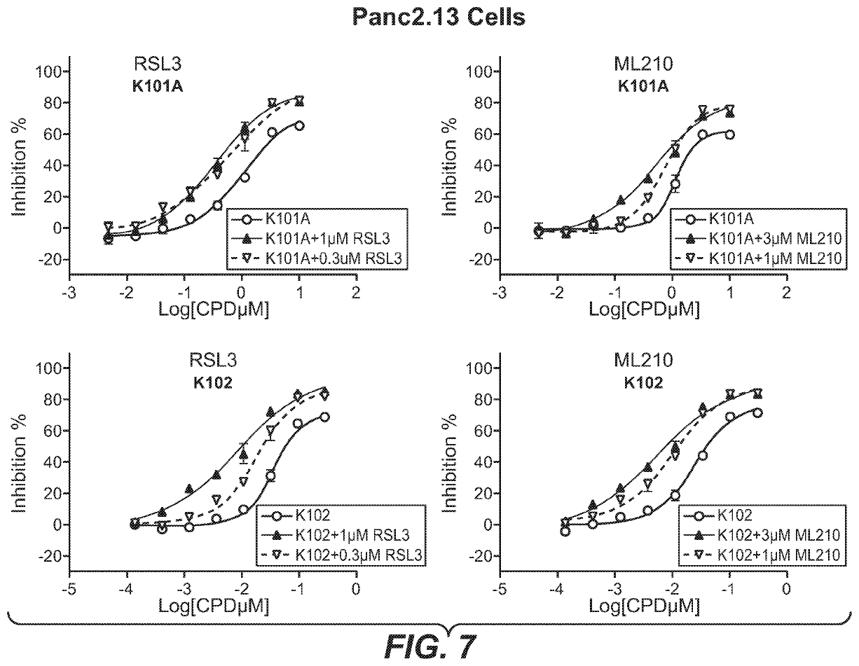

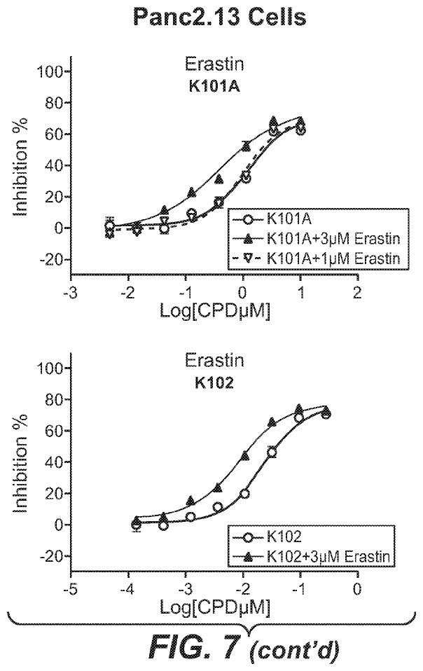

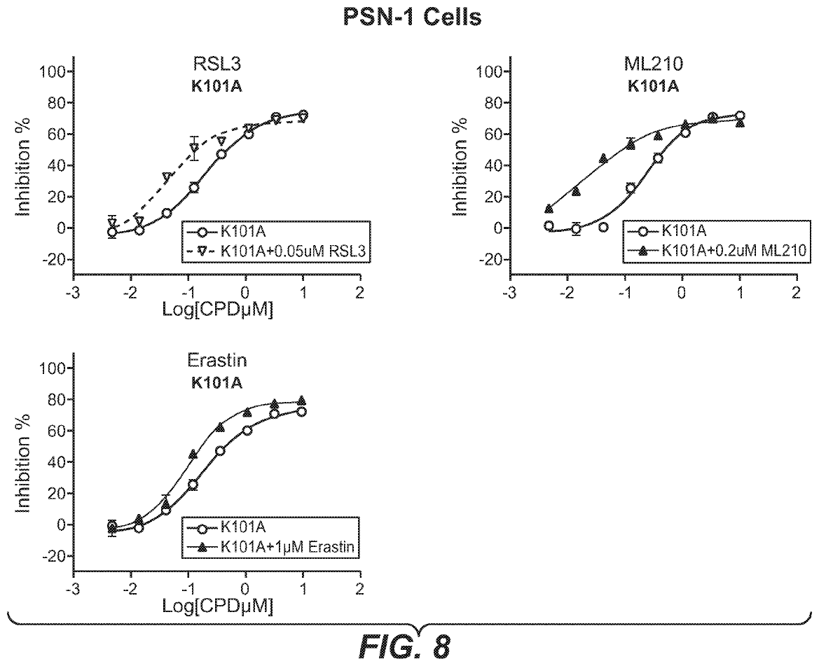

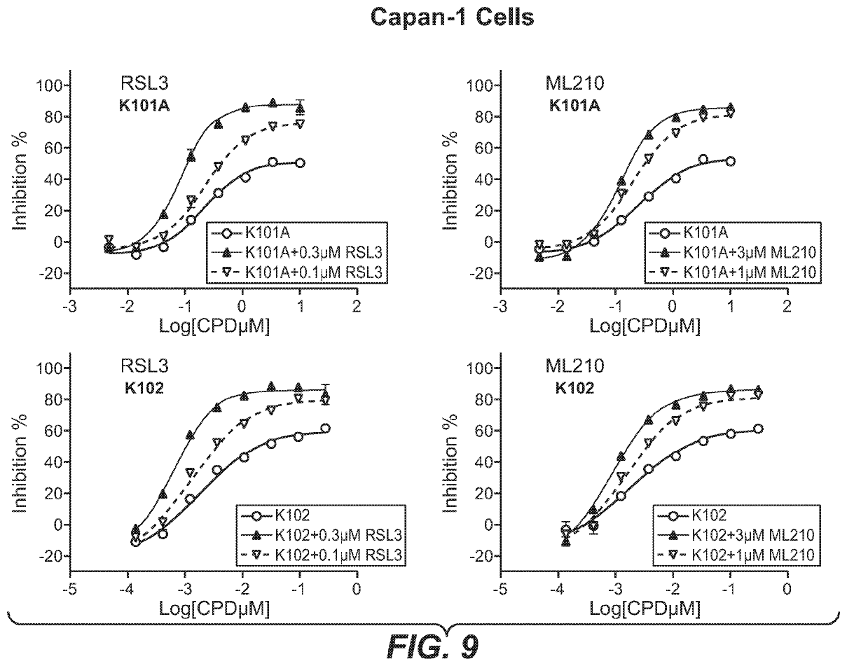

Similar combination experiments as in Example 2 were performed in pancreatic cell lines Panc1, Panc2.13, PSN-1, Capan-1, and KP-4. As shown in FIG. 6, RSL3 or ML210 synergized with K101A or K102 to block proliferation of Panc1 cells. In addition to increasing the potency of K101A or K102, RSL3 or ML210 also increased the % of top inhibition by K101A or K102 from 50-60% to >80%. No synergistic effects were observed when K101A or K102 was combined with Erastin (up to 1 μM) or Auranofin (up to 1 μM). As shown in FIG. 7, RSL3, ML210, or Erastin synergized with K101A or K102 to block proliferation of Panc2.13 cells. RSL3 or ML210 increased the potency of K101A or K102 and the % of top inhibition by K101A or K102 from ˜60% to ˜80% whereas Erastin only increased the potency of K101A or K102. Other ferroptosis inducers such as Auranofin, Sulfasalazine, and Arteminisin related compounds did not synergize with the PKC activators. As shown in FIG. 8, RSL3, ML210, or Erastin synergized with K101A to block proliferation of PSN-1 cells whereas Dihydroartemisinin showed no synergy with the PKC activators. As shown in FIG. 9, RSL3 or ML210 synergized strongly with K101A or K102 to block proliferation of Capan-1 cells. RSL3 or ML210 increased the potency of K101A or K102 as well as the % of top inhibition from 40-50% to ˜80%. Other ferroptosis inducers such as Erastin, Auranofin, and Arteminisin related compounds did not synergize with the PKC activators. In conclusion, GPX4 inhibitor RSL3 or ML210 seems to have strong synergy with the PKC activators to block cell proliferation in multiple pancreatic cell lines whereas the cystine/glutamate antiporter system Xc⁻ inhibitor Erastin has shown synergy with some but not all pancreatic cell lines tested.

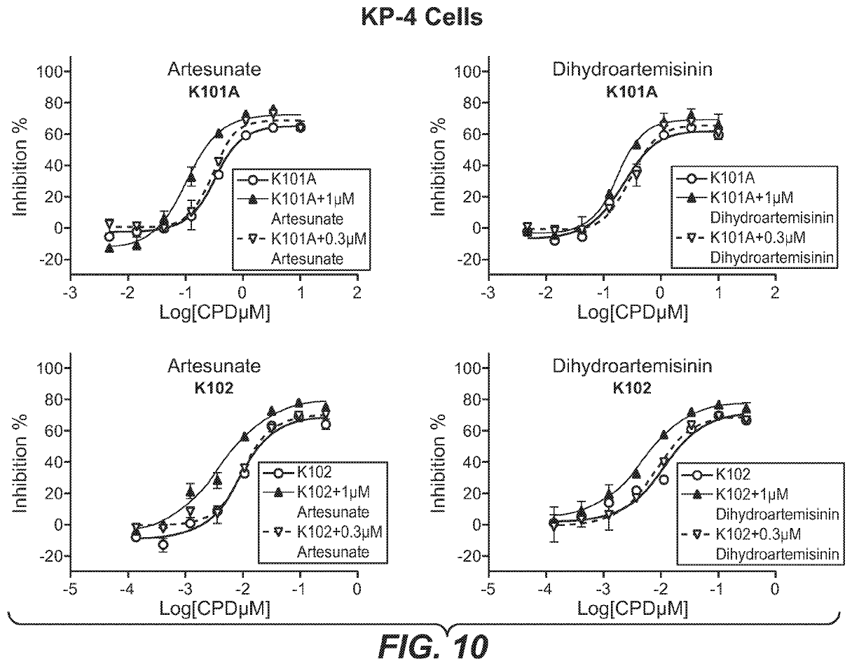

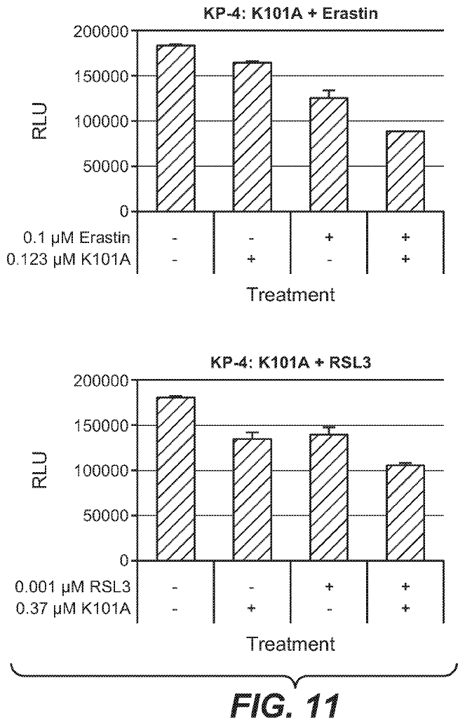

Results of combination experiments in KP-4 cells are shown in FIG. 10 and FIG. 11. Artesunate or Dihydroartemisinin had moderate/minor synergy with the PKC activator compound K101A or K102. Interestingly, RSL3 and Erastin showed only additive effects whereas ML210 and Auranofin showed no synergy when combined with K101A or K102 in KP-4 cells. This may be related to the fact that RSL3, ML210, Erastin, or Auranofin alone could induce ferroptosis at much lower concentration quite effectively in KP-4 cells than in other pancreatic cells, as shown in Example 1.

### Example 4: Combination of Ferroptosis Inducer and PKC Activator on Proliferation of Lymphoma Cells

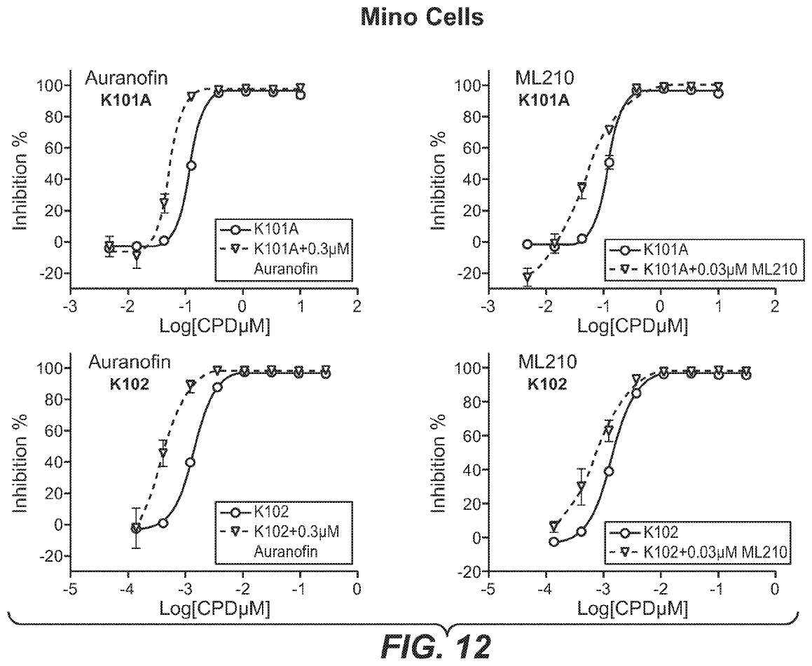

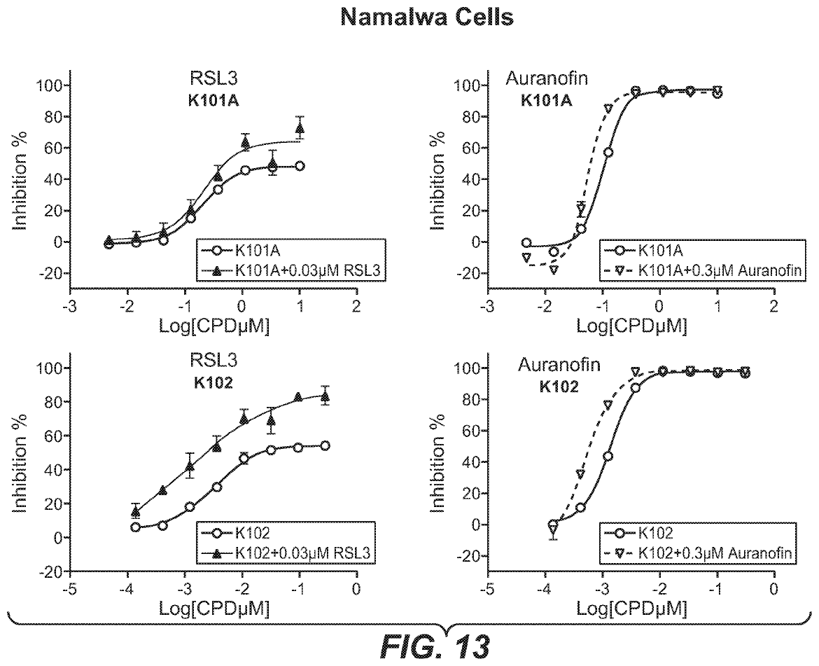

As shown in Example 1, lymphoma cell lines are sensitive to ferroptosis inducers. In addition, we identified that lymphoma cells are quite sensitive to anti-proliferation effect of PKC activators (see International application No. PCT/US2016/61711, incorporated herein by reference). Therefore, it is of interest to test combination treatment of both classes of compounds in lymphoma cells. Similar combination experiments as in Example 2 were performed on lymphoma cell lines Mino and Namalwa, and the results are shown in FIG. 12 and FIG. 13, respectively. Interestingly, Auranofin, which had not shown synergy with PKC activators in other cell lines, demonstrated moderate synergy in Mino and minor synergy in Namalwa when combined with K101A or K102. In Mino cell line, ML210 also showed minor synergy when combined with K101A or K102. In Namalwa cell line, addition of RSL3 to K101A or K102 improved potency and % of top inhibition of these PKC activators.

### Example 5: Effect of Ferroptosis Inhibitor on Cell Death Induced by Ferroptosis Inducer in KP-4 and A549

To investigate if cell death induced by ferroptosis inducers is different in sensitive and insensitive cells, similar cell viability experiment was performed as described in Example 1 with slight modifications to study effect of ferroptosis inhibitor or apoptosis inhibitor on RSL3 or Erastin-induced cell death in KP-4 or A549 cells. Ferroptosis inhibitors include: Ferrostatin-1 and Liproxstatin-1, inhibitors of ROS accumulation; Deferoxamine, an iron chelator; and PD146176, a 15-lipoxygenase inhibitor (see, e.g., Hofman et al., 2016, J Med Chem. March 10; 59(5):2041-53; Yang et al., 2016 Cell 26(3):165-176). The apoptosis inhibitor chosen was ZVAD (see, e.g. Wu et al., 2011, Cell Death and Differentiation 18:26-37). Ferroptosis inhibitors and ZVAD were first titrated as a single agent as shown in Example 1. Then, for each ferroptosis inhibitor, a concentration that had no anti-proliferative effect was identified to combine with a ferroptosis inducer.

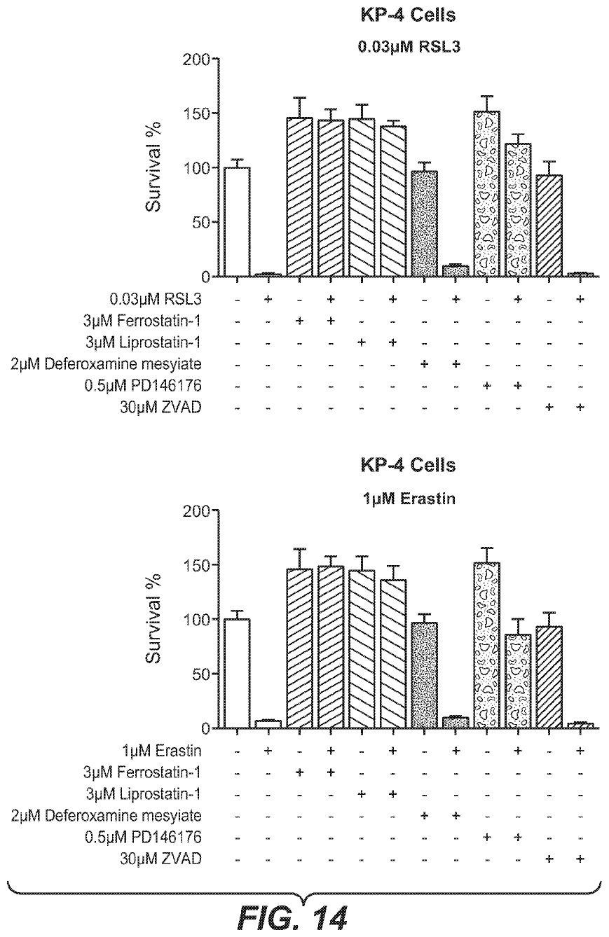

As shown in FIG. 14, treatment of KP-4 cells with RSL3 at 0.0304 or Erastin at 1 μM alone reduced cell viability by >90% compared to DMSO. Thus, KP-4 is quite sensitive to ferroptosis inducers. Co-treatment with ferroptosis inhibitors, such as Ferrostatin-1 at 3 μM or Liproxstatin-1 at 304 completely abrogated cell death induced by RSL3 or Erastin. While the iron chelator Deferoxamine at 204 did not rescue RSL3- or Erastin-induced cell death, another ferroptosis inhibitor PD146176 partially reversed RSL3- or Erastin-induced cell death at 0.504. We noted that some ferroptosis inhibitors stimulated cell growth in Kp-4 cells. However, the apoptosis inhibitor ZVAD at 30 μM did not rescue RSL3- or Erastin-induced cell death. Thus, we conclude that in sensitive cell lines such as KP-4, cell death induced by RSL3 or Erastin, which can only be reversed by a number of ferroptosis inhibitors, is ferroptosis but not apoptosis.

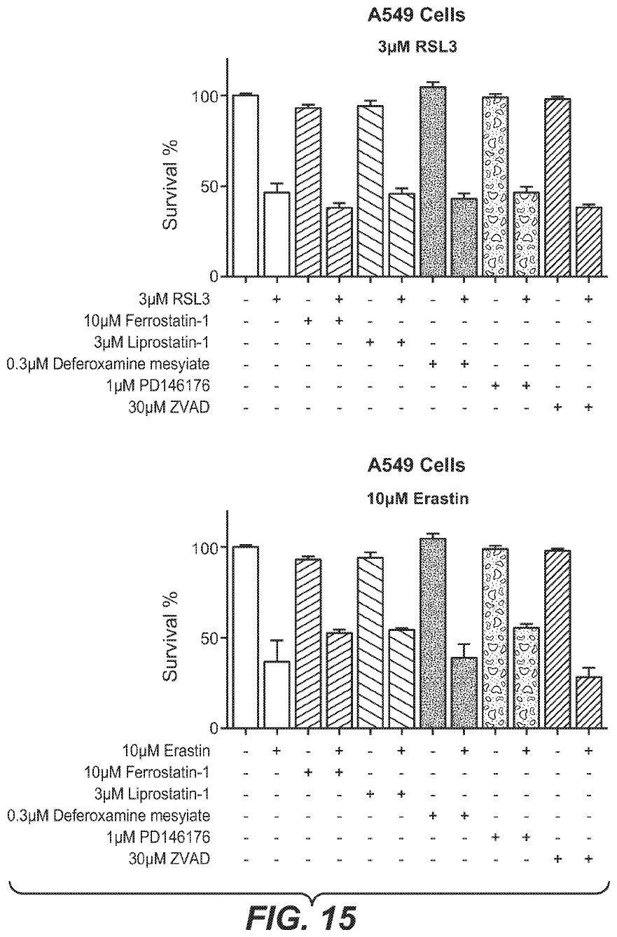

In A549 cells, RSL3 at 304 or Erastin at 10 μM reduced cell viability by 50-60% compared to DMSO. In contrast to the results from KP-4 cells, none of the ferroptosis inhibitors could reverse the effect of RSL3 or Erastin on cell viability when A549 cells were treated in combination (FIG. 15). As expected, the apoptosis inhibitor ZVAD at 3004 did not rescue RSL3- or Erastin-induced cell death. Therefore, in an insensitive cell line A549, cell death induced by RSL3 or Erastin at higher concentrations, not rescued by ferroptosis or apoptosis inhibitors, is neither ferroptosis nor apoptosis.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of treating cancer comprising administering to a subject in need thereof a therapeutically effective amount of a ferroptosis inducer.

2. The method of claim 1, wherein the cancer treated is adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer, intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer (gastric), testicular cancer, throat cancer, thyroid cancer, uterine cancer, urinary tract cancer, vaginal cancer, sarcoma or soft tissue carcinoma.

3. The method of claim 2, wherein the hematologic cancer is a leukemia or lymphoma.

4. The method of claim 3, wherein the leukemia is selected from acute lymphoblastic leukemia (ALL), acute myeloid leukemia (AML), chronic lymphocytic leukemia (CLL), chronic myelogenous leukemia (CML), Hairy Cell chronic myelogenous leukemia (CML), and multiple myeloma.

5. The method of claim 3, wherein the lymphoma is selected from Hodgkin's lymphoma, Non-Hodgkin's lymphoma, Burkitt's lymphoma.

6. The method of claim 2, wherein the cancer treated is pancreatic cancer.

7. The method of claim 6, wherein the pancreatic cancer is pancreatic adenocarcinoma or metastatic pancreatic cancer.

8. The method of claim 7, wherein the pancreatic adenocarcinoma is stage 1, stage II, stage III, or stage IV pancreatic adenocarcinoma.

9. The method of claim 2, wherein the cancer treated is lung cancer.

10. The method of claim 9, wherein the lung cancer is small cell lung cancer or non-small cell lung cancer.

11. The method of claim 10, wherein the non-small cell lung cancer is an adenocarcinoma, squamous cell carcinoma, or large cell carcinoma.

12. The method of claim 10, wherein the lung cancer is metastatic lung cancer.

13. The method of any one of claims 1 to 12, wherein the cancer is resistant to cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and erlotinib.

14. The method of any one of claims 1 to 12, wherein the cancer is resistant to ionizing radiation.

15. The method of any one of claims 1 to 12, wherein the cancer for treatment with the ferroptosis inducer has an identified activating or oncogenic RAS activity.

16. The method of claim 15, wherein the cancer for treatment with the ferroptosis inducer is identified as having an activating or oncogenic K-RAS, H-RAS or N-RAS activity.

17. The method of any one of claims 1 to 16, the ferroptosis inducer is a compound of formula (I): 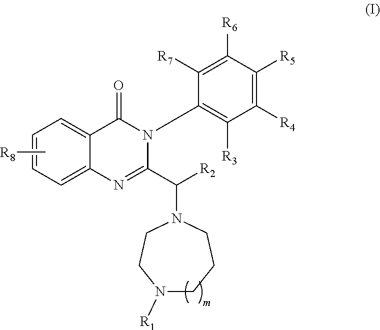
or an N-oxide, hydrate, solvate, or pharmaceutically acceptable salt thereof,
wherein
R₁ selected from the group consisting of H, C₁₋₄ alkyl, arylC₁₋₄ alkyl, aryl, heteroaryl, acyl, alkylsulfonyl, and arylsulfonyl, or R₁ is —NRₐR_{b}, wherein Rₐ and R_{b} are each independently selected from H, C₁₋₄ alkyl, arylC₁₋₄alkyl, aryl, heteroaryl, acyl, alkylsulfonyl, and arylsulfonyl;
R₂ is selected from the group consisting of H, C₁₋₈alkyl, —NRₐR_{b}, C₁₋₈alkyl-OR₃, 3- to 8-membered carbocyclic or heterocyclic, aryl, heteroaryl, and arylC₁₋₄alkyl;
R₃, R₄, R₅, R₆ and R₇ are each independently selected from the group consisting of H, halo, C₁₋₈alkyl, C₁₋₈alkylamino, C₁₋₈alkyloxy, C₂₋₈alkenyl, C₂₋₈ alkynyl, amide, amine, carbamate, carbonate, carboxy, acyl, ether, heterocycloalkyl, and arylalkyl; and
R₈ is selected from the group consisting of H, halo, NH₂, C₁₋₆ alkyl, C₁₋₄ alkyloxy, carbonyl, aryl, heteroaryl, C₃₋₈ cycloalkyl, and C₃₋₈ heterocycloalkyl; and
m is 0 or 1.

18. The method of any one of claims 1 to 16, wherein the ferroptosis inducer is a compound of formula (la): 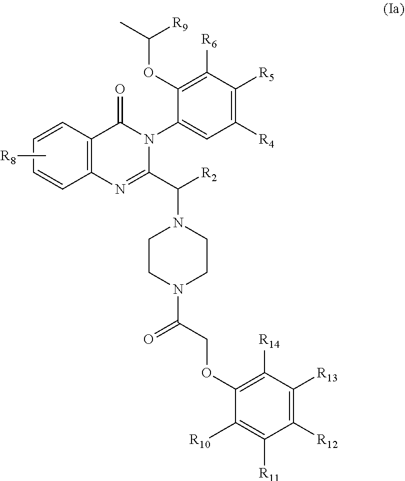
or an N-oxide, hydrate, solvate, or pharmaceutically acceptable salt thereof,
wherein
R₂ is selected from the group consisting of H, C₁₋₈ alkyl, —N(Rₐ)(R_{b}), C₁₋₈alkyl-OR₃, 3- to 8-membered carbocyclic or heterocyclic, aryl, heteroaryl, and arylC₁₋₄alkyl;
R₄, R₅, and R₆ are each independently selected from the group consisting of H, halo, C₁₋₈alkyl, C₁₋₈alkylamino, C₁₋₈alkyloxy, C₂₋₈ alkenyl, C₂₋₈ alkynyl, amide, amine, carbamate, carbonate, carboxy, acyl, ether, heteroalkyl, and arylalkyl;
R₈ is selected from group consisting of H, halo, NH₂, C₁₋₆ alkyl, C₁₋₄ alkyloxy, carbonyl, aryl, heteroaryl, C₃₋₈ cycloalkyl, and C₃₋₈ heterocycloalkyl;
R₉ is H or C₁₋₄ alkyl;
R₁₀, R₁₁, R₁₂, R₁₃, and R₁₄ are independently selected from the group consisting of H, halo, C₁₋₄alkyl, C₁₋₄ alkylamino, acyl, and alkylsulfonyl; and
Rₐ and R_{b} are each independently selected from the group consisting of H, C₁₋₄ alkyl, arylC₁₋₄alkyl, aryl, heteroaryl, acyl, alkylsulfonyl, and arylsulfonyl.

19. The method of any one of claims 1 to 16, wherein the ferroptosis inducer is a compound of formula (IV): 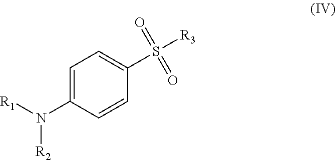
Or an N-oxide, hydrate, solvate, or pharmaceutically acceptable salt thereof,
Wherein,
R₁ and R₂ are H or together form N=phenyl, wherein the phenyl is substituted with a carboxyl group and/or a hydroxyl; and
R₃ is a —NH-pyridyl.

20. The method of any one of claims 1 to 16, wherein the ferroptosis inducer is a compound of formula (V): 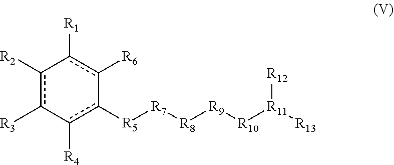
or an N-oxide, hydrate, solvate, or pharmaceutically acceptable salt thereof,
wherein
R₁, R₂, R₃, R₄, and R₆ are independently selected from the group consisting of H, halo, CF₃, OCF₃, C₁₋₄ alkyl, and CN;
R₅ is selected from the group consisting of no atom, NH, O, and C₁₋₄ alkyl;
R₇ is selected from the group consisting of no atom, carbonyl, thiocarbonyl, and sulfonyl;
R₈ is NH or no atom;
R₉ is an aryl or heteroaryl optionally substituted with hydroxy, C₁₋₄ alkyl, halo, and combinations thereof;
R₁₀ is no atom, O, or C₁₋₄ alkyl;
R₁₁ is selected from the group consisting of no atom, aryl, heterocyclyl, and heteroaryl optionally substituted with a halo or a C₁₋₄ alkyl;
R₁₂ is selected from the group consisting of no atom, amide, CN, heteroaryl, O, C₁₋₄ alkyloxy, and amine;
R₁₃ is selected from the group consisting of no atom, C₁₋₄ alkyloxy, amine, CN, carboxy, and carbocyclyl optionally substituted with one or more of C₁₋₄ alkyl and OH; and
the dotted line (- - - -) is an optional double bond.

21. The method of claim 20, wherein the ferroptosis inducer is a compound of formula (Vc): 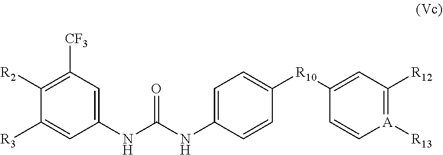
or a hydrate, solvate, or pharmaceutically acceptable salt thereof,
wherein
R₂ and R₃ are independently selected from the group consisting of H, halo, and CF₃;
R₁₀ is O or C₁₋₄ alkyl;
A is C or N;
B is O or S;
R₁₂ is selected from the group consisting of no atom, amide, CN, and heteroaryl; and
R₁₃ is selected from the group consisting of no atom or CN.

22. The method of any one of claims 1 to 16, wherein the ferroptosis inducer is a compound of formula (VI): 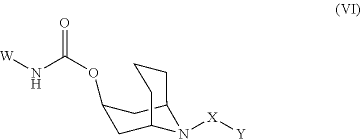
or a hydrate, solvate, or pharmaceutically acceptable salt thereof,
wherein
W is an optionally substituted C₆₋₁₀aryl or C₅₋₁₀heteroaryl;
X is a inking moiety; and
Y is a ferroptosis inducing moiety selected from erastin, erastin-A, erastin-B, desmethyl erastin and a synthetic erastin mimetic.

23. The method of any one of claims 1 to 16, wherein the ferroptosis inducer is a compound of formula (VII): 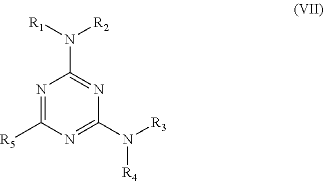
or a hydrate, solvate, or pharmaceutically acceptable salt thereof
wherein
R₁ and R₂ are each independently H or C₁₋₄alkyl;
R₃ and R₄ are each independently H, C₁₋₄ alkyl, C₂₋₄ alkenyl, optionally substituted aryl or fused aryl; and
R₅ is C₁₋₄alkyl, haloC₁₋₄alkyl; C₁₋₄ alkylamino; or di-C₁₋₄ alkylamino.

24. The method of any one of claims 1 to 16, wherein the ferroptosis inducer is a compound of structural formula (VIII): 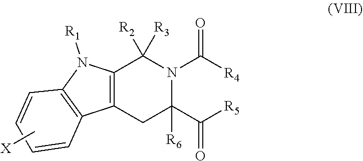
or a hydrate, solvate, or pharmaceutically acceptable salt thereof,
wherein
R₁, R₂, R₃, R₄, R₅, and R₆ are independently selected from the group consisting of H, C₁₋₈alkyloxy, 3- to 8-membered carbocyclic, 3- to 8-membered heterocyclyl, 3- to 8-membered aryl, or 3- to 8-membered heteroaryl, carboxylate, ester, amide, carbohydrate, amino acid, acyl, alkyloxy-substituted acyl, alditol, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether, wherein each alkyl, alkyloxy, carbocyclic, heterocyclyl, aryl, heteroaryl, carboxylate, ester, amide, carbohydrate, amino acid, acyl, alkyloxy-substituted acyl, alditol, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether is optionally substituted with at least one substituent;
R₇ is selected from the group consisting of H, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl may be optionally substituted with at least one substituent;
R₈ is selected from the group consisting of H, C₁₋₈alkyl, C₂₋₈alkenyl, C₂₋₈alkynyl, aryl, carbocycle, heteroaryl, heterocyclyl, alkylaryl, alkylheteroaryl, alkylheterocyclyl, and heteroaryl, wherein each alkyl, alkenyl, alkynyl, aryl, carbocycle, heteroaryl, heterocycle, alkylaryl, alkylheteroaryl, alkylheterocyclyl, and heteroaryl may be optionally substituted with at least one substituent; and
X is 0-4 substituents on the ring to which it is attached, wherein X is selected from the group consisting of H, cyano, oxo, nitro, acyl, acylamino, halo, hydroxy, amino acid, amine, amide, carbamate, ester, ether, carboxylic acid, thio, thioalkyl, thioester, thioether, C₁₋₈alkyl, C₁₋₈alkoxy, C₁₋₈alkenyl, arylC₁₋₈alkyl, 3- to 8-membered carbocyclic, 3- to 8-membered heterocyclyl, 3- to 8-membered aryl, 3- to 8-membered heteroaryl, sulfate, sulfonamide, sulfoxide, sulfonate, sulfone, alkylsulfonyl, and arylsulfonyl.

25. The method of any one of claims 1 to 16, wherein the ferroptosis inducer is a compound of structural formula (VIIId): 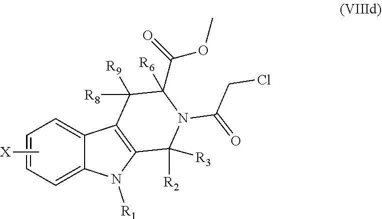
or a hydrate, solvate, or pharmaceutically acceptable salt thereof
wherein
R₂ and R₃ are independently selected from H, methyl, methyl benzoate, propargyl, and phenyl, wherein at least one of R₂ and R₃ is other than H;
R₁, R₆, R₈, and R₉ are independently selected from H, halo, C₁₋₈alkyl, C₁₋₈alkoxy, 3- to 8-membered carbocyclic, 3- to 8-membered heterocyclic, 3- to 8-membered aryl, or 3- to 8-membered heteroaryl, carboxylate, ester, ether, amide, amino acid, acyl, alkoxy-substituted acyl, NR₇R₈, OC(R₇)₂COOH, SC(R₇)₂COOH, NHCHR₇COOH, COR₈, CO₂R₈, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, ether, amide, amino acid, acyl, alkoxy-substituted acyl, NR₇R₈, OC(R₇)₂COOH, SC(R₇)₂COOH, NHCHR₇COOH, COR₈, CO₂%, sulfate, sulfonamide, sulfoxide, sulfonate, sulfone, thioalkyl, thioester, and thioether is optionally substituted with at least one substituent;
R₇ is selected from the group consisting of H, carbocycle, aryl, heteroaryl, heterocyclyl, alkylaryl, alkylheteroaryl, and alkylheterocyclyl, wherein each alkyl, carbocycle, aryl, heteroaryl, heterocyclyl, alkylaryl, alkylheteroaryl, and alkylheterocyclyl is optionally substituted with at least one substituent; and
X is 0-4 substituents on the ring to which it is attached, wherein X is selected from the group consisting of H, cyano, oxo, nitro, acyl, acylamino, halo, hydroxy, amino acid, amine, amide, carbamate, ester, ether, carboxylic acid, thio, thioalkyl, thioester, thioether, C₁₋₈ alkyl, C₁₋₈ alkoxy, C₁₋₈alkenyl, arylC₁₋₈alkyl, 3- to 8-membered carbocyclic, 3- to 8-membered heterocyclyl, 3- to 8-membered aryl, 3- to 8-membered heteroaryl, sulfate, sulfonamide, sulfoxide, sulfonate, sulfone, alkylsulfonyl, and arylsulfonyl.

26. The method of any one of claims 1 to 16, wherein the ferroptosis inducer is a compound of formula (XII): 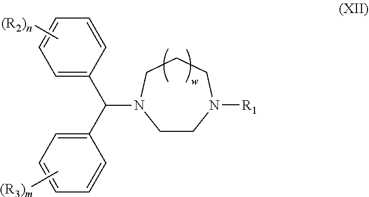
or a hydrate, solvate, or pharmaceutically acceptable salt thereof,
wherein,
R₁ is selected from the group consisting of C₁₋₄ alkyl, C₂₋₄alkenyl, optionally substituted heterocyclyl, optionally substituted aryl, optionally substituted heteroaryl, optionally substituted aryl C₁₋₄alkyl, optionally substituted arylC₂₋₄ alkenyl, optionally substituted heteroarylC₁₋₄ alkyl, optionally substituted heteroarylC₂₋₄ alkenyl, optionally substituted heterocyclylcarbonyl, optionally substituted arylcarbonyl, optionally substituted heteroarylcarbonyl, carboxyC₁₋₆ heteroalkyl, and hydroxyC₁₋₆heteroalkyl, wherein the substitution when present is selected from the group consisting of halo, amino, C₁₋₄ alkylamino, di-C₁₋₄ alkylamino, C₂₋₄ alkenylamino, di-C₂₋₄alkenylamino, nitro, sulfonyl, aminosulfonyl, C₁₋₄ alkyl, heteroaryl, and heterocyclyl;
R₂ and R₃ are independently Cl or F;
n and m are independently 0, 1 or 2; and
w is 0 or 1.

27. The method of any one of claims 1 to 16, wherein the ferroptosis inducer is a compound of formula (XIII): 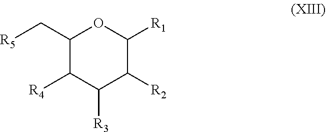
or a hydrate, solvate, or pharmaceutically acceptable salt thereof
wherein,
R₁, R₂, R₃, R₄ and R₅ are each independently selected from the group consisting of H, OH, C₁₋₄alkyloxy, C₁₋₄alkylcarbonyloxy, —NRₐR_{b}, —S—Au═P(R₆)₃, and —S—Au—R₇; wherein
each R₆ is independently an optionally substituted C₁₋₄ alkyl;
R₇ is 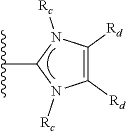
wherein
each R_{c} is independently selected from the group consisting of H, C₂₋₄alkenyl, optionally substituted phenylC₁₋₄ alkyl, and optionally substituted phenyl, wherein the optional substitution on the phenyl is —C₁₋₄ alkyl on one or more of the carbon atoms of the phenyl;
each R_{d} is H, C₁₋₄alkyl, or the two R_{d} together form an optionally substituted phenyl;
Rₐ and R_{b} are each independently selected from the group consisting of H and C₁₋₄alkylcarbonyl; and
at least one of R₁, R₂, R₃, R₄ and R₅ is —S—Au═P(R₆)₃ or —S—Au—R₇.

28. The method of any one of claims 1 to 16, wherein the ferroptosis inducer is a compound of formula (XIV): 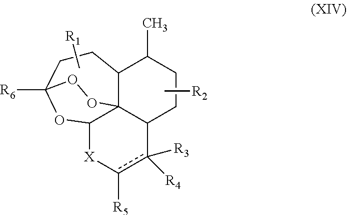
or a hydrate, solvate, or pharmaceutically acceptable salt thereof
wherein [symbol]
is a single or double bond;
R₁, R₂, R₃, R₄, and R₆ are each independently selected from the group consisting of H, halogen, —CF₃, ═CH₂, —OR₇, —NR₇R₈, —(CH₂)ₙCOOR₇, —(CH₂)ₙC(═O)R₇, —(CH₂)ₙCONR₇R₇, C₁₋₆ alkyl, C₂₋₆ alkenyl, C₂₋₆ alkynyl, C₃₋₇ cycloalkyl, C₃₋₇ heterocycloalkyl, aryl and heteroaryl;
R₅ is selected from the group consisting of H, halogen, ═O, —OR₇, —NR₇R₈, —(CH₂)ₙCF₃, —(CH₂)ₙCHF₂, —(CH₂)ₙC(═O)R₇, —O(CH₂)ₙCOOR₇, —OC(═O)(CH₂)ₙCOOR₇, —OC(═O)R₇, C₁₋₆ alkyl, C₂₋₆alkenyl, C₂₋₆ alkynyl, C₃₋₇ cycloalkyl, C₃₋₇ heterocycloalkyl, aryl and heteroaryl;
X is O or —NR₇;
wherein
R₇ is selected from the group consisting of H, optionally substituted C₁₋₆ alkyl, C₂₋₆ alkenyl, and C₂₋₆ alkynyl; and
R₈ is selected from the group consisting of H or an optionally substituted C₁₋₆ alkyl, C₂₋₆alkenyl, C₂₋₆ alkynyl, cycloalkyl, aryl, heteroaryl, and arylalkyl; or
R₇ and R₈ together with the nitrogen atom to which R₇ and R₈ are attached form an optionally substituted heterocyclic ring, wherein the heterocyclic atom is N, O or S.

29. The method of any one of claims 1 to 16, wherein the ferroptosis inducer is a compound of formula (XV): 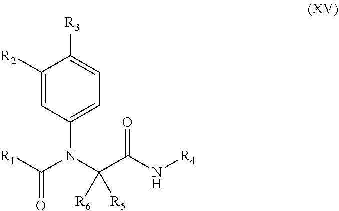
or a hydrate, solvate, or pharmaceutically acceptable salt thereof,
wherein
R₁ is an optionally substituted C₁₋₄ alkyl, optionally substituted C₁₋₄ alkyl-O—, optionally substituted C₁₋₄ alkyl-O—C₁₋₄ alkyl, or optionally substituted heteroaryl, wherein the substitution when present is selected from OH, halo, and thio;
R₂ is H, halo, or C₁₋₄alkyl-O—;
R₃ is H or C₁₋₄ alkyl-O—;
R₄ is optionally substituted cycloalkylC₁₋₄ alkyl, optionally substituted arylC₁₋₄ alkyl, optionally substituted heteroarylC₁₋₄ alkyl, or optionally substituted aryl-S(O)₂C₁₋₄ alkyl, wherein the substitution when present is OH, halo or C₁₋₄alkyl;
R₅ is H or C₁₋₄ alkyl; and
R₆ is H or optionally substituted cycloalkyl, heterocycloalkyl, C₁₋₄ alkyl, wherein the substitution when present is selected from halo or C₁₋₄ alkyl.

30. The method of claim 29, wherein
R₁ is HOCH₂—, ClCH₂—, C₁₋₄ alkyl, CH₃O—, CH₃—O—CH₂—, or thiopyrimidine;
R₂ is H, Cl, or CH₃O—;
R₃ is H or CH₃O—;
R₄ is C₃₋₆ cycloalkylC₁₋₄ alkyl, phenylC₁₋₄ alkyl, heteroarylC₁₋₄ alkyl, or 4-methylphenyl-S(O)₂C₁₋₄alkyl;
R₅ is H or CH₃—; and
R₆ is H, CH₃—, or 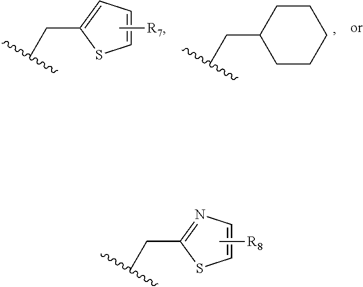
wherein
R₇ is H or halo; and
R₈ is H or C₁₋₄ alkyl.

31. The method of any one of claims 1 to 30, further comprising adjunctively administering a therapeutically effective amount of radiation therapy to treat the cancer.

32. The method of claim 31, wherein the ferroptosis inducer is administered to the subject in need thereof prior to, concurrently with, or subsequent to the radiation therapy.

33. The method of claim 31, wherein the radiation therapy is fractionated.

34. The method of any one of claims 1 to 30, further comprising adjunctively administering a therapeutically effective amount of a second therapeutic agent to treat the cancer.

35. The method of claim 34, wherein the ferroptosis inducer is administered to the subject in need thereof prior to, concurrently with, or subsequent to the administering of the second therapeutic agent.

36. The method of claim 34, wherein the second therapeutic agent comprises a chemotherapeutic agent.

37. The method of claim 36, wherein the chemotherapeutic agent is selected from an alkylating agent, antibiotic agent, antimetabolic agent, topoisomerase inhibiting agent, antimicrotubule agent, hormonal agent, antiangiogenic agent, differentiation inducing agents, cell growth arrest inducing agent, apoptosis inducing agent, and cytotoxic agent.

38. The method of claim 36, wherein the chemotherapeutic agent is selected from afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, and vindesine.

39. The method of claim 36, wherein the second therapeutic agent comprises a diterpenoid PKC activator.

40. The method of claim 39, wherein the diterpenoid PKC activator is a compound of formula (PI): 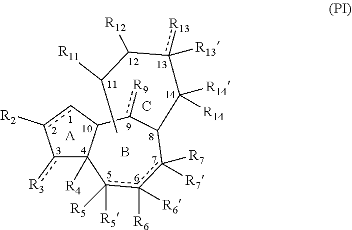
or an enantiomer, hydrate, solvate, or pharmaceutically acceptable salt thereof
wherein
Ring C is attached to Ring B at carbon atom 9 or 10;
R₂ is selected from H or lower alkyl;
R₃ is H, or O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl, —S(O)₂R_{b}, —S(O)₂OR_{b}, or —P(O)(OR_{b})₂;
R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, —S(O)₂R_{b}, —S(O)₂OR_{b}, and —P(O)(OR_{b})₂;
R₅′ and R₆′ are H, or R₅′ and R₆′ together form a bond or are bonded to a common oxygen atom to form an epoxide;
R₆ is —NR_{b}R_{b}, —NHC(O)R_{b}, —SR_{b}, SOR_{b}, —S(O)₂R_{b}, —S(O)₂OR_{b}, —P(O)(OR_{b})₂, —SeR_{b}, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —C₁₋₄ alkyl-O—R_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, —S(O)₂R_{b}, —S(O)₂OR_{b}, —P(O)(OR_{b}), or R_{d} is a promoiety which is hydrolyzable under biological conditions to yield an -alkyl-OH.
R₆′ and R₇′ are H, or R₆′ and R₇′ together form a bond or are bonded to a common oxygen atom to form an epoxide;
R₇ is H or OH;
R₉ is H, oxo, or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl, or R₉′, is an O atom which is bonded to an optionally substituted common C atom bonded to R₁₃′ and R₁₄′, wherein R₁₃′ and R₁₄′ each is an O atom;
R₁₁ is lower alkyl;
R₁₂ is H, halo, —NR_{b}R_{b}, —NHC(O)R_{b}, —SR_{b}, SOR_{B}, —S(O)₂R_{b}, —S(O)₂OR_{b}, —P(O)(OR_{b})₂, —SeR_{b}, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or R₁₂ is —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, —S(O)₂R_{b}, —S(O)₂OR_{b}, and —P(O)(OR_{b})₂;
R₁₃ is H, halo, oxo, —NR_{b}R_{b}, —NHC(O)R_{b}, —SR_{b}, SOR_{B}, —S(O)₂R_{b}, —S(O)₂OR_{b}, —P(O)(OR_{b})₂, —SeR_{b}, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —OR_{b}, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, —S(O)₂R_{b}, —S(O)₂OR_{b}, and —P(O)(OR_{b})₂;
R₁₃′ and R₁₄′ are independently H, OH, or are bonded to a common carbon atom to form a cyclopropyl ring, wherein the cyclopropyl ring is optionally mono- or disubstituted with OH, halo, —NR_{b}R_{b}, —NHC(O)R_{b}, —SR_{b}, SOR_{B}, —S(O)₂R_{b}, —S(O)₂OR_{b}, and —OP(O)(OR_{b})₂, —SeR_{b}, optionally substituted alkyl, optionally substituted cycloalkyl, optionally substituted heteroalkyl, optionally substituted aryl, optionally substituted heteroaryl, optionally substituted alkyloxy, optionally substituted alkenyloxy, optionally substituted alkynyloxy, optionally substituted cycloalkyloxy, optionally substituted cycloalkenyloxy, optionally substituted heterocycloalkyloxy, optionally substituted aryloxy, optionally substituted heteroaryloxy, optionally substituted arylalkyloxy, optionally substituted arylalkenyloxy, optionally substituted heteroarylalkyloxy, optionally substituted heteroarylalkenyloxy, optionally substituted alkylcarbonyloxy, optionally substituted alkenylcarbonyloxy, optionally substituted alkynylcarbonyloxy, optionally substituted arylcarbonyloxy, optionally substituted heteroarylcarbonyloxy, optionally substituted arylalkylcarbonyloxy, optionally substituted arylalkenylcarbonyloxy, optionally substituted heteroarylalkylcarbonyloxy, optionally substituted heteroarylalkenylcarbonyloxy, optionally substituted carboxyalkylcarbonyloxy, optionally substituted amino acid carbonyloxy, carbamate, phosphine, phosphoramide, phosphoramidite, phosphoramidate, phosphonate, sulfonamide, amide, guanidine, urea; or a progroup which is hydrolysable under biological conditions to yield an -alkyl-OH group, or R₁₃′ and R₁₄′ are each an O atom which is bonded to an optionally substituted common C atom bonded to R₉, wherein R₉ is an O atom;
R₁₄ is H, OH or optionally substituted alkenyl;
wherein each R_{b} is independently H, optionally substituted alkyl, optionally substituted alkenyl, alkynyl, optionally substituted aryl, optionally substituted heteroaryl, optionally substituted arylalkyl, or optionally substituted heteroarylalkyl; and
the dashed line (- - - - -) represents an optional bond.

41. The method of claim 40, wherein the PKC activator is a compound of formula (PII): 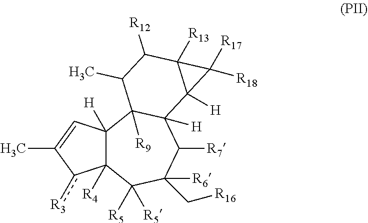
or an enantiomer, hydrate, solvate, or pharmaceutically acceptable salt thereof,
wherein
R₃ is O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;
R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₅′ and R₆′ are H, or R₅′ and R₆′ together form a bond or are bonded to a common oxygen atom to form an epoxide;
R₆′ and R₇′ are H, or R₆′ and R₇′ together form a bond or are bonded to a common oxygen atom to form an epoxide;
R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl;
R₁₂ is H, halo, or —OR_{g}, wherein R_{g} is H, optionally substituted alkyl, optionally substituted alkenyl, optionally substituted alkynyl, optionally substituted cycloalkyl, optionally substituted aryl, optionally substituted heteroaryl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₁₃ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₁₆ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group at R₁₆; and
R₁₇ and R₁₈ are each independently H, OH, amino, thiol, sulfanyl, sulfinyl, sulfonyl, optionally substituted alkyl, optionally substituted cycloalkyl, optionally substituted heteroalkyl, optionally substituted aryl, optionally substituted heteroaryl, optionally substituted alkyloxy, optionally substituted alkenyloxy, optionally substituted aryloxy, optionally substituted arylalkyloxy, optionally substituted alkylcarbonyloxy, optionally substituted alkenylcarbonyloxy, optionally substituted arylcarbonyloxy, optionally substituted arylalkylcarbonyloxy, phosphine, phosphate, phosphoramide, phosphoramidite, phosphoramidate, phosphonate, sulfate, sulfonate, sulfonamide, sulfone, sulfite, amide, guanidine, or urea.

42. The method of claim 41, wherein the PKC activator comprises the compound of formula (PIIa): 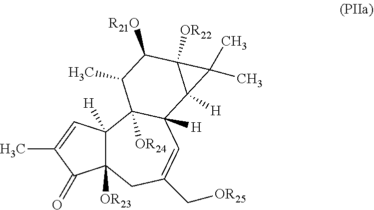
or an enantiomer, hydrate, solvate, or pharmaceutically acceptable salt thereof,
wherein
R₂₁, R₂₂, R₂₃, and R₂₄ are each independently H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and
R₂₅ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R₂₅ is a promoiety which is hydrolyzable under biological conditions to yield an —OH group at the C20 carbon atom.

43. The method of claim 41, wherein the PKC activator comprises a compound of formula (PIIb): 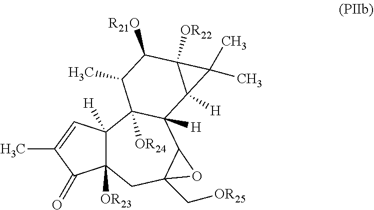
or an enantiomer, hydrate, solvate, or pharmaceutically acceptable salt thereof,
wherein,
R₂₁, R₂₂, R₂₃, R₂₄ and R₂₅ are as defined for formula (PIIa).

44. The method of claim 40, wherein the PKC activator is compound of formula (PIII): 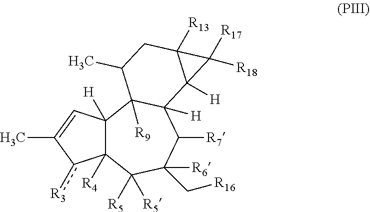
or an enantiomer, hydrate, solvate, or pharmaceutically acceptable salt thereof,
wherein
R₃ is O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;
R₄ and R₅ are independently H, halo, cyano, or R₄ is —OR_{c}, wherein R_{c} is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₅′ and R₆′ are H, or R₅′ and R₆′ together form a bond or are bonded to a common oxygen atom to form an epoxide;
R₆′ and R₇′ are H, or R₆′ and R₇′ together form a bond or are bonded to a common oxygen atom to form an epoxide;
R₉ is H or —OR_{f}, wherein R_{f} is H, an optionally substituted alkyl, optionally substituted aryl, optionally substituted heteroaryl, optionally substituted alkylcarbonyl; optionally substituted arylcarbonyl, optionally substituted heteroarylcarbonyl, optionally substituted arylalkylcarbonyl, optionally substituted heteroarylalkylcarbonyl, or optionally substituted arylalkyloxycarbonyl;
R₁₃ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₁₆ is H, halo, or —O—R_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group—at the C20 carbon atom; and
R₁₇ and R₁₈ are each independently H, OH, amino, thiol, sulfanyl, sulfinyl, sulfonyl, optionally substituted alkyl, optionally substituted cycloalkyl, optionally substituted heteroalkyl, optionally substituted aryl, optionally substituted heteroaryl, optionally substituted alkyloxy, optionally substituted alkenyloxy, optionally substituted alkynyloxy, optionally substituted cycloalkyloxy, optionally substituted cycloalkenyloxy, optionally substituted heterocycloalkyloxy, optionally substituted aryloxy, optionally substituted heteroaryloxy, optionally substituted arylalkyloxy, optionally substituted arylalkenyloxy, optionally substituted heteroarylalkyloxy, optionally substituted heteroarylalkenyloxy, optionally substituted alkylcarbonyloxy, optionally substituted alkenylcarbonyloxy, optionally substituted alkynylcarbonyloxy, optionally substituted arylcarbonyloxy, optionally substituted heteroarylcarbonyloxy, optionally substituted arylalkylcarbonyloxy, optionally substituted arylalkenylcarbonyloxy, optionally substituted heteroarylalkylcarbonyloxy, optionally substituted heteroarylalkenylcarbonyloxy, optionally substituted carboxyalkylcarbonyloxy, optionally substituted amino acid carbonyloxy, phosphine, phosphate, phosphoramide, phosphoramidite, phosphoramidate, phosphonate, sulfate, sulfonate, sulfonamide, sulfone, sulfite, amide, guanidine, urea, or a progroup which is hydrolyzable under biological conditions to yield an -alkyl-OH group.

45. The method of claim 44, wherein the PKC activator comprises a compound of formula (PIIIa) or (PIIIb): 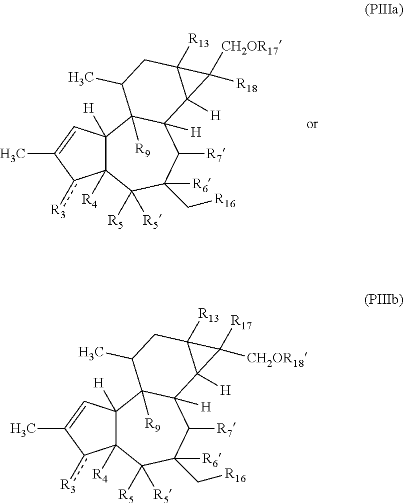
or an enantiomer, hydrate, solvate, or pharmaceutically acceptable salt thereof,
wherein
R₃; R₄, R₅, R₅′ R₆′, R₇′, R₉, R₁₃, and R₁₆ are as defined for formula (PIII);
R₁₇ or R₁₈ is H, OH, amino, thiol, sulfanyl, sulfinyl, sulfonyl, optionally substituted alkyl, optionally substituted cycloalkyl, optionally substituted heteroalkyl, optionally substituted aryl, optionally substituted heteroaryl, optionally substituted alkyloxy, optionally substituted aryloxy, optionally substituted arylalkyloxy, phosphine, phosphate, phosphoramide, phosphoramidite, phosphoramidate, phosphonate, sulfate, sulfonate, sulfonamide, sulfone, sulfite, amide, guanidine, or urea; and
R₁₇′ or R₁₈′ is H, optionally substituted alkyl, optionally substituted cycloalkyl, optionally substituted heteroalkyl, optionally substituted aryl, optionally substituted heteroaryl, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a progroup which is hydrolyzable under biological conditions to yield an —OH group.

46. The method of claim 44, wherein the PKC activator comprises a compound of formula (PIIIc): 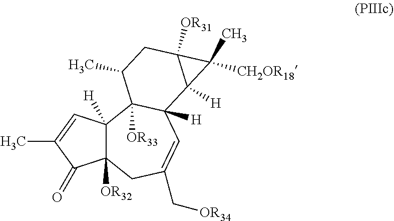
or an enantiomer, hydrate, solvate, or pharmaceutically acceptable salt thereof,
wherein,
R₁₈′ is H, optionally substituted alkyl, optionally substituted cycloalkyl, optionally substituted heteroalkyl, optionally substituted aryl, optionally substituted heteroaryl, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a promoiety which is hydrolyzable under biological conditions to yield an —OH group;
R₃₁, R₃₂, and R₃₃ are each independently H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and
R₃₄ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R₃₄ is a promoiety which is hydrolyzable under biological conditions to yield an —OH group—at the C20 carbon atom.

47. The method of claim 44, wherein the PKC activator comprises a compound of formula (PIIId): 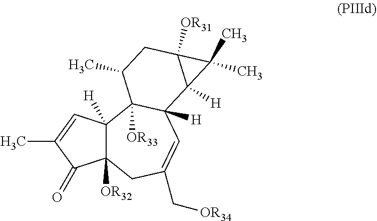
or an enantiomer, hydrate, solvate, or pharmaceutically acceptable salt thereof,
wherein
R₃₁, R₃₂, and R₃₃ are each independently H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and
R₃₄ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R₃₄ is a promoiety which is hydrolyzable under biological conditions to yield an —OH group—at the C20 carbon atom.

48. The method of claim 44, the PKC activator comprises a compound of formula (PIIIe): 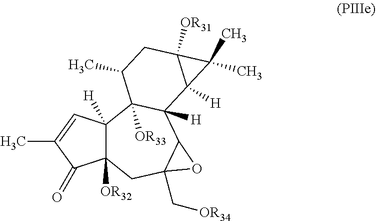
or an enantiomer, hydrate, solvate, or pharmaceutically acceptable salt thereof,
wherein,
R₃₁, R₃₂, R₃₃, and R₃₄ are as defined for formula (PIIIc).

49. The method of claim 40, wherein the PKC activator comprises a compound of formula (PIV): 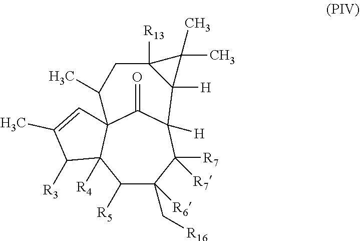
or an enantiomer, hydrate, solvate, or pharmaceutically acceptable salt thereof,
wherein
R₃ is O, S or N double bonded to the ring carbon, or R₃ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;
R₄ and R₅ are independently H, halo, cyano, or R₄ is —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₆′ and R₇′ are H, or R₆′ and R₇′ together form a bond or are bonded to a common oxygen atom to form an epoxide;
R₇ is H or OH;
R₁₃ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and
R₁₆ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a promoiety which is hydrolyzable under biological conditions to yield an —OH group at R₁₆.

50. The method of claim 49, wherein the PKC activator comprises a compound of formula (PIVa): 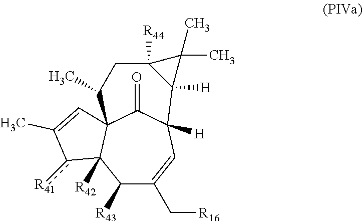
or an enantiomer, hydrate, solvate, or pharmaceutically acceptable salt thereof,
wherein
R₄₁ is O double bonded to the ring carbon, or R₄₁ is —ORₐ, wherein Rₐ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl;
R₄₂ and R₄₃ are independently H, halo, or —OR_{c}, wherein R, is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₄₄ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and
R₁₆ is H, halo, or —OR_{d}, wherein R_{d} is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or R_{d} is a biohydrolyzable promoiety which is hydrolyzable under biological conditions to yield an —OH group at R₁₆.

51. The method of claim 50, wherein the PKC activator comprises a compound of formula (PIVb): 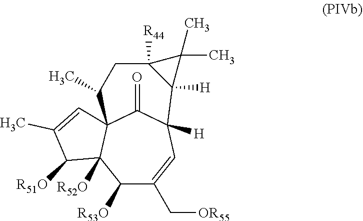
or an enantiomer, hydrate, solvate, or pharmaceutically acceptable salt thereof,
wherein
R₄₄ is H, halo, carbamate, phosphine, phosphoramide, phosphoramidite, phosphoramidate, phosphonate, sulfonamide, amide, guanidine, urea, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₅₁ is H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted heteroarylalkylcarbonyl, arylalkenylcarbonyl, optionally substituted heteroarylalkenylcarbonyl; and
R₅₂ and R₅₃ are independently H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl; and
R₅₅ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a promoiety which is hydrolyzable under biological conditions to yield an —OH group at the C20 carbon atom.

52. The method of claim 51, wherein the PKC activator comprises a compound of formula (PIVc): 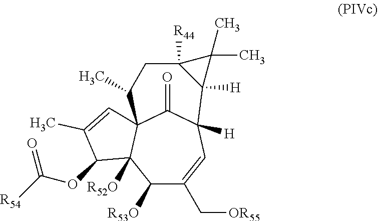
or an enantiomer, hydrate, solvate, or pharmaceutically acceptable salt thereof,
wherein
R₄₄ is H or —ORₕ, wherein Rₕ is H, optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₅₂ and R₅₃ are independently H, an optionally substituted alkyl, optionally substituted alkenyl, optionally substituted alkynyl, optionally substituted cycloalkyl, optionally substituted aryl, optionally substituted heteroaryl, optionally substituted arylalkyl, optionally substituted heteroarylalkyl, optionally substituted alkylcarbonyl, optionally substituted alkenylcarbonyl, optionally substituted cycloalkylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl;
R₅₄ is H, an optionally substituted alkyl, optionally substituted alkenyl, optionally substituted cycloalkyl, optionally substituted aryl, optionally substituted heteroaryl, optionally substituted arylalkyl, optionally substituted arylalkenyl, optionally substituted heteroarylalkyl, optionally substituted heteroarylalkenyl; and
R₅₅ is H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, optionally substituted arylalkenyl, optionally substituted heteroarylalkyl, optionally substituted heteroarylalkenyl, optionally substituted alkylcarbonyl, optionally substituted alkenylcarbonyl, optionally substituted alkynylcarbonyl, optionally substituted arylcarbonyl, optionally substituted heteroarylcarbonyl, optionally substituted arylalkylcarbonyl, optionally substituted arylalkenylcarbonyl, optionally substituted heteroarylalkylcarbonyl, optionally substituted heteroarylalkenylcarbonyl, optionally substituted carboxyalkylcarbonyl, optionally substituted amino acid carbonyl, or a promoiety which is hydrolyzable under biological conditions to yield an —OH group at the C20 carbon atom.

53. The method of claim 50, wherein the PKC activator comprises a compound of formula (PIVd): 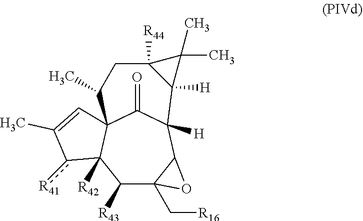
or an enantiomer, hydrate, solvate, or pharmaceutically acceptable salt thereof,
wherein
R₄₁, R₄₂, R₄₃, R₄₄ and R₁₆ are as defined for formula (PIVa).

54. The method of claim 39, wherein the PKC activator comprises a compound of formula (PVI): 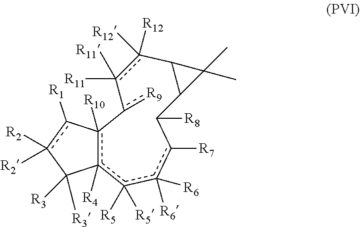
or an enantiomer, hydrate, solvate, or pharmaceutically acceptable salt thereof,
wherein
R₁ is H, or R₁ with R₂′ is a bond, or is bonded to a common O atom with R₂′ to form a epoxide ring;
R₂ is CH₃—, HOCH₂—, CH₃(O)COCH₂—; CH₃(O)COCH₂—, or phenyl(O)COCH₂—;
R₂′ is H, OH, CH₃, or R₂′ with R₁ is a bond, or is bonded to a common O atom with R₁ to form an epoxide ring;
R₃ is OH, C₁₋₆alkyl (O)CO—; C₂₋₆alkenyl(O)CO—, phenyl(O)CO—; phenylC₁₋₄ alkyl(O)CO—, phenylC₂₋₄alkenyl(O)CO—; pyrrolidinyl(O)CO—; or R₃ with R₃′ is ═O,
R₃′ is H, or R₃′ with R₃ is ═O;
R₄ is H, or R₄ with R₁₀ or with R₅′ is a bond, or is bonded to a common O atom with R₁₀ to form an epoxide ring;
R₅ is H, —CH₃, OH, CH₃(O)CO—, C₁₋₄alkyl-O—, or phenylC₂₋₄ alkenyl(O)CO—,
R₅′ is H, or R₅′ with R₄ or R₆′ is a bond, or is bonded to a common O atom with R₆′ to form an epoxide ring;
R₆ is H, CH₃—, CH₂— which is bonded to a common O atom with R₆′ to form an epoxide ring, or R₆ with R₆′ is ═CH₂;
R₆′; is H, OH, CH₃(O)CO—, or R₆′ with R₅′ or R₇ is a bond, or is bonded to a common O atom with R₅′ to form the epoxide ring;
R₇ is H, OH, CH₃(O)CO—, phenyl(O)CO—, C₂₋₈ alkenyl-(O)CO—, or phenylC₁₋₄alkyl-(O)CO—;
R₈ is H, OH, C₁₋₆ alkyl-O—, C₁₋₄alkyl(O)CO—, C₂₋₆ alkenyl(O)CO—, or phenyl(O)CO—;
R₉ is OH, ═O or ═CH₂;
R₁₀ is H, OH, CH₃(O)CO—, phenylC₂₋₄alkenyl(O)CO—, or R₁₀ with R₄ is a bond, or is bonded to a common O atom with R₄ to form the epoxide ring;
R₁₁ is H, CH₃—, CH₂OH, or R₁₁ with R₁₁′ is ═CH₂,
R₁₁′ is H, or R₁₁′ with R₁₂′ is a bond;
R₁₂ is H, OH, C₁₋₆ alkyl(O)CO—, C₂₋₆ alkenyl(O)CO—; and
R₁₂′ is H, OH, or R₁₂′ with R₁₁′ is a bond; and [symbol]
represents an optional double bond, with the proviso that when a double bond is present between C4 and C10 atoms, there is no double bond between the C4 and C5 atoms;
when a double bond is present between C4 and C5 atoms, there is no double bond between the C4 and C10 atoms and the C5 and C6 atoms;
when a double bond is present between C5 and C6 atoms, there is no double bond between the C4 and C5 atoms and C6 and C7 atoms; and
when a double bond is present between C6 and C7 atoms, there is no double bond between the C5 and C6 atoms.
